SEQUENCE LISTING (170 sequences)

SEQ ID NO: 1 - protein, 9 aa, Artificial Sequence
  Synthetic Polypeptide
PFPQPELPY

SEQ ID NO: 2 - protein, 9 aa, Artificial Sequence
  Synthetic Polypeptide
PQPELPYPQ

SEQ ID NO: 3 - protein, 9 aa, Artificial Sequence
  Synthetic Polypeptide
PFPQPEQPF

SEQ ID NO: 4 - protein, 9 aa, Artificial Sequence
  Synthetic Polypeptide
PQPEQPFPW

SEQ ID NO: 5 - protein, 9 aa, Artificial Sequence
  Synthetic Polypeptide
EQPIPEQPQ

SEQ ID NO: 6 - protein, 9 aa, Artificial Sequence
  Synthetic Polypeptide
PIPEQPQPY

SEQ ID NO: 7 - protein, 9 aa, Artificial Sequence
  Synthetic Polypeptide
PFPQPEQPI

SEQ ID NO: 8 - protein, 9 aa, Artificial Sequence
  Synthetic Polypeptide
PQPEQPIPV

SEQ ID NO: 9 - protein, 9 aa, Artificial Sequence
  Synthetic Polypeptide
EQPIPVQPE

SEQ ID NO: 10 - protein, 9 aa, Artificial Sequence
  Synthetic Polypeptide
PFPQPEQPT

SEQ ID NO: 11 - protein, 9 aa, Artificial Sequence
  Synthetic Polypeptide
PQPEQPTPI

SEQ ID NO: 12 - protein, 9 aa, Artificial Sequence
  Synthetic Polypeptide
EQPTPIQPE

SEQ ID NO: 13 - protein, 9 aa, Artificial Sequence
  Synthetic Polypeptide
PQPEQPFPL

SEQ ID NO: 14 - protein, 9 aa, Artificial Sequence
  Synthetic Polypeptide
EQPFPLQPE

SEQ ID NO: 15 - protein, 9 aa, Artificial Sequence
  Synthetic Polypeptide
PQPEQPFSQ

SEQ ID NO: 16 - protein, 9 aa, Artificial Sequence
  Synthetic Polypeptide
PYPEQPQPF

SEQ ID NO: 17 - protein, 9 aa, Artificial Sequence
  Synthetic Polypeptide
EGSFQPSQE

SEQ ID NO: 18 - protein, 9 aa, Artificial Sequence
  Synthetic Polypeptide
QGYYPTSPQ

SEQ ID NO: 19 - protein, 9 aa, Artificial Sequence
  Synthetic Polypeptide
EQPEQPFPE

SEQ ID NO: 20 - protein, 9 aa, Artificial Sequence
  Synthetic Polypeptide
EQPFPEQPQ

SEQ ID NO: 21 - protein, 9 aa, Artificial Sequence
  Synthetic Polypeptide
PFPEQPEQI

SEQ ID NO: 22 - protein, 9 aa, Artificial Sequence
  Synthetic Polypeptide
PFSEQEQPV

SEQ ID NO: 23 - protein, 9 aa, Artificial Sequence
  Synthetic Polypeptide
EQPFPEQPI

SEQ ID NO: 24 - protein, 9 aa, Artificial Sequence
  Synthetic Polypeptide
PFPEQPIPE

SEQ ID NO: 25 - protein, 9 aa, Artificial Sequence
  Synthetic Polypeptide
PYPQPELPY

SEQ ID NO: 26 - protein, 9 aa, Artificial Sequence
  Synthetic Polypeptide
PQPELPYPY

SEQ ID NO: 27 - protein, 9 aa, Artificial Sequence
  Synthetic Polypeptide
PQPYPEQPQ

SEQ ID NO: 28 - protein, 15 aa, Artificial Sequence
  Synthetic Polypeptide
LQPFPQPELPYPQPQ

SEQ ID NO: 29 - protein, 14 aa, Artificial Sequence
  Synthetic Polypeptide
QPFPQPEQPFPWQP

SEQ ID NO: 30 - protein, 15 aa, Artificial Sequence
  Synthetic Polypeptide
PEQPIPEQPQPYPQQ

SEQ ID NO: 31 - protein, 17 aa, Artificial Sequence
  Synthetic Polypeptide
QPFPQPEQPIPVQPEQS

SEQ ID NO: 32 - protein, 17 aa, Artificial Sequence
  Synthetic Polypeptide
QPFPQPEQPTPIQPEQP

SEQ ID NO: 33 - protein, 17 aa, Artificial Sequence
  Synthetic Polypeptide
QPFPQPEQPFPLQPEQP

SEQ ID NO: 34 - protein, 13 aa, Artificial Sequence
  Synthetic Polypeptide
QPFPQPEQPFSQQ

SEQ ID NO: 35 - protein, 14 aa, Artificial Sequence
  Synthetic Polypeptide
PQPYPEQPQPFPQQ

SEQ ID NO: 36 - protein, 15 aa, Artificial Sequence
  Synthetic Polypeptide
QPFPEQPEQIIPQQP

SEQ ID NO: 37 - protein, 14 aa, Artificial Sequence
  Synthetic Polypeptide
SGEGSFQPSQENPQ

SEQ ID NO: 38 - protein, 14 aa, Artificial Sequence
  Synthetic Polypeptide
GQQGYYPTSPQQSG

SEQ ID NO: 39 - protein, 14 aa, Artificial Sequence
  Synthetic Polypeptide
PEQPEQPFPEQPQQ

SEQ ID NO: 40 - protein, 14 aa, Artificial Sequence
  Synthetic Polypeptide
QPPFSEQEQPVLPQ

SEQ ID NO: 41 - protein, 18 aa, Artificial Sequence
  Synthetic Polypeptide
PEQPFPEQPIPEQPQPYP

SEQ ID NO: 42 - protein, 14 aa, Artificial Sequence
  Synthetic Polypeptide
QPYPQPELPYPQPQ

SEQ ID NO: 43 - protein, 14 aa, Artificial Sequence
  Synthetic Polypeptide
QPFPQPELPYPYPQ

SEQ ID NO: 44 - protein, 15 aa, Artificial Sequence
  Synthetic Polypeptide
PQEQPFPEQPIPEQP

SEQ ID NO: 45 - protein, 15 aa, Artificial Sequence
  Synthetic Polypeptide
QPQPYPEQPQPFPQQ

SEQ ID NO: 46 - protein, 12 aa, Artificial Sequence
  Synthetic Polypeptide
PFPQPELPYPQP

SEQ ID NO: 47 - protein, 12 aa, Artificial Sequence
  Synthetic Polypeptide
PFPQPEQPFPWQ

SEQ ID NO: 48 - protein, 12 aa, Artificial Sequence
  Synthetic Polypeptide
EQPIPEQPQPYP

SEQ ID NO: 49 - protein, 12 aa, Artificial Sequence
  Synthetic Polypeptide
PFPQPEQPIPVQ

SEQ ID NO: 50 - protein, 12 aa, Artificial Sequence
  Synthetic Polypeptide
PEQPIPVQPEQS

SEQ ID NO: 51 - protein, 12 aa, Artificial Sequence
  Synthetic Polypeptide
PFPQPEQPTPIQ

SEQ ID NO: 52 - protein, 12 aa, Artificial Sequence
  Synthetic Polypeptide
PEQPTPIQPEQP

SEQ ID NO: 53 - protein, 12 aa, Artificial Sequence
  Synthetic Polypeptide
PFPQPEQPFPLQ

SEQ ID NO: 54 - protein, 12 aa, Artificial Sequence
  Synthetic Polypeptide
PEQPFPLQPEQP

SEQ ID NO: 55 - protein, 12 aa, Artificial Sequence
  Synthetic Polypeptide
GEGSFQPSQENP

SEQ ID NO: 56 - protein, 12 aa, Artificial Sequence
  Synthetic Polypeptide
QQGYYPTSPQQS

SEQ ID NO: 57 - protein, 12 aa, Artificial Sequence
  Synthetic Polypeptide
PEQPEQPFPEQP

SEQ ID NO: 58 - protein, 12 aa, Artificial Sequence
  Synthetic Polypeptide
PPFSEQEQPVLP

SEQ ID NO: 59 - protein, 12 aa, Artificial Sequence
  Synthetic Polypeptide
PYPQPELPYPQP

SEQ ID NO: 60 - protein, 12 aa, Artificial Sequence
  Synthetic Polypeptide
EQPFPEQPIPEQ

SEQ ID NO: 61 - protein, 12 aa, Artificial Sequence
  Synthetic Polypeptide
PQPYPEQPQPFP

SEQ ID NO: 62 - protein, 10 aa, Artificial Sequence
  Synthetic Polypeptide
PFPQPEQPIP

SEQ ID NO: 63 - protein, 11 aa, Artificial Sequence
  Synthetic Polypeptide
PFPEQPEQIIP

SEQ ID NO: 64 - protein, 12 aa, Artificial Sequence
  Synthetic Polypeptide
EQPEQPFPEQPQ

SEQ ID NO: 65 - protein, 11 aa, Artificial Sequence
  Synthetic Polypeptide
PFPQPEQPTPI

SEQ ID NO: 66 - protein, 16 aa, Artificial Sequence
  Synthetic Polypeptide
XLQPFPQPELPYPQPQ

SEQ ID NO: 67 - protein, 15 aa, Artificial Sequence
  Synthetic Polypeptide
XQPFPQPEQPFPWQP

SEQ ID NO: 68 - protein, 16 aa, Artificial Sequence
  Synthetic Polypeptide
XPEQPIPEQPQPYPQQ

SEQ ID NO: 69 - protein, 19 aa, Artificial Sequence
  Synthetic Polypeptide
XPEQPFPEQPIPEQPQPYP

SEQ ID NO: 70 - protein, 18 aa, Artificial Sequence
  Synthetic Polypeptide
XQPFPQPEQPIPVQPEQS

SEQ ID NO: 71 - protein, 17 aa, Artificial Sequence
  Synthetic Polypeptide
XQPFPQPEQPTPIQPEQ

SEQ ID NO: 72 - protein, 18 aa, Artificial Sequence
  Synthetic Polypeptide
XQPFPQPEQPFPLQPEQP

SEQ ID NO: 73 - protein, 14 aa, Artificial Sequence
  Synthetic Polypeptide
XQPFPQPEQPFSQQ

SEQ ID NO: 74 - protein, 15 aa, Artificial Sequence
  Synthetic Polypeptide
XPQPYPEQPQPFPQQ

SEQ ID NO: 75 - protein, 15 aa, Artificial Sequence
  Synthetic Polypeptide
XQPYPQPELPYPQPQ

SEQ ID NO: 76 - protein, 15 aa, Artificial Sequence
  Synthetic Polypeptide
XQPFPQPELPYPYPQ

SEQ ID NO: 77 - protein, 15 aa, Artificial Sequence
  Synthetic Polypeptide
XSGEGSFQPSQENPQ

SEQ ID NO: 78 - protein, 15 aa, Artificial Sequence
  Synthetic Polypeptide
XGQQGYYPTSPQQSG

SEQ ID NO: 79 - protein, 15 aa, Artificial Sequence
  Synthetic Polypeptide
XPEQPEQPFPEQPQQ

SEQ ID NO: 80 - protein, 16 aa, Artificial Sequence
  Synthetic Polypeptide
XQPFPEQPEQIIPQQP

SEQ ID NO: 81 - protein, 15 aa, Artificial Sequence
  Synthetic Polypeptide
XQPPFSEQEQPVLPQ

SEQ ID NO: 82 - protein, 16 aa, Artificial Sequence
  Synthetic Polypeptide
XPQEQPFPEQPIPEQP

SEQ ID NO: 83 - protein, 16 aa, Artificial Sequence
  Synthetic Polypeptide
XQPQPYPEQPQPFPQQ

SEQ ID NO: 84 - protein, 13 aa, Artificial Sequence
  Synthetic Polypeptide
XPFPQPELPYPQP

SEQ ID NO: 85 - protein, 13 aa, Artificial Sequence
  Synthetic Polypeptide
XPFPQPEQPFPWQ

SEQ ID NO: 86 - protein, 13 aa, Artificial Sequence
  Synthetic Polypeptide
XEQPIPEQPQPYP

SEQ ID NO: 87 - protein, 13 aa, Artificial Sequence
  Synthetic Polypeptide
XPFPQPEQPIPVQ

SEQ ID NO: 88 - protein, 13 aa, Artificial Sequence
  Synthetic Polypeptide
XPEQPIPVQPEQS

SEQ ID NO: 89 - protein, 13 aa, Artificial Sequence
  Synthetic Polypeptide
XPFPQPEQPTPIQ

SEQ ID NO: 90 - protein, 13 aa, Artificial Sequence
  Synthetic Polypeptide
XPEQPTPIQPEQP

SEQ ID NO: 91 - protein, 13 aa, Artificial Sequence
  Synthetic Polypeptide
XPFPQPEQPFPLQ

SEQ ID NO: 92 - protein, 13 aa, Artificial Sequence
  Synthetic Polypeptide
XPEQPFPLQPEQP

SEQ ID NO: 93 - protein, 13 aa, Artificial Sequence
  Synthetic Polypeptide
XGEGSFQPSQENP

SEQ ID NO: 94 - protein, 13 aa, Artificial Sequence
  Synthetic Polypeptide
XQQGYYPTSPQQS

SEQ ID NO: 95 - protein, 13 aa, Artificial Sequence
  Synthetic Polypeptide
XPEQPEQPFPEQP

SEQ ID NO: 96 - protein, 13 aa, Artificial Sequence
  Synthetic Polypeptide
XPPFSEQEQPVLP

SEQ ID NO: 97 - protein, 13 aa, Artificial Sequence
  Synthetic Polypeptide
XPYPQPELPYPQP

SEQ ID NO: 98 - protein, 13 aa, Artificial Sequence
  Synthetic Polypeptide
XEQPFPEQPIPEQ

SEQ ID NO: 99 - protein, 13 aa, Artificial Sequence
  Synthetic Polypeptide
XPQPYPEQPQPFP

SEQ ID NO: 100 - protein, 18 aa, Artificial Sequence
  Synthetic Polypeptide
XQPFPQPEQPTPIQPEQP

SEQ ID NO: 101 - protein, 9 aa, Artificial Sequence
  Synthetic Polypeptide
PFPQPDLPY

SEQ ID NO: 102 - protein, 9 aa, Artificial Sequence
  Synthetic Polypeptide
PQPDLPYPQ

SEQ ID NO: 103 - protein, 9 aa, Artificial Sequence
  Synthetic Polypeptide
PFPQPDQPF

SEQ ID NO: 104 - protein, 9 aa, Artificial Sequence
  Synthetic Polypeptide
PQPDQPFPW

SEQ ID NO: 105 - protein, 9 aa, Artificial Sequence
  Synthetic Polypeptide
PIPDQPQPY

SEQ ID NO: 106 - protein, 10 aa, Artificial Sequence
  Synthetic Polypeptide
PFPQPDQPIP

SEQ ID NO: 107 - protein, 9 aa, Artificial Sequence
  Synthetic Polypeptide
DQPIPVQPD

SEQ ID NO: 108 - protein, 11 aa, Artificial Sequence
  Synthetic Polypeptide
PFPQPDQPTPI

SEQ ID NO: 109 - protein, 9 aa, Artificial Sequence
  Synthetic Polypeptide
DQPTPIQPD

SEQ ID NO: 110 - protein, 9 aa, Artificial Sequence
  Synthetic Polypeptide
PQPDQPFPL

SEQ ID NO: 111 - protein, 9 aa, Artificial Sequence
  Synthetic Polypeptide
DQPFPLQPD

SEQ ID NO: 112 - protein, 9 aa, Artificial Sequence
  Synthetic Polypeptide
PFPQPDQPF

SEQ ID NO: 113 - protein, 9 aa, Artificial Sequence
  Synthetic Polypeptide
PQPDQPFSQ

SEQ ID NO: 114 - protein, 9 aa, Artificial Sequence
  Synthetic Polypeptide
PYPDQPQPF

SEQ ID NO: 115 - protein, 11 aa, Artificial Sequence
  Synthetic Polypeptide
PFPDQPDQIIP

SEQ ID NO: 116 - protein, 9 aa, Artificial Sequence
  Synthetic Polypeptide
DGSFQPSQD

SEQ ID NO: 117 - protein, 12 aa, Artificial Sequence
  Synthetic Polypeptide
DQPDQPFPDQPQ

SEQ ID NO: 118 - protein, 9 aa, Artificial Sequence
  Synthetic Polypeptide
PFSDQDQPV

SEQ ID NO: 119 - protein, 9 aa, Artificial Sequence
  Synthetic Polypeptide
DQPFPDQPI

SEQ ID NO: 120 - protein, 9 aa, Artificial Sequence
  Synthetic Polypeptide
PIPDQPQPY

SEQ ID NO: 121 - protein, 9 aa, Artificial Sequence
  Synthetic Polypeptide
PQPDLPYPQ

SEQ ID NO: 122 - protein, 9 aa, Artificial Sequence
  Synthetic Polypeptide
PYPQPDLPY

SEQ ID NO: 123 - protein, 9 aa, Artificial Sequence
  Synthetic Polypeptide
PFPQPDLPY

SEQ ID NO: 124 - protein, 9 aa, Artificial Sequence
  Synthetic Polypeptide
PQPDLPYPY

SEQ ID NO: 125 - protein, 9 aa, Artificial Sequence
  Synthetic Polypeptide
DQPIPDQPQ

SEQ ID NO: 126 - protein, 9 aa, Artificial Sequence
  Synthetic Polypeptide
PFPQPDQPI

SEQ ID NO: 127 - protein, 9 aa, Artificial Sequence
  Synthetic Polypeptide
PQPDQPIPV

SEQ ID NO: 128 - protein, 9 aa, Artificial Sequence
  Synthetic Polypeptide
DQPIPVQPE

SEQ ID NO: 129 - protein, 9 aa, Artificial Sequence
  Synthetic Protein
PFPQPDQPT

SEQ ID NO: 130 - protein, 9 aa, Artificial Sequence
  Synthetic Polypeptide
PQPDQPTPI

SEQ ID NO: 131 - protein, 9 aa, Artificial Sequence
  Synthetic Polypeptide
DQPQQPFPD

SEQ ID NO: 132 - protein, 9 aa, Artificial Sequence
  Synthetic Polypeptide
DQPDQPFPQ

SEQ ID NO: 133 - protein, 9 aa, Artificial Sequence
  Synthetic Polypeptide
DQPFPDQPQ

SEQ ID NO: 134 - protein, 9 aa, Artificial Sequence
  Synthetic Polypeptide
PFPDQPDQI

SEQ ID NO: 135 - protein, 9 aa, Artificial Sequence
  Synthetic Polypeptide
PFPDQPIPD

SEQ ID NO: 136 - protein, 9 aa, Artificial Sequence
  Synthetic Polypeptide
PQPYPDQPQ

SEQ ID NO: 137 - protein, 9 aa, Artificial Sequence
  Synthetic Polypeptide
PFPQPQLPY

SEQ ID NO: 138 - protein, 9 aa, Artificial Sequence
  Synthetic Polypeptide
PFPQPDLPY

SEQ ID NO: 139 - protein, 9 aa, Artificial Sequence
  Synthetic Polypeptide
PQPQLPYPQ

SEQ ID NO: 140 - protein, 9 aa, Artificial Sequence
  Synthetic Polypeptide
PFPQPQQPF

SEQ ID NO: 141 - protein, 9 aa, Artificial Sequence
  Synthetic Polypeptide
PQPQQPFPW

SEQ ID NO: 142 - protein, 9 aa, Artificial Sequence
  Synthetic Polypeptide
PIPQQPQPY

SEQ ID NO: 143 - protein, 10 aa, Artificial Sequence
  Synthetic Polypeptide
PFPQPQQPIP

SEQ ID NO: 144 - protein, 9 aa, Artificial Sequence
  Synthetic Polypeptide
QQPIPVQPQ

SEQ ID NO: 145 - protein, 11 aa, Artificial Sequence
  Synthetic Polypeptide
PFPQPQQPTPI

SEQ ID NO: 146 - protein, 9 aa, Artificial Sequence
  Synthetic Polypeptide
QQPTPIQPQ

SEQ ID NO: 147 - protein, 9 aa, Artificial Sequence
  Synthetic Polypeptide
PQPQQPFPL

SEQ ID NO: 148 - protein, 9 aa, Artificial Sequence
  Synthetic Polypeptide
QQPFPLQPQ

SEQ ID NO: 149 - protein, 9 aa, Artificial Sequence
  Synthetic Polypeptide
PQPQQPFSQ

SEQ ID NO: 150 - protein, 9 aa, Artificial Sequence
  Synthetic Polypeptide
PYPQQPQPF

SEQ ID NO: 151 - protein, 11 aa, Artificial Sequence
  Synthetic Polypeptide
PFPQQPQQIIP

SEQ ID NO: 152 - protein, 9 aa, Artificial Sequence
  Synthetic Polypeptide
QGSFQPSQQ

SEQ ID NO: 153 - protein, 12 aa, Artificial Sequence
  Synthetic Polypeptide
QQPQQPFPQQPQ

SEQ ID NO: 154 - protein, 9 aa, Artificial Sequence
  Synthetic Polypeptide
PFSQQQQPV

SEQ ID NO: 155 - protein, 12 aa, Artificial Sequence
  Synthetic Polypeptide
QQQPFPQQPIPQ

SEQ ID NO: 156 - protein, 9 aa, Artificial Sequence
  Synthetic Polypeptide
QQPFPQQPI

SEQ ID NO: 157 - protein, 9 aa, Artificial Sequence
  Synthetic Polypeptide
PYPQPQLPY

SEQ ID NO: 158 - protein, 9 aa, Artificial Sequence
  Synthetic Polypeptide
PQPQLPYPY

SEQ ID NO: 159 - protein, 9 aa, Artificial Sequence
  Synthetic Polypeptide
QQPIPQQPQ

SEQ ID NO: 160 - protein, 9 aa, Artificial Sequence
  Synthetic Polypeptide
PFPQPQQPI

SEQ ID NO: 161 - protein, 9 aa, Artificial Sequence
  Synthetic Polypeptide
PQPQQPIPV

SEQ ID NO: 162 - protein, 9 aa, Artificial Sequence
  Synthetic Polypeptide
PFPQPQQPT

SEQ ID NO: 163 - protein, 9 aa, Artificial Sequence
  Synthetic Polypeptide
PQPQQPTPI

SEQ ID NO: 164 - protein, 9 aa, Artificial Sequence
  Synthetic Polypeptide
QQPQQPFPQ

SEQ ID NO: 165 - protein, 9 aa, Artificial Sequence
  Synthetic Polypeptide
QQPQQPFPQ

SEQ ID NO: 166 - protein, 9 aa, Artificial Sequence
  Synthetic Polypeptide
QQPFPQQPQ

SEQ ID NO: 167 - protein, 9 aa, Artificial Sequence
  Synthetic Polypeptide
PFPQQPQQI

SEQ ID NO: 168 - protein, 9 aa, Artificial Sequence
  Synthetic Polypeptide
PFPQQPIPQ

SEQ ID NO: 169 - protein, 9 aa, Artificial Sequence
  Synthetic Polypeptide
PQPQLPYPY

SEQ ID NO: 170 - protein, 9 aa, Artificial Sequence
  Synthetic Polypeptide
PQPYPQQPQ

(19) United States
(12) Patent Application Publication
(10) Pub. No.: US 2017/0042991 A1
(43) Pub. Date: Feb. 16, 2017

(54) COMPOSITIONS COMPRISING GLUTEN PEPTIDES AND USES THEREOF

(71) Applicant: ImmusanT, Inc., Cambridge, MA (US)

(72) Inventor: Robert P. Anderson, Shrewsbury, MA (US)

(73) Assignee: ImmusanT, Inc., Cambridge, MA (US)

(21) Appl. No.: 15/306,136

(22) PCT Filed: Apr. 24, 2015
(86) PCT No.: PCT/US2015/027530

§ 371 Date: Oct. 24, 2016

Related U.S. Application Data
(60) Provisional application No. 61/983,989, filed on Apr. 24, 2014.
(60) Provisional application No. 61/983,981, filed on Apr. 24, 2014.
(60) Provisional application No. 61/983,993, filed on Apr. 24, 2014.
(60) Provisional application No. 61/984,028, filed on Apr. 24, 2014.
(60) Provisional application No. 61/984,043, filed on Apr. 25, 2014.
(60) Provisional application No. 62/009,090, filed on Jun. 6, 2014.
(60) Provisional application No. 62/009,146, filed on Jun. 6, 2014.
(60) Provisional application No. 62/011,561, filed on Jun. 12, 2014.
(60) Provisional application No. 62/011,508, filed on Jun. 12, 2014.
(60) Provisional application No. 62/011,493, filed on Jun. 12, 2014.
(60) Provisional application No. 62/011,540, filed on Jun. 12, 2014.
(60) Provisional application No. 62/011,566, filed on Jun. 12, 2014.
(60) Provisional application No. 62/011,794, filed on Jun. 13, 2014.
(60) Provisional application No. 62/014,373, filed on Jun. 19, 2014.
(60) Provisional application No. 62/014,401, filed on Jun. 19, 2014.
(60) Provisional application No. 62/014,681, filed on Jun. 19, 2014.
(60) Provisional application No. 62/014,666, filed on Jun. 19, 2014.
(60) Provisional application No. 62/014,676, filed on Jun. 19, 2014.
(60) Provisional application No. 62/043,395, filed on Aug. 28, 2014.
(60) Provisional application No. 62/043,386, filed on Aug. 28, 2014.
(60) Provisional application No. 62/043,390, filed on Aug. 28, 2014.
(60) Provisional application No. 62/057,152, filed on Sep. 29, 2014.
(60) Provisional application No. 62/057,163, filed on Sep. 29, 2014.
(60) Provisional application No. 62/082,832, filed on Nov. 21, 2014.
(60) Provisional application No. 62/116,052, filed on Feb. 13, 2015.
(60) Provisional application No. 62/116,027, filed on Feb. 13, 2015.
(60) Provisional application No. 62/116,002, filed on Feb. 13, 2015.
(60) Provisional application No. 62/115,925, filed on Feb. 13, 2015.
(60) Provisional application No. 62/115,897, filed on Feb. 13, 2015.
(60) Provisional application No. 62/115,963, filed on Feb. 13, 2015.

(51) Int. Cl. A61K 39/00; G01N 33/564; C07K 7/06

(52) U.S. Cl. CPC A61K 39/0008; C07K 7/06; G01N 33/564; G01N 2800/06; G01N 2333/57; G01N 2333/521

(57) ABSTRACT

Provided herein are compositions, methods, and kits related to compositions comprising at least one gluten peptide. In some aspects, compositions, methods, and kits useful for subjects having Celiac disease.

### RELATED APPLICATIONS

This application claims the benefit under 35 U.S.C. §119(e) of U.S. provisional application No. 61/983,981, filed Apr. 24, 2014, U.S. provisional application No. 62/011,561, filed Jun. 12, 2014, U.S. provisional application No. 62/014,676, filed Jun. 19, 2014, U.S. provisional application No. 62/057,152, filed Sep. 29, 2014, U.S. provisional application No. 62/115,925, filed Feb. 13, 2015, U.S. provisional application No. 61/984,028, filed Apr. 24, 2014, U.S. provisional application No. 61/984,043, filed Apr. 25, 2014, U.S. provisional application No. 62/011,566, filed Jun. 12, 2014, U.S. provisional application No. 62/014,681, filed Jun. 19, 2014, U.S. provisional application No. 62/057,163, filed Sep. 29, 2014, U.S. provisional application No. 62/115,897, filed Feb. 13, 2015, U.S. provisional application No. 61/983,989, filed Apr. 24, 2014, U.S. provisional application No. 62/014,666, filed Jun. 19, 2014, U.S. provisional application No. 62/009,146, filed Jun. 6, 2014, U.S. provisional application No. 62/043,386, filed Aug. 28, 2014, U.S. provisional application No. 62/115,963, filed Feb. 13, 2015, U.S. provisional application No. 61/983,993, filed Apr. 24, 2014, U.S. provisional application No. 62/011,508, filed Jun. 12, 2014, U.S. provisional application No. 62/116,052, filed Feb. 13, 2015, U.S. provisional application No. 62/043,395, filed Aug. 28, 2014, U.S. provisional application No. 62/082,832, filed Nov. 21, 2014, U.S. provisional application No. 62/009,090, filed Jun. 6, 2014, U.S. provisional application No. 62/014,373, filed Jun. 19, 2014, U.S. provisional application No. 62/043,390, filed Aug. 28, 2014, U.S. provisional application No. 62/116,002, filed Feb. 13, 2015, U.S. provisional application No. 62/011,493, filed Jun. 12, 2014, U.S. provisional application No. 62/011,794, filed Jun. 13, 2014, U.S. provisional application No. 62/014,401, filed Jun. 19, 2014, U.S. provisional application No. 62/116,027, filed Feb. 13, 2015, and U.S. provisional application No. 62/011,540, filed Jun. 12, 2014, the contents of each of which are incorporated by reference herein in their entirety.

### BACKGROUND

Celiac disease is an autoimmune disorder of the small intestine that occurs in people of all ages. Celiac disease causes damage to the villi of the small intestine due to an inappropriate immune response to gluten peptides, leading to malabsorption and an increased risk of intestinal cancer. The only currently approved treatment for Celiac disease is a gluten free diet.

### SUMMARY

Celiac disease generally occurs in individuals who possess HLA-DQA and HLA-DQB susceptibility alleles encoding HLA-DQ2.5 (DQA1*05 and DQB1*02), DQ2.2 (DQA1*02 and DQB1*02) or DQ8 (DQA1*03 and DQB1*0302). As described herein, compositions were designed and optimized to contain multiple T cell epitopes that were DQ2.5, DQ2.2, and/or DQ8-restricted. It was found that these compositions produced a robust response in blood samples from subjects with Celiac disease.

Accordingly, aspects of the disclosure relate to compositions comprising at least one of these peptides and methods of use related thereto.

In some aspects, the disclosure relates to a composition comprising at least one (e.g., one, two, three, four, five, six, seven, eight, nine, ten, eleven, twelve, thirteen, fourteen, fifteen, or sixteen) peptide(s), the at least one peptide comprising at least one (e.g., one, two, three, four, five, six, seven, eight, nine, ten, eleven, twelve, thirteen, fourteen, fifteen, sixteen, seventeen, eighteen, nineteen, twenty, twenty-one, twenty-two, twenty-three or more) amino acid sequence(s) selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), PQPEQPFSQ (SEQ ID NO: 15), PYPEQPQPF (SEQ ID NO: 16), EGSFQPSQE (SEQ ID NO: 17), QGYYPTSPQ (SEQ ID NO: 18), EQPEQPFPE (SEQ ID NO: 19), EQPFPEQPQ (SEQ ID NO: 20), PFPEQPEQI (SEQ ID NO: 21), PFSEQEQPV (SEQ ID NO: 22), EQPFPEQPI (SEQ ID NO: 23), PFPEQPIPE (SEQ ID NO: 24), PYPQPELPY (SEQ ID NO: 25), PQPELPYPY (SEQ ID NO: 26), and PQPYPEQPQ (SEQ ID NO: 27). In some embodiments, the composition comprises at least one (e.g., one, two, three, four, five, six, seven, eight, nine, ten, eleven, twelve, thirteen, fourteen, fifteen, or sixteen) peptide(s) comprising at least four (e.g., four, five, six, seven, eight, nine, ten, eleven, twelve, thirteen, fourteen, fifteen, sixteen, seventeen, eighteen, nineteen, twenty, twenty-one, twenty-two, or twenty-three) amino acid sequences selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), PQPEQPFSQ (SEQ ID NO: 15), PYPEQPQPF (SEQ ID NO: 16), EGSFQPSQE (SEQ ID NO: 17), QGYYPTSPQ (SEQ ID NO: 18), EQPEQPFPE (SEQ ID NO: 19), EQPFPEQPQ (SEQ ID NO: 20), PFPEQPEQI (SEQ ID NO: 21), PFSEQEQPV (SEQ ID NO: 22), EQPFPEQPI (SEQ ID NO: 23), PFPEQPIPE (SEQ ID NO: 24), PYPQPELPY (SEQ ID NO: 25), PQPELPYPY (SEQ ID NO: 26), and PQPYPEQPQ (SEQ ID NO: 27). In some embodiments, the compositions comprises at least one (e.g., one, two, three, four, five, six, seven, eight, nine, ten, eleven, twelve, thirteen, fourteen, fifteen, or sixteen) peptide(s) comprising the amino acid sequences PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6) and at least one (e.g., one, two, three, four, five, six, seven, eight, nine, ten, eleven, twelve, thirteen, fourteen, fifteen, sixteen, seventeen, eighteen, nineteen, twenty, twenty-one or more) further amino acid sequence(s) selected from PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), PQPEQPFSQ (SEQ ID NO: 15), PYPEQPQPF (SEQ ID NO: 16), EGSFQPSQE (SEQ ID NO: 17), QGYYPTSPQ (SEQ ID NO: 18), EQPEQPFPE (SEQ ID NO: 19), EQPFPEQPQ (SEQ ID NO: 20), PFPEQPEQI (SEQ ID NO: 21), PFSEQEQPV (SEQ ID NO: 22), EQPFPEQPI (SEQ ID NO: 23), PFPEQPIPE (SEQ ID NO: 24), PYPQPELPY (SEQ ID NO: 25), PQPELPYPY (SEQ ID NO: 26), and PQPYPEQPQ (SEQ ID NO: 27). In some embodiments, the composition comprises at least one peptide comprising the amino acid sequences EQPFPEQPI (SEQ ID NO: 23), PFPEQPIPE (SEQ ID NO: 24), EQPIPEQPQ (SEQ ID NO: 5), and PIPEQPQPY (SEQ ID NO: 6) (e.g., the composition comprises at least one peptide comprising the amino acid sequence PEQPFPEQPIPEQPQPYP (SEQ ID NO: 41)).

In some embodiments, the composition comprises (or consists of) at least one (e.g., one, two, three, four, five, six, seven, eight, nine, ten, eleven, twelve, thirteen, fourteen, fifteen, or sixteen) peptide(s) selected from:

(a) a first peptide comprising the amino acid sequence PFPQPELPY (SEQ ID NO: 1) and the amino acid sequence PQPELPYPQ (SEQ ID NO: 2);

(b) a second peptide comprising the amino acid sequence PFPQPEQPF (SEQ ID NO: 3) and the amino acid sequence PQPEQPFPW (SEQ ID NO: 4);

(c) a third peptide comprising the amino acid sequence EQPIPEQPQ (SEQ ID NO: 5) and the amino acid sequence PIPEQPQPY (SEQ ID NO: 6);

(d) a fourth peptide comprising the amino acid sequence PFPQPEQPI (SEQ ID NO: 7), the amino acid sequence PQPEQPIPV (SEQ ID NO: 8), and the amino acid sequence EQPIPVQPE (SEQ ID NO: 9);

(e) a fifth peptide comprising the amino acid sequence PFPQPEQPT (SEQ ID NO: 10), the amino acid sequence PQPEQPTPI (SEQ ID NO: 11), and the amino acid sequence EQPTPIQPE (SEQ ID NO: 12);

(f) a sixth peptide comprising the amino acid sequence PFPQPEQPF (SEQ ID NO: 3), the amino acid sequence PQPEQPFPL (SEQ ID NO: 13), and the amino acid sequence EQPFPLQPE (SEQ ID NO: 14);

(g) a seventh peptide comprising the amino acid sequence PFPQPEQPF (SEQ ID NO: 3) and the amino acid sequence PQPEQPFSQ (SEQ ID NO: 15);

(h) an eighth peptide comprising the amino acid sequence PYPEQPQPF (SEQ ID NO: 16);

(i) a ninth peptide comprising the amino acid sequence PFPEQPEQI (SEQ ID NO: 21);

(j) a tenth peptide comprising the amino acid sequence EGSFQPSQE (SEQ ID NO: 17);

(k) an eleventh peptide comprising the amino acid sequence QGYYPTSPQ (SEQ ID NO: 18);

(l) a twelfth peptide comprising the amino acid sequence EQPEQPFPE (SEQ ID NO: 19) and the amino acid sequence EQPFPEQPQ (SEQ ID NO: 20);

(m) a thirteenth peptide comprising the amino acid sequence PFSEQEQPV (SEQ ID NO: 22);

(n) a fourteenth peptide comprising the amino acid sequence EQPFPEQPI (SEQ ID NO: 23), the amino acid sequence PFPEQPIPE (SEQ ID NO: 24), the amino acid sequence EQPIPEQPQ (SEQ ID NO: 5), and the amino acid sequence PIPEQPQPY (SEQ ID NO: 6);

(o) a fifteenth peptide comprising the amino acid sequence PQPELPYPQ (SEQ ID NO: 2) and the amino acid sequence PYPQPELPY (SEQ ID NO: 25);

(p) a sixteenth peptide comprising the amino acid sequence PFPQPELPY (SEQ ID NO: 1) and the amino acid sequence PQPELPYPY (SEQ ID NO: 26);

(q) a seventeenth peptide comprising the amino acid sequence EQPFPEQPI (SEQ ID NO: 23) and the amino acid sequence PFPEQPIPE (SEQ ID NO: 24); and

(r) an eighteenth peptide comprising the amino acid sequence PQPYPEQPQ (SEQ ID NO: 27) and the amino acid sequence PYPEQPQPF (SEQ ID NO: 16).

In some embodiments,

(a) the first peptide comprises the amino acid sequence LQPFPQPELPYPQPQ (SEQ ID NO: 28);

(b) the second peptide comprises the amino acid sequence QPFPQPEQPFPWQP (SEQ ID NO: 29);

(c) the third peptide comprises the amino acid sequence PEQPIPEQPQPYPQQ (SEQ ID NO: 30);

(d) the fourth peptide comprises the amino acid sequence QPFPQPEQPIPVQPEQS (SEQ ID NO: 31);

(e) the fifth peptide comprises the amino acid sequence QPFPQPEQPTPIQPEQP (SEQ ID NO: 32);

(f) the sixth peptide comprises the amino acid sequence QPFPQPEQPFPLQPEQP (SEQ ID NO: 33);

(g) the seventh peptide comprises the amino acid sequence QPFPQPEQPFSQQ (SEQ ID NO: 34);

(h) the eighth peptide comprises the amino acid sequence PQPYPEQPQPFPQQ (SEQ ID NO: 35);

(i) the ninth peptide comprises the amino acid sequence QPFPEQPEQIIPQQP (SEQ ID NO: 36);

(j) the tenth peptide comprises the amino acid sequence SGEGSFQPSQENPQ (SEQ ID NO: 37);

(k) the eleventh peptide comprises the amino acid sequence GQQGYYPTSPQQSG (SEQ ID NO: 38);

(l) the twelfth peptide comprises the amino acid sequence PEQPEQPFPEQPQQ (SEQ ID NO: 39);

(m) the thirteenth peptide comprises the amino acid sequence QPPFSEQEQPVLPQ (SEQ ID NO: 40);

(n) the fourteenth peptide comprises the amino acid sequence PEQPFPEQPIPEQPQPYP (SEQ ID NO: 41);

(o) the fifteenth peptide comprises the amino acid sequence QPYPQPELPYPQPQ (SEQ ID NO: 42);

(p) the sixteenth peptide comprises the amino acid sequence QPFPQPELPYPYPQ (SEQ ID NO: 43);

(q) the seventeenth peptide comprises the amino acid sequence PQEQPFPEQPIPEQP (SEQ ID NO: 44); and

(r) the eighteenth peptide comprises the amino acid sequence QPQPYPEQPQPFPQQ (SEQ ID NO: 45).

In some embodiments, the composition comprises at least four (e.g., at least four, at least five, at least six, at least seven, at least eight, at least nine, at least ten, at least eleven, at least twelve, at least thirteen, at least fourteen, at least fifteen, or at least sixteen) of any of the peptides described herein. In some embodiments, the composition comprises (or consists of) (i) the first, second, and third peptides or the second, fourteenth, fifteenth, and sixteenth peptides; and (ii) at least one of the fourth, fifth, sixth, seventh, eighth, ninth, tenth, eleventh, twelfth, thirteenth, sixteenth, seventeenth, and eighteenth peptides. In some embodiments, the composition comprises (or consists of) at least two of the fourth, fifth, sixth, seventh, eighth, ninth, tenth, eleventh, twelfth, thirteenth, sixteenth, seventeenth, and eighteenth peptides. In some embodiments, the composition comprises (or consists of) at least three of the fourth, fifth, sixth, seventh, eighth, ninth, tenth, eleventh, twelfth, thirteenth, sixteenth, seventeenth, and eighteenth peptides. In some embodiments, the composition comprises (or consists of) at least four of the fourth, fifth, sixth, seventh, eighth, ninth, tenth, eleventh, twelfth, thirteenth, sixteenth, seventeenth, and eighteenth peptides. In some embodiments, the composition comprises (or consists of) at least five of the fourth, fifth, sixth, seventh, eighth, ninth, tenth, eleventh, twelfth, thirteenth, sixteenth, seventeenth, and eighteenth peptides. In some embodiments, the composition comprises (or consists of) at least six of the fourth, fifth, sixth, seventh, eighth, ninth, tenth, eleventh, twelfth, thirteenth, sixteenth, seventeenth, and eighteenth peptides. In some embodiments, the composition comprises (or consists of) at least seven of the fourth, fifth, sixth, seventh, eighth, ninth, tenth, eleventh, twelfth, thirteenth, sixteenth, seventeenth, and eighteenth peptides. In some embodiments, the composition comprises (or consists of) at least eight of the fourth, fifth, sixth, seventh, eighth, ninth, tenth, eleventh, twelfth, thirteenth, sixteenth, seventeenth, and eighteenth peptides. In some embodiments, the composition comprises (or consists of) at least nine of the fourth, fifth, sixth, seventh, eighth, ninth, tenth, eleventh, twelfth, thirteenth, sixteenth, seventeenth, and eighteenth peptides. In some embodiments, the composition comprises (or consists of) at least ten of the fourth, fifth, sixth, seventh, eighth, ninth, tenth, eleventh, twelfth, thirteenth, sixteenth, seventeenth, and eighteenth peptides.

In some embodiments, the composition comprises (or consists of) the first, second, third, fourth, fifth, sixth, seventh, eighth, ninth, tenth, eleventh, twelfth, and thirteenth peptides. In some embodiments, the composition comprises (or consists of) the second, fourth, fifth, sixth, seventh, eighth, ninth, tenth, eleventh, twelfth, thirteenth, fourteenth, fifteenth, and sixteenth peptides. In some embodiments, the composition comprises (or consists of) the first, second, third, fourth, fifth, sixth, tenth, eleventh, twelfth, thirteenth, fifteenth, seventeenth, and eighteenth peptides.

In some embodiments of any one of the compositions provided herein, at least one of the peptides comprises an N-terminal pyroglutamate and/or a C-terminal amide group. In some embodiments, each of the peptides comprises an N-terminal pyroglutamate and/or a C-terminal amide group.

In some embodiments of any one of the compositions provided herein, each of the peptides is less than full-length gluten. In some embodiments of any one of the compositions provided herein, each of the peptides is independently between 8 to 50 amino acids in length. In some embodiments, each of the peptides is independently between 10 to 30 amino acids in length. In some embodiments, each of the peptides is independently between 14 to 20 amino acids in length.

In some embodiments, the composition comprises at least one peptide, the at least one peptide comprising at least one amino acid sequence selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), EGSFQPSQE (SEQ ID NO: 17), QGYYPTSPQ (SEQ ID NO: 18), EQPEQPFPE (SEQ ID NO: 19), PFSEQEQPV (SEQ ID NO: 22), PYPQPELPY (SEQ ID NO: 25), EQPFPEQPI (SEQ ID NO: 23), PFPEQPIPE (SEQ ID NO: 24), PYPEQPQPF (SEQ ID NO: 16), and PQPYPEQPQ (SEQ ID NO: 27). In some embodiments, the composition comprises at least one (e.g., two, three, four, five, six, seven, eight, nine, ten, eleven, twelve, thirteen, fourteen, fifteen, or sixteen) peptide(s) comprising at least four (e.g., four, five, six, seven, eight, nine, ten, eleven, twelve, thirteen, fourteen, fifteen, sixteen, seventeen, eighteen, nineteen, twenty, twenty-one, twenty-two, or twenty-three) amino acid sequences selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), EGSFQPSQE (SEQ ID NO: 17), QGYYPTSPQ (SEQ ID NO: 18), EQPEQPFPE (SEQ ID NO: 19), PFSEQEQPV (SEQ ID NO: 22), PYPQPELPY (SEQ ID NO: 25), EQPFPEQPI (SEQ ID NO: 23), PFPEQPIPE (SEQ ID NO: 24), PYPEQPQPF (SEQ ID NO: 16), and PQPYPEQPQ (SEQ ID NO: 27).

In some embodiments, the composition comprises at least one of:

(a) a first peptide comprising the amino acid sequence PFPQPELPY (SEQ ID NO: 1) and the amino acid sequence PQPELPYPQ (SEQ ID NO: 2);

(b) a second peptide comprising the amino acid sequence PFPQPEQPF (SEQ ID NO: 3) and the amino acid sequence PQPEQPFPW (SEQ ID NO: 4);

(c) a third peptide comprising the amino acid sequence EQPIPEQPQ (SEQ ID NO: 5) and the amino acid sequence PIPEQPQPY (SEQ ID NO: 6);

(d) a fourth peptide comprising the amino acid sequence PFPQPEQPI (SEQ ID NO: 7) and the amino acid sequence PQPEQPIPV (SEQ ID NO: 8);

(e) a fifth peptide comprising the amino acid sequence EQPIPVQPE (SEQ ID NO: 9); (f) a sixth peptide comprising the amino acid sequence PFPQPEQPT (SEQ ID NO: 10) and the amino acid sequence PQPEQPTPI (SEQ ID NO: 11);

(g) a seventh peptide comprising the amino acid sequence EQPTPIQPE (SEQ ID NO: 12);

(h) an eighth peptide comprising the amino acid sequence PQPEQPFPL (SEQ ID NO: 13);

(i) a ninth peptide comprising the amino acid sequence EQPFPLQPE (SEQ ID NO: 14);

(j) a tenth peptide comprising the amino acid sequence EGSFQPSQE (SEQ ID NO: 17);

(k) a eleventh peptide comprising the amino acid sequence QGYYPTSPQ (SEQ ID NO: 18);

(l) a twelfth peptide comprising the amino acid sequence EQPEQPFPE (SEQ ID NO: 19);

(m) a thirteenth peptide comprising the amino acid sequence PFSEQEQPV (SEQ ID NO: 22);

(n) a fourteenth peptide comprising the amino acid sequence PYPQPELPY (SEQ ID NO: 25);

(o) a fifteenth peptide comprising the amino acid sequence EQPFPEQPI (SEQ ID NO: 23) and the amino acid sequence PFPEQPIPE (SEQ ID NO: 24); and

(p) a sixteenth peptide comprising the amino acid sequence PYPEQPQPF (SEQ ID NO: 16) and the amino acid sequence PQPYPEQPQ (SEQ ID NO: 27).

In some embodiments:

(a) the first peptide comprises the amino acid sequence PFPQPELPYPQP (SEQ ID NO: 46);

(b) the second peptide comprises the amino acid sequence PFPQPEQPFPWQ (SEQ ID NO: 47);

(c) the third peptide comprises the amino acid sequence EQPIPEQPQPYP (SEQ ID NO: 48);

(d) the fourth peptide comprises the amino acid sequence PFPQPEQPIPVQ (SEQ ID NO: 49);

(e) the fifth peptide comprises the amino acid sequence PEQPIPVQPEQS (SEQ ID NO: 50);

(f) the sixth peptide comprises the amino acid sequence PFPQPEQPTPIQ (SEQ ID NO: 51);

(g) the seventh peptide comprises the amino acid sequence PEQPTPIQPEQP (SEQ ID NO: 52);

(h) the eighth peptide comprises the amino acid sequence PFPQPEQPFPLQ (SEQ ID NO: 53);

(i) the ninth peptide comprises the amino acid sequence PEQPFPLQPEQP (SEQ ID NO: 54);

(j) the tenth peptide comprises the amino acid sequence GEGSFQPSQENP (SEQ ID NO: 55);

(k) the eleventh peptide comprises the amino acid sequence QQGYYPTSPQQS (SEQ ID NO: 56);

(l) the twelfth peptide comprises the amino acid sequence PEQPEQPFPEQP (SEQ ID NO: 57);

(m) the thirteenth peptide comprises the amino acid sequence PPFSEQEQPVLP (SEQ ID NO: 58);

(n) the fourteenth peptide comprises the amino acid sequence PYPQPELPYPQP (SEQ ID NO: 59);

(o) the fifteenth peptide comprises the amino acid sequence EQPFPEQPIPEQ (SEQ ID NO: 60); and

(p) the sixteenth peptide comprises the amino acid sequence PQPYPEQPQPFP (SEQ ID NO: 61).

In some embodiments, the composition comprises (or consists of) at least four (e.g., four, five, six, seven, eight, nine, ten, eleven, twelve, thirteen, fourteen, fifteen or sixteen) of the peptides. In some embodiments, the composition comprises (or consists of) the peptides in (a)-(p).

In some embodiments of any one of the compositions provided, at least one of the peptides comprises an N-terminal pyroglutamate and/or a C-terminal amide group. In some embodiments of any one of the compositions provided, each of the peptides comprises an N-terminal pyroglutamate and/or a C-terminal amide group. In some embodiments of any one of the compositions provided herein, each of the peptides is less than full-length gluten. In some embodiments of any one of the compositions provided herein, each of the peptides is independently between 8 to 50 amino acids in length. In some embodiments, each of the peptides is independently between 10 to 30 amino acids in length. In some embodiments, each of the peptides is independently between 12 to 30 amino acids in length. In some embodiments, each of the peptides is 13 amino acids in length.

In some embodiments of any one of the compositions provided, the peptides in the composition each consist of the recited amino acid sequence(s).

In some embodiments of any one of the compositions provided herein, the composition further comprises a pharmaceutically acceptable carrier. In some embodiments of any one of the compositions provided herein, at least one of the peptides is bound to a) an HLA molecule, or b) a fragment of an HLA molecule, capable of binding the peptide.

Other aspects of the disclosure relate to a composition comprising one or more polynucleotides encoding the peptides of any one of the compositions described herein.

Other aspects of the disclosure relate to an isolated antigen presenting cell comprising any one of the compositions described herein.

Yet other aspects of the disclosure relate to a kit comprising any one of the compositions described herein and means to detect binding of one or more of the peptides in the composition to T cells. In some embodiments, the means to detect binding of one or more of the peptides in the composition to T cells is an antibody specific for a cytokine. In some embodiments, the cytokine is selected from IFN-gamma or IP-10.

Other aspects of the disclosure relate to a method for treating Celiac disease in a subject, the method comprising administering to a subject having Celiac disease an effective amount of any one of the compositions described herein or an antigen presenting cell described herein. In some embodiments, the subject is HLA-DQ2.2 positive and/or HLA-DQ8 positive. In some embodiments, the subject is HLA-DQ2.5 positive and either HLA-DQ2.2 positive or HLA-DQ8 positive.

Other aspects of the disclosure relate to a method for identifying a subject as having or at risk of having Celiac disease, the method comprising determining a T cell response to any one of the compositions described herein or an antigen presenting cell described herein in a sample comprising a T cell from the subject; and assessing whether or not the subject has or is at risk of having Celiac disease.

In some embodiments, the assessing comprises identifying the subject as (i) having or at risk of having Celiac disease if the T cell response to the composition is elevated compared to a control T cell response, or (ii) not having or not at risk of having Celiac disease if the T cell response to the composition is reduced compared to the control T cell response or the same as the control T cell response.

In some embodiments, the step of determining comprises contacting the sample with the composition and measuring a T cell response to the composition. In some embodiments, measuring a T cell response to the composition comprises measuring a level of a cytokine in the sample. In some embodiments, the cytokine is IFN-gamma or IP-10. In some embodiments, measuring comprises an enzyme-linked immunosorbent assay (ELISA), an enzyme-linked immunosorbent spot (ELISpot) assay, or a multiplex bead-based immunoassay. In some embodiments, the sample comprises whole blood or peripheral blood mononuclear cells.

In some embodiments, any one of the methods further comprises administering a composition comprising wheat, rye, or barley, or one or more peptides thereof, to the subject prior to determining the T cell response. In some embodiments, the composition comprising wheat, rye, or barley, or one or more peptides thereof, is administered to the subject more than once prior to determining the T cell response. In some embodiments, the composition comprising wheat, rye, or barley, or one or more peptides thereof, is administered to the subject at least once a day for three days. In some embodiments, the sample comprising the T cell is obtained from the subject after the administration of the composition comprising wheat, rye, or barley, or one or more peptides thereof. In some embodiments, the composition comprising wheat, rye, or barley, or one or more peptides thereof, is administered to the subject via oral administration. In some embodiments, the composition comprising wheat, rye, or barley, or one or more peptides thereof, is a foodstuff. In some embodiments, the sample is obtained from the subject 6 days after the oral administration.

In some embodiments any one of the methods provided further comprises treating the subject if identified as having or at risk of having Celiac disease or providing information to the subject about a treatment.

In some embodiments, any one of the methods provided further comprises a step of recommending a gluten-free diet if the subject is identified as having or at risk of having Celiac disease or providing information to the subject about such a diet.

In some embodiments, the subject is HLA-DQ2.2 positive and/or HLA-DQ8 positive. In some embodiments, the subject is HLA-DQ2.5 positive and either HLA-DQ2.2 positive or HLA-DQ8 positive.

In some embodiments of any one of the compositions, an antigen presenting cell, any one of the methods or any one of the kits described herein, the composition comprises at least one peptide selected from:

(a) a first peptide comprising the amino acid sequence PFPQPELPY (SEQ ID NO: 1) and the amino acid sequence PQPELPYPQ (SEQ ID NO: 2);

(b) a second peptide comprising the amino acid sequence PFPQPEQPF (SEQ ID NO: 3) and the amino acid sequence PQPEQPFPW (SEQ ID NO: 4);

(c) a third peptide comprising the amino acid sequence EQPIPEQPQ (SEQ ID NO: 5) and the amino acid sequence PIPEQPQPY (SEQ ID NO: 6);

(d) a fourth peptide comprising the amino acid sequence PFPQPEQPI (SEQ ID NO: 7), the amino acid sequence PQPEQPIPV (SEQ ID NO: 8), and the amino acid sequence EQPIPVQPE (SEQ ID NO: 9);

(e) a fifth peptide comprising the amino acid sequence PFPQPEQPT (SEQ ID NO: 10), the amino acid sequence PQPEQPTPI (SEQ ID NO: 11), and the amino acid sequence EQPTPIQPE (SEQ ID NO: 12);

(f) a sixth peptide comprising the amino acid sequence PFPQPEQPF (SEQ ID NO: 3), the amino acid sequence PQPEQPFPL (SEQ ID NO: 13), and the amino acid sequence EQPFPLQPE (SEQ ID NO: 14);

(g) a seventh peptide comprising the amino acid sequence PFPQPEQPF (SEQ ID NO: 3) and the amino acid sequence PQPEQPFSQ (SEQ ID NO: 15);

(h) an eighth peptide comprising the amino acid sequence PYPEQPQPF (SEQ ID NO: 16);

(i) a ninth peptide comprising the amino acid sequence PFPEQPEQI (SEQ ID NO: 21);

(j) a tenth peptide comprising the amino acid sequence EGSFQPSQE (SEQ ID NO: 17);

(k) an eleventh peptide comprising the amino acid sequence QGYYPTSPQ (SEQ ID NO: 18);

(l) a twelfth peptide comprising the amino acid sequence EQPEQPFPE (SEQ ID NO: 19) and the amino acid sequence EQPFPEQPQ (SEQ ID NO: 20);

(m) a thirteenth peptide comprising the amino acid sequence PFSEQEQPV (SEQ ID NO: 22);

(n) a fourteenth peptide comprising the amino acid sequence EQPFPEQPI (SEQ ID NO: 23), the amino acid sequence PFPEQPIPE (SEQ ID NO: 24), the amino acid sequence EQPIPEQPQ (SEQ ID NO: 5), and the amino acid sequence PIPEQPQPY (SEQ ID NO: 6);

(o) a fifteenth peptide comprising the amino acid sequence PQPELPYPQ (SEQ ID NO: 2) and the amino acid sequence PYPQPELPY (SEQ ID NO: 25);

(p) a sixteenth peptide comprising the amino acid sequence PFPQPELPY (SEQ ID NO: 1) and the amino acid sequence PQPELPYPY (SEQ ID NO: 26);

(q) a seventeenth peptide comprising the amino acid sequence EQPFPEQPI (SEQ ID NO: 23) and the amino acid sequence PFPEQPIPE (SEQ ID NO: 24); and

(r) an eighteenth peptide comprising the amino acid sequence PQPYPEQPQ (SEQ ID NO: 27) and the amino acid sequence PYPEQPQPF (SEQ ID NO: 16).

In some embodiments,

(a) the first peptide comprises the amino acid sequence LQPFPQPELPYPQPQ (SEQ ID NO: 28);

(b) the second peptide comprises the amino acid sequence QPFPQPEQPFPWQP (SEQ ID NO: 29);

(c) the third peptide comprises the amino acid sequence PEQPIPEQPQPYPQQ (SEQ ID NO: 30);

(d) the fourth peptide comprises the amino acid sequence QPFPQPEQPIPVQPEQS (SEQ ID NO: 31);

(e) the fifth peptide comprises the amino acid sequence QPFPQPEQPTPIQPEQP (SEQ ID NO: 32);

(f) the sixth peptide comprises the amino acid sequence QPFPQPEQPFPLQPEQP (SEQ ID NO: 33);

(g) the seventh peptide comprises the amino acid sequence QPFPQPEQPFSQQ (SEQ ID NO: 34);

(h) the eighth peptide comprises the amino acid sequence PQPYPEQPQPFPQQ (SEQ ID NO: 35);

(i) the ninth peptide comprises the amino acid sequence QPFPEQPEQIIPQQP (SEQ ID NO: 36);

(j) the tenth peptide comprises the amino acid sequence SGEGSFQPSQENPQ (SEQ ID NO: 37);

(k) the eleventh peptide comprises the amino acid sequence GQQGYYPTSPQQSG (SEQ ID NO: 38);

(l) the twelfth peptide comprises the amino acid sequence PEQPEQPFPEQPQQ (SEQ ID NO: 39);

(m) the thirteenth peptide comprises the amino acid sequence QPPFSEQEQPVLPQ (SEQ ID NO: 40);

(n) the fourteenth peptide comprises the amino acid sequence PEQPFPEQPIPEQPQPYP (SEQ ID NO: 41);

(o) the fifteenth peptide comprises the amino acid sequence QPYPQPELPYPQPQ (SEQ ID NO: 42);

(p) the sixteenth peptide comprises the amino acid sequence QPFPQPELPYPYPQ (SEQ ID NO: 43);

(q) the seventeenth peptide comprises the amino acid sequence PQEQPFPEQPIPEQP (SEQ ID NO: 44); and

(r) the eighteenth peptide comprises the amino acid sequence QPQPYPEQPQPFPQQ (SEQ ID NO: 45).

In some embodiments, the composition comprises (or consists of) the first, second, third, fourth, fifth, sixth, seventh, eighth, ninth, tenth, eleventh, twelfth, and thirteenth peptides. In some embodiments, the composition comprises (or consists of) the second, fourth, fifth, sixth, seventh, eighth, ninth, tenth, eleventh, twelfth, thirteenth, fourteenth, fifteenth, and sixteenth peptides. In some embodiments, the composition comprises (or consists of) the first, second, third, fourth, fifth, sixth, tenth, eleventh, twelfth, thirteenth, fifteenth, seventeenth, and eighteenth peptides.

In some embodiments of any one of the compositions, an antigen presenting cell, any one of the methods or any one of the kits described herein, the composition comprises at least one peptide selected from:

(a) a first peptide comprising the amino acid sequence PFPQPELPY (SEQ ID NO: 1) and the amino acid sequence PQPELPYPQ (SEQ ID NO: 2);

(b) a second peptide comprising the amino acid sequence PFPQPEQPF (SEQ ID NO: 3) and the amino acid sequence PQPEQPFPW (SEQ ID NO: 4);

(c) a third peptide comprising the amino acid sequence EQPIPEQPQ (SEQ ID NO: 5) and the amino acid sequence PIPEQPQPY (SEQ ID NO: 6);

(d) a fourth peptide comprising the amino acid sequence PFPQPEQPI (SEQ ID NO: 7) and the amino acid sequence PQPEQPIPV (SEQ ID NO: 8);

(e) a fifth peptide comprising the amino acid sequence EQPIPVQPE (SEQ ID NO: 9);

(f) a sixth peptide comprising the amino acid sequence PFPQPEQPT (SEQ ID NO: 10) and the amino acid sequence PQPEQPTPI (SEQ ID NO: 11);

(g) a seventh peptide comprising the amino acid sequence EQPTPIQPE (SEQ ID NO: 12);

(h) an eighth peptide comprising the amino acid sequence PQPEQPFPL (SEQ ID NO: 13);

(i) a ninth peptide comprising the amino acid sequence EQPFPLQPE (SEQ ID NO: 14);

(j) a tenth peptide comprising the amino acid sequence EGSFQPSQE (SEQ ID NO: 17);

(k) a eleventh peptide comprising the amino acid sequence QGYYPTSPQ (SEQ ID NO: 18);

(l) a twelfth peptide comprising the amino acid sequence EQPEQPFPE (SEQ ID NO: 19);

(m) a thirteenth peptide comprising the amino acid sequence PFSEQEQPV (SEQ ID NO: 22);

(n) a fourteenth peptide comprising the amino acid sequence PYPQPELPY (SEQ ID NO: 25);

(o) a fifteenth peptide comprising the amino acid sequence EQPFPEQPI (SEQ ID NO: 23) and the amino acid sequence PFPEQPIPE (SEQ ID NO: 24); and

(p) a sixteenth peptide comprising the amino acid sequence PYPEQPQPF (SEQ ID NO: 16) and the amino acid sequence PQPYPEQPQ (SEQ ID NO: 27).

In some embodiments:

(a) the first peptide comprises the amino acid sequence PFPQPELPYPQP (SEQ ID NO: 46);

(b) the second peptide comprises the amino acid sequence PFPQPEQPFPWQ (SEQ ID NO: 47);

(c) the third peptide comprises the amino acid sequence EQPIPEQPQPYP (SEQ ID NO: 48);

(d) the fourth peptide comprises the amino acid sequence PFPQPEQPIPVQ (SEQ ID NO: 49);

(e) the fifth peptide comprises the amino acid sequence PEQPIPVQPEQS (SEQ ID NO: 50);

(f) the sixth peptide comprises the amino acid sequence PFPQPEQPTPIQ (SEQ ID NO: 51);

(g) the seventh peptide comprises the amino acid sequence PEQPTPIQPEQP (SEQ ID NO: 52);

(h) the eighth peptide comprises the amino acid sequence PFPQPEQPFPLQ (SEQ ID NO: 53);

(i) the ninth peptide comprises the amino acid sequence PEQPFPLQPEQP (SEQ ID NO: 54);

(j) the tenth peptide comprises the amino acid sequence GEGSFQPSQENP (SEQ ID NO: 55);

(k) the eleventh peptide comprises the amino acid sequence QQGYYPTSPQQS (SEQ ID NO: 56);

(l) the twelfth peptide comprises the amino acid sequence PEQPEQPFPEQP (SEQ ID NO: 57);

(m) the thirteenth peptide comprises the amino acid sequence PPFSEQEQPVLP (SEQ ID NO: 58);

(n) the fourteenth peptide comprises the amino acid sequence PYPQPELPYPQP (SEQ ID NO: 59);

(o) the fifteenth peptide comprises the amino acid sequence EQPFPEQPIPEQ (SEQ ID NO: 60); and

(p) the sixteenth peptide comprises the amino acid sequence PQPYPEQPQPFP (SEQ ID NO: 61).

In some embodiments, the composition comprises (or consists of) at least four (e.g., four, five, six, seven, eight, nine, ten, eleven, twelve, thirteen, fourteen, fifteen or sixteen) of the peptides. In some embodiments, the composition comprises (or consists of) the peptides in (a)-(p).

In some embodiments of any one of the compositions provided, at least one of the peptides comprises an N-terminal pyroglutamate and/or a C-terminal amide group. In some embodiments of any one of the compositions provided, each of the peptides comprises an N-terminal pyroglutamate and/or a C-terminal amide group. In some embodiments of any one of the compositions provided herein, each of the peptides is less than full-length gluten. In some embodiments of any one of the compositions provided herein, each of the peptides is independently between 8 to 50 amino acids in length. In some embodiments, each of the peptides is independently between 10 to 30 amino acids in length. In some embodiments, each of the peptides is independently between 12 to 30 amino acids in length. In some embodiments, each of the peptides is 13 amino acids in length.

In some embodiments, a composition comprises (or consists of) any one of the peptide pools as described in the examples provided. In some embodiments, a composition comprising the epitopes of any one of the peptide pools of the examples is provided. In some embodiments of any one of the compositions, the peptides or epitopes are in equimolar amounts.

In other aspects, the disclosure relates to a composition comprising at least one peptide selected from:

(a) a first peptide comprising the amino acid sequence PFPQPELPY (SEQ ID NO: 1) and the amino acid sequence PQPELPYPQ (SEQ ID NO: 2);

(b) a second peptide comprising the amino acid sequence PFPQPEQPF (SEQ ID NO: 3) and the amino acid sequence PQPEQPFPW (SEQ ID NO: 4);

(c) a third peptide comprising the amino acid sequence PIPEQPQPY (SEQ ID NO: 6); (d) a fourth peptide comprising the amino acid sequence PFPQPEQPIP (SEQ ID NO: 62) and the amino acid sequence EQPIPVQPE (SEQ ID NO: 9);

(e) a fifth peptide comprising the amino acid sequence PFPQPEQPTPI (SEQ ID NO: 65) and the amino acid sequence EQPTPIQPE (SEQ ID NO: 12);

(f) a sixth peptide comprising the amino acid sequence PQPEQPFPL (SEQ ID NO: 13) and the amino acid sequence EQPFPLQPE (SEQ ID NO: 14);

(g) a seventh peptide comprising the amino acid sequence PFPQPEQPF (SEQ ID NO: 3) and the amino acid sequence PQPEQPFSQ (SEQ ID NO: 15);

(h) an eighth peptide comprising the amino acid sequence PYPEQPQPF (SEQ ID NO: 16);

(i) a ninth peptide comprising the amino acid sequence PFPEQPEQIIP (SEQ ID NO: 63);

(j) a tenth peptide comprising the amino acid sequence EGSFQPSQE (SEQ ID NO: 17);

(k) an eleventh peptide comprising the amino acid sequence QGYYPTSPQ (SEQ ID NO: 18);

(l) a twelfth peptide comprising the amino acid sequence EQPEQPFPEQPQ (SEQ ID NO: 64);

(m) a thirteenth peptide comprising the amino acid sequence PFSEQEQPV (SEQ ID NO: 22);

(n) a fourteenth peptide comprising the amino acid sequence EQPFPEQPI (SEQ ID NO: 23) and PIPEQPQPY (SEQ ID NO: 6);

(o) a fifteenth peptide comprising the amino acid sequence PQPELPYPQ (SEQ ID NO: 2) and the amino acid sequence PYPQPELPY (SEQ ID NO: 25);

(p) a sixteenth peptide comprising the amino acid sequence PFPQPELPY (SEQ ID NO: 1) and the amino acid sequence PQPELPYPY (SEQ ID NO: 26); and

(q) a seventeenth peptide comprising the amino acid sequence EQPFPEQPI (SEQ ID NO: 23).

In some embodiments, (a) the first peptide comprises the amino acid sequence LQPFPQPELPYPQPQ (SEQ ID NO: 28); (b) the second peptide comprises the amino acid sequence QPFPQPEQPFPWQP (SEQ ID NO: 29); (c) the third peptide comprises the amino acid sequence PEQPIPEQPQPYPQQ (SEQ ID NO: 30); (d) the fourth peptide comprises the amino acid sequence QPFPQPEQPIPVQPEQS (SEQ ID NO: 31); (e) the fifth peptide comprises the amino acid sequence QPFPQPEQPTPIQPEQP (SEQ ID NO: 32); (f) the sixth peptide comprises the amino acid sequence QPFPQPEQPFPLQPEQP (SEQ ID NO: 33); (g) the seventh peptide comprises the amino acid sequence QPFPQPEQPFSQQ (SEQ ID NO: 34); (h) the eighth peptide comprises the amino acid sequence PQPYPEQPQPFPQQ (SEQ ID NO: 35); (i) the ninth peptide comprises the amino acid sequence QPFPEQPEQIIPQQP (SEQ ID NO: 36); (j) the tenth peptide comprises the amino acid sequence SGEGSFQPSQENPQ (SEQ ID NO: 37); (k) the eleventh peptide comprises the amino acid sequence GQQGYYPTSPQQSG (SEQ ID NO: 38); (1) the twelfth peptide comprises the amino acid sequence PEQPEQPFPEQPQQ (SEQ ID NO: 39); (m) the thirteenth peptide comprises the amino acid sequence QPPFSEQEQPVLPQ (SEQ ID NO: 40); (n) the fourteenth peptide comprises the amino acid sequence PEQPFPEQPIPEQPQPYP (SEQ ID NO: 41); (o) the fifteenth peptide comprises the amino acid sequence QPYPQPELPYPQPQ (SEQ ID NO: 42); (p) the sixteenth peptide comprises the amino acid sequence QPFPQPELPYPYPQ (SEQ ID NO: 43); and (q) the seventeenth peptide comprises the amino acid sequence EQPFPEQPI (SEQ ID NO: 23).

In some embodiments, the composition comprises at least four of the peptides described herein. In some embodiments, the composition comprises (i) the first, second, and third peptides or the second, fourteenth, fifteenth, and sixteenth peptides; and (ii) at least one of the fourth, fifth, sixth, seventh, eighth, ninth, tenth, eleventh, twelfth, and thirteenth peptides. In some embodiments, the composition comprises at least two of the fourth, fifth, sixth, seventh, eighth, ninth, tenth, eleventh, twelfth, and thirteenth peptides. In some embodiments, the composition comprises (or consists of) at least three of the fourth, fifth, sixth, seventh, eighth, ninth, tenth, eleventh, twelfth, and thirteenth peptides. In some embodiments, the composition comprises (or consists of) at least four of the fourth, fifth, sixth, seventh, eighth, ninth, tenth, eleventh, twelfth, and thirteenth peptides. In some embodiments, the composition comprises (or consists of) at least five of the fourth, fifth, sixth, seventh, eighth, ninth, tenth, eleventh, twelfth, and thirteenth peptides. In some embodiments, the composition comprises (or consists of) at least six of the fourth, fifth, sixth, seventh, eighth, ninth, tenth, eleventh, twelfth, and thirteenth peptides. In some embodiments, the composition comprises (or consists of) at least seven of the fourth, fifth, sixth, seventh, eighth, ninth, tenth, eleventh, twelfth, and thirteenth peptides. In some embodiments, the composition comprises (or consists of) at least eight of the fourth, fifth, sixth, seventh, eighth, ninth, tenth, eleventh, twelfth, and thirteenth peptides. In some embodiments, the composition comprises (or consists of) at least nine of the fourth, fifth, sixth, seventh, eighth, ninth, tenth, eleventh, twelfth, and thirteenth peptides.

In some embodiments, the composition comprises (or consists of) the first, second, third, fourth, fifth, sixth, seventh, eighth, ninth, tenth, eleventh, twelfth, and thirteenth peptides. In some embodiments, the composition comprises (or consists of) the second, fourth, fifth, sixth, seventh, eighth, ninth, tenth, eleventh, twelfth, thirteenth, fourteenth, fifteenth, and sixteenth peptides. In some embodiments, the composition comprises (or consists of) the first, second, third, fourth, fifth, sixth, eighth, ninth, tenth, eleventh, twelfth, and thirteenth peptides. In some embodiments, the composition comprises (or consists of) the second, fourth, fifth, sixth, eighth, ninth, tenth, eleventh, twelfth, thirteenth, fourteenth, fifteenth, and sixteenth peptides.

In some embodiments of any one of the compositions provided herein, at least one of the peptides comprises an N-terminal pyroglutamate and/or a C-terminal amide group. In some embodiments of any one of the compositions provided herein, each of the peptides comprises an N-terminal pyroglutamate and/or a C-terminal amide group.

In some embodiments of any one of the compositions provided herein, each of the peptides is less than full-length gluten. In some embodiments of any one of the compositions provided herein, each of the peptides is independently between 8 to 50 amino acids in length. In some embodiments of any one of the compositions provided herein, each of the peptides is independently between 10 to 30 amino acids in length. In some embodiments of any one of the compositions provided herein, each of the peptides is independently between 14 to 20 amino acids in length.

In some embodiments of any one of the compositions provided herein, the composition further comprises a pharmaceutically acceptable carrier. In some embodiments of any one of the compositions provided herein, at least one of the peptides is bound to a) an HLA molecule, or b) a fragment of an HLA molecule, capable of binding the peptide.

Other aspects of the disclosure relate to a composition comprising one or more polynucleotides encoding the peptides of any one of the compositions described herein. Other aspects of the disclosure relate to an isolated antigen presenting cell comprising any one of the compositions described herein.

Yet other aspects of the disclosure relate to a kit comprising any one of the compositions described herein and means to detect binding of one or more of the peptides in the composition to T cells. In some embodiments, the means to detect binding of one or more of the peptides in the composition to T cells is an antibody specific for a cytokine. In some embodiments, the cytokine is selected from IFN-gamma or IP-10.

Other aspects of the disclosure relate to a method for treating Celiac disease in a subject, the method comprising administering to a subject having Celiac disease an effective amount of any one of the compositions described herein or an antigen presenting cell described herein. In some embodiments, the subject is HLA-DQ2.2 positive and/or HLA-DQ8 positive. In some embodiments, the subject is HLA-DQ2.5 positive and either HLA-DQ2.2 positive or HLA-DQ8 positive.

Other aspects of the disclosure relate to a method for identifying a subject as having or at risk of having Celiac disease, the method comprising determining a T cell response to any one of the compositions described herein or an antigen presenting cell described herein in a sample comprising a T cell from the subject; and assessing whether or not the subject has or is at risk of having Celiac disease.

In some embodiments, the assessing comprises identifying the subject as (i) having or at risk of having Celiac disease if the T cell response to the composition is elevated compared to a control T cell response, or (ii) not having or not at risk of having Celiac disease if the T cell response to the composition is reduced compared to the control T cell response or the same as the control T cell response.

In some embodiments, the step of determining comprises contacting the sample with the composition and measuring a T cell response to the composition. In some embodiments, measuring a T cell response to the composition comprises measuring a level of a cytokine in the sample. In some embodiments, the cytokine is IFN-gamma or IP-10. In some embodiments, measuring comprises an enzyme-linked immunosorbent assay (ELISA), an enzyme-linked immunosorbent spot (ELISpot) assay, or a multiplex bead-based immunoassay. In some embodiments, the sample comprises whole blood or peripheral blood mononuclear cells.

In some embodiments, any one of the methods provided herein further comprises administering a composition comprising wheat, rye, or barley, or one or more peptides thereof, to the subject prior to determining the T cell response. In some embodiments, the composition comprising wheat, rye, or barley, or one or more peptides thereof, is administered to the subject more than once prior to determining the T cell response. In some embodiments, the composition comprising wheat, rye, or barley, or one or more peptides thereof, is administered to the subject at least once a day for three days. In some embodiments, the sample comprising the T cell is obtained from the subject after the administration of the composition comprising wheat, rye, or barley, or one or more peptides thereof. In some embodiments, the composition comprising wheat, rye, or barley, or one or more peptides thereof, is administered to the subject via oral administration. In some embodiments, the composition comprising wheat, rye, or barley, or one or more peptides thereof, is a foodstuff. In some embodiments, the sample is obtained from the subject 6 days after the oral administration.

In some embodiments, any one of the methods provided herein further comprises treating the subject if identified as having or at risk of having Celiac disease or providing information to the subject about a treatment.

In some embodiments, any one of the methods provided herein further comprises a step of recommending a gluten-free diet if the subject is identified as having or at risk of having Celiac disease or providing information to the subject about such a diet.

In some embodiments of any one of the methods provided herein, the method further comprises recording the level(s), the result(s) of the assessing and/or the treatment, or suggestion for treatment, based on the assessing.

In some embodiments, the subject is HLA-DQ2.2 positive and/or HLA-DQ8 positive. In some embodiments, the subject is HLA-DQ2.5 positive and either HLA-DQ2.2 positive or HLA-DQ8 positive.

In some embodiments of any one of the compositions, an antigen presenting cell, any one of the methods or any one of the kits described herein, the composition comprises at least one peptide selected from:

(a) a first peptide comprising the amino acid sequence PFPQPELPY (SEQ ID NO: 1) and the amino acid sequence PQPELPYPQ (SEQ ID NO: 2);

(b) a second peptide comprising the amino acid sequence PFPQPEQPF (SEQ ID NO: 3) and the amino acid sequence PQPEQPFPW (SEQ ID NO: 4);

(c) a third peptide comprising the amino acid sequence PIPEQPQPY (SEQ ID NO: 6);

(d) a fourth peptide comprising the amino acid sequence PFPQPEQPIP (SEQ ID NO: 62) and the amino acid sequence EQPIPVQPE (SEQ ID NO: 9);

(e) a fifth peptide comprising the amino acid sequence PFPQPEQPTPI (SEQ ID NO: 65) and the amino acid sequence EQPTPIQPE (SEQ ID NO: 12);

(f) a sixth peptide comprising the amino acid sequence PQPEQPFPL (SEQ ID NO: 13) and the amino acid sequence EQPFPLQPE (SEQ ID NO: 14);

(g) a seventh peptide comprising the amino acid sequence PFPQPEQPF (SEQ ID NO: 3) and the amino acid sequence PQPEQPFSQ (SEQ ID NO: 15);

(h) an eighth peptide comprising the amino acid sequence PYPEQPQPF (SEQ ID NO: 16);

(i) a ninth peptide comprising the amino acid sequence PFPEQPEQIIP (SEQ ID NO: 63);

(j) a tenth peptide comprising the amino acid sequence EGSFQPSQE (SEQ ID NO: 17);

(k) an eleventh peptide comprising the amino acid sequence QGYYPTSPQ (SEQ ID NO: 18);

(l) a twelfth peptide comprising the amino acid sequence EQPEQPFPEQPQ (SEQ ID NO: 64);

(m) a thirteenth peptide comprising the amino acid sequence PFSEQEQPV (SEQ ID NO: 22);

(n) a fourteenth peptide comprising the amino acid sequence EQPFPEQPI (SEQ ID NO: 23) and PIPEQPQPY (SEQ ID NO: 6);

(o) a fifteenth peptide comprising the amino acid sequence PQPELPYPQ (SEQ ID NO: 2) and the amino acid sequence PYPQPELPY (SEQ ID NO: 25);

(p) a sixteenth peptide comprising the amino acid sequence PFPQPELPY (SEQ ID NO: 1) and the amino acid sequence PQPELPYPY (SEQ ID NO: 26); and

(q) a seventeenth peptide comprising the amino acid sequence EQPFPEQPI (SEQ ID NO: 23). In some embodiments, (a) the first peptide comprises the amino acid sequence LQPFPQPELPYPQPQ (SEQ ID NO: 28); (b) the second peptide comprises the amino acid sequence QPFPQPEQPFPWQP (SEQ ID NO: 29); (c) the third peptide comprises the amino acid sequence PEQPIPEQPQPYPQQ (SEQ ID NO: 30); (d) the fourth peptide comprises the amino acid sequence QPFPQPEQPIPVQPEQS (SEQ ID NO: 31); (e) the fifth peptide comprises the amino acid sequence QPFPQPEQPTPIQPEQP (SEQ ID NO: 32); (f) the sixth peptide comprises the amino acid sequence QPFPQPEQPFPLQPEQP (SEQ ID NO: 33); (g) the seventh peptide comprises the amino acid sequence QPFPQPEQPFSQQ (SEQ ID NO: 34); (h) the eighth peptide comprises the amino acid sequence PQPYPEQPQPFPQQ (SEQ ID NO: 35); (i) the ninth peptide comprises the amino acid sequence QPFPEQPEQIIPQQP (SEQ ID NO: 36); (j) the tenth peptide comprises the amino acid sequence SGEGSFQPSQENPQ (SEQ ID NO: 37); (k) the eleventh peptide comprises the amino acid sequence GQQGYYPTSPQQSG (SEQ ID NO: 38); (1) the twelfth peptide comprises the amino acid sequence PEQPEQPFPEQPQQ (SEQ ID NO: 39); (m) the thirteenth peptide comprises the amino acid sequence QPPFSEQEQPVLPQ (SEQ ID NO: 40); (n) the fourteenth peptide comprises the amino acid sequence PEQPFPEQPIPEQPQPYP (SEQ ID NO: 41); (o) the fifteenth peptide comprises the amino acid sequence QPYPQPELPYPQPQ (SEQ ID NO: 42); (p) the sixteenth peptide comprises the amino acid sequence QPFPQPELPYPYPQ (SEQ ID NO: 43); and (q) the seventeenth peptide comprises the amino acid sequence EQPFPEQPI (SEQ ID NO: 23). In some embodiments, the composition comprises (or consists of) the first, second, third, fourth, fifth, sixth, seventh, eighth, ninth, tenth, eleventh, twelfth, and thirteenth peptides. In some embodiments, the composition comprises (or consists of) the second, fourth, fifth, sixth, seventh, eighth, ninth, tenth, eleventh, twelfth, thirteenth, fourteenth, fifteenth, and sixteenth peptides. In some embodiments, the composition comprises (or consists of) the first, second, third, fourth, fifth, sixth, eighth, ninth, tenth, eleventh, twelfth, and thirteenth peptides. In some embodiments, the composition comprises (or consists of) the second, fourth, fifth, sixth, eighth, ninth, tenth, eleventh, twelfth, thirteenth, fourteenth, fifteenth, and sixteenth peptides.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-C are graphs showing levels of whole blood plasma IP-10 and interferon-gamma (IFNγ or IFNg) in exemplary subjects 1, 2, and 3 having Celiac disease after the blood was contacted with individual gluten peptides. Blood was collected from the subjects six days after commencing oral gluten challenge. The X-axis for each graph shows, from left to right as pairs of bars, peptide 1, peptide 2, peptide 3, peptide 4, peptide 2, peptide 5, peptide 6, peptide 7, peptide 8, peptide 9, peptide 10, peptide 11, peptide 15, peptide 12, peptide 13, peptide 14, and peptide 16. The sequences of these peptides are provided in Table 1.

FIGS. 2A-C are graphs showing levels of whole blood plasma IP-10 and interferon-gamma (IFNγ or IFNg) in exemplary subjects 4, 5, and 6 having Celiac disease after the blood was contacted with individual gluten peptides. Blood was collected from the subjects six days after commencing oral gluten challenge. The X-axis for each graph shows, from left to right as pairs of bars, peptide 1, peptide 2, peptide 3, peptide 4, peptide 2, peptide 5, peptide 6, peptide 7, peptide 8, peptide 9, peptide 10, peptide 11, peptide 15, peptide 12, peptide 13, peptide 14, and peptide 16. The sequences of these peptides are provided in Table 1.

FIGS. 3A-D are graphs showing levels of whole blood plasma IP-10 and interferon-gamma (IFNγ or IFNg) in exemplary subjects 7, 8, 9, and 10 having Celiac disease after the blood was contacted with individual gluten peptides. Blood was collected from the subjects six days after commencing oral gluten challenge. The X-axis for each graph shows, from left to right as pairs of bars, peptide 1, peptide 2, peptide 3, peptide 4, peptide 2, peptide 5, peptide 6, peptide 7, peptide 8, peptide 9, peptide 10, peptide 11, peptide 15, peptide 12, peptide 13, peptide 14, and peptide 16. The sequences of these peptides are provided in Table 1.

FIGS. 4A-C are graphs that show the levels of IP-10, IFNγ, and the number of IFNγ SFUs (spot forming units) in the blood of exemplary subject 1. The blood was contacted with medium (negative control), CEF (human CMV, EBV and influenza virus, positive control), peptide pool 1, peptide pool 2, peptide pool 3, or total gluten peptide pool. For each graph in FIGS. 4A-C and also in each graph in FIGS. 5A-13C, the X-axis labels from left to right are: Day 0 medium, Day 0 CEF, Day 6 medium, Day 6 CEF, Day 0 medium, Day 0 Pool 1 3×10 ug/ml, Day 6 medium, Day 6 pool 1 3×50 ug/mL, Day 6 pool 1 3×20 ug/mL, Day 6 pool 1 3×20 ug/mL, Day 6 pool 1 3×5 ug/mL, Day 0 medium, Day 0 Pool 2 13×5 ug/ml, Day 6 medium, Day 6 pool 2 13×25 ug/mL, Day 6 pool 2 13×10 ug/mL, Day 6 pool 2 13×5 ug/mL, Day 6 pool 2 13×2.5 ug/mL, Day 0 medium, Day 0 Pool 2 14×5 ug/ml, Day 6 medium, Day 6 pool 2 14×25 ug/mL, Day 6 pool 2 14×10 ug/mL, Day 6 pool 2 14×5 ug/mL, and Day 6 pool 2 14×2.5 ug/mL.

FIGS. 5A-C are graphs that show the levels of IP-10, IFNγ, and the number of IFNγ SFUs (spot forming units) in the blood of exemplary subject 2. The blood was contacted with medium (negative control), CEF (human CMV, EBV and influenza virus, positive control), peptide pool 1, peptide pool 2, peptide pool 3, or total gluten peptide pool.

FIGS. 6A-C are graphs that show the levels of IP-10, IFNγ, and the number of IFNγ SFUs (spot forming units) in the blood of exemplary subject 3. The blood was contacted with medium (negative control), CEF (human CMV, EBV and influenza virus, positive control), peptide pool 1, peptide pool 2, peptide pool 3, or total gluten peptide pool.

FIGS. 7A-C are graphs that show the levels of IP-10, IFNγ, and the number of IFNγ SFUs (spot forming units) in the blood of exemplary subject 4. The blood was contacted with medium (negative control), CEF (human CMV, EBV and influenza virus, positive control), peptide pool 1, peptide pool 2, peptide pool 3, or total gluten peptide pool.

FIGS. 8A-C are graphs that show the levels of IP-10, IFNγ, and the number of IFNγ SFUs (spot forming units) in the blood of exemplary subject 5. The blood was contacted with medium (negative control), CEF (human CMV, EBV and influenza virus, positive control), peptide pool 1, peptide pool 2, peptide pool 3, or total gluten peptide pool.

FIGS. 9A-C are graphs that show the levels of IP-10, IFNγ, and the number of IFNγ SFUs (spot forming units) in the blood of exemplary subject 6. The blood was contacted with medium (negative control), CEF (human CMV, EBV and influenza virus, positive control), peptide pool 1, peptide pool 2, peptide pool 3, or total gluten peptide pool.

FIGS. 10A-C are graphs that show the levels of IP-10, IFNγ, and the number of IFNγ SFUs (spot forming units) in the blood of exemplary subject 7. The blood was contacted with medium (negative control), CEF (human CMV, EBV and influenza virus, positive control), peptide pool 1, peptide pool 2, peptide pool 3, or total gluten peptide pool.

FIGS. 11A-C are graphs that show the levels of IP-10, IFNγ, and the number of IFNγ SFUs (spot forming units) in the blood of exemplary subject 8. The blood was contacted with medium (negative control), CEF (human CMV, EBV and influenza virus, positive control), peptide pool 1, peptide pool 2, peptide pool 3, or total gluten peptide pool.

FIGS. 12A-C are graphs that show the levels of IP-10, IFNγ, and the number of IFNγ SFUs (spot forming units) in the blood of exemplary subject 9. The blood was contacted with medium (negative control), CEF (human CMV, EBV and influenza virus, positive control), peptide pool 1, peptide pool 2, peptide pool 3, or total gluten peptide pool.

FIGS. 13A-C are graphs that show the levels of IP-10, IFNγ, and the number of IFNγ SFUs (spot forming units) in the blood of exemplary subject 10. The blood was contacted with medium (negative control), CEF (human CMV, EBV and influenza virus, positive control), peptide pool 1, peptide pool 2, peptide pool 3, or total gluten peptide pool.

FIGS. 14A-D are graphs that show IFNγ spot forming units (SFU) in an ELISpot of PBMCs in samples collected from subjects 6 days after commencing a 3 day oral gluten challenge.

FIGS. 15A-D are graphs that show IFNγ spot forming units (SFU) in an ELISpot of PBMCs in samples collected from subjects 6 days after commencing a 3 day oral gluten challenge.

FIGS. 16A and B are graphs that show IFNγ spot forming units (SFU) in an ELISpot of PBMCs in samples collected from subjects 6 days after commencing a 3 day oral gluten challenge.

FIGS. 17A-C are graphs that show responses to gluten peptide pools in cytokine release assays before (filled-in circles) and 6-days after (open circles) commencing oral gluten challenge in 10 HLA-DQ2.5+ subjects with celiac disease to medium only (Nil), P3 10 μg/mL, P14 5 μM, P13 5 μM, P71 10 μg/mL (P71), and CEF 1 μg/mL. Linked symbols represent individual subject data: spot forming units (SFU) per million PBMC in ELISpot assay, or the ratio of plasma cytokine concentration in whole blood incubated with antigen to medium only (stimulation index). Day-0 vs Day-6: *p<0.05 **p<0.01 by 2-tail Wilcoxon paired rank sum test.

FIGS. 18A-L are graphs that show Day-6 IFNγ ELISpot, whole blood (WB) IFNγ and IP-10 dose-responses to gluten peptide pools by subjects normalized against their response to P3 50 μg/mL. FIGS. 18A-D show ELISpot results normalized after subtraction of response to medium only for each of six subjects whose response to P3 50 μg/mL was at least 10 SFU per 1.2 million PBMC (3 wells) above medium only. Data shown are median +/−range from six subjects. FIGS. 18E-H show whole blood IFNγ release for seven subjects whose stimulation index to P3 50 μg/mL was SI>1.5. FIGS. 18I-L show whole blood IP-10 release for four subjects whose responses to P3 50 μg/mL were less than the maximal detectable limit, IP-10 levels in the other six subjects were all at or above the limit of quantitation. Statistical significance compared to P3 50 μg/mL for all 10 subjects assessed for IFNγ ELISpot and whole blood release are indicated by *p<0.05 or **p<0.01 (two-tail “Wilcoxon matched-pairs signed rank test”). Statistical significance of IP-10 responses were not formally tested due there being only four informative data sets.

FIGS. 19A-J are graphs that show several individual subject's measured plasma concentrations of IFNγ and IP-10 (pg/mL) before subtraction of response to medium alone in cytokine bead assay. Plasma was separated following 24 h whole blood incubation with individual gluten peptides (5 uM) or pools of peptides. Each graph is for blood collected six days after commencing oral gluten challenge for each of 10 subjects. r² values are for data points that were below the maximum level of quantitation for IP-10 is 10,000 pg/mL. FIGS. 19A-J are graphs for each of subjects 1-10, respectively.

FIGS. 20A-H are graphs that show the stimulation index and net concentration of IFNγ (FIGS. 20A-D) and IP-10 (FIGS. 20E-H) in plasma after subtraction of response to medium only in whole blood collected before (filled-in circles) and 6-days after (open circles) commencing oral gluten challenge in 10 HLADQ2.5+ subjects with celiac disease. Blood was incubated with one of four different peptide pools: (FIGS. 20A and E) P3 10 μg/mL, (FIGS. 20B and F) P14 5 μM, (FIGS. 20C and G) P13 5 μM, or (FIGS. 20D and H) P71 10 μg/mL.

FIGS. 21A-D are graphs that show IFNγ and IP-10 (pg/mL) in plasma from whole bloods samples incubated with medium alone. IFNγ and IP-10 measured in plasma from replicate blood samples collected on Day-6 in separate cytokine bead assay plates (inter-assay variation), or from blood collected before and after oral gluten challenge that was assessed in the same cytokine bead assay (temporal change). Ten subjects were studied on Day-0 and Day-6. Three sets of triplicate blood samples were incubated with medium and one set of triplicates was incubated in each of the duplicate plates on Day-6. One set of triplicate blood samples was incubated with medium on Day-0. Except for one plate, each blood sample incubated with medium yielded one plasma sample that was assessed in a single well in the cytokine bead assay. For the duplicate plates, corresponding wells were pooled. In one cytokine bead assay plate, IFNγ was measured in three triplicate plasmas from Day-6 and in one triplicate from Day-0. A further triplicate plasma sample from Day-6 was assessed in a second cytokine bead assay plate performed on the same day. Data points represent the mean of triplicates derived from three blood incubations.

FIG. 22 is a graph that shows IFNγ and IP-10 (pg/mL) in plasma from blood incubated with medium alone from 10 subjects. Plasma levels for both analytes were assessed in one set of triplicate blood incubations on Day-0 and from two sets of triplicate whole blood samples collected on Day-6. Each point represents the mean of triplicates.

FIG. 23 is a graph that shows the fold-change in IP-10 concentration in blood contacted with peptide pool 1, 3, or 4 compared to blood incubated with PBS alone.

### DETAILED DESCRIPTION

Celiac disease (CD, also sometimes referred to as coeiac disease, c(o)eliac sprue, non-tropical sprue, endemic sprue, gluten enteropathy or gluten-sensitive enteropathy, and gluten intolerance) is an autoimmune disorder of the small intestine caused by ingestion of gluten-containing foods that occurs in people of all ages, ranging from middle infancy onward, and affects approximately 1% of people in Europe and North America. Untreated Celiac disease is associated with increased risk of adenocarcinoma (small intestine cancer) and lymphoma of the small bowel (enteropathy-associated T-cell lymphoma), as well as other complications, such as ulcerative jejunitis (ulcer formation of the small bowel) and structuring (narrowing as a result of scarring with obstruction of the bowel).

Celiac disease generally occurs in genetically susceptible individuals who possess either HLA-DQ2 encoded by HLA-DQA1*05 and HLA-DQB1*02 (accounting for about 90% of individuals), variants of HLA-DQ2, or HLA-DQ8. Without wishing to be bound by theory, such individuals are thought to mount an inappropriate HLA-DQ2- and/or DQ8-restricted CD4⁺ T cell-mediated immune response to peptides derived from the aqueous-insoluble proteins of wheat flour, gluten, and related proteins in rye and barley (herein referred to as gluten peptides). Such individuals are thought to respond to different T cell epitopes, depending on the susceptibility alleles (e.g., HLA-DQ2.5+ subjects respond to different T cell epitopes than HLA-DQ8+ subjects).

As described herein, compositions designed to contain multiple T cell epitopes that are HLA-DQ2.5-, DQ2.2- and/or DQ8-restricted are provided. These compositions induced robust T cell responses in samples from subjects with Celiac disease. Accordingly, aspects of the disclosure relate to compositions, and methods and kits related to these compositions.

### Gluten Peptides and Compositions Containing Gluten Peptides

As used herein the term “gluten peptide” includes any peptide comprising a sequence derived from, or encompassed within, one or more of gluten proteins alpha (α), beta (β), gamma (γ) and omega (ω) gliadins, and low and high molecular weight (LMW and HMW) glutenins in wheat, B, C and D hordeins in barley, β, γ and omega secalins in rye, and optionally avenins in oats, including deamidated variants thereof containing one or more glutamine to glutamate substitutions. In some embodiments, the gluten peptide(s) stimulate a CD4+ T cell specific response.

In some embodiments, a gluten peptide may comprise or consist of one or more T cell epitope sequences selected from: PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), PQPEQPFSQ (SEQ ID NO: 15), PYPEQPQPF (SEQ ID NO: 16), EGSFQPSQE (SEQ ID NO: 17), QGYYPTSPQ (SEQ ID NO: 18), EQPEQPFPE (SEQ ID NO: 19), EQPFPEQPQ (SEQ ID NO: 20), PFPEQPEQI (SEQ ID NO: 21), PFSEQEQPV (SEQ ID NO: 22), EQPFPEQPI (SEQ ID NO: 23), PFPEQPIPE (SEQ ID NO: 24), PYPQPELPY (SEQ ID NO: 25), PQPELPYPY (SEQ ID NO: 26), and PQPYPEQPQ (SEQ ID NO: 27). In some embodiments, a gluten peptide may comprise or consist of the T cell epitope sequences PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), and PIPEQPQPY (SEQ ID NO: 6) and at least one further amino acid sequence selected from PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), PQPEQPFSQ (SEQ ID NO: 15), PYPEQPQPF (SEQ ID NO: 16), EGSFQPSQE (SEQ ID NO: 17), QGYYPTSPQ (SEQ ID NO: 18), EQPEQPFPE (SEQ ID NO: 19), EQPFPEQPQ (SEQ ID NO: 20), PFPEQPEQI (SEQ ID NO: 21), PFSEQEQPV (SEQ ID NO: 22), EQPFPEQPI (SEQ ID NO: 23), PFPEQPIPE (SEQ ID NO: 24), PYPQPELPY (SEQ ID NO: 25), PQPELPYPY (SEQ ID NO: 26), and PQPYPEQPQ (SEQ ID NO: 27). In some embodiments, a gluten peptide may comprise or consist of the T cell epitope sequences EQPFPEQPI (SEQ ID NO: 23), PFPEQPIPE (SEQ ID NO: 24), EQPIPEQPQ (SEQ ID NO: 5), and PIPEQPQPY (SEQ ID NO: 6). In some embodiments, a gluten peptide may include one or more T cell epitope sequences selected from: PFPQPELPY (SEQ ID NO: 1), PQPELPYPQ (SEQ ID NO: 2), PFPQPEQPF (SEQ ID NO: 3), PQPEQPFPW (SEQ ID NO: 4), PIPEQPQPY (SEQ ID NO: 6), PFPQPEQPIP (SEQ ID NO: 62), EQPIPVQPE (SEQ ID NO: 9), PFPQPEQPTPI (SEQ ID NO: 65), EQPTPIQPE (SEQ ID NO: 12), PQPEQPFPL (SEQ ID NO: 13), EQPFPLQPE (SEQ ID NO: 14), PFPQPEQPF (SEQ ID NO: 3), PQPEQPFSQ (SEQ ID NO: 15), PYPEQPQPF (SEQ ID NO: 16), PFPEQPEQIIP (SEQ ID NO: 63), EGSFQPSQE (SEQ ID NO: 17), QGYYPTSPQ (SEQ ID NO: 18), EQPEQPFPEQPQ (SEQ ID NO: 64), PFSEQEQPV (SEQ ID NO: 22), EQPFPEQPI (SEQ ID NO: 23), PIPEQPQPY (SEQ ID NO: 6), PQPELPYPQ (SEQ ID NO: 2), PYPQPELPY (SEQ ID NO: 25), PFPQPELPY (SEQ ID NO: 1), and PQPELPYPY (SEQ ID NO: 26). Preferably, in some embodiments, the gluten peptides are at least 8 or 9 amino acids in length.

In some embodiments, the gluten peptide is selected from:

(a) a first peptide comprising the amino acid sequence PFPQPELPY (SEQ ID NO: 1) and the amino acid sequence PQPELPYPQ (SEQ ID NO: 2);

(b) a second peptide comprising the amino acid sequence PFPQPEQPF (SEQ ID NO: 3) and the amino acid sequence PQPEQPFPW (SEQ ID NO: 4);

(c) a third peptide comprising the amino acid sequence EQPIPEQPQ (SEQ ID NO: 5) and the amino acid sequence PIPEQPQPY (SEQ ID NO: 6);

(d) a fourth peptide comprising the amino acid sequence PFPQPEQPI (SEQ ID NO: 7), the amino acid sequence PQPEQPIPV (SEQ ID NO: 8), and the amino acid sequence EQPIPVQPE (SEQ ID NO: 9);

(e) a fifth peptide comprising the amino acid sequence PFPQPEQPT (SEQ ID NO: 10), the amino acid sequence PQPEQPTPI (SEQ ID NO: 11), and the amino acid sequence EQPTPIQPE (SEQ ID NO: 12);

(f) a sixth peptide comprising the amino acid sequence PFPQPEQPF (SEQ ID NO: 3), the amino acid sequence PQPEQPFPL (SEQ ID NO: 13), and the amino acid sequence EQPFPLQPE (SEQ ID NO: 14);

(g) a seventh peptide comprising the amino acid sequence PFPQPEQPF (SEQ ID NO: 3) and the amino acid sequence PQPEQPFSQ (SEQ ID NO: 15);

(h) an eighth peptide comprising the amino acid sequence PYPEQPQPF (SEQ ID NO: 16);

(i) a ninth peptide comprising the amino acid sequence PFPEQPEQI (SEQ ID NO: 21);

(j) a tenth peptide comprising the amino acid sequence EGSFQPSQE (SEQ ID NO: 17);

(k) an eleventh peptide comprising the amino acid sequence QGYYPTSPQ (SEQ ID NO: 18);

(l) a twelfth peptide comprising the amino acid sequence EQPEQPFPE (SEQ ID NO: 19) and the amino acid sequence EQPFPEQPQ (SEQ ID NO: 20);

(m) a thirteenth peptide comprising the amino acid sequence PFSEQEQPV (SEQ ID NO: 22);

(n) a fourteenth peptide comprising the amino acid sequence EQPFPEQPI (SEQ ID NO: 23), the amino acid sequence PFPEQPIPE (SEQ ID NO: 24), the amino acid sequence EQPIPEQPQ (SEQ ID NO: 5), and the amino acid sequence PIPEQPQPY (SEQ ID NO: 6);

(o) a fifteenth peptide comprising the amino acid sequence PQPELPYPQ (SEQ ID NO: 2) and the amino acid sequence PYPQPELPY (SEQ ID NO: 25);

(p) a sixteenth peptide comprising the amino acid sequence PFPQPELPY (SEQ ID NO: 1) and the amino acid sequence PQPELPYPY (SEQ ID NO: 26);

(q) a seventeenth peptide comprising the amino acid sequence EQPFPEQPI (SEQ ID NO: 23) and the amino acid sequence PFPEQPIPE (SEQ ID NO: 24); and

(r) an eighteenth peptide comprising the amino acid sequence PQPYPEQPQ (SEQ ID NO: 27) and the amino acid sequence PYPEQPQPF (SEQ ID NO: 16). In some embodiments, any one or more of the peptides herein comprises an N-terminal pyroglutamate and/or a C-terminal amide group.

In some embodiments, the gluten peptide is selected from:

(i) a first peptide comprising the amino acid sequence PFPQPELPY (SEQ ID NO: 1) and the amino acid sequence PQPELPYPQ (SEQ ID NO: 2);

(ii) a second peptide comprising the amino acid sequence PFPQPEQPF (SEQ ID NO: 3) and the amino acid sequence PQPEQPFPW (SEQ ID NO: 4);

(iii) a third peptide comprising the amino acid sequence PIPEQPQPY (SEQ ID NO: 6);

(iv) a fourth peptide comprising the amino acid sequence PFPQPEQPIP (SEQ ID NO: 62) and the amino acid sequence EQPIPVQPE (SEQ ID NO: 9);

(v) a fifth peptide comprising the amino acid sequence PFPQPEQPTPI (SEQ ID NO: 65) and the amino acid sequence EQPTPIQPE (SEQ ID NO: 12);

(vi) a sixth peptide comprising the amino acid sequence PQPEQPFPL (SEQ ID NO: 13) and the amino acid sequence EQPFPLQPE (SEQ ID NO: 14);

(vii) a seventh peptide comprising the amino acid sequence PFPQPEQPF (SEQ ID NO: 3) and the amino acid sequence PQPEQPFSQ (SEQ ID NO: 15);

(viii) an eighth peptide comprising the amino acid sequence PYPEQPQPF (SEQ ID NO: 16);

(ix) a ninth peptide comprising the amino acid sequence PFPEQPEQIIP (SEQ ID NO: 63);

(x) a tenth peptide comprising the amino acid sequence EGSFQPSQE (SEQ ID NO: 17);

(xi) an eleventh peptide comprising the amino acid sequence QGYYPTSPQ (SEQ ID NO: 18);

(xii) a twelfth peptide comprising the amino acid sequence EQPEQPFPEQPQ (SEQ ID NO: 64);

(xiii) a thirteenth peptide comprising the amino acid sequence PFSEQEQPV (SEQ ID NO: 22);

(xiv) a fourteenth peptide comprising the amino acid sequence EQPFPEQPI (SEQ ID NO: 23) and PIPEQPQPY (SEQ ID NO: 6);

(xv) a fifteenth peptide comprising the amino acid sequence PQPELPYPQ (SEQ ID NO: 2) and the amino acid sequence PYPQPELPY (SEQ ID NO: 25);

(xvi) a sixteenth peptide comprising the amino acid sequence PFPQPELPY (SEQ ID NO: 1) and the amino acid sequence PQPELPYPY (SEQ ID NO: 26); and

(xvii) a seventeenth peptide comprising the amino acid sequence EQPFPEQPI (SEQ ID NO: 23).

In some embodiments, the gluten peptide is selected from:

(i) a first peptide comprising the amino acid sequence LQPFPQPELPYPQPQ (SEQ ID NO: 28);

(ii) a second peptide comprising the amino acid sequence QPFPQPEQPFPWQP (SEQ ID NO: 29);

(iii) a third peptide comprising the amino acid sequence PEQPIPEQPQPYPQQ (SEQ ID NO: 30);

(iv) a fourth peptide comprising the amino acid sequence QPFPQPEQPIPVQPEQS (SEQ ID NO: 31);

(v) a fifth peptide comprising the amino acid sequence QPFPQPEQPTPIQPEQP (SEQ ID NO: 32);

(vi) a sixth peptide comprising the amino acid sequence QPFPQPEQPFPLQPEQP (SEQ ID NO: 33);

(vii) a seventh peptide comprising the amino acid sequence QPFPQPEQPFSQQ (SEQ ID NO: 34);

(viii) an eighth peptide comprising the amino acid sequence PQPYPEQPQPFPQQ (SEQ ID NO: 35);

(ix) a ninth peptide comprising the amino acid sequence QPFPEQPEQIIPQQP (SEQ ID NO: 36);

(x) a tenth peptide comprising the amino acid sequence SGEGSFQPSQENPQ (SEQ ID NO: 37);

(xi) an eleventh peptide comprising the amino acid sequence GQQGYYPTSPQQSG (SEQ ID NO: 38);

(xii) a twelfth peptide comprising the amino acid sequence PEQPEQPFPEQPQQ (SEQ ID NO: 39);

(xiii) a thirteenth peptide comprising the amino acid sequence QPPFSEQEQPVLPQ (SEQ ID NO: 40);

(xiv) a fourteenth peptide comprising the amino acid sequence PEQPFPEQPIPEQPQPYP (SEQ ID NO: 41);

(xv) a fifteenth peptide comprising the amino acid sequence QPYPQPELPYPQPQ (SEQ ID NO: 42);

(xvi) a sixteenth peptide comprising the amino acid sequence QPFPQPELPYPYPQ (SEQ ID NO: 43); and

(xvii) a seventeenth peptide comprising the amino acid sequence EQPFPEQPI (SEQ ID NO: 23). In some embodiments, any one of the peptides herein comprises an N-terminal pyroglutamate and/or a C-terminal amide group.

In some embodiments, a gluten peptide may include one or more T cell epitope sequences selected from: PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), EGSFQPSQE (SEQ ID NO: 17), QGYYPTSPQ (SEQ ID NO: 18), EQPEQPFPE (SEQ ID NO: 19), PFSEQEQPV (SEQ ID NO: 22), PYPQPELPY (SEQ ID NO: 25), EQPFPEQPI (SEQ ID NO: 23), PFPEQPIPE (SEQ ID NO: 24), PYPEQPQPF (SEQ ID NO: 16), and PQPYPEQPQ (SEQ ID NO: 27).

In some embodiments, the gluten peptide is selected from:

(a) a first peptide comprising the amino acid sequence PFPQPELPY (SEQ ID NO: 1) and the amino acid sequence PQPELPYPQ (SEQ ID NO: 2);

(b) a second peptide comprising the amino acid sequence PFPQPEQPF (SEQ ID NO: 3) and the amino acid sequence PQPEQPFPW (SEQ ID NO: 4);

(c) a third peptide comprising the amino acid sequence EQPIPEQPQ (SEQ ID NO: 5) and the amino acid sequence PIPEQPQPY (SEQ ID NO: 6);

(d) a fourth peptide comprising the amino acid sequence PFPQPEQPI (SEQ ID NO: 7) and the amino acid sequence PQPEQPIPV (SEQ ID NO: 8);

(e) a fifth peptide comprising the amino acid sequence EQPIPVQPE (SEQ ID NO: 9);

(f) a sixth peptide comprising the amino acid sequence PFPQPEQPT (SEQ ID NO: 10) and the amino acid sequence PQPEQPTPI (SEQ ID NO: 11);

(g) a seventh peptide comprising the amino acid sequence EQPTPIQPE (SEQ ID NO: 12);

(h) an eighth peptide comprising the amino acid sequence PQPEQPFPL (SEQ ID NO: 13);

(i) a ninth peptide comprising the amino acid sequence EQPFPLQPE (SEQ ID NO: 14);

(j) a tenth peptide comprising the amino acid sequence EGSFQPSQE (SEQ ID NO: 17);

(k) a eleventh peptide comprising the amino acid sequence QGYYPTSPQ (SEQ ID NO: 18);

(l) a twelfth peptide comprising the amino acid sequence EQPEQPFPE (SEQ ID NO: 19);

(m) a thirteenth peptide comprising the amino acid sequence PFSEQEQPV (SEQ ID NO: 22);

(n) a fourteenth peptide comprising the amino acid sequence PYPQPELPY (SEQ ID NO: 25);

(o) a fifteenth peptide comprising the amino acid sequence EQPFPEQPI (SEQ ID NO: 23) and the amino acid sequence PFPEQPIPE (SEQ ID NO: 24); and

(p) a sixteenth peptide comprising the amino acid sequence PYPEQPQPF (SEQ ID NO: 16) and the amino acid sequence PQPYPEQPQ (SEQ ID NO: 27). In some embodiments, any one or more of the peptides herein comprises an N-terminal pyroglutamate and/or a C-terminal amide group.

Exemplary gluten peptides and methods for synthesizing or obtaining such peptides are known in the art and described herein (see, e.g., PCT Publication Nos.: WO/2001/025793, WO/2003/104273, WO/2005/105129, and WO/2010/060155, which are incorporated herein by reference in their entirety, including specifically the aforementioned peptides and methods). A gluten peptide can be recombinantly and/or synthetically produced. In some embodiments, a gluten peptide is chemically synthesized, e.g., using a method known in the art. Non-limiting examples of peptide synthesis include liquid-phase synthesis and solid-phase synthesis. In some embodiments, a gluten peptide is produced by enzymatic digestion, e.g., by enzymatic digestion of a larger polypeptide into short peptides.

In some embodiments, one or more glutamate residues of a gluten peptide may be generated by tissue transglutaminase (tTG) deamidation activity upon one or more glutamine residues of the gluten peptide. This deamidation of glutamine to glutamate can cause the generation of gluten peptides that can bind to HLA-DQ2 or -DQ8 molecules with high affinity. This reaction may occur in vitro by contacting the gluten peptide composition with tTG outside of the subject or in vivo following administration through deamidation via tTG in the body. Deamidation of a peptide may also be accomplished by synthesizing a peptide de novo with glutamate residues in place of one or more glutamine residues, and thus deamidation does not necessarily require use of tTG. For example, PFPQPQLPY (SEQ ID NO: 137) could become PFPQPELPY (SEQ ID NO: 1) after processing by tTG. Conservative substitution of E with D is also contemplated herein for any one of the peptides herein (e.g., PFPQPELPY (SEQ ID NO: 1) could become PFPQPDLPY (SEQ ID NO: 138)). Exemplary peptides including an E to D substitution include peptides comprising or consisting of one or more of the sequences selected from PFPQPDLPY (SEQ ID NO: 101), PQPDLPYPQ (SEQ ID NO: 102), PFPQPDQPF (SEQ ID NO: 103), PQPDQPFPW (SEQ ID NO: 104), PIPDQPQPY (SEQ ID NO: 105), PFPQPDQPIP (SEQ ID NO: 106), DQPIPVQPD (SEQ ID NO: 107), PFPQPDQPTPI (SEQ ID NO: 108), DQPTPIQPD (SEQ ID NO: 109), PQPDQPFPL (SEQ ID NO: 110), DQPFPLQPD (SEQ ID NO: 111), PFPQPDQPF (SEQ ID NO: 112), PQPDQPFSQ (SEQ ID NO: 113), PYPDQPQPF (SEQ ID NO: 114), PFPDQPDQIIP (SEQ ID NO: 115), DGSFQPSQD (SEQ ID NO: 116), DQPDQPFPDQPQ (SEQ ID NO: 117), PFSDQDQPV (SEQ ID NO: 118), DQPFPDQPI (SEQ ID NO: 119), PIPDQPQPY (SEQ ID NO: 120), PQPDLPYPQ (SEQ ID NO: 121), PYPQPDLPY (SEQ ID NO: 122), PFPQPDLPY (SEQ ID NO: 123), and PQPDLPYPY (SEQ ID NO: 124). Other exemplary peptides including an E to D substitution include peptides comprising or consisting of one or more of the sequences selected from PFPQPDLPY (SEQ ID NO: 123), PQPDLPYPQ (SEQ ID NO: 121), PFPQPDQPF (SEQ ID NO: 103), PQPDQPFPW (SEQ ID NO: 104), DQPIPDQPQ (SEQ ID NO: 125), PIPDQPQPY (SEQ ID NO: 105), PFPQPDQPI (SEQ ID NO: 126), PQPDQPIPV (SEQ ID NO: 127), DQPIPVQPE (SEQ ID NO: 128), PFPQPDQPT (SEQ ID NO: 129), PQPDQPTPI (SEQ ID NO: 130), DQPTPIQPD (SEQ ID NO: 109), PQPDQPFPL (SEQ ID NO: 110), DQPFPLQPD (SEQ ID NO: 111), PQPDQPFSQ (SEQ ID NO: 113), PYPDQPQPF (SEQ ID NO: 114), DGSFQPSQD (SEQ ID NO: 116), DQPQQPFPD (SEQ ID NO: 131), DQPDQPFPQ (SEQ ID NO: 132), DQPFPDQPQ (SEQ ID NO: 133), PFPDQPDQI (SEQ ID NO: 134), PFSDQDQPV (SEQ ID NO: 118), DQPFPDQPI (SEQ ID NO: 119), PFPDQPIPD (SEQ ID NO: 135), PYPQPDLPY (SEQ ID NO: 122), PQPDLPYPY (SEQ ID NO: 124), and PQPYPDQPQ (SEQ ID NO: 136). Such substituted peptides can be the gluten peptides of any one of the methods and compositions provided herein.

In some embodiments, it may be desirable to utilize the non-deamidated forms of such peptides, e.g., if the peptides are contained within a composition for administration to a subject where tissue transglutaminase will act in situ (see, e.g., Øyvind Molberg, Stephen McAdam, Knut E. A. Lundin, Christel Kristiansen, Helene Arentz-Hansen, Kjell Kett and Ludvig M. Sollid. T cells from celiac disease lesions recognize gliadin epitopes deamidated in situ by endogenous tissue transglutaminase. Eur. J. Immunol. 2001. 31: 1317-1323). Accordingly, gluten peptides that have not undergone deamidation are also contemplated herein (e.g., gluten peptides comprising or consisting of one or more of the sequences selected from: PFPQPQLPY (SEQ ID NO: 137), PQPQLPYPQ (SEQ ID NO: 139), PFPQPQQPF (SEQ ID NO: 140), PQPQQPFPW (SEQ ID NO: 141), PIPQQPQPY (SEQ ID NO: 142), PFPQPQQPIP (SEQ ID NO: 143), QQPIPVQPQ (SEQ ID NO: 144), PFPQPQQPTPI (SEQ ID NO: 145), QQPTPIQPQ (SEQ ID NO: 146), PQPQQPFPL (SEQ ID NO: 147), QQPFPLQPQ (SEQ ID NO: 148), PFPQPQQPF (SEQ ID NO: 140), PQPQQPFSQ (SEQ ID NO: 149), PYPQQPQPF (SEQ ID NO: 150), PFPQQPQQIIP (SEQ ID NO: 151), QGSFQPSQQ (SEQ ID NO: 152), QQPQQPFPQQPQ (SEQ ID NO: 153), PFSQQQQPV (SEQ ID NO: 154), QQPFPQQPI (SEQ ID NO: 156), PYPQPQLPY (SEQ ID NO: 157), and PQPQLPYPY (SEQ ID NO: 158) or gluten peptides comprising or consisting of one or more of the sequences selected from: PFPQPQLPY (SEQ ID NO: 137), PQPQLPYPQ (SEQ ID NO: 139), PFPQPQQPF (SEQ ID NO: 140), PQPQQPFPW (SEQ ID NO: 141), QQPIPQQPQ (SEQ ID NO: 159), PIPQQPQPY (SEQ ID NO: 142), PFPQPQQPI (SEQ ID NO: 160), PQPQQPIPV (SEQ ID NO: 161), QQPIPVQPQ (SEQ ID NO: 144), PFPQPQQPT (SEQ ID NO: 162), PQPQQPTPI (SEQ ID NO: 163), QQPTPIQPQ (SEQ ID NO: 146), PQPQQPFPL (SEQ ID NO: 147), QQPFPLQPQ (SEQ ID NO: 148), PQPQQPFSQ (SEQ ID NO: 149), PYPQQPQPF (SEQ ID NO: 150), QGSFQPSQQ (SEQ ID NO: 152), QGYYPTSPQ (SEQ ID NO: 18), QQPQQPFPQ (SEQ ID NO: 164), QQPQQPFPQ (SEQ ID NO: 165), QQPFPQQPQ (SEQ ID NO: 166), PFPQQPQQI (SEQ ID NO: 167), PFSQQQQPV (SEQ ID NO: 154), QQPFPQQPI (SEQ ID NO: 156), PFPQQPIPQ (SEQ ID NO: 168), PYPQPQLPY (SEQ ID NO: 157), PQPQLPYPY (SEQ ID NO: 169), and PQPYPQQPQ (SEQ ID NO: 170).

A gluten peptide may also be an analog of any one of the peptides described herein. Preferably, in some embodiments the analog is recognized by a CD4+ T cell that recognizes one or more of the epitopes listed herein. Exemplary analogs comprise a peptide that has a sequence that is, e.g., 75%, 80%, 85%, 90%, 95%, 96%, 97%, 98%, or 99% homologous to the epitopes specifically recited herein. In some embodiments, the analogs comprise a peptide that is, e.g., 75%, 80%, 85%, 90%, 95%, 96%, 97%, 98%, or 99% homologous to the peptides specifically recited herein. Analogs may also be a variant of any one of the peptides provided, such variants can include conservative amino acid substitutions, e.g., E to D substitution. The length of the peptide may vary. In some embodiments of any one of the compositions, peptides, methods, kits, or antigen presenting cells provided herein, peptides are, e.g., 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids in length. In some embodiments of any one of the compositions, peptides, methods, kits, or antigen presenting cells provided herein, peptides are, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100 or fewer amino acids in length. In some embodiments of any one of the compositions, peptides, methods, kits, or antigen presenting cells provided herein, peptides are, e.g., 4-100, 4-50, 4-40, 4-30, or 4-20 amino acids in length. In some embodiments of any one of the compositions, peptides, methods, kits, or antigen presenting cells provided herein, peptides are 4-20, 5-20, 6-20, 7-20, 8-20, 9-20, 10-20, 11-20, 12-20, 13-20, 14-20, or 15-20 amino acids in length. In some embodiments of any one of the compositions, peptides, methods, kits, or antigen presenting cells provided herein, peptides are e.g., 5-30, 10-30, 15-30 or 20-30 amino acids in length. In some embodiments of any one of the compositions, peptides, methods, kits, or antigen presenting cells provided herein, peptides are 4-50, 5-50, 6-50, 7-50, 8-50, 9-50, 10-50, 11-50, 12-50, 13-50, 14-50, or 15-50 amino acids in length. In some embodiments of any one of the compositions, peptides, methods, kits, or antigen presenting cells provided herein, peptides are 8-30 amino acids in length.

In some embodiments, a composition comprising one or one or more gluten peptide(s) is contemplated. In some embodiments, the composition comprises at least one (e.g., 1, 2, 3, 4, 5, 7, 8, 9, 10, 11, 12, 13, 14, 15, or more) peptide, the at least one peptide comprising at least one (e.g., 1, 2, 3, 4, 5, 7, 8, 9, 10, 11, 12, 13, 14, 15, or more) amino acid sequence(s) selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), PQPEQPFSQ (SEQ ID NO: 15), PYPEQPQPF (SEQ ID NO: 16), EGSFQPSQE (SEQ ID NO: 17), QGYYPTSPQ (SEQ ID NO: 18), EQPEQPFPE (SEQ ID NO: 19), EQPFPEQPQ (SEQ ID NO: 20), PFPEQPEQI (SEQ ID NO: 21), PFSEQEQPV (SEQ ID NO: 22), EQPFPEQPI (SEQ ID NO: 23), PFPEQPIPE (SEQ ID NO: 24), PYPQPELPY (SEQ ID NO: 25), PQPELPYPY (SEQ ID NO: 26), and PQPYPEQPQ (SEQ ID NO: 27).

In some embodiments, the composition comprises at least one (e.g., 1, 2, 3, 4, 5, 7, 8, 9, 10, 11, 12, 13, 14, 15, or more) peptide, the at least one peptide comprising at least one (e.g., 1, 2, 3, 4, 5, 7, 8, 9, 10, 11, 12, 13, 14, 15, or more) amino acid sequence(s) selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), PIPEQPQPY (SEQ ID NO: 6), PFPQPEQPIP (SEQ ID NO: 62), EQPIPVQPE (SEQ ID NO: 9), PFPQPEQPTPI (SEQ ID NO: 65), EQPTPIQPE (SEQ ID NO: 12), PQPEQPFPL (SEQ ID NO: 13), EQPFPLQPE (SEQ ID NO: 14), PFPQPEQPF (SEQ ID NO: 3), PQPEQPFSQ (SEQ ID NO: 15), PYPEQPQPF (SEQ ID NO: 16), PFPEQPEQIIP (SEQ ID NO: 63), EGSFQPSQE (SEQ ID NO: 17), QGYYPTSPQ (SEQ ID NO: 18), EQPEQPFPEQPQ (SEQ ID NO: 64), PFSEQEQPV (SEQ ID NO: 22), EQPFPEQPI (SEQ ID NO: 23), PIPEQPQPY (SEQ ID NO: 6), PQPELPYPQ (SEQ ID NO: 2), PYPQPELPY (SEQ ID NO: 25), PFPQPELPY (SEQ ID NO: 1), PQPELPYPY (SEQ ID NO: 26), and EQPFPEQPI (SEQ ID NO: 23).

In some embodiments, the composition comprises at least one of:

(a) a first peptide comprising the amino acid sequence PFPQPELPY (SEQ ID NO: 1) and the amino acid sequence PQPELPYPQ (SEQ ID NO: 2);

(b) a second peptide comprising the amino acid sequence PFPQPEQPF (SEQ ID NO: 3) and the amino acid sequence PQPEQPFPW (SEQ ID NO: 4);

(c) a third peptide comprising the amino acid sequence EQPIPEQPQ (SEQ ID NO: 5) and the amino acid sequence PIPEQPQPY (SEQ ID NO: 6);

(d) a fourth peptide comprising the amino acid sequence PFPQPEQPI (SEQ ID NO: 7), the amino acid sequence PQPEQPIPV (SEQ ID NO: 8), and the amino acid sequence EQPIPVQPE (SEQ ID NO: 9);

(e) a fifth peptide comprising the amino acid sequence PFPQPEQPT (SEQ ID NO: 10), the amino acid sequence PQPEQPTPI (SEQ ID NO: 11), and the amino acid sequence EQPTPIQPE (SEQ ID NO: 12);

(f) a sixth peptide comprising the amino acid sequence PFPQPEQPF (SEQ ID NO: 3), the amino acid sequence PQPEQPFPL (SEQ ID NO: 13), and the amino acid sequence EQPFPLQPE (SEQ ID NO: 14);

(g) a seventh peptide comprising the amino acid sequence PFPQPEQPF (SEQ ID NO: 3) and the amino acid sequence PQPEQPFSQ (SEQ ID NO: 15);

(h) an eighth peptide comprising the amino acid sequence PYPEQPQPF (SEQ ID NO: 16);

(i) a ninth peptide comprising the amino acid sequence PFPEQPEQI (SEQ ID NO: 21);

(j) a tenth peptide comprising the amino acid sequence EGSFQPSQE (SEQ ID NO: 17);

(k) an eleventh peptide comprising the amino acid sequence QGYYPTSPQ (SEQ ID NO: 18);

(l) a twelfth peptide comprising the amino acid sequence EQPEQPFPE (SEQ ID NO: 19) and the amino acid sequence EQPFPEQPQ (SEQ ID NO: 20);

(m) a thirteenth peptide comprising the amino acid sequence PFSEQEQPV (SEQ ID NO: 22);

(n) a fourteenth peptide comprising the amino acid sequence EQPFPEQPI (SEQ ID NO: 23), the amino acid sequence PFPEQPIPE (SEQ ID NO: 24), the amino acid sequence EQPIPEQPQ (SEQ ID NO: 5), and the amino acid sequence PIPEQPQPY (SEQ ID NO: 6);

(o) a fifteenth peptide comprising the amino acid sequence PQPELPYPQ (SEQ ID NO: 2) and the amino acid sequence PYPQPELPY (SEQ ID NO: 25);

(p) a sixteenth peptide comprising the amino acid sequence PFPQPELPY (SEQ ID NO: 1) and the amino acid sequence PQPELPYPY (SEQ ID NO: 26);

(q) a seventeenth peptide comprising the amino acid sequence EQPFPEQPI (SEQ ID NO: 23) and the amino acid sequence PFPEQPIPE (SEQ ID NO: 24); and

(r) an eighteenth peptide comprising the amino acid sequence PQPYPEQPQ (SEQ ID NO: 27) and the amino acid sequence PYPEQPQPF (SEQ ID NO: 16).

In some embodiments,

(a) the first peptide comprises the amino acid sequence LQPFPQPELPYPQPQ (SEQ ID NO: 28);

(b) the second peptide comprises the amino acid sequence QPFPQPEQPFPWQP (SEQ ID NO: 29);

(c) the third peptide comprises the amino acid sequence PEQPIPEQPQPYPQQ (SEQ ID NO: 30);

(d) the fourth peptide comprises the amino acid sequence QPFPQPEQPIPVQPEQS (SEQ ID NO: 31);

(e) the fifth peptide comprises the amino acid sequence QPFPQPEQPTPIQPEQP (SEQ ID NO: 32);

(f) the sixth peptide comprises the amino acid sequence QPFPQPEQPFPLQPEQP (SEQ ID NO: 33);

(g) the seventh peptide comprises the amino acid sequence QPFPQPEQPFSQQ (SEQ ID NO: 34);

(h) the eighth peptide comprises the amino acid sequence PQPYPEQPQPFPQQ (SEQ ID NO: 35);

(i) the ninth peptide comprises the amino acid sequence QPFPEQPEQIIPQQP (SEQ ID NO: 36);

(j) the tenth peptide comprises the amino acid sequence SGEGSFQPSQENPQ (SEQ ID NO: 37);

(k) the eleventh peptide comprises the amino acid sequence GQQGYYPTSPQQSG (SEQ ID NO: 38);

(l) the twelfth peptide comprises the amino acid sequence PEQPEQPFPEQPQQ (SEQ ID NO: 39);

(m) the thirteenth peptide comprises the amino acid sequence QPPFSEQEQPVLPQ (SEQ ID NO: 40);

(n) the fourteenth peptide comprises the amino acid sequence PEQPFPEQPIPEQPQPYP (SEQ ID NO: 41);

(o) the fifteenth peptide comprises the amino acid sequence QPYPQPELPYPQPQ (SEQ ID NO: 42);

(p) the sixteenth peptide comprises the amino acid sequence QPFPQPELPYPYPQ (SEQ ID NO: 43);

(q) the seventeenth peptide comprises the amino acid sequence PQEQPFPEQPIPEQP (SEQ ID NO: 44); and

(r) the eighteenth peptide comprises the amino acid sequence QPQPYPEQPQPFPQQ (SEQ ID NO: 45).

In some embodiments, the composition comprises at least one of:

(i) a first peptide comprising the amino acid sequence PFPQPELPY (SEQ ID NO: 1) and the amino acid sequence PQPELPYPQ (SEQ ID NO: 2);

(ii) a second peptide comprising the amino acid sequence PFPQPEQPF (SEQ ID NO: 3) and the amino acid sequence PQPEQPFPW (SEQ ID NO: 4);

(iii) a third peptide comprising the amino acid sequence PIPEQPQPY (SEQ ID NO: 6);

(iv) a fourth peptide comprising the amino acid sequence PFPQPEQPIP (SEQ ID NO: 62) and the amino acid sequence EQPIPVQPE (SEQ ID NO: 9);

(v) a fifth peptide comprising the amino acid sequence PFPQPEQPTPI (SEQ ID NO: 65) and the amino acid sequence EQPTPIQPE (SEQ ID NO: 12);

(vi) a sixth peptide comprising the amino acid sequence PQPEQPFPL (SEQ ID NO: 13) and the amino acid sequence EQPFPLQPE (SEQ ID NO: 14);

(vii) a seventh peptide comprising the amino acid sequence PFPQPEQPF (SEQ ID NO: 3) and the amino acid sequence PQPEQPFSQ (SEQ ID NO: 15);

(viii) an eighth peptide comprising the amino acid sequence PYPEQPQPF (SEQ ID NO: 16);

(ix) a ninth peptide comprising the amino acid sequence PFPEQPEQIIP (SEQ ID NO: 63);

(x) a tenth peptide comprising the amino acid sequence EGSFQPSQE (SEQ ID NO: 17);

(xi) an eleventh peptide comprising the amino acid sequence QGYYPTSPQ (SEQ ID NO: 18);

(xii) a twelfth peptide comprising the amino acid sequence EQPEQPFPEQPQ (SEQ ID NO: 64);

(xiii) a thirteenth peptide comprising the amino acid sequence PFSEQEQPV (SEQ ID NO: 22);

(xiv) a fourteenth peptide comprising the amino acid sequence EQPFPEQPI (SEQ ID NO: 23) and PIPEQPQPY (SEQ ID NO: 6);

(xv) a fifteenth peptide comprising the amino acid sequence PQPELPYPQ (SEQ ID NO: 2) and the amino acid sequence PYPQPELPY (SEQ ID NO: 25);

(xvi) a sixteenth peptide comprising the amino acid sequence PFPQPELPY (SEQ ID NO: 1) and the amino acid sequence PQPELPYPY (SEQ ID NO: 26); and

(xvii) a seventeenth peptide comprising the amino acid sequence EQPFPEQPI (SEQ ID NO: 23).

In some embodiments,

(i) the first peptide comprises the amino acid sequence LQPFPQPELPYPQPQ (SEQ ID NO: 28);

(ii) the second peptide comprises the amino acid sequence QPFPQPEQPFPWQP (SEQ ID NO: 29);

(iii) the third peptide comprises the amino acid sequence PEQPIPEQPQPYPQQ (SEQ ID NO: 30);

(iv) the fourth peptide comprises the amino acid sequence QPFPQPEQPIPVQPEQS (SEQ ID NO: 31);

(v) the fifth peptide comprises the amino acid sequence QPFPQPEQPTPIQPEQP (SEQ ID NO: 32);

(vi) the sixth peptide comprises the amino acid sequence QPFPQPEQPFPLQPEQP (SEQ ID NO: 33);

(vii) the seventh peptide comprises the amino acid sequence QPFPQPEQPFSQQ (SEQ ID NO: 34);

(viii) the eighth peptide comprises the amino acid sequence PQPYPEQPQPFPQQ (SEQ ID NO: 35);

(ix) the ninth peptide comprises the amino acid sequence QPFPEQPEQIIPQQP (SEQ ID NO: 36);

(x) the tenth peptide comprises the amino acid sequence SGEGSFQPSQENPQ (SEQ ID NO: 37);

(xi) the eleventh peptide comprises the amino acid sequence GQQGYYPTSPQQSG (SEQ ID NO: 38);

(xii) the twelfth peptide comprises the amino acid sequence PEQPEQPFPEQPQQ (SEQ ID NO: 39);

(xiii) the thirteenth peptide comprises the amino acid sequence QPPFSEQEQPVLPQ (SEQ ID NO: 40);

(xiv) the fourteenth peptide comprises the amino acid sequence PEQPFPEQPIPEQPQPYP (SEQ ID NO: 41);

(xv) the fifteenth peptide comprises the amino acid sequence QPYPQPELPYPQPQ (SEQ ID NO: 42);

(xvi) the sixteenth peptide comprises the amino acid sequence QPFPQPELPYPYPQ (SEQ ID NO: 43); and

(xvii) the seventeenth peptide comprises the amino acid sequence EQPFPEQPI (SEQ ID NO: 23).

“First”, “second”, “third”, etc. are not meant to imply an order of use or importance, unless specifically stated otherwise. In some embodiments, the peptides are each individually 8-50 amino acids in length.

In some embodiments, the composition comprises at least one, at least two, at least three, at least four, at least five, at least six, at least seven, at least eight, at least nine, at least ten, at least eleven, at least twelve, at least thirteen, at least fourteen, at least fifteen or more of any of the peptides provided herein. In some embodiments, the composition comprises (i) the first, second, and third peptides or the second, fourteenth, fifteenth, and sixteenth peptides; and (ii) at least one (e.g., at least two, at least three, at least four, at least five, at least six, at least seven, at least eight, at least nine, or at least ten) of the fourth, fifth, sixth, seventh, eighth, ninth, tenth, eleventh, twelfth, and thirteenth peptides. In some embodiments, the composition comprises (i) the first, second, and third peptides or the second, fourteenth, fifteenth, and sixteenth peptides; and (ii) at least one (e.g., at least two, at least three, at least four, at least five, at least six, at least seven, at least eight, at least nine, or at least ten) of the fourth, fifth, sixth, seventh, eighth, ninth, tenth, eleventh, twelfth, thirteenth, sixteenth, seventeenth, and eighteenth peptides. In some embodiments, the composition comprises the first, second, third, fourth, fifth, sixth, seventh, eighth, ninth, tenth, eleventh, twelfth, and thirteenth peptides. In some embodiments, the composition comprises the second, fourth, fifth, sixth, seventh, eighth, ninth, tenth, eleventh, twelfth, thirteenth, fourteenth, fifteenth, and sixteenth peptides. In some embodiments, the composition comprises the first, second, third, fourth, fifth, sixth, eighth, ninth, tenth, eleventh, twelfth, and thirteenth peptides. In some embodiments, the composition comprises the second, fourth, fifth, sixth, eighth, ninth, tenth, eleventh, twelfth, thirteenth, fourteenth, fifteenth, and sixteenth peptides. In some embodiments, the composition comprises the second, third, fourth, fifth, sixth, seventh, eighth, ninth, tenth, eleventh, twelfth, thirteenth, fifteenth, sixteenth, and seventeenth peptides. In some embodiments, the composition comprises the second, third, fourth, fifth, sixth, eighth, ninth, tenth, eleventh, twelfth, thirteenth, fifteenth, sixteenth, and seventeenth peptides. In some embodiments, the composition comprises the first, second, third, fourth, fifth, sixth, tenth, eleventh, twelfth, thirteenth, fifteenth, seventeenth, and eighteenth peptides. In some embodiments, at least one of the peptides comprises an N-terminal pyroglutamate and/or a C-terminal amide group. In some embodiments, each of the peptides comprises an N-terminal pyroglutamate and/or a C-terminal amide group.

In some embodiments of any one of the compositions provided herein, the composition comprises at least one peptide, the at least one peptide comprising at least one amino acid sequence selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), EGSFQPSQE (SEQ ID NO: 17), QGYYPTSPQ (SEQ ID NO: 18), EQPEQPFPE (SEQ ID NO: 19), PFSEQEQPV (SEQ ID NO: 22), PYPQPELPY (SEQ ID NO: 25), EQPFPEQPI (SEQ ID NO: 23), PFPEQPIPE (SEQ ID NO: 24), PYPEQPQPF (SEQ ID NO: 16), and PQPYPEQPQ (SEQ ID NO: 27). In some embodiments of any one of the compositions provided herein, the composition comprises at least one (e.g., two, three, four, five, six, seven, eight, nine, ten, eleven, twelve, thirteen, fourteen, fifteen, or sixteen) peptide comprising at least four (e.g., four, five, six, seven, eight, nine, ten, eleven, twelve, thirteen, fourteen, fifteen, sixteen, seventeen, eighteen, nineteen, twenty, twenty-one, twenty-two, or twenty-three) amino acid sequences selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), EGSFQPSQE (SEQ ID NO: 17), QGYYPTSPQ (SEQ ID NO: 18), EQPEQPFPE (SEQ ID NO: 19), PFSEQEQPV (SEQ ID NO: 22), PYPQPELPY (SEQ ID NO: 25), EQPFPEQPI (SEQ ID NO: 23), PFPEQPIPE (SEQ ID NO: 24), PYPEQPQPF (SEQ ID NO: 16), and PQPYPEQPQ (SEQ ID NO: 27).

In some embodiments of any one of the compositions provided herein, the composition comprises at least one of:

(a) a first peptide comprising the amino acid sequence PFPQPELPY (SEQ ID NO: 1) and the amino acid sequence PQPELPYPQ (SEQ ID NO: 2);

(b) a second peptide comprising the amino acid sequence PFPQPEQPF (SEQ ID NO: 3) and the amino acid sequence PQPEQPFPW (SEQ ID NO: 4);

(c) a third peptide comprising the amino acid sequence EQPIPEQPQ (SEQ ID NO: 5) and the amino acid sequence PIPEQPQPY (SEQ ID NO: 6);

(d) a fourth peptide comprising the amino acid sequence PFPQPEQPI (SEQ ID NO: 7) and the amino acid sequence PQPEQPIPV (SEQ ID NO: 8);

(e) a fifth peptide comprising the amino acid sequence EQPIPVQPE (SEQ ID NO: 9);

(f) a sixth peptide comprising the amino acid sequence PFPQPEQPT (SEQ ID NO: 10) and the amino acid sequence PQPEQPTPI (SEQ ID NO: 11);

(g) a seventh peptide comprising the amino acid sequence EQPTPIQPE (SEQ ID NO: 12);

(h) an eighth peptide comprising the amino acid sequence PQPEQPFPL (SEQ ID NO: 13);

(i) a ninth peptide comprising the amino acid sequence EQPFPLQPE (SEQ ID NO: 14);

(j) a tenth peptide comprising the amino acid sequence EGSFQPSQE (SEQ ID NO: 17);

(k) a eleventh peptide comprising the amino acid sequence QGYYPTSPQ (SEQ ID NO: 18);

(l) a twelfth peptide comprising the amino acid sequence EQPEQPFPE (SEQ ID NO: 19);

(m) a thirteenth peptide comprising the amino acid sequence PFSEQEQPV (SEQ ID NO: 22);

(n) a fourteenth peptide comprising the amino acid sequence PYPQPELPY (SEQ ID NO: 25);

(o) a fifteenth peptide comprising the amino acid sequence EQPFPEQPI (SEQ ID NO: 23) and the amino acid sequence PFPEQPIPE (SEQ ID NO: 24); and

(p) a sixteenth peptide comprising the amino acid sequence PYPEQPQPF (SEQ ID NO: 16) and the amino acid sequence PQPYPEQPQ (SEQ ID NO: 27).

In some embodiments:

(a) the first peptide comprises the amino acid sequence PFPQPELPYPQP (SEQ ID NO: 46);

(b) the second peptide comprises the amino acid sequence PFPQPEQPFPWQ (SEQ ID NO: 47);

(c) the third peptide comprises the amino acid sequence EQPIPEQPQPYP (SEQ ID NO: 48);

(d) the fourth peptide comprises the amino acid sequence PFPQPEQPIPVQ (SEQ ID NO: 49);

(e) the fifth peptide comprises the amino acid sequence PEQPIPVQPEQS (SEQ ID NO: 50);

(f) the sixth peptide comprises the amino acid sequence PFPQPEQPTPIQ (SEQ ID NO: 51);

(g) the seventh peptide comprises the amino acid sequence PEQPTPIQPEQP (SEQ ID NO: 52);

(h) the eighth peptide comprises the amino acid sequence PFPQPEQPFPLQ (SEQ ID NO: 53);

(i) the ninth peptide comprises the amino acid sequence PEQPFPLQPEQP (SEQ ID NO: 54);

(j) the tenth peptide comprises the amino acid sequence GEGSFQPSQENP (SEQ ID NO: 55);

(k) the eleventh peptide comprises the amino acid sequence QQGYYPTSPQQS (SEQ ID NO: 56);

(l) the twelfth peptide comprises the amino acid sequence PEQPEQPFPEQP (SEQ ID NO: 57);

(m) the thirteenth peptide comprises the amino acid sequence PPFSEQEQPVLP (SEQ ID NO: 58);

(n) the fourteenth peptide comprises the amino acid sequence PYPQPELPYPQP (SEQ ID NO: 59);

(o) the fifteenth peptide comprises the amino acid sequence EQPFPEQPIPEQ (SEQ ID NO: 60); and

(p) the sixteenth peptide comprises the amino acid sequence PQPYPEQPQPFP (SEQ ID NO: 61).

In some embodiments of any one of the compositions provided herein, the composition comprises at least four (e.g., five, six, seven, eight, nine, ten, eleven, twelve, thirteen, fourteen, fifteen or sixteen) of the peptides. In some embodiments of any one of the compositions provided herein, the composition comprises (or consists of) the peptides in (a)-(p). In some embodiments of any one of the compositions provided herein, at least one of the peptides comprises an N-terminal pyroglutamate and/or a C-terminal amide group. In some embodiments of any one of the methods provided herein, each of the peptides comprises an N-terminal pyroglutamate and/or a C-terminal amide group.

Modifications to a gluten peptide are also contemplated herein. This modification may occur during or after translation or synthesis (for example, by farnesylation, prenylation, myristoylation, glycosylation, palmitoylation, acetylation, phosphorylation (such as phosphotyrosine, phosphoserine or phosphothreonine), amidation, pyrolation, derivatisation by known protecting/blocking groups, proteolytic cleavage, linkage to an antibody molecule or other cellular ligand, and the like). Any of the numerous chemical modification methods known within the art may be utilized including, but not limited to, specific chemical cleavage by cyanogen bromide, trypsin, chymotrypsin, papain, V8 protease, NaBH4, acetylation, formylation, oxidation, reduction, metabolic synthesis in the presence of tunicamycin, etc. The phrases “protecting group” and “blocking group” as used herein, refers to modifications to the peptide which protect it from undesirable chemical reactions, particularly chemical reactions in vivo. Examples of such protecting groups include esters of carboxylic acids and boronic acids, ethers of alcohols and acetals, and ketals of aldehydes and ketones. Examples of suitable groups include acyl protecting groups such as, for example, furoyl, formyl, adipyl, azelayl, suberyl, dansyl, acetyl, theyl, benzoyl, trifluoroacetyl, succinyl and methoxysuccinyl; aromatic urethane protecting groups such as, for example, benzyloxycarbonyl (Cbz); aliphatic urethane protecting groups such as, for example, t-butoxycarbonyl (Boc) or 9-fluorenylmethoxy-carbonyl (FMOC); pyroglutamate and amidation. Many other modifications providing increased potency, prolonged activity, ease of purification, and/or increased half-life are known to the person skilled in the art.

The peptides may comprise one or more modifications, which may be natural post-translation modifications or artificial modifications. The modification may provide a chemical moiety (typically by substitution of a hydrogen, for example, of a C—H bond), such as an amino, acetyl, acyl, carboxy, hydroxy or halogen (for example, fluorine) group, or a carbohydrate group. Typically, the modification is present on the N- and/or C-terminal. Furthermore, one or more of the peptides may be PEGylated, where the PEG (polyethyleneoxy group) provides for enhanced lifetime in the blood stream. One or more of the peptides may also be combined as a fusion or chimeric protein with other proteins, or with specific binding agents that allow targeting to specific moieties on a target cell.

A gluten peptide may also be chemically modified at the level of amino acid side chains, of amino acid chirality, and/or of the peptide backbone.

Particular changes can be made to a gluten peptide to improve resistance to degradation or optimize solubility properties or otherwise improve bioavailability compared to the parent gluten peptide, thereby providing gluten peptides having similar or improved therapeutic, diagnostic and/or pharmacokinetic properties. A preferred such modification includes the use of an N-terminal acetyl group or pyroglutamate and/or a C-terminal amide. Such modifications have been shown in the art to significantly increase the half-life and bioavailability of the peptides compared to the parent peptides having a free N- and C-terminus (see, e.g., PCT Publication No.: WO/2010/060155). In some embodiments, any one of the gluten peptides comprise an N-terminal acetyl group or pyroglutamate group, and/or a C-terminal amide group. In some embodiments, the first, second and/or third peptides described herein comprise an N-terminal acetyl group or pyroglutamate group, and/or a C-terminal amide group. In some embodiments, the first, second, third, fourth, fifth, sixth, seventh, eighth, ninth, tenth, eleventh, twelfth, and/or thirteenth peptides described herein comprise an N-terminal acetyl group or pyroglutamate group, and/or a C-terminal amide group. In some embodiments, the second, fourth, fifth, sixth, seventh, eighth, ninth, tenth, eleventh, twelfth, thirteenth, fourteenth, fifteenth, and/or sixteenth peptides described herein comprise an N-terminal acetyl group or pyroglutamate group, and/or a C-terminal amide group. In some embodiments, the first, second, third, fourth, fifth, sixth, eighth, ninth, tenth, eleventh, twelfth, and/or thirteenth peptides described herein comprise an N-terminal acetyl group or pyroglutamate group, and/or a C-terminal amide group. In some embodiments, the second, fourth, fifth, sixth, eighth, ninth, tenth, eleventh, twelfth, thirteenth, fourteenth, fifteenth, and/or sixteenth peptides described herein comprise an N-terminal acetyl group or pyroglutamate group, and/or a C-terminal amide group. In some embodiments, the first, second, third, fourth, fifth, sixth, tenth, eleventh, twelfth, thirteenth, fifteenth, seventeenth, and eighteenth peptides described herein comprise an N-terminal acetyl group or pyroglutamate group, and/or a C-terminal amide group.

### Peptide Production

The peptides described herein can be prepared in any suitable manner. For example, the peptides can be recombinantly and/or synthetically produced.

The peptides may be synthesised by standard chemistry techniques, including synthesis by an automated procedure using a commercially available peptide synthesiser. In general, peptides may be prepared by solid-phase peptide synthesis methodologies which may involve coupling each protected amino acid residue to a resin support, preferably a 4-methylbenzhydrylamine resin, by activation with dicyclohexylcarbodiimide to yield a peptide with a C-terminal amide. Alternatively, a chloromethyl resin (Merrifield resin) may be used to yield a peptide with a free carboxylic acid at the C-terminal. After the last residue has been attached, the protected peptide-resin is treated with hydrogen fluoride to cleave the peptide from the resin, as well as deprotect the side chain functional groups. Crude product can be further purified by gel filtration, high pressure liquid chromatography (HPLC), partition chromatography, or ion-exchange chromatography.

If desired, and as outlined above, various groups may be introduced into the peptide of the composition during synthesis or during expression, which allow for linking to other molecules or to a surface. For example, cysteines can be used to make thioethers, histidines for linking to a metal ion complex, carboxyl groups for forming amides or esters, amino groups for forming amides, and the like.

The peptides may also be produced using cell-free translation systems. Standard translation systems, such as reticulocyte lysates and wheat germ extracts, using RNA as a template; whereas “coupled” and “linked” systems start with DNA templates, which are transcribed into RNA then translated.

Alternatively, the peptides may be produced by transfecting host cells with expression vectors that comprise polynucleotide(s) that encode one or more peptides. For recombinant production, a recombinant construct comprising a sequence which encodes one or more of the peptides is introduced into host cells by conventional methods such as calcium phosphate transfection, DEAE-dextran mediated transfection, microinjection, cationic lipid-mediated transfection, electroporation, transduction, scrape lading, ballistic introduction or infection.

One or more of the peptides may be expressed in suitable host cells, such as, for example, mammalian cells (for example, COS, CHO, BHK, 293 HEK, VERO, HeLa, HepG2, MDCK, W138, or NIH 3T3 cells), yeast (for example, Saccharomyces or Pichia), bacteria (for example, E. coli, P. pastoris, or B. subtilis), insect cells (for example, baculovirus in Sf9 cells) or other cells under the control of appropriate promoters using conventional techniques. Following transformation of the suitable host strain and growth of the host strain to an appropriate cell density, the cells are harvested by centrifugation, disrupted by physical or chemical means, and the resulting crude extract retained for further purification of the peptide or variant thereof.

Suitable expression vectors include, for example, chromosomal, non-chromosomal and synthetic polynucleotides, for example, derivatives of SV40, bacterial plasmids, phage DNAs, yeast plasmids, vectors derived from combinations of plasmids and phage DNAs, viral DNA such as vaccinia viruses, adenovirus, adeno-associated virus, lentivirus, canary pox virus, fowl pox virus, pseudorabies, baculovirus, herpes virus and retrovirus. The polynucleotide may be introduced into the expression vector by conventional procedures known in the art.

The polynucleotide which encodes one or more peptides may be operatively linked to an expression control sequence, i.e., a promoter, which directs mRNA synthesis. Representative examples of such promoters include the LTR or SV40 promoter, the E. coli lac or trp, the phage lambda PL promoter and other promoters known to control expression of genes in prokaryotic or eukaryotic cells or in viruses. The expression vector may also contain a ribosome binding site for translation initiation and a transcription terminator. The expression vectors may also include an origin of replication and a selectable marker, such as the ampicillin resistance gene of E. coli to permit selection of transformed cells, i.e., cells that are expressing the heterologous polynucleotide. The nucleic acid molecule encoding one or more of the peptides may be incorporated into the vector in frame with translation initiation and termination sequences.

One or more of the peptides can be recovered and purified from recombinant cell cultures (i.e., from the cells or culture medium) by well-known methods including ammonium sulphate or ethanol precipitation, acid extraction, anion or cation exchange chromatography, phosphocellulose chromatography, hydrophobic interaction chromatography, affinity chromatography, hydroxyapatite chromatography, lectin chromatography, and HPLC. Well known techniques for refolding proteins may be employed to regenerate active conformation when the peptide is denatured during isolation and or purification.

To produce a glycosylated peptide, it is preferred that recombinant techniques be used. To produce a glycosylated peptide, it is preferred that mammalian cells such as, COS-7 and Hep-G2 cells be employed in the recombinant techniques.

The peptides can also be prepared by cleavage of longer peptides, especially from food extracts.

Pharmaceutically acceptable salts of the peptides can be synthesised from the peptides which contain a basic or acid moiety by conventional chemical methods. Generally, the salts are prepared by reacting the free base or acid with stoichiometric amounts or with an excess of the desired salt-forming inorganic or organic acid or base in a suitable solvent. In some embodiments, the pharmaceutically acceptable salt is a trifluoroacetate (TFA) salt or an acetate salt.

In some embodiments, a composition described herein further comprises a pharmaceutically acceptable carrier. The term “pharmaceutically acceptable carrier” refers to molecular entities and compositions that do not produce an allergic, toxic or otherwise adverse reaction when administered to a subject, particularly a mammal, and more particularly a human. The pharmaceutically acceptable carrier may be solid or liquid. Useful examples of pharmaceutically acceptable carriers include, but are not limited to, diluents, excipients, solvents, surfactants, suspending agents, buffering agents, lubricating agents, adjuvants, vehicles, emulsifiers, absorbents, dispersion media, coatings, stabilizers, protective colloids, adhesives, thickeners, thixotropic agents, penetration agents, sequestering agents, isotonic and absorption delaying agents that do not affect the activity of the active agents of the pharmaceutical composition. The carrier can be any of those conventionally used and is limited only by chemico-physical considerations, such as solubility and lack of reactivity with the active agent, and by the route of administration. Suitable carriers for the pharmaceutical composition include those conventionally used, for example, water, saline, aqueous dextrose, lactose, Ringer's solution, a buffered solution, hyaluronan, glycols, starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like. Liposomes may also be used as carriers. Other carriers are well known in the art (see, e.g., Remington: The Science and Practice of Pharmacy, 21st Ed. Lippincott Williams & Wilkins, 2005).

### Identification

In some aspects, the disclosure relates to methods for identifying (e.g., diagnosing) a subject as having or at risk of having Celiac disease.

In some embodiments, the method comprises determining a T cell response to any one of the compositions provided, such as a composition comprising at least one (e.g., at least four) peptide as described herein in a sample comprising a T cell from a subject and identifying the subject as (i) having or at risk of having Celiac disease if the T cell response to the peptide described herein is elevated compared to a control T cell response, or (ii) not having or not at risk of having Celiac disease if the T cell response to the peptide described herein is reduced compared to the control T cell response or the same as the control T cell response.

In some embodiments, the composition comprises the first, second, third, fourth, fifth, sixth, seventh, eighth, ninth, tenth, eleventh, twelfth, and thirteenth peptides described herein. In some embodiments, the composition comprises the second, fourth, fifth, sixth, seventh, eighth, ninth, tenth, eleventh, twelfth, thirteenth, fourteenth, fifteenth, and sixteenth peptides described herein. In some embodiments, the composition comprises the first, second, third, fourth, fifth, sixth, eighth, ninth, tenth, eleventh, twelfth, and thirteenth peptides described herein. In some embodiments, the composition comprises the second, fourth, fifth, sixth, eighth, ninth, tenth, eleventh, twelfth, thirteenth, fourteenth, fifteenth, and sixteenth peptides described herein. In some embodiments, the composition comprises the second, third, fourth, fifth, sixth, seventh, eighth, ninth, tenth, eleventh, twelfth, thirteenth, fifteenth, sixteenth, and seventeenth peptides described herein. In some embodiments, the composition comprises the second, third, fourth, fifth, sixth, eighth, ninth, tenth, eleventh, twelfth, thirteenth, fifteenth, sixteenth, and seventeenth peptides described herein. In some embodiments, the composition comprises the first, second, third, fourth, fifth, sixth, tenth, eleventh, twelfth, thirteenth, fifteenth, seventeenth, and eighteenth peptides described herein.

T cells responses and methods of measuring T cell responses are described herein. In some embodiments, the step of determining comprises contacting the sample with a composition comprising at least one (e.g., at least four) peptides as described herein and measuring a T cell response to the composition. Without wishing to be bound by theory, it is believed that the peptides described herein serve as an active component causing the activation and/or mobilization of CD4+ T cells in a subject who has Celiac disease. Thus, in some embodiments, the T cell or T cell response referred to in any one of the methods provided is a CD4+ T cell or CD4+ T cell response. In some embodiments, the subject has or is at risk of having Celiac disease.

In some embodiments, any one of the methods described herein further comprises performing a challenge as described herein.

In some embodiments, any one of the methods described herein further comprises performing other testing, particularly if the subject is identified as having or at risk of having Celiac disease. Other testing is described herein.

In some embodiments, any one of the methods described herein comprises a step of providing a treatment to a subject identified as having or being at risk of having Celiac disease. In some embodiments, any one of the methods described herein comprises a step of providing information to the subject about a treatment. In some embodiments, any one of the methods described herein comprises a step of recommending a gluten free diet, or providing information about such a diet, if the subject is identified as having or at risk of having Celiac disease. Information can be given orally or in written form, such as with written materials. Written materials may be in an electronic form. In some embodiments, treatment comprises administration of any one of the compositions as described herein, such as a composition comprising at least one (e.g., at least four) peptides described herein.

In some embodiments, any one of the methods described herein further comprises recording whether or not the subject has celiac disease based on the assessing. In some embodiments, any one of the methods described herein further comprises transmitting, such as to a database, whether or not the subject has celiac disease based on the assessing. The transmitting may be accomplished, e.g., via a computer or network of computers.

### T Cell Responses and Measurement Thereof

Aspects of the disclosure relate to a determination or measurement of a T cell response in a sample comprising T cells from a subject. In some embodiments, a composition comprising wheat, rye, and/or barley, or one or more of the peptides described herein (e.g., as a challenge described herein), is administered to a subject and, preferably, is capable of activating a CD4+ T cell in a subject, e.g., a subject with Celiac disease. The term “activate” or “activating” or “activation” in relation to a CD4+ T cell refers to the presentation by an MHC molecule of an epitope on one cell to an appropriate T cell receptor on a second CD4+ T cell, together with binding of a co-stimulatory molecule by the CD4+ T cell, thereby eliciting a “T cell response”, in this example a CD4+ T cell response. Such a T cell response can be measured ex vivo, e.g., by measuring a T cell response in a sample comprising T cells from the subject.

As described herein, an elevated T cell response, such as an elevated CD4+ T cell response, from a sample comprising T cells from a subject, e.g., after administration of a composition comprising wheat, rye, and/or barley or one or more of the peptides described herein, compared to a control T cell response can correlate with the presence or absence of Celiac disease in the subject. Accordingly, aspects of the disclosure relate to methods that comprise determining or measuring a T cell response in a sample comprising T cells from a subject, e.g., having or suspected of having Celiac disease.

In some embodiments, measuring a T cell response in a sample comprising T cells from a subject comprises contacting the sample with a composition comprising at least one (e.g., at least four) gluten peptides as described herein. For example, whole blood or PBMCs obtained from a subject who has been exposed to gluten (e.g., by a challenge as described herein or by administration of one or more peptides described herein) may be contacted with the composition comprising the peptides in order to stimulate T cells in the whole blood sample or PBMCs.

Measuring a T cell response can be accomplished using any assay known in the art (see, e.g., Molecular Cloning: A Laboratory Manual, M. Green and J. Sambrook, Fourth Edition, Cold Spring Harbor Laboratory Press, Cold Spring Harbor, N.Y., 2012; Current Protocols in Molecular Biology, F. M. Ausubel, et al., Current Edition, John Wiley & Sons, Inc., New York). In some embodiments, measuring a T cell response comprises an MHC Class II tetramer assay, such as flow cytometry with MHC Class II tetramer staining (see, e.g., Raki M, Fallang L E, Brottveit M, Bergseng E, Quarsten H, Lundin K E, Sollid L M: Tetramer visualization of gut-homing gluten-specific T cells in the peripheral blood of Celiac disease patients. Proceedings of the National Academy of Sciences of the United States of America 2007; Anderson R P, van Heel D A, Tye-Din J A, Barnardo M, Salio M, Jewell D P, Hill A V: T cells in peripheral blood after gluten challenge in coeliac disease. Gut 2005, 54(9):1217-1223; Brottveit M, Raki M, Bergseng E, Fallang L E, Simonsen B, Lovik A, Larsen S, Loberg E M, Jahnsen F L, Sollid L M et al: Assessing possible Celiac disease by an HLA-DQ2-gliadin Tetramer Test. The American journal of gastroenterology 2011, 106(7):1318-1324; and Anderson R P, Degano P, Godkin A J, Jewell D P, Hill A V: In vivo antigen challenge in Celiac disease identifies a single transglutaminase-modified peptide as the dominant A-gliadin T cell epitope. Nature Medicine 2000, 6(3):337-342).

In some embodiments, measuring a T cell response in a sample comprising T cells from a subject comprises measuring a level of at least one cytokine in the sample. In some embodiments, measuring a T cell response in a sample comprising T cells from a subject comprises contacting the sample with any one of the compositions provided, such as a composition comprising at least one (e.g., at least four) gluten peptides as described herein, and measuring a level of at least one cytokine in the sample. In some embodiments, the at least one cytokine is at least one pro-inflammatory cytokine such as IL-2, IFN-γ, IL-4, IL-5, IP-10, IL-13, or IL-17, e.g., by monocytes or granulocytes, as a result of secretion of these cytokines. In some embodiments, the at least one cytokine is IFN-γ or IP-10. In some embodiments, the at least one cytokine is IP-10. In some embodiments, the at least one cytokine is IFN-γ.

Interferon-γ (IFN-γ, also called IFNG, IFG, and IFI) is a dimerized soluble cytokine of the type II class of interferons. IFN-γ typically binds to a heterodimeric receptor consisting of Interferon γ receptor 1 (IFNGR1) and Interferon γ receptor 2 (IFNGR2). IFN-γ can also bind to the glycosaminoglycan heparan sulfate (HS). IFN-γ is produced predominantly by natural killer (NK) and natural killer T (NKT) cells as part of the innate immune response, and by CD4 Th1 and CD8 cytotoxic T lymphocyte (CTL) effector T cells once antigen-specific immunity develops in a subject. In humans, the IFN-γ protein is encoded by the IFNG gene. The Genbank number for the human IFNG gene is 3458. Exemplary Genbank mRNA transcript IDs and protein IDs for IFN-γ are NM_000619.2 and NP_000610.2, respectively.

IFN-γ inducible protein-10 (IP-10, also referred to as C-X-C motif chemokine 10, CXCL10, small-inducible cytokine B10, SCYB10, C7, IFI10, crg-2, gIP-10, or mob-1) is a protein that in humans is encoded by the CXCL10 gene. IP-10 is a small cytokine belonging to the CXC chemokine family and binds to the chemokine receptor CXCR3. The Genbank ID number for the human CXCL10 gene is 3627. Exemplary Genbank mRNA transcript IDs and protein IDs for IP-10 are NM_001565.3 and NP_001556.2, respectively.

In some embodiments, measuring a T cell response comprises measuring a level of at least one cytokine. Levels of at least one cytokine include levels of cytokine RNA, e.g., mRNA, and/or levels of cytokine protein. In a preferred embodiment, levels of the at least one cytokine are protein levels.

Assays for detecting cytokine RNA include, but are not limited to, Northern blot analysis, RT-PCR, sequencing technology, RNA in situ hybridization (using e.g., DNA or RNA probes to hybridize RNA molecules present in the sample), in situ RT-PCR (e.g., as described in Nuovo G J, et al. Am J Surg Pathol. 1993, 17: 683-90; Komminoth P, et al. Pathol Res Pract. 1994, 190: 1017-25), and oligonucleotide microarray (e.g., by hybridization of polynucleotide sequences derived from a sample to oligonucleotides attached to a solid surface (e.g., a glass wafer with addressable location, such as Affymetrix microarray (Affymetrix®, Santa Clara, Calif.)). Designing nucleic acid binding partners, such as probes, is well known in the art. In some embodiments, the nucleic acid binding partners bind to a part of or an entire nucleic acid sequence of at least one cytokine, e.g., IFN-γ or IP-10, the sequence(s) being identifiable using the Genbank IDs described herein.

Assays for detecting protein levels include, but are not limited to, immunoassays (also referred to herein as immune-based or immuno-based assays, e.g., Western blot, ELISA, and ELISpot assays), Mass spectrometry, and multiplex bead-based assays. Binding partners for protein detection can be designed using methods known in the art and as described herein. In some embodiments, the protein binding partners, e.g., antibodies, bind to a part of or an entire amino acid sequence of at least one cytokine, e.g., IFN-γ or IP-10, the sequence(s) being identifiable using the Genbank IDs described herein. Other examples of protein detection and quantitation methods include multiplexed immunoassays as described for example in U.S. Pat. Nos. 6,939,720 and 8,148,171, and published U.S. Patent Application No. 2008/0255766, and protein microarrays as described for example in published U.S. Patent Application No. 2009/0088329.

Any suitable binding partner is contemplated herein. In some embodiments, the binding partner is any molecule that binds specifically to a cytokine as provided herein. A molecule is said to exhibit “specific binding” if it reacts or associates more frequently, more rapidly, with greater duration and/or with greater affinity with a particular target than it does with alternative targets. As described herein, “binds specifically”, when referring to a protein, means that the molecule is more likely to bind to a portion of or the entirety of a protein to be measured than to a portion of or the entirety of another protein. In some embodiments, the binding partner is an antibody or antigen-binding fragment thereof, such as Fab, F(ab)2, Fv, single chain antibodies, Fab and sFab fragments, F(ab′)2, Fd fragments, scFv, or dAb fragments. Methods for producing antibodies and antigen-binding fragments thereof are well known in the art (see, e.g., Sambrook et al, “Molecular Cloning: A Laboratory Manual” (2nd Ed.), Cold Spring Harbor Laboratory Press (1989); Lewin, “Genes IV”, Oxford University Press, New York, (1990), and Roitt et al., “Immunology” (2nd Ed.), Gower Medical Publishing, London, New York (1989), WO2006/040153, WO2006/122786, and WO2003/002609). Binding partners also include other peptide molecules and aptamers that bind specifically. Methods for producing peptide molecules and aptamers are well known in the art (see, e.g., published US Patent Application No. 2009/0075834, U.S. Pat. Nos. 7,435,542, 7,807,351, and 7,239,742). In some embodiments, the binding partner is any molecule that binds specifically to an mRNA (e.g., IFN-γ or IP-10 mRNA). As described herein, “binds specifically to an mRNA” means that the molecule is more likely to bind to a portion of or the entirety of the mRNA to be measured (e.g., by complementary base-pairing) than to a portion of or the entirety of another mRNA or other nucleic acid. In some embodiments, the binding partner that binds specifically to an mRNA is a nucleic acid, e.g., a probe.

In some embodiments, measuring a level of at least one cytokine comprises a multiplex bead-based assay. An exemplary multiplex bead-based assay involves use of magnetic beads that are internally dyed with fluorescent dyes to produce a specific spectral address. Binding partners (e.g., antibodies) are conjugated to the surface of beads to capture the at least one cytokine. The sample is loaded into a 96-well plate containing the beads and the sample is incubated to allow binding of the at least one cytokine to the beads. A second biotinylated binding partner for the at least one cytokine is added after the at least one cytokine binds to the beads. A streptavidin-conjugated detectable label is then bound to the biotin. Light emitting diodes are used to illuminate the samples, causing the fluorescent dyes in the beads to fluoresce, as well as the detectable label to fluoresce. The concentration of the at least one cytokine is then determined based on the level of fluorescence. An exemplary system for running a multiplex bead-based assay is the MAGPIX® system available from Luminex® Corporation (see, e.g., U.S. Pat. No. 8,031,918, U.S. Pat. No. 8,296,088, U.S. Pat. No. 8,274,656, U.S. Pat. No. 8,532,351, U.S. Pat. No. 8,542,897, U.S. Pat. No. 6,514,295, U.S. Pat. No. 6,599,331, U.S. Pat. No. 6,632,526, U.S. Pat. No. 6,929,859, U.S. Pat. No. 7,445,844, U.S. Pat. No. 7,718,262, U.S. Pat. No. 8,283,037, and U.S. Pat. No. 8,568,881, all of which are incorporated by reference herein, and in particular the systems provided herein).

In some embodiments, measuring a level of at least one cytokine comprises an enzyme-linked immunosorbent assay (ELISA) or enzyme-linked immunosorbent spot (ELISpot) assay. ELISA and ELISpot assays are well known in the art (see, e.g., U.S. Pat. Nos. 5,939,281, 6,410,252, and 7,575,870; Czerkinsky C, Nilsson L, Nygren H, Ouchterlony O, Tarkowski A (1983) “A solid-phase enzyme-linked immunospot (ELISPOT) assay for enumeration of specific antibody-secreting cells”. J Immunol Methods 65 (1-2): 109-121 and Lequin R (2005). “Enzyme immunoassay (EIA)/enzyme-linked immunosorbent assay (ELISA)”. Clin. Chem. 51(12): 2415-8).

An exemplary ELISA involves at least one binding partner, e.g., an antibody or antigen-binding fragment thereof, with specificity for the at least one cytokine, e.g., IFN-γ or IP-10. The sample with an unknown amount of the at least one cytokine can be immobilized on a solid support (e.g., a polystyrene microtiter plate) either non-specifically (via adsorption to the surface) or specifically (via capture by another binding partner specific to the same at least one cytokine, as in a “sandwich” ELISA). After the antigen is immobilized, the binding partner for the at least one cytokine is added, forming a complex with the immobilized at least one cytokine. The binding partner can be attached to a detectable label as described herein (e.g., a fluorophor or an enzyme), or can itself be detected by an agent that recognizes the at least one cytokine binding partner that is attached to a detectable label as described herein (e.g., a fluorophor or an enzyme). If the detectable label is an enzyme, a substrate for the enzyme is added, and the enzyme elicits a chromogenic or fluorescent signal by acting on the substrate. The detectable label can then be detected using an appropriate machine, e.g., a fluorimeter or spectrophotometer, or by eye.

An exemplary ELISpot assay involves a binding agent for the at least one cytokine (e.g., an anti-IFN-γ antibody) that is coated aseptically onto a PVDF (polyvinylidene fluoride)-backed microplate. Cells of interest (e.g., peripheral blood mononuclear cells) are plated out at varying densities, along with one or more peptides as described herein, and allowed to incubate for a period of time (e.g., about 24 hours). The at least one cytokine secreted by activated cells is captured locally by the binding partner for the at least one cytokine on the high surface area PVDF membrane. After the at least one cytokine is immobilized, a second binding partner for the at least one cytokine is added, forming a complex with the immobilized at least one cytokine. The binding partner can be linked to a detectable label (e.g., a fluorophor or an enzyme), or can itself be detected by an agent that recognizes the binding partner for the at least one cytokine (e.g., a secondary antibody) that is linked to a detectable label (e.g., a fluorophor or an enzyme). If the detectable label is an enzyme, a substrate for the enzyme is added, and the enzyme elicits a chromogenic or fluorescent signal by acting on the substrate. The detectable label can then be detected using an appropriate machine, e.g., a fluorimeter or spectrophotometer, or by eye.

In some embodiments, a level of at least one cytokine is measured using an ELISA. As an exemplary method, a composition comprising at least one (e.g., at least four) gluten peptides as described herein is dried onto the inner wall of a blood collection tube. A negative control tube containing no antigen is provided. A positive control tube containing a mitogen is also provided. Blood from a subject is drawn into each of the three tubes. Each tube is agitated to ensure mixing. The tubes are then incubated at 37 degrees Celsius, preferably immediately after blood draw or at least within about 16 hours of collection. After incubation, the cells are separated from the plasma by centrifugation. The plasma is then loaded into an ELISA plate for detection of levels of at least one cytokine (e.g., IFN-γ) present in the plasma. A standard ELISA assay as described above can then be used to detect the levels of the at least one cytokine present in each plasma sample. In some embodiments, a T cell response measurement in a sample obtained from the subject after a challenge as described herein is detected using any one of the methods above or any other appropriate method and is then compared to a control T cell response, e.g., a T cell response measurement in a sample obtained before challenge or a T cell response measurement in a sample from a control subject or subjects. Exemplary control T cell responses include, but are not limited to, a T cell response in a sample obtained from a diseased subject(s) (e.g., subject(s) with Celiac disease), a healthy subject(s) (e.g., subject(s) without Celiac disease) or a T cell response in a sample obtained from a subject before or during a challenge as described herein. In some embodiments, a control T cell response is measured using any one of the methods above or any other appropriate methods. In some embodiments, the same method is used to measure a T cell response in the sample of the subject and the control sample.

In some embodiments, a T cell response is compared to a control T cell response. In some embodiments, if the control T cell response is a T cell response in a sample from a healthy control subject or subjects, then an elevated T cell response compared to the control T cell response is indicative that the subject has or is at risk of having Celiac disease while a reduced or equal T cell response compared to the control T cell response is indicative that the subject does not have or is not at risk of having Celiac disease. In some embodiments, if the control T cell response is a T cell response in a sample from the subject before a challenge as described herein, then an elevated T cell response compared to the control T cell response is indicative that the subject has or is at risk of having Celiac disease while a reduced or equal T cell response compared to the control T cell response is indicative that the subject does not have or is not at risk of having Celiac disease.

An elevated T cell response includes a response that is, for example, 1%, 5%, 10%, 20%, 30%, 40%, 50%, 60%, 70%, 80%, 90%, 100%, 150%, 200%, 300%, 400%, 500% or more above a control T cell response. A reduced T cell response includes a response that is, for example, 1%, 5%, 10%, 20%, 30%, 40%, 50%, 60%, 70%, 80%, 90%, 100%, 150%, 200%, 300%, 400%, 500% or more below a control T cell response.

In some embodiments, a second control T cell response is contemplated. In some embodiments, the second control T cell response is a negative control T cell response. Exemplary negative controls include, but are not limited to, a T cell response in a sample that has been contacted with a non-T cell-activating peptide (e.g., a peptide not recognized by T cells present in a sample from a subject), such as a non-CD4⁺-T cell-activating peptide, or a T cell response in a sample that has not been contacted with a T cell-activating peptide (e.g., contacting the sample with a saline solution containing no peptides), such as a CD4+ T cell-activating peptide. Such a second control T cell response can be measured using any one of the methods above or any other appropriate methods. In some embodiments, the second control T cell response is a positive control T cell response. Exemplary positive controls include, but are not limited to, a T cell response in a sample that has been contacted with a mitogen (e.g., phytohaemagglutinin, concanavalin A, lipopolysaccharide, or pokeweed mitogen). Positive and/or negative controls may be used to determine that an assay, such as an ELISA or ELISpot assay, is not defective or contaminated.

### Challenge

In some embodiments, any one of the methods provided herein comprise a challenge or a sample obtained from a subject before, during, or after a challenge. Generally, a challenge comprises administering to the subject a composition comprising wheat, rye, or barley, or a peptide thereof (e.g., a composition comprising a wheat gliadin, a rye secalin, or a barley hordein, or one or more peptides thereof), in some form for a defined period of time in order to activate the immune system of the subject, e.g., through activation of wheat-, rye- and/or barley-reactive T cells and/or mobilization of such T cells in the subject. Methods of challenges, e.g., gluten challenges, are well known in the art and include oral, submucosal, supramucosal, and rectal administration of peptides or proteins (see, e.g., Can J Gastroenterol. 2001. 15(4):243-7. In vivo gluten challenge in celiac disease. Ellis H J, Ciclitira P J; Mol Diagn Ther. 2008. 12(5):289-98. Celiac disease: risk assessment, diagnosis, and monitoring. Setty M, Hormaza L, Guandalini S; Gastroenterology. 2009; 137(6):1912-33. Celiac disease: from pathogenesis to novel therapies. Schuppan D, Junker Y, Barisani D; J Dent Res. 2008; 87(12):1100-1107. Orally based diagnosis of celiac disease: current perspectives. Pastore L, Campisi G, Compilato D, and Lo Muzio L; Gastroenterology. 2001; 120:636-651. Current Approaches to Diagnosis and Treatment of Celiac Disease: An Evolving Spectrum. Fasano A and Catassi C; Clin Exp Immunol. 2000; 120:38-45. Local challenge of oral mucosa with gliadin in patients with coeliac disease. Lahteenoja M, Maki M, Viander M, Toivanen A, Syrjanen S; Clin Exp Immunol. 2000; 120:10-11. The mouth—an accessible region for gluten challenge. Ellis H and Ciclitira P; Clinical Science. 2001; 101:199-207. Diagnosing coeliac disease by rectal gluten challenge: a prospective study based on immunopathology, computerized image analysis and logistic regression analysis. Ensari A, Marsh M, Morgan S, Lobley R, Unsworth D, Kounali D, Crowe P, Paisley J, Moriarty K, and Lowry J; Gut. 2005; 54:1217-1223. T cells in peripheral blood after gluten challenge in coeliac disease. Anderson R, van Heel D, Tye-Din J, Barnardo M, Salio M, Jewell D, and Hill A; and Nature Medicine. 2000; 6(3):337-342. In vivo antigen challenge in celiac disease identifies a single transglutaminase-modified peptide as the dominant A-gliadin T-cell epitope. Anderson R, Degano P, Godkin A, Jewell D, and Hill A). Traditionally, a challenge lasts for several weeks (e.g., 4 weeks or more) and involves high doses of orally administered peptides or proteins (usually in the form of baked foodstuff that includes the peptides or proteins). Some studies suggest that a shorter challenge, e.g., through use of as little as 3 days of oral challenge, is sufficient to activate and/or mobilize reactive T-cells (Anderson R, van Heel D, Tye-Din J, Barnardo M, Salio M, Jewell D, and Hill A; and Nature Medicine. 2000; 6(3):337-342. In vivo antigen challenge in celiac disease identifies a single transglutaminase-modified peptide as the dominant A-gliadin T-cell epitope. Anderson R, Degano P, Godkin A, Jewell D, and Hill A). Any such methods of challenge that are capable of activating the immune system of the subject, e.g., by activating wheat-, rye- or barley-reactive T-cells and/or mobilizing such T cells into blood are contemplated herein.

In some embodiments, the challenge comprises administering a composition comprising wheat, barley and/or rye, or one or more peptides thereof. In some embodiments, the wheat is wheat flour, the barely is barley flour, and the rye is rye flour. In some embodiments, the challenge comprises administering a composition comprising a wheat gliadin, a barley hordein and/or a rye secalin, or one or more peptides thereof, to the subject prior to determining a T cell response as described herein.

In some embodiments, the composition is administered to the subject more than once prior to determining the T cell response, and a sample is obtained from the subject after administration of the composition. In some embodiments, administration is daily for 3 days. In some embodiments, the sample is obtained from the subject 6 days after administration of the composition. In some embodiments, the subject has been on a gluten-free diet for at least 4 weeks prior to commencing the challenge.

In some embodiments, administration is oral. Suitable forms of oral administration include foodstuffs (e.g., baked goods such as breads, cookies, cakes, etc.), tablets, troches, lozenges, aqueous or oily suspensions, dispersible powders or granules, emulsions, hard or soft capsules, or syrups or elixirs. Compositions intended for oral use may be prepared according to methods known to the art for the manufacture of pharmaceutical compositions or foodstuffs and such compositions may contain one or more agents including, for example, sweetening agents, flavoring agents, coloring agents and preserving agents in order to provide pharmaceutically elegant and palatable preparations.

In some embodiments, a sample is obtained from a subject before, during, and/or after a challenge as described herein. In some embodiments, the sample is a sample comprising a T cell, e.g., a whole blood sample or PBMCs. In some embodiments, the sample is contacted with a composition comprising at least one (e.g., at least four) gluten peptides as described herein. In some embodiments, a T cell response in the sample is measured as described herein.

### Treatment and Compositions Comprising Pharmaceutically Acceptable Carrier

Other aspects of the disclosure relate to treatment of subjects having or at risk of having Celiac disease. In some embodiments, the subject to be treated is one identified as having or at risk of having Celiac disease by any one of the methods described herein, e.g., by evaluating a T cell response. In some embodiments, the method comprises a step where information regarding treatment is provided to the subject. Such information can be given orally or in written form, such as with written materials. Written materials may be in an electronic form.

In some embodiments, a method of treatment comprises administering an effective amount of any one of the compositions provided, such as a composition comprising at least one (e.g., at least four) gluten peptides as described herein to a subject having or at risk of having Celiac disease. In some embodiments, the composition is a composition described in the Examples provided. Modifications to such peptides, e.g., an N-terminal pyro-glutamate and/or C-terminal amide, are contemplated and described herein.

As used herein, the terms “treat”, “treating”, and “treatment” include abrogating, inhibiting, slowing, or reversing the progression of a disease, or ameliorating or preventing a clinical symptom of the disease (for example, Celiac disease). Treatment may include induction of immune tolerance (for example, to gluten or peptides thereof), modification of the cytokine secretion profile of the subject and/or induction of suppressor T cell subpopulations to secrete cytokines. Thus, in some embodiments, a subject treated according to the disclosure preferably is able to eat at least wheat, rye, and barley without a significant T cell response which would normally lead to symptoms of Celiac disease. In some embodiments, an effective amount of a treatment is administered. The term “effective amount” means the amount of a treatment sufficient to provide the desired therapeutic or physiological effect when administered under appropriate or sufficient conditions.

Treatments may be administrated using any method known in the art. Pharmaceutical compositions suitable for each administration route are well known in the art (see, e.g., Remington: The Science and Practice of Pharmacy, 21st Ed. Lippincott Williams & Wilkins, 2005). In some embodiments, a treatment, e.g., a composition described herein, is administered via intradermal injection.

The peptides may be in a salt form, preferably, a pharmaceutically acceptable salt form. “A pharmaceutically acceptable salt form” includes the conventional non-toxic salts or quaternary ammonium salts of a peptide, for example, from non-toxic organic or inorganic acids. Conventional non-toxic salts include, for example, those derived from inorganic acids such as hydrochloride, hydrobromic, sulphuric, sulfonic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

Compositions may include a pharmaceutically acceptable carrier. Pharmaceutical carriers are described herein.

The composition may be in the form of a sterile injectable aqueous or oleagenous suspension. In some embodiments, the composition is formulated as a sterile, injectable solution. This suspension or solution may be formulated according to known methods using those suitable dispersing or wetting agents and suspending agents which have been mentioned above. The sterile injectable preparation may be a suspension in a non-toxic parenterally-acceptable diluent or solvent, for example as a solution in 1,3-butanediol. Among the acceptable carriers that may be employed are water, Ringer's solution and isotonic sodium chloride solution. In some embodiments, the composition is formulated as a sterile, injectable solution, wherein the solution is a sodium chloride solution (e.g., sodium chloride 0.9% USP). In some embodiments, the composition is formulated as a bolus for intradermal injection. Examples of appropriate delivery mechanisms for intradermal administration include, but are not limited to, implants, depots, syringes, needles, capsules, and osmotic pumps.

It is especially advantageous to formulate the active agent in a dosage unit form for ease of administration and uniformity of dosage. “Dosage unit form” as used herein refers to physically discrete units suited as unitary dosages for the subject to be treated; each unit containing a predetermined quantity of active agent calculated to produce the desired therapeutic effect in association with the required pharmaceutical carrier. The specification for the dosage unit forms are dictated by and directly dependent on the unique characteristics of the active agent and the particular therapeutic effect to be achieved, and the limitations inherent in the art of compounding such an active agent for the treatment of subjects. Alternatively, the compositions may be presented in multi-dose form. Examples of dosage units include sealed ampoules and vials and may be stored in a freeze-dried condition requiring only the addition of the sterile liquid carrier immediately prior to use.

The actual amount administered (or dose or dosage) and the rate and time-course of administration will depend on the nature and severity of the condition being treated as well as the characteristics of the subject to be treated (weight, age, etc.). Prescription of treatment, for example, decisions on dosage, timing, frequency, etc., is within the responsibility of general practitioners or specialists (including human medical practitioner, veterinarian or medical scientist) and typically takes account of the disorder to be treated, the condition of the subject, the site of delivery, the method of administration and other factors known to practitioners. Examples of techniques and protocols can be found in, e.g., Remington: The Science and Practice of Pharmacy, 21st Ed. Lippincott Williams & Wilkins, 2005. Effective amounts may be measured from ng/kg body weight to g/kg body weight per minute, hour, day, week or month. Dosage amounts may vary from, e.g., 10 ng/kg to up to 100 mg/kg of mammal body weight or more per day, preferably about 1 μg/kg/day to 10 mg/kg/day, depending upon the route of administration.

Toxicity and therapeutic efficacy of the agent can be determined by standard pharmaceutical procedures in cell cultures or experimental animals by determining the IC50 and the maximal tolerated dose. The data obtained from these cell culture assays and animal studies can be used to formulate a range suitable for humans.

### Other Testing

In some embodiments of any one of the methods provided, methods described herein further comprise other testing of a subject (e.g., based on the results of the methods described herein). As used herein, “other testing” describes use of at least one additional diagnostic method in addition to the methods provided herein. Any diagnostic method or combinations thereof for Celiac disease is contemplated as other testing. Exemplary other testing includes, but is not limited to, intestinal biopsy, serology (measuring the levels of one or more antibodies present in the serum), genotyping (see, e.g., Walker-Smith J A, et al. Arch Dis Child 1990), and measurement of a T cell response. Such other testing may be performed as part of the methods described herein or after the methods described herein (e.g., as a companion diagnostic), or before use of the methods described herein (e.g., as a first-pass screen to eliminate certain subjects before use of the methods described herein, e.g., eliminating those that do not have one or more HLA-DQA and HLA-DQB susceptibility alleles).

Detection of serum antibodies (serology) is contemplated. The presence of such serum antibodies can be detected using methods known to those of skill in the art, e.g., by ELISA, histology, cytology, immunofluorescence or western blotting. Such antibodies include, but are not limited to: IgA anti-endomysial antibody (IgA EMA), IgA anti-tissue transglutaminase antibody (IgA tTG), IgA anti-deamidated gliadin peptide antibody (IgA DGP), and IgG anti-deamidated gliadin peptide antibody (IgG DGP).

IgA EMA: IgA endomysial antibodies bind to endomysium, the connective tissue around smooth muscle, producing a characteristic staining pattern that is visualized by indirect immunofluorescence. The target antigen has been identified as tissue transglutaminase (tTG or transglutaminase 2). IgA endomysial antibody testing is thought to be moderately sensitive and highly specific for untreated (active) Celiac disease.

IgA tTG: The antigen is tTG. Anti-tTG antibodies are thought to be highly sensitive and specific for the diagnosis of Celiac disease. Enzyme-linked immunosorbent assay (ELISA) tests for IgA anti-tTG antibodies are now widely available and are easier to perform, less observer-dependent, and less costly than the immunofluorescence assay used to detect IgA endomysial antibodies. The diagnostic accuracy of IgA anti-tTG immunoassays has been improved further by the use of human tTG in place of the nonhuman tTG preparations used in earlier immunoassay kits. Kits for IgA tTG are commercially available (INV 708760, 704525, and 704520, INOVA Diagnostics, San Diego, Calif.).

Deamidated gliadin peptide-IgA (DGP-IgA) and deamidated gliadin peptide-IgG (DGP-IgG) are also contemplated herein and can be evaluated with commercial kits (INV 708760, 704525, and 704520, INOVA Diagnostics, San Diego, Calif.).

Genetic testing (genotyping) is also contemplated. Subjects can be tested for the presence of the HLA-DQA and HLA-DQB susceptibility alleles encoding HLA-DQ2.5 (DQA1*05 and DQB1*02), DQ2.2 (DQA1*02 and DQB1*02) or DQ8 (DQA1*03 and DQB1*0302). Exemplary sequences that encode the DQA and DQB susceptibility alleles include HLA-DQA1*0501 (Genbank accession number: AF515813.1) HLA-DQA1*0505 (AH013295.2), HLA-DQB1*0201 (AY375842.1) or HLA-DQB1*0202 (AY375844.1). Methods of genetic testing are well known in the art (see, e.g., Bunce M, et al. Phototyping: comprehensive DNA typing for HLA-A, B, C, DRB1, DRB3, DRB4, DRB5 & DQB1 by PCR with 144 primer mixes utilizing sequence-specific primers (PCR-SSP). Tissue Antigens 46, 355-367 (1995); Olerup O, Aldener A, Fogdell A. HLA-DQB1 and DQA1 typing by PCR amplification with sequence-specific primers in 2 hours. Tissue antigens 41, 119-134 (1993); Mullighan C G, Bunce M, Welsh K I. High-resolution HLA-DQB1 typing using the polymerase chain reaction and sequence-specific primers. Tissue-Antigens. 50, 688-92 (1997); Koskinen L, Romanos J, Kaukinen K, Mustalahti K, Korponay-Szabo I, et al. (2009) Cost-effective HLA typing with tagging SNPs predicts celiac disease risk haplotypes in the Finnish, Hungarian, and Italian populations. Immunogenetics 61: 247-256; and Monsuur A J, de Bakker P I, Zhernakova A, Pinto D, Verduijn W, et al. (2008) Effective detection of human leukocyte antigen risk alleles in celiac disease using tag single nucleotide polymorphisms. PLoS ONE 3: e2270). Subjects that have one or more copies of a susceptibility allele are considered to be positive for that allele. Detection of the presence of susceptibility alleles can be accomplished by any nucleic acid assay known in the art, e.g., by polymerase chain reaction (PCR) amplification of DNA extracted from the patient followed by hybridization with sequence-specific oligonucleotide probes or using leukocyte-derived DNA (Koskinen L, Romanos J, Kaukinen K, Mustalahti K, Korponay-Szabo I, Barisani D, Bardella M T, Ziberna F, Vatta S, Szeles G et al: Cost-effective HLA typing with tagging SNPs predicts Celiac disease risk haplotypes in the Finnish, Hungarian, and Italian populations. Immunogenetics 2009, 61(4):247-256; Monsuur A J, de Bakker P I, Zhernakova A, Pinto D, Verduijn W, Romanos J, Auricchio R, Lopez A, van Heel D A, Crusius J B et al: Effective detection of human leukocyte antigen risk alleles in Celiac disease using tag single nucleotide polymorphisms. PLoS ONE 2008, 3(5):e2270).

T cell response tests are also contemplated as other testing. In some embodiments, a T cell response test comprises contacting a sample comprising a T cell with a gluten peptide and measuring a T cell response in the sample. In some embodiments, a T cell response is measured by measuring a level of IFN-γ, where an increased level of IFN-γ compared to a control level (e.g., a level of IFN-γ in a sample that has not been contacted with a gluten peptide) may identify a subject as having Celiac disease. T cell response tests are known in the art (see, e.g., PCT Publication Nos.: WO/2001/025793, WO/2003/104273, WO/2005/105129, and WO/2010/060155).

### Subjects

A subject may include any subject that is suspected of having Celiac disease. Preferably, the subject is a human. In some embodiments, the subject has one or more HLA-DQA and HLA-DQB susceptibility alleles encoding HLA-DQ2.5 (DQA1*05 and DQB1*02), HLA-DQ2.2 (DQA1*02 and DQB1*02) or HLA-DQ8 (DQA1*03 and DQB1*0302). In some embodiments, the subject is HLA-DQ2.5 positive (i.e., has both susceptibility alleles DQA1*05 and DQB1*02). In some embodiments, the subject is HLA-DQ2.2 positive (i.e., has both susceptibility alleles DQA1*02 and DQB1*02). In some embodiments, the subject is HLA-DQ8 positive (i.e., has both susceptibility alleles DQA1*03 and DQB1*0302). In some embodiments, the subject is HLA-DQ2.2 positive and HLA-DQ2.5 positive. In some embodiments, the subject is HLA-DQ8 positive and HLA-DQ2.5 positive. In some embodiments, the subject is HLA-DQ2.2 positive and HLA-DQ8 positive. In some embodiments, a subject may have a family member that has one or more HLA-DQA and HLA-DQB susceptibility alleles encoding HLA-DQ2.5 (DQA1*05 and DQB1*02), HLA-DQ2.2 (DQA1*02 and DQB1*02) or HLA-DQ8 (DQA1*03 and DQB1*0302). The presence of susceptibility alleles can be detected by any nucleic acid detection method known in the art, e.g., by polymerase chain reaction (PCR) amplification of DNA extracted from the patient followed by hybridization with sequence-specific oligonucleotide probes. In some embodiments of any one of the methods provided herein, the subject is on a gluten-free diet.

### Samples

Samples, as used herein, refer to biological samples taken or derived from a subject, e.g., a subject having or suspected of having Celiac disease. Examples of samples include tissue samples or fluid samples. Examples of fluid samples are whole blood, plasma, serum, and other bodily fluids that comprise T cells. In some embodiments, the sample comprises T cells. In some embodiments, the sample comprises T cells and monocytes and/or granulocytes. In some embodiments, the sample comprising T cells comprises whole blood or peripheral blood mononuclear cells (PBMCs). The T cell may be a CD4+ T cell, e.g., a gluten-reactive CD4+ T cell. In some embodiments, any one of the methods described herein comprise obtaining or providing the sample. In some embodiments, a first sample and second sample are contemplated. In some embodiments, the first sample is obtained from a subject before administration of any one of the compositions provided, such as a composition comprising at least one (e.g., at least four) peptides described herein or a challenge described herein. In some embodiments, the second sample is obtained from a subject after administration of the composition or after a challenge described herein. Additional samples, e.g., third, fourth, fifth, etc., are also contemplated if additional measurements of a T cell response are desired. Such additional samples may be obtained from the subject at any time, e.g., before or after administration of any one of the compositions provided, such as a composition comprising at least one (e.g., at least four) peptides described herein or a challenge described herein.

### Controls and Control Subjects

In some embodiments, any one of the methods provided herein comprise measuring or use of a control T cell response. In some embodiments, the control T cell response is a T cell response in a sample from the subject, e.g., before or during a challenge as described herein.

In some embodiments, the control T cell response is a T cell response in a sample obtained from a control subject (or subjects). In some embodiments, a control subject has one or more HLA-DQA and HLA-DQB susceptibility alleles encoding HLA-DQ2.5 (DQA1*05 and DQB1*02), DQ2.2 (DQA1*02 and DQB1*02) or DQ8 (DQA1*03 and DQB1*0302) described herein but does not have Celiac disease. In some embodiments, a control subject does not have any of the HLA-DQA and HLA-DQB susceptibility alleles encoding HLA-DQ2.5 (DQA1*05 and DQB1*02), DQ2.2 (DQA1*02 and DQB1*02) or DQ8 (DQA1*03 and DQB1*0302) described herein. In some embodiments, a control subject is a healthy individual not having or suspected of having Celiac disease. In some embodiments, control subjects are a population of subjects. In some embodiments, a control level is a pre-determined value from a control subject or subjects, such that the control level need not be measured every time the methods described herein are performed.

### Polynucleotides, Antigen Presenting Cells, and HLA Molecules

The one or more peptides may be encoded by one or more polynucleotides. Thus, at least some of the one or more peptides may be transcribed and translated, e.g., from a single polynucleotide as a single polypeptide chain.

The composition may also comprise a mixture of peptides and polynucleotides that encode the peptides.

The overall length of each constituent polynucleotide may be, for example, 21 to 150 nucleotides, such as, 21, 22, 23, 24, 25, 26, 27, 28, 29, 30, 31, 32, 33, 34, 35, 36, 37, 38, 39, 40, 41, 42, 43, 44, 45, 50, 60, 70, 80, 90, 100, 110, 120, 130, 140, or 150 nucleotides.

Analogues of the polynucleotides are also contemplated. Analogues include polynucleotides that vary by one or more nucleotides from a reference polynucleotide. For example, an analogue can comprise a substitution of one or more naturally occurring nucleotides with a nucleotide analogue (such as the morpholine ring), methylated nucleotide, internucleotide modifications such as uncharged linkages (for example, methyl phosphonates, phosphotriesters, phosphoamidates, carbamates, etc.), charged linkages (for example, phosphorothioates, phosphorodithioates, etc.), pendent moieties (for example, polypeptides), intercalators (for example, acridine, psoralen, etc.), chelators, alkylators and modified linkages (for example, α-anomeric nucleic acids, etc.). Polynucleotides encoding one or more of the peptides may be provided in a vector.

A polynucleotide encoding one or more of the peptides defined herein can be used for the recombinant production of the peptides using techniques well known in the art. Alternatively, the polynucleotide can be used to treat a subject having Celiac disease.

A polynucleotide of the disclosure includes a DNA sequence that can be derived from one or more of the peptides, bearing in mind the degeneracy of codon usage. This is well known in the art, as is knowledge of codon usage in different expression hosts, which is helpful in optimizing the recombinant expression of the peptides.

When the polynucleotide is used for the recombinant production of one or more of the peptides, the polynucleotide may include the coding sequence for the peptides alone or the coding sequence for the peptides in reading frame with other coding sequences, such as those encoding a leader or secretory sequence, a pre-, or pro- or prepro-protein sequence, linker peptide sequence, or other fusion peptide portions. For example, a marker sequence which facilitates purification of the fused peptide can be encoded. In certain embodiments, the marker sequence is a hexa-histidine peptide, as provided in the pQE vector (Qiagen, Inc.), or is an HA tag, or is glutathione-S-transferase. The polynucleotide may also contain non-coding 5′ and 3′ sequences, such as transcribed, non-translated sequences, splicing and polyadenylation signals, ribosome binding sites and sequences that stabilise mRNA.

Antigen presenting cells (APCs) are also contemplated herein. In some embodiments, an antigen presenting cell comprising a composition, peptide, or polynucleotide as described herein is contemplated. The composition, peptide, or polynucleotide defined herein may be delivered by loading APCs with, for example, at least one peptide described herein and/or a polynucleotide encoding one or more thereof.

In some embodiments, the APCs are selected from the group consisting of dendritic cells, macrophages, B-lymphocytes and liver sinusoidal endothelial cells that express MHC class II molecules shared with the MHC phenotype of the subject. For example, the APCs may express HLA-DQ2 (for example, HLA DQA1*05 and HLA DQB1*02) and/or HLA DQ8. The APCs employed for this purpose may be isolated from the subject to whom they are to be delivered after loading, or they may be obtained from an allo-matched subject.

By “loading” an APC it is meant that the APC is incubated or transfected with one or more peptides or a polynucleotide encoding one or more thereof. Loading an APC can be achieved by using conventional nucleic acid transfection methods, such as lipid-mediated transfection, electroporation, or calcium phosphate transfection.

In some embodiments, one or more peptides described herein are bound to a) an HLA molecule, or b) a fragment of an HLA molecule, capable of binding the peptide(s). In some embodiments, the HLA molecule is a heterodimer of an HLA-DQA protein encoded by HLA-DQA1*05, DQA1*02, or DQA1*03, and an HLA-DQB protein encoded by HLA-DQB1*02, or DQB1*0302. In some embodiments, the fragment of an HLA molecule is a fragment of a heterodimer of an HLA-DQA protein encoded by HLA-DQA1*05, DQA1*02, or DQA1*03, and an HLA-DQB protein encoded by HLA-DQB1*02, or DQB1*0302.

### Kits

Other aspects of this disclosure relate to kits. In some embodiments, the kit comprises any one of the compositions as described herein.

In some embodiments, the composition comprises at least one peptide selected from:

(a) a first peptide comprising the amino acid sequence PFPQPELPY (SEQ ID NO: 1) and the amino acid sequence PQPELPYPQ (SEQ ID NO: 2);

(b) a second peptide comprising the amino acid sequence PFPQPEQPF (SEQ ID NO: 3) and the amino acid sequence PQPEQPFPW (SEQ ID NO: 4);

(c) a third peptide comprising the amino acid sequence EQPIPEQPQ (SEQ ID NO: 5) and the amino acid sequence PIPEQPQPY (SEQ ID NO: 6);

(d) a fourth peptide comprising the amino acid sequence PFPQPEQPI (SEQ ID NO: 7), the amino acid sequence PQPEQPIPV (SEQ ID NO: 8), and the amino acid sequence EQPIPVQPE (SEQ ID NO: 9);

(e) a fifth peptide comprising the amino acid sequence PFPQPEQPT (SEQ ID NO: 10), the amino acid sequence PQPEQPTPI (SEQ ID NO: 11), and the amino acid sequence EQPTPIQPE (SEQ ID NO: 12);

(f) a sixth peptide comprising the amino acid sequence PFPQPEQPF (SEQ ID NO: 3), the amino acid sequence PQPEQPFPL (SEQ ID NO: 13), and the amino acid sequence EQPFPLQPE (SEQ ID NO: 14);

(g) a seventh peptide comprising the amino acid sequence PFPQPEQPF (SEQ ID NO: 3) and the amino acid sequence PQPEQPFSQ (SEQ ID NO: 15);

(h) an eighth peptide comprising the amino acid sequence PYPEQPQPF (SEQ ID NO: 16);

(i) a ninth peptide comprising the amino acid sequence PFPEQPEQI (SEQ ID NO: 21);

(j) a tenth peptide comprising the amino acid sequence EGSFQPSQE (SEQ ID NO: 17);

(k) an eleventh peptide comprising the amino acid sequence QGYYPTSPQ (SEQ ID NO: 18);

(l) a twelfth peptide comprising the amino acid sequence EQPEQPFPE (SEQ ID NO: 19) and the amino acid sequence EQPFPEQPQ (SEQ ID NO: 20);

(m) a thirteenth peptide comprising the amino acid sequence PFSEQEQPV (SEQ ID NO: 22);

(n) a fourteenth peptide comprising the amino acid sequence EQPFPEQPI (SEQ ID NO: 23), the amino acid sequence PFPEQPIPE (SEQ ID NO: 24), the amino acid sequence EQPIPEQPQ (SEQ ID NO: 5), and the amino acid sequence PIPEQPQPY (SEQ ID NO: 6);

(o) a fifteenth peptide comprising the amino acid sequence PQPELPYPQ (SEQ ID NO: 2) and the amino acid sequence PYPQPELPY (SEQ ID NO: 25);

(p) a sixteenth peptide comprising the amino acid sequence PFPQPELPY (SEQ ID NO: 1) and the amino acid sequence PQPELPYPY (SEQ ID NO: 26);

(q) a seventeenth peptide comprising the amino acid sequence EQPFPEQPI (SEQ ID NO: 23) and the amino acid sequence PFPEQPIPE (SEQ ID NO: 24); and

(r) an eighteenth peptide comprising the amino acid sequence PQPYPEQPQ (SEQ ID NO: 27) and the amino acid sequence PYPEQPQPF (SEQ ID NO: 16).

In some embodiments,

(a) the first peptide comprises the amino acid sequence LQPFPQPELPYPQPQ (SEQ ID NO: 28);

(b) the second peptide comprises the amino acid sequence QPFPQPEQPFPWQP (SEQ ID NO: 29);

(c) the third peptide comprises the amino acid sequence PEQPIPEQPQPYPQQ (SEQ ID NO: 30);

(d) the fourth peptide comprises the amino acid sequence QPFPQPEQPIPVQPEQS (SEQ ID NO: 31);

(e) the fifth peptide comprises the amino acid sequence QPFPQPEQPTPIQPEQP (SEQ ID NO: 32);

(f) the sixth peptide comprises the amino acid sequence QPFPQPEQPFPLQPEQP (SEQ ID NO: 33);

(g) the seventh peptide comprises the amino acid sequence QPFPQPEQPFSQQ (SEQ ID NO: 34);

(h) the eighth peptide comprises the amino acid sequence PQPYPEQPQPFPQQ (SEQ ID NO: 35);

(i) the ninth peptide comprises the amino acid sequence QPFPEQPEQIIPQQP (SEQ ID NO: 36);

(j) the tenth peptide comprises the amino acid sequence SGEGSFQPSQENPQ (SEQ ID NO: 37);

(k) the eleventh peptide comprises the amino acid sequence GQQGYYPTSPQQSG (SEQ ID NO: 38);

(l) the twelfth peptide comprises the amino acid sequence PEQPEQPFPEQPQQ (SEQ ID NO: 39);

(m) the thirteenth peptide comprises the amino acid sequence QPPFSEQEQPVLPQ (SEQ ID NO: 40);

(n) the fourteenth peptide comprises the amino acid sequence PEQPFPEQPIPEQPQPYP (SEQ ID NO: 41);

(o) the fifteenth peptide comprises the amino acid sequence QPYPQPELPYPQPQ (SEQ ID NO: 42);

(p) the sixteenth peptide comprises the amino acid sequence QPFPQPELPYPYPQ (SEQ ID NO: 43);

(q) the seventeenth peptide comprises the amino acid sequence PQEQPFPEQPIPEQP (SEQ ID NO: 44); and

(r) the eighteenth peptide comprises the amino acid sequence QPQPYPEQPQPFPQQ (SEQ ID NO: 45).

In some embodiments, the composition comprises at least one of:

(i) a first peptide comprising the amino acid sequence PFPQPELPY (SEQ ID NO: 1) and the amino acid sequence PQPELPYPQ (SEQ ID NO: 2);

(ii) a second peptide comprising the amino acid sequence PFPQPEQPF (SEQ ID NO: 3) and the amino acid sequence PQPEQPFPW (SEQ ID NO: 4);

(iii) a third peptide comprising the amino acid sequence PIPEQPQPY (SEQ ID NO: 6);

(iv) a fourth peptide comprising the amino acid sequence PFPQPEQPIP (SEQ ID NO: 62) and the amino acid sequence EQPIPVQPE (SEQ ID NO: 9);

(v) a fifth peptide comprising the amino acid sequence PFPQPEQPTPI (SEQ ID NO: 65) and the amino acid sequence EQPTPIQPE (SEQ ID NO: 12);

(vi) a sixth peptide comprising the amino acid sequence PQPEQPFPL (SEQ ID NO: 13) and the amino acid sequence EQPFPLQPE (SEQ ID NO: 14);

(vii) a seventh peptide comprising the amino acid sequence PFPQPEQPF (SEQ ID NO: 3) and the amino acid sequence PQPEQPFSQ (SEQ ID NO: 15);

(viii) an eighth peptide comprising the amino acid sequence PYPEQPQPF (SEQ ID NO: 16);

(ix) a ninth peptide comprising the amino acid sequence PFPEQPEQIIP (SEQ ID NO: 63);

(x) a tenth peptide comprising the amino acid sequence EGSFQPSQE (SEQ ID NO: 17);

(xi) an eleventh peptide comprising the amino acid sequence QGYYPTSPQ (SEQ ID NO: 18);

(xii) a twelfth peptide comprising the amino acid sequence EQPEQPFPEQPQ (SEQ ID NO: 64);

(xiii) a thirteenth peptide comprising the amino acid sequence PFSEQEQPV (SEQ ID NO: 22);

(xiv) a fourteenth peptide comprising the amino acid sequence EQPFPEQPI (SEQ ID NO: 23) and PIPEQPQPY (SEQ ID NO: 6);

(xv) a fifteenth peptide comprising the amino acid sequence PQPELPYPQ (SEQ ID NO: 2) and the amino acid sequence PYPQPELPY (SEQ ID NO: 25);

(xvi) a sixteenth peptide comprising the amino acid sequence PFPQPELPY (SEQ ID NO: 1) and the amino acid sequence PQPELPYPY (SEQ ID NO: 26); and

(xvii) a seventeenth peptide comprising the amino acid sequence EQPFPEQPI (SEQ ID NO: 23).

In some embodiments,

(i) the first peptide comprises the amino acid sequence LQPFPQPELPYPQPQ (SEQ ID NO: 28);

(ii) the second peptide comprises the amino acid sequence QPFPQPEQPFPWQP (SEQ ID NO: 29);

(iii) the third peptide comprises the amino acid sequence PEQPIPEQPQPYPQQ (SEQ ID NO: 30);

(iv) the fourth peptide comprises the amino acid sequence QPFPQPEQPIPVQPEQS (SEQ ID NO: 31);

(v) the fifth peptide comprises the amino acid sequence QPFPQPEQPTPIQPEQP (SEQ ID NO: 32);

(vi) the sixth peptide comprises the amino acid sequence QPFPQPEQPFPLQPEQP (SEQ ID NO: 33);

(vii) the seventh peptide comprises the amino acid sequence QPFPQPEQPFSQQ (SEQ ID NO: 34);

(viii) the eighth peptide comprises the amino acid sequence PQPYPEQPQPFPQQ (SEQ ID NO: 35);

(ix) the ninth peptide comprises the amino acid sequence QPFPEQPEQIIPQQP (SEQ ID NO: 36);

(x) the tenth peptide comprises the amino acid sequence SGEGSFQPSQENPQ (SEQ ID NO: 37);

(xi) the eleventh peptide comprises the amino acid sequence GQQGYYPTSPQQSG (SEQ ID NO: 38);

(xii) the twelfth peptide comprises the amino acid sequence PEQPEQPFPEQPQQ (SEQ ID NO: 39);

(xiii) the thirteenth peptide comprises the amino acid sequence QPPFSEQEQPVLPQ (SEQ ID NO: 40);

(xiv) the fourteenth peptide comprises the amino acid sequence PEQPFPEQPIPEQPQPYP (SEQ ID NO: 41);

(xv) the fifteenth peptide comprises the amino acid sequence QPYPQPELPYPQPQ (SEQ ID NO: 42);

(xvi) the sixteenth peptide comprises the amino acid sequence QPFPQPELPYPYPQ (SEQ ID NO: 43); and

(xvii) the seventeenth peptide comprises the amino acid sequence EQPFPEQPI (SEQ ID NO: 23).

In some embodiments, the composition comprises the first, second, third, fourth, fifth, sixth, seventh, eighth, ninth, tenth, eleventh, twelfth, and thirteenth peptides. In some embodiments, the composition comprises the second, fourth, fifth, sixth, seventh, eighth, ninth, tenth, eleventh, twelfth, thirteenth, fourteenth, fifteenth, and sixteenth peptides. In some embodiments, the composition comprises the first, second, third, fourth, fifth, sixth, eighth, ninth, tenth, eleventh, twelfth, and thirteenth peptides. In some embodiments, the composition comprises the second, fourth, fifth, sixth, eighth, ninth, tenth, eleventh, twelfth, thirteenth, fourteenth, fifteenth, and sixteenth peptides. In some embodiments, the composition comprises the second, third, fourth, fifth, sixth, seventh, eighth, ninth, tenth, eleventh, twelfth, thirteenth, fifteenth, sixteenth, and seventeenth peptides. In some embodiments, the composition comprises the second, third, fourth, fifth, sixth, eighth, ninth, tenth, eleventh, twelfth, thirteenth, fifteenth, sixteenth, and seventeenth peptides. In some embodiments, the composition comprises the first, second, third, fourth, fifth, sixth, tenth, eleventh, twelfth, thirteenth, fifteenth, seventeenth, and eighteenth peptides.

In some embodiments of any one of the kits provided herein, the composition comprises at least one of:

(a) a first peptide comprising the amino acid sequence PFPQPELPY (SEQ ID NO: 1) and the amino acid sequence PQPELPYPQ (SEQ ID NO: 2);

(b) a second peptide comprising the amino acid sequence PFPQPEQPF (SEQ ID NO: 3) and the amino acid sequence PQPEQPFPW (SEQ ID NO: 4);

(c) a third peptide comprising the amino acid sequence EQPIPEQPQ (SEQ ID NO: 5) and the amino acid sequence PIPEQPQPY (SEQ ID NO: 6);

(d) a fourth peptide comprising the amino acid sequence PFPQPEQPI (SEQ ID NO: 7) and the amino acid sequence PQPEQPIPV (SEQ ID NO: 8);

(e) a fifth peptide comprising the amino acid sequence EQPIPVQPE (SEQ ID NO: 9);

(f) a sixth peptide comprising the amino acid sequence PFPQPEQPT (SEQ ID NO: 10) and the amino acid sequence PQPEQPTPI (SEQ ID NO: 11);

(g) a seventh peptide comprising the amino acid sequence EQPTPIQPE (SEQ ID NO: 12);

(h) an eighth peptide comprising the amino acid sequence PQPEQPFPL (SEQ ID NO: 13);

(i) a ninth peptide comprising the amino acid sequence EQPFPLQPE (SEQ ID NO: 14);

(j) a tenth peptide comprising the amino acid sequence EGSFQPSQE (SEQ ID NO: 17);

(k) a eleventh peptide comprising the amino acid sequence QGYYPTSPQ (SEQ ID NO: 18);

(l) a twelfth peptide comprising the amino acid sequence EQPEQPFPE (SEQ ID NO: 19);

(m) a thirteenth peptide comprising the amino acid sequence PFSEQEQPV (SEQ ID NO: 22);

(n) a fourteenth peptide comprising the amino acid sequence PYPQPELPY (SEQ ID NO: 25);

(o) a fifteenth peptide comprising the amino acid sequence EQPFPEQPI (SEQ ID NO: 23) and the amino acid sequence PFPEQPIPE (SEQ ID NO: 24); and

(p) a sixteenth peptide comprising the amino acid sequence PYPEQPQPF (SEQ ID NO: 16) and the amino acid sequence PQPYPEQPQ (SEQ ID NO: 27).

In some embodiments:

(a) the first peptide comprises the amino acid sequence PFPQPELPYPQP (SEQ ID NO: 46);

(b) the second peptide comprises the amino acid sequence PFPQPEQPFPWQ (SEQ ID NO: 47);

(c) the third peptide comprises the amino acid sequence EQPIPEQPQPYP (SEQ ID NO: 48);

(d) the fourth peptide comprises the amino acid sequence PFPQPEQPIPVQ (SEQ ID NO: 49);

(e) the fifth peptide comprises the amino acid sequence PEQPIPVQPEQS (SEQ ID NO: 50);

(f) the sixth peptide comprises the amino acid sequence PFPQPEQPTPIQ (SEQ ID NO: 51);

(g) the seventh peptide comprises the amino acid sequence PEQPTPIQPEQP (SEQ ID NO: 52);

(h) the eighth peptide comprises the amino acid sequence PFPQPEQPFPLQ (SEQ ID NO: 53);

(i) the ninth peptide comprises the amino acid sequence PEQPFPLQPEQP (SEQ ID NO: 54);

(j) the tenth peptide comprises the amino acid sequence GEGSFQPSQENP (SEQ ID NO: 55);

(k) the eleventh peptide comprises the amino acid sequence QQGYYPTSPQQS (SEQ ID NO: 56);

(l) the twelfth peptide comprises the amino acid sequence PEQPEQPFPEQP (SEQ ID NO: 57);

(m) the thirteenth peptide comprises the amino acid sequence PPFSEQEQPVLP (SEQ ID NO: 58);

(n) the fourteenth peptide comprises the amino acid sequence PYPQPELPYPQP (SEQ ID NO: 59);

(o) the fifteenth peptide comprises the amino acid sequence EQPFPEQPIPEQ (SEQ ID NO: 60); and

(p) the sixteenth peptide comprises the amino acid sequence PQPYPEQPQPFP (SEQ ID NO: 61).

In some embodiments of any one of the kits provided herein, the composition comprises at least four (e.g., five, six, seven, eight, nine, ten, eleven, twelve, thirteen, fourteen, fifteen or sixteen) of the peptides. In some embodiments of any one of the kits provided herein, the composition comprises (or consists of) the peptides in (a)-(p). In some embodiments of any one of the kits provided herein, at least one of the peptides comprises an N-terminal pyroglutamate and/or a C-terminal amide group. In some embodiments of any one of the kits provided herein, each of the peptides comprises an N-terminal pyroglutamate and/or a C-terminal amide group.

In some embodiments of any one of the kits, the kit further comprises means to detect binding of one or more of the peptides in the composition to T cells. In some embodiments of any one of the kits, the kit further comprises means for administering the composition to a subject, e.g., a needle.

In some embodiments of any one of the kits, the means to detect binding of one or more of the peptides in the composition to T cells is a binding partner (e.g., an antibody) specific for a cytokine, e.g., IFN-gamma or IP-10. Binding partners are described herein. In some embodiments, the kit further comprises an agent that recognizes the binding partner. In some embodiments, the kit further comprises a container for blood. In some embodiments, the composition is contained within the container (e.g., dried onto the wall of the container).

In some embodiments of any one of the kits, the kit comprises a first and second binding partner for the cytokine. Binding partners are described herein. In some embodiments of any one of the kits, the first and second binding partners are antibodies or antigen binding fragments thereof. In some embodiments of any one of the kits, the second binding partner is bound to a surface. The second binding partner may be bound to the surface covalently or non-covalently. The second binding partner may be bound directly to the surface, or may be bound indirectly, e.g., through a linker. Examples of linkers, include, but are not limited to, carbon-containing chains, polyethylene glycol (PEG), nucleic acids, monosaccharide units, and peptides. The surface can be made of any material, e.g., metal, plastic, paper, or any other polymer, or any combination thereof. In some embodiments of any one of the kits, the first binding partner for the cytokine is washed over the cytokine bound to the second binding partner (e.g., as in a sandwich ELISA). The first binding partner may comprise a detectable label, or an agent that recognizes the first binding partner for the cytokine (e.g., a secondary antibody) may comprise a detectable label.

Any suitable agent that recognizes a binding partner for the cytokine is contemplated. In some embodiments of any one of the kits, the binding partner is any molecule that binds specifically to the binding partner for the cytokine. In some embodiments of any one of the kits, the agent is an antibody (e.g., a secondary antibody) or antigen-binding fragment thereof, such as Fab, F(ab)2, Fv, single chain antibodies, Fab and sFab fragments, F(ab′)2, Fd fragments, scFv, or dAb fragments. Agents also include other peptide molecules and aptamers that bind specifically to a binding partner for the cytokine. In some embodiments of any one of the kits, the binding partner for the cytokine comprises a biotin moiety and the agent is a composition that binds to the biotin moiety (e.g., an avidin or streptavidin).

In some embodiments of any one of the kits, the binding partner for the cytokine comprises a detectable label. Any suitable detectable label is contemplated. Detectable labels include any composition detectable by spectroscopic, photochemical, biochemical, immunochemical, chemical, or other physical means, e.g., an enzyme, a radioactive label, a fluorophore, an electron dense reagent, biotin, digoxigenin, or a hapten. Such detectable labels are well-known in the art are detectable through use of, e.g., an enzyme assay, a chromogenic assay, a luminometric assay, a fluorogenic assay, or a radioimmune assay. The reaction conditions to perform detection of the detectable label depend upon the detection method selected.

In some embodiments of any one of the kits, the kit further comprises a negative control, e.g., a composition that does not comprise a gluten peptide, e.g., a saline solution or cell culture medium. In some embodiments, the kit further comprises a positive control, e.g., a composition comprising the cytokine at a known concentration.

In some embodiments of any one of the kits, the kit comprises any combination of the components mentioned above.

In some embodiments of any one of the kits, the kit further comprises instructions for use of the composition. In some embodiments, the instructions include a method as described herein. Instructions can be in any suitable form, e.g., as a printed insert or a label.

### General Techniques and Definitions

Unless specifically defined otherwise, all technical and scientific terms used herein shall be taken to have the same meaning as commonly understood by one of ordinary skill in the art (e.g., in cell culture, molecular genetics, immunology, immunohistochemistry, protein chemistry, and biochemistry).

Unless otherwise indicated, techniques utilized in the present disclosure are standard procedures, well known to those skilled in the art. Such techniques are described and explained throughout the literature in sources such as, J. Sambrook et al., Molecular Cloning: A Laboratory Manual, Cold Spring Harbour Laboratory Press (2012); T. A. Brown (editor), Essential Molecular Biology: A Practical Approach, Volumes 1 and 2, IRL Press (2000 and 2002); D. M. Glover and B. D. Hames (editors), Current Protocols in Molecular Biology, Greene Pub. Associates and Wiley-Interscience (1988, including all updates until present); Edward A. Greenfield (editor) Antibodies: A Laboratory Manual, Cold Spring Harbour Laboratory, (2013); and J. E. Coligan et al. (editors), Current Protocols in Immunology, John Wiley & Sons (including all updates until present).

In any one aspect or embodiment provided herein “comprising” may be replaced with “consisting essentially of” or “consisting of”.

### EXAMPLES

### Example 1

### Methods

HLA-DQ2.5-positive celiac disease subjects on a gluten-free diet were used in this study. Blood was collected immediately before and 6 days after commencing a 3-day oral gluten challenge. Whole blood or PBMCs were incubated with pools or single peptides derived from gluten or recall antigens. Negative control samples were contacted with medium only (no peptides). Positive control samples were contacted with CEF (human CMV, EBV and influenza virus) peptide pools. IFNγ and IP-10 levels were measured in plasma from the whole blood that was incubated in 96-well plates with peptides or peptide pools. Plasma cytokine/chemokine levels were measured by MAGPIX® multiplex bead assay (IFNγ and IP-10) or by ELISA (IFNγ), and PBMC separated from the same blood sample were incubated in overnight IFNγ ELISpot assays.

The individual peptides used are shown in Table 1 (pE=pyroglutamate).

TABLE 1
Individual peptides

| Peptide | | | |
|---|---|---|---|
| Identifier | Sequence | Epitopes | Restriction |
| 1 | (pE)LQPFPQPELPYPQPQ- | PFPQPELPY | DQ2.5 |
| | amide (SEQ ID NO: 66) | (SEQ ID NO: 1), | |
| | | PQPELPYPQ | |
| | | (SEQ ID NO: 2) | |
| 2 | (pE)QPFPQPEQPFPWQP- | PFPQPEQPF | DQ2.5 |
| | amide (SEQ ID NO: 67) | (SEQ ID NO: 3), | |
| | | PQPEQPFPW | |
| | | (SEQ ID NO: 4) | |
| 3 | (pE)PEQPEPEQPQPYPQQ- | PIPEQPQPY | DQ2.5 |
| | amide (SEQ ID NO: 68) | (SEQ ID NO: 6) | |
| 4 | (pE)PEQPFPEQPIPEQPQPYP- | EQPFPEQPI | DQ2.5 |
| | amide (SEQ ID NO: 69) | (SEQ ID NO: 23), | |
| | | PIPEQPQPY | |
| | | (SEQ ID NO: 6) | |
| 5 | (pE)QPFPQPEQPIPVQPEQS- | PFPQPEQPIP | DQ2.5/2.5 and 8/8 |
| | amide (SEQ ID NO: 70) | (SEQ ID NO: 62), | |
| | | EQPIPVQPE | |
| | | (SEQ ID NO: 9) | |
| 6 | (pE)QPFPQPEQPTPIQPEQP- | PFPQPEQPTPI | DQ2.5/2.5 and 8/8 |
| | amide (SEQ ID NO: 71) | (SEQ ID NO: 65), | |
| | | EQPTPIQPE | |
| | | (SEQ ID NO: 12) | |
| 7 | (pE)QPFPQPEQPFPLQPEQP- | PQPEQPFPL | DQ2.5/2.5 and 8/8 |
| | amide (SEQ ID NO: 72) | (SEQ ID NO: 13), | |
| | | EQPFPLQPE | |
| | | (SEQ ID NO: 14) | |
| 8 | (pE)QPFPQPEQPFSQQ- | PFPQPEQPF | DQ2.5 |
| | amide (SEQ ID NO: 73) | (SEQ ID NO: 3), | |
| | | PQPEQPFSQ | |
| | | (SEQ ID NO: 15) | |
| 9 | (pE)PQPYPEQPQPFPQQ- | PYPEQPQPF | DQ2.5 |
| | amide (SEQ ID NO: 74) | (SEQ ID NO: 16) | |
| 10 | (pE)QPYPQPELPYPQPQ- | PQPELPYPQ | DQ2.5 |
| | amide (SEQ ID NO: 75) | (SEQ ID NO: 2), | |
| | | PYPQPELPY | |
| | | (SEQ ID NO: 25) | |
| 11 | (pE)QPFPQPELPYPYPQ- | PFPQPELPY | DQ2.5 |
| | amide (SEQ ID NO: 76) | (SEQ ID NO: 1), | |
| | | PQPELPYPY | |
| | | (SEQ ID NO: 26) | |
| 12 | (pE)SGEGSFQPSQENPQ- | EGSFQPSQE | DQ8/2.5 and 8/8 |
| | amide (SEQ ID NO: 77) | (SEQ ID NO: 17) | |
| 13 | (pE)GQQGYYPTSPQQSG- | QGYYPTSPQ | DQ2.5/2.5 and 8/8 |
| | amide (SEQ ID NO: 78) | (SEQ ID NO: 18) | |
| 14 | (pE)PEQPEQPFPEQPQQ- | EQPEQPFPEQPQ | DQ2.5/2.5 and |
| | amide (SEQ ID NO: 79) | (SEQ ID NO: 64) | 8/8/2.2 and 8 |
| 15 | (pE)QPFPEQPEQIIPQQP- | PFPEQPEQIIP | DQ2.5 |
| | amide (SEQ ID NO: 80) | (SEQ ID NO: 63) | |
| 16 | (pE)QPPFSEQEQPVLPQ- | PFSEQEQPV | DQ2.2 |
| | amide (SEQ ID NO: 81) | (SEQ ID NO: 22) | |

The peptide pools used are shown in Tables 2-4.

TABLE 2
Peptide pool 1-3 peptides (pE = pyroglutamate)

| Identi- | | | Restric- |
|---|---|---|---|
| fier | Sequence | Epitopes | tion |
| 1 | (pE)LQPFPQPELPY | PFPQPELPY | DQ2.5 |
| | PQPQ-amide | (SEQ ID NO: 1), | |
| | (SEQ ID NO: 66) | PQPELPYPQ | |
| | | (SEQ ID NO: 2) | |
| 2 | (pE)QPFPQPEQPFP | PFPQPEQPF | DQ2.5 |
| | WQP-amide | (SEQ ID NO: 3), | |
| | (SEQ ID NO: 67) | PQPEQPFPW | |
| | | (SEQ ID NO: 4) | |
| 3 | (pE)PEQPIPEQPQP | PIPEQPQPY | DQ2.5 |
| | YPQQ-amide | (SEQ ID NO: 6) | |
| | (SEQ ID NO: 68) | | |

TABLE 3
Peptide pool 2-13 peptides (pE = pyroglutamate)

| Identifier | Sequence | Epitopes | Restriction |
|---|---|---|---|
| 1 | (pE)LQPFPQPELPYPQPQ- | PFPQPELPY | DQ2.5 |
| | amide (SEQ ID NO: 66) | (SEQ ID NO: 1), | |
| | | PQPELPYPQ | |
| | | (SEQ ID NO: 2) | |
| 2 | (pE)QPFPQPEQPFPWQP- | PFPQPEQPF | DQ2.5 |
| | amide (SEQ ID NO: 67) | (SEQ ID NO: 3), | |
| | | PQPEQPFPW | |
| | | (SEQ ID NO: 4) | |
| 3 | (pE)PEQPIPEQPQPYPQQ- | PIPEQPQPY | DQ2.5 |
| | amide (SEQ ID NO: 68) | (SEQ ID NO: 6) | |
| 5 | (pE)QPFPQPEQPIPVQPEQS- | PFPQPEQPIP | DQ2.5/2.5 and 8/8 |
| | amide (SEQ ID NO: 70) | (SEQ ID NO: 62), | |
| | | EQPIPVQPE | |
| | | (SEQ ID NO: 9) | |
| 6 | (pE)QPFPQPEQPTPIQPEQP- | PFPQPEQPTPI | DQ2.5/2.5 and 8/8 |
| | amide (SEQ ID NO: 71) | (SEQ ID NO: 65), | |
| | | EQPTPIQPE | |
| | | (SEQ ID NO: 12) | |
| 7 | (pE)QPFPQPEQPFPLQPEQP- | PQPEQPFPL | DQ2.5/2.5 and 8/8 |
| | amide (SEQ ID NO: 72) | (SEQ ID NO: 13), | |
| | | EQPFPLQPE | |
| | | (SEQ ID NO: 14) | |
| 8 | (pE)QPFPQPEQPFSQQ- | PFPQPEQPF | DQ2.5 |
| | amide (SEQ ID NO: 73) | (SEQ ID NO: 3), | |
| | | PQPEQPFSQ | |
| | | (SEQ ID NO: 15) | |
| 9 | (pE)PQPYPEQPQPFPQQ- | PYPEQPQPF | DQ2.5 |
| | amide (SEQ ID NO: 74) | (SEQ ID NO: 16) | |
| 15 | (pE)QPFPEQPEQIIPQQP- | PFPEQPEQIIP | DQ2.5 |
| | amide (SEQ ID NO: 80) | (SEQ ID NO: 63) | |
| 12 | (pE)SGEGSFQPSQENPQ- | EGSFQPSQE | DQ8/2.5 and 8/8 |
| | amide (SEQ ID NO: 77) | (SEQ ID NO: 17) | |
| 13 | (pE)GQQGYYPTSPQQSG- | QGYYPTSPQ | DQ2.5/2.5 and 8/8 |
| | amide (SEQ ID NO: 78) | (SEQ ID NO: 18) | |
| 14 | (pE)PEQPEQPFPEQPQQ- | EQPEQPFPEQPQ | DQ2.5/2.5 and |
| | amide (SEQ ID NO: 79) | (SEQ ID NO: 64) | 8/8/2.2 and 8 |
| 16 | (pE)QPPFSEQEQPVLPQ- | PFSEQEQPV | DQ2.2 |
| | amide (SEQ ID NO: 81) | (SEQ ID NO: 22) | |

TABLE 4
Peptide pool 3-14 peptides (pE = pyroglutamate)

| Identifier | Sequence | Epitopes | Restriction |
|---|---|---|---|
| 2 | (pE)QPFPQPEQPFPWQP- | PFPQPEQPF | DQ2.5 |
| | amide (SEQ ID NO: 67) | (SEQ ID NO: 3), | |
| | | PQPEQPFPW | |
| | | (SEQ ID NO: 4) | |
| 5 | (pE)QPFPQPEQPIPVQPEQS- | PFPQPEQPIP | DQ2.5/2.5 and |
| | amide (SEQ ID NO: 70) | (SEQ ID NO: 62), | 8/8 |
| | | EQPIPVQPE | |
| | | (SEQ ID NO: 9) | |
| 6 | (pE)QPFPQPEQPTPIQPEQP- | PFPQPEQPTPI | DQ2.5/2.5 and |
| | amide (SEQ ID NO: 71) | (SEQ ID NO: 65), | 8/8 |
| | | EQPTPIQPE | |
| | | (SEQ ID NO: 12) | |
| 7 | (pE)QPFPQPEQPFPLQPEQP- | PQPEQPFPL | DQ2.5/2.5 and |
| | amide (SEQ ID NO: 72) | (SEQ ID NO: 13), | 8/8 |
| | | EQPFPLQPE | |
| | | (SEQ ID NO: 14) | |
| 8 | (pE)QPFPQPEQPFSQQ-amide | PFPQPEQPF | DQ2.5 |
| | (SEQ ID NO: 73) | (SEQ ID NO: 3), | |
| | | PQPEQPFSQ | |
| | | (SEQ ID NO: 15) | |
| 9 | (pE)PQPYPEQPQPFPQQ- | PYPEQPQPF | DQ2.5 |
| | amide (SEQ ID NO: 74) | (SEQ ID NO: 16) | |
| 15 | (pE)QPFPEQPEQIIPQQP- | PFPEQPEQIIP | DQ2.5 |
| | amide (SEQ ID NO: 80) | (SEQ ID NO: 63) | |
| 12 | (pE)SGEGSFQPSQENPQ- | EGSFQPSQE | DQ8/2.5 and 8/8 |
| | amide (SEQ ID NO: 77) | (SEQ ID NO: 17) | |
| 13 | (pE)GQQGYYPTSPQQSG- | QGYYPTSPQ | DQ2.5/2.5 and |
| | amide (SEQ ID NO: 78) | (SEQ ID NO: 18) | 8/8 |
| 14 | (pE)PEQPEQPFPEQPQQ- | EQPEQPFPEQPQ | DQ2.5/2.5 and |
| | amide (SEQ ID NO: 79) | (SEQ ID NO: 64) | 8/8/2.2 and 8 |
| 16 | (pE)QPPFSEQEQPVLPQ- | PFSEQEQPV | DQ2.2 |
| | amide (SEQ ID NO: 81) | (SEQ ID NO: 22) | |
| 4 | (pE)PEQPFPEQPIPEQPQPYP- | EQPFPEQPI | DQ2.5 |
| | NH2 (SEQ ID NO: 69) | (SEQ ID NO: 23), | |
| | | PIPEQPQPY | |
| | | (SEQ ID NO: 6) | |
| 10 | (pE)QPYPQPELPYPQPQ- | PQPELPYPQ | DQ2.5 |
| | NH2 (SEQ ID NO: 75) | (SEQ ID NO: 2), | |
| | | PYPQPELPY | |
| | | (SEQ ID NO: 25) | |
| 11 | (pE)QPFPQPELPYPYPQ-NH2 | PFPQPELPY | DQ2.5 |
| | (SEQ ID NO: 76) | (SEQ ID NO: 1), | |
| | | PQPELPYPY | |
| | | (SEQ ID NO: 26) | |

Each peptide in the above pools was designed to include at least one T cell epitope. The peptide pools were provided such that equimolar amounts of each peptide were present in each pool. A total gluten pool including 71 peptides capturing the majority of T cell epitopes in gluten was used as a control to simulate total gluten.

### Results

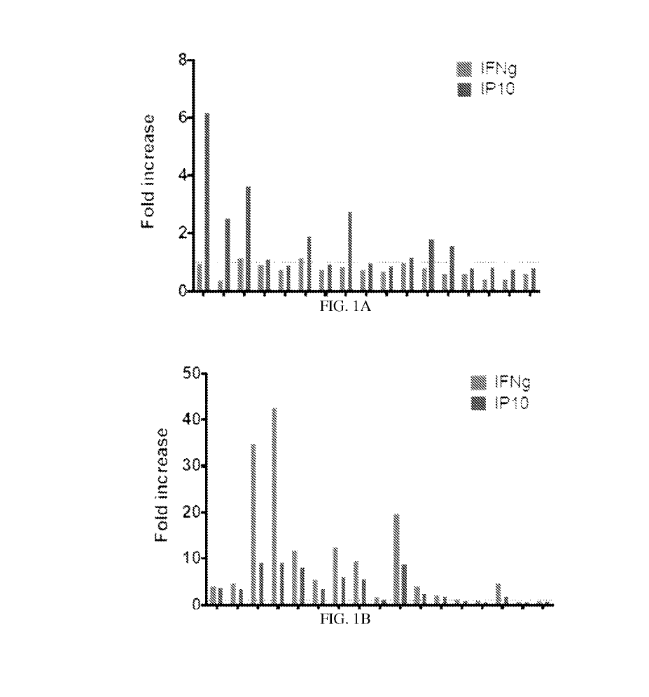

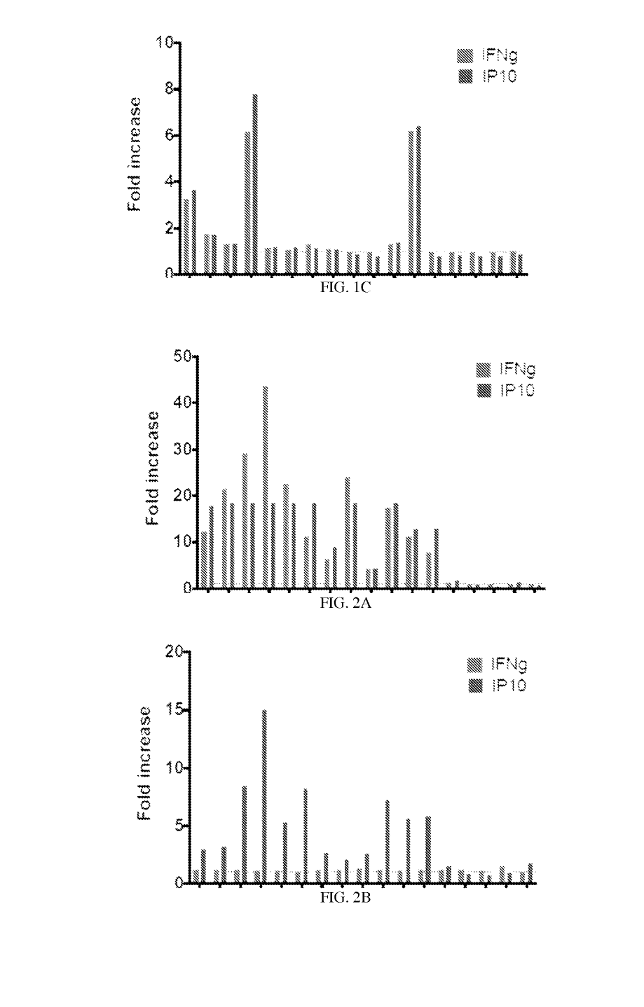

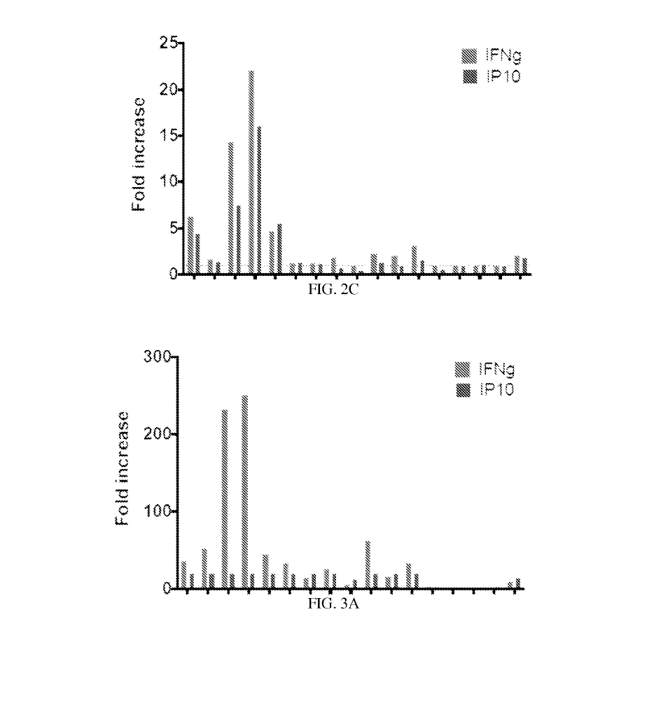

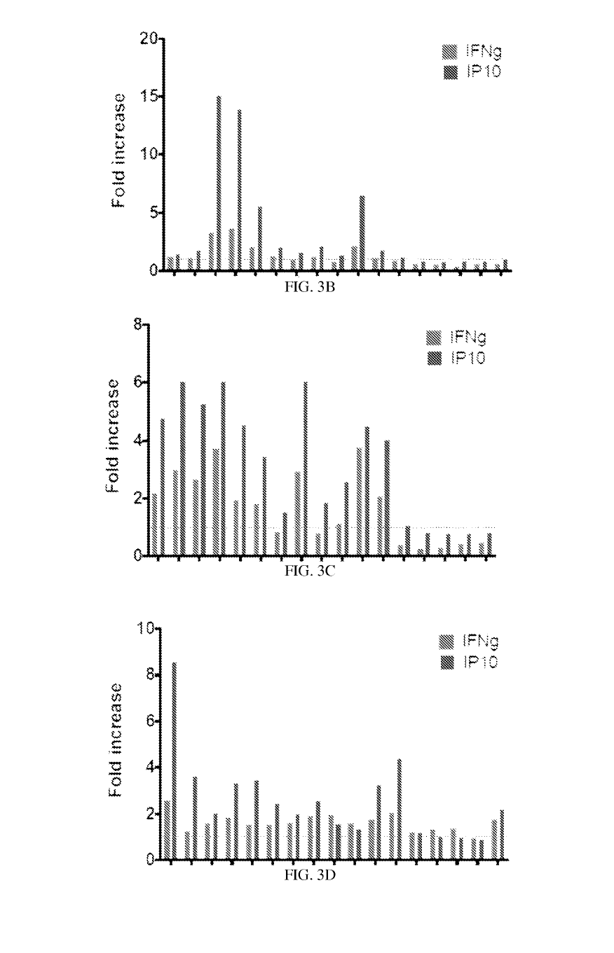

Individual peptides containing several different T cell epitopes were tested to identify candidates for inclusion in peptide pools. It was found that subjects had variable responses to the peptides (FIGS. 1 and 2), meaning that multiple peptides were preferred for a composition to be effective for the majority of DQ2.5+ subjects and also to be effective for Celiac disease subjects having other genetic backgrounds (e.g., DQ2.2 and DQ8+ subjects). Thus, 13 or 14 peptides from the individual peptides were pooled together in order to cover the variable responses observed in the subjects. In other words, these pools were designed to contain T cell epitopes that were restricted by HLA-DQ2.2, DQ2.5, and DQ8+ and stimulatory for the majority of circulating gluten-reactive T cells in patients with celiac disease positive for HLA-DQ2.2, DQ2.5, and/or DQ8. Thus, the majority of Celiac disease subjects are expected respond to the peptide pools. Peptide pool 1, which contained only HLA-DQ2.5-restricted epitopes, was used for comparison purposes, as this peptide pool had been shown previously to effectively induce T cell responses in a majority of subjects with DQ2.5+ Celiac disease.

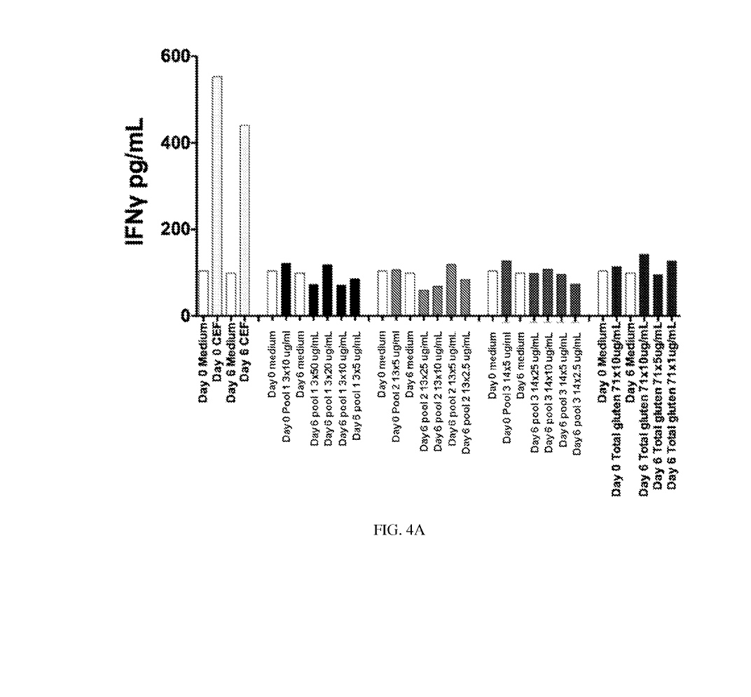

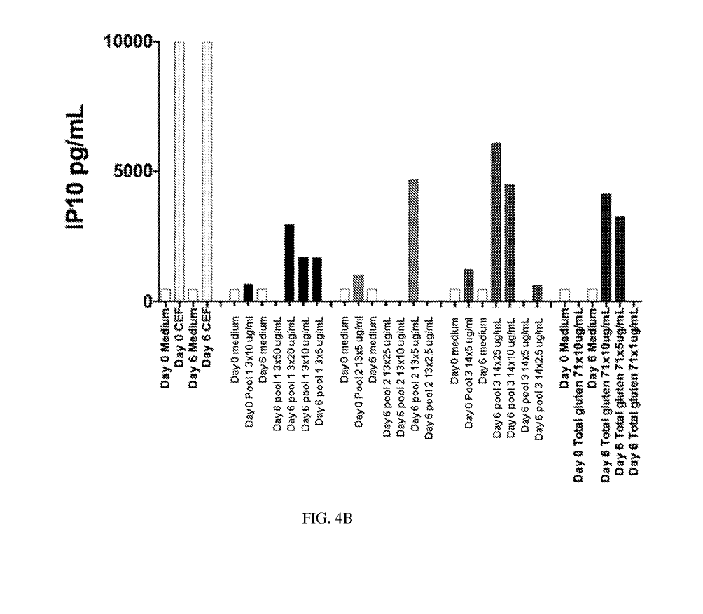

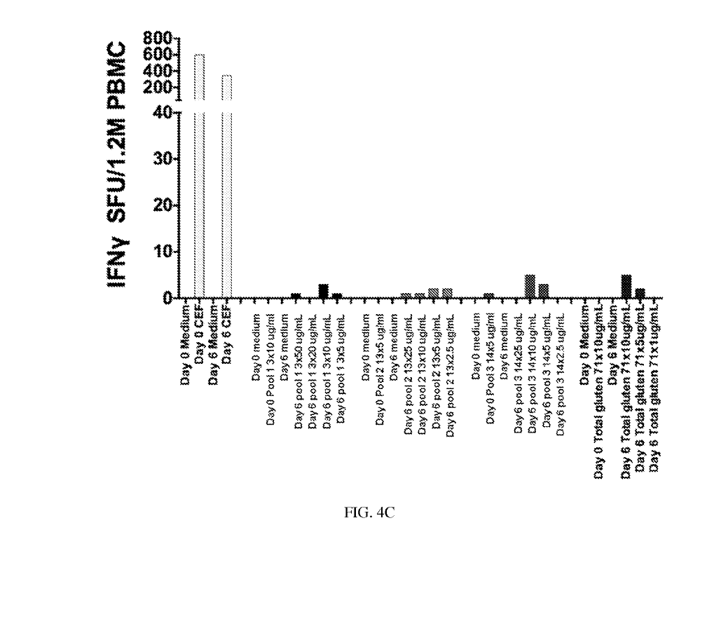

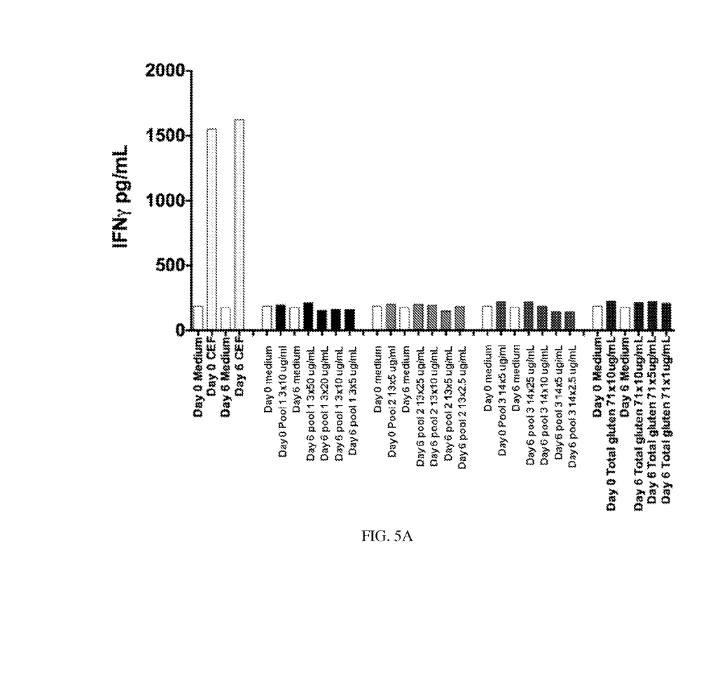

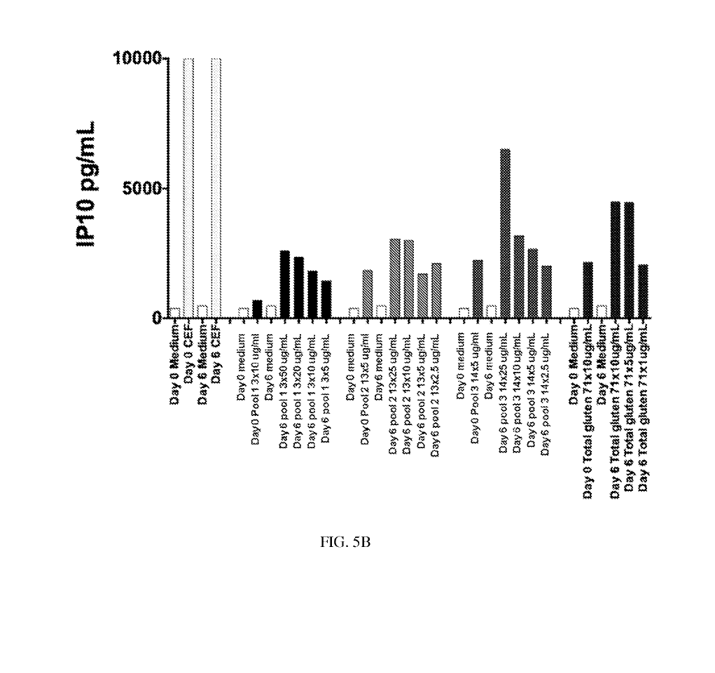

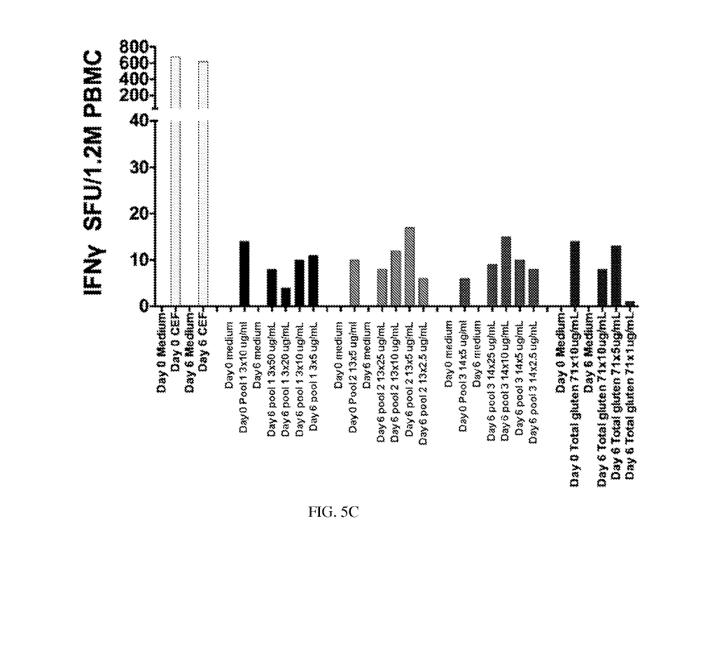

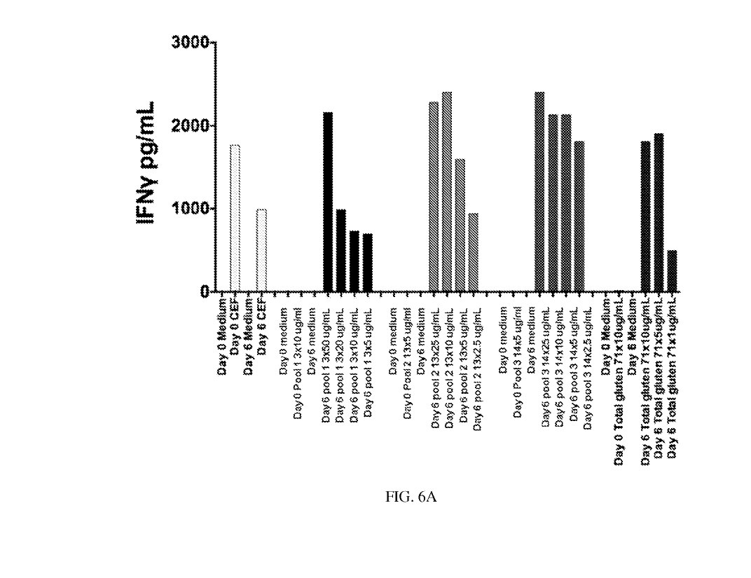

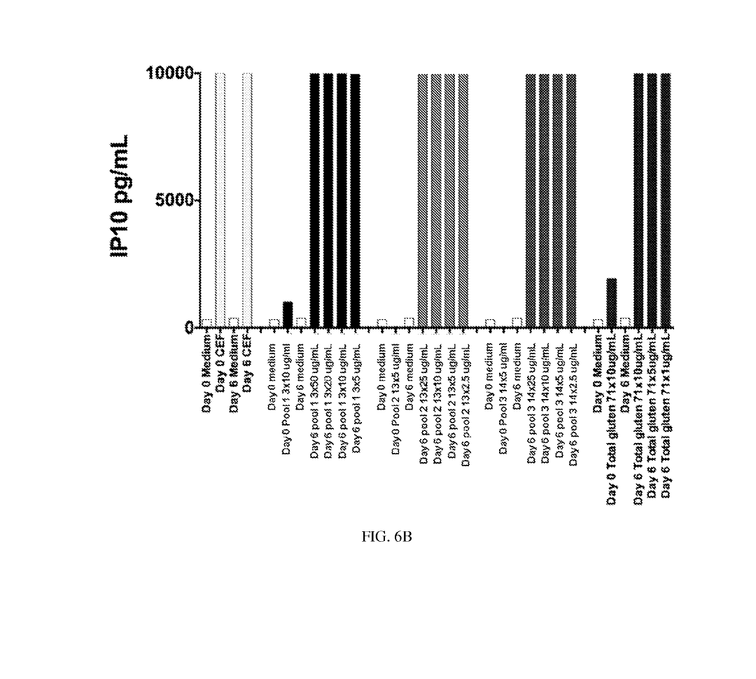

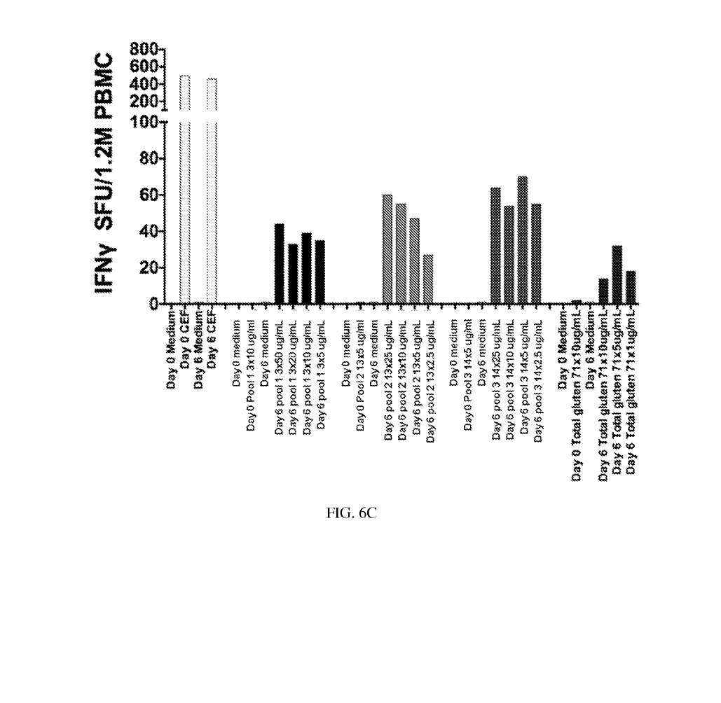

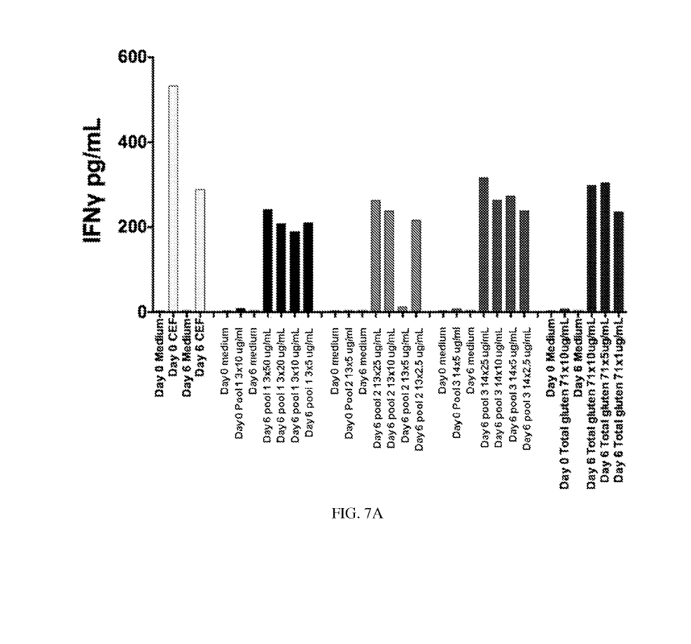

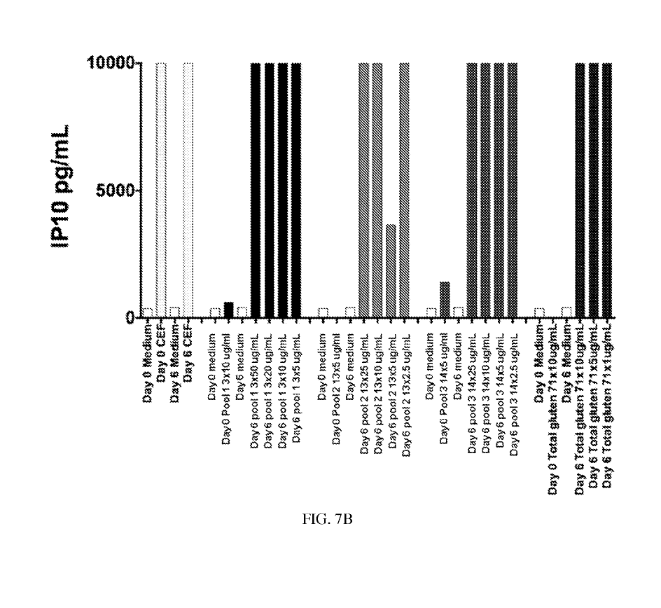

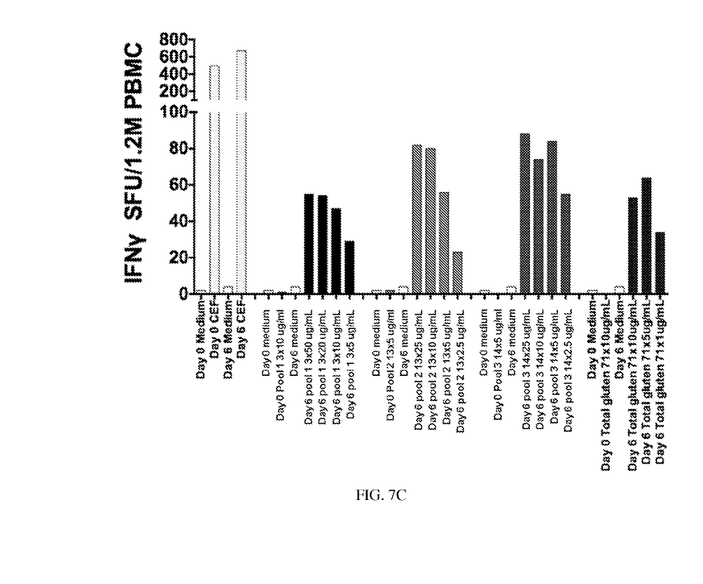

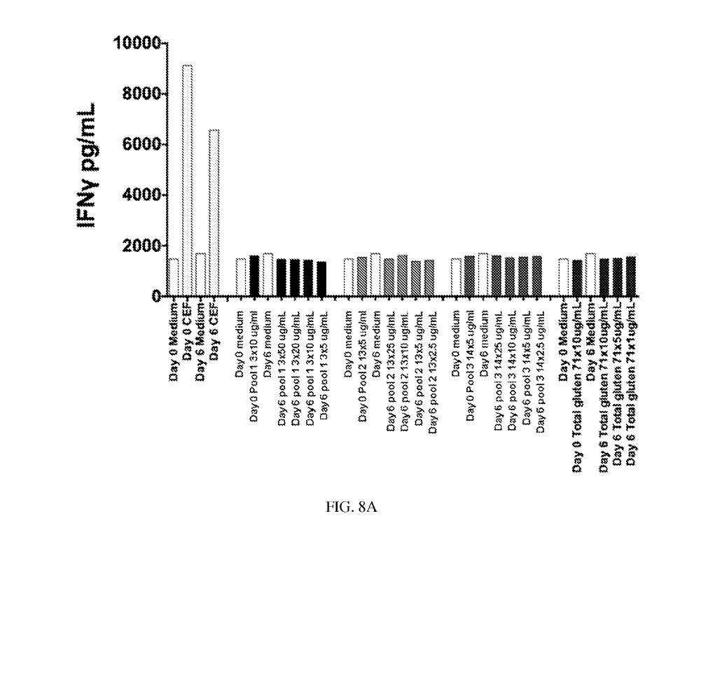

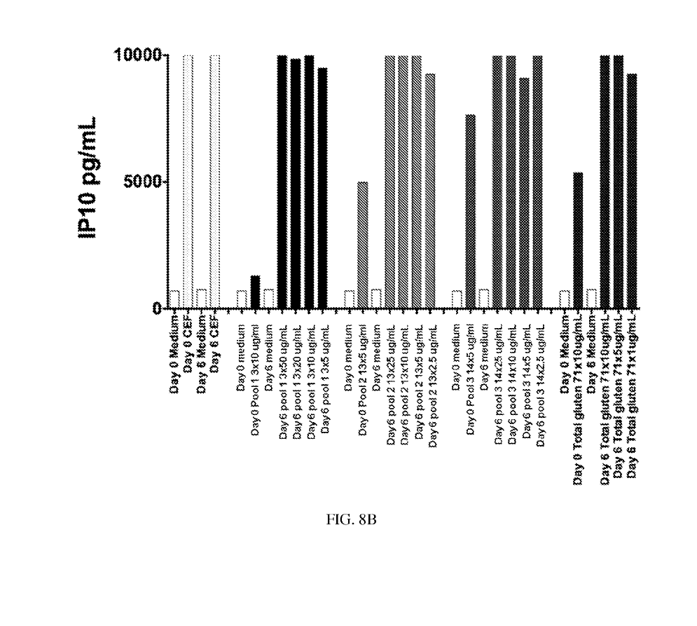

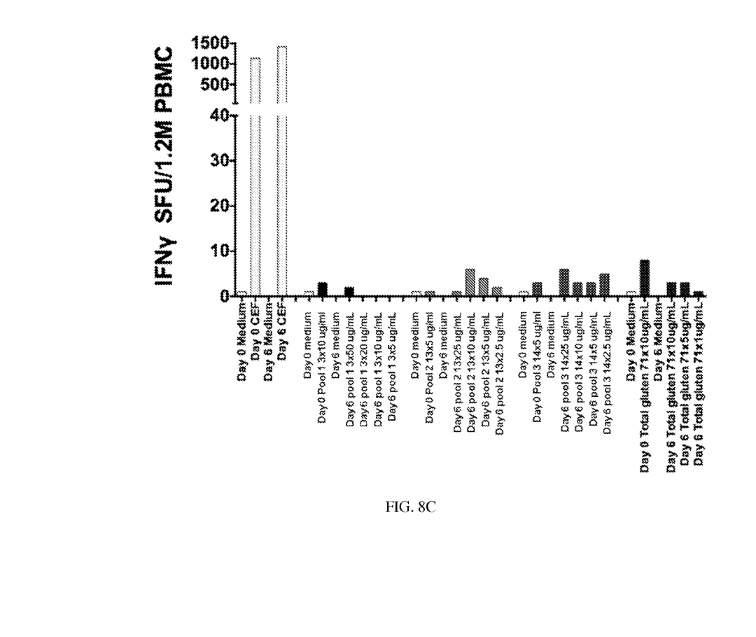

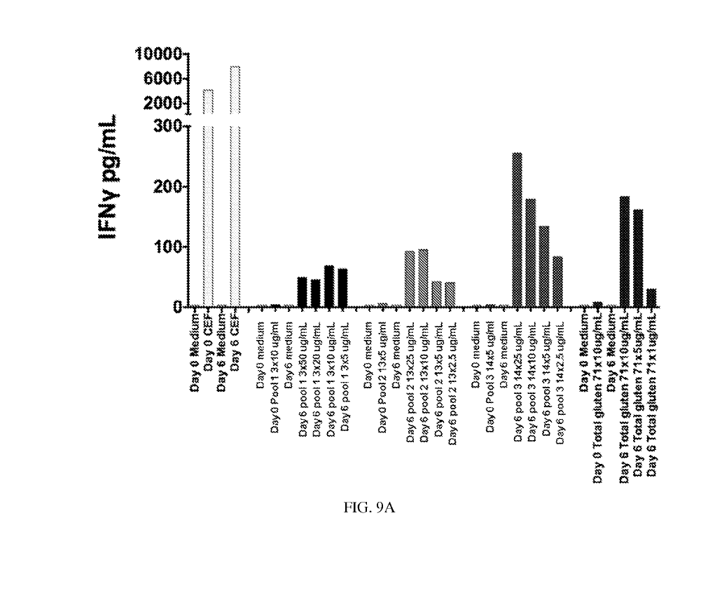

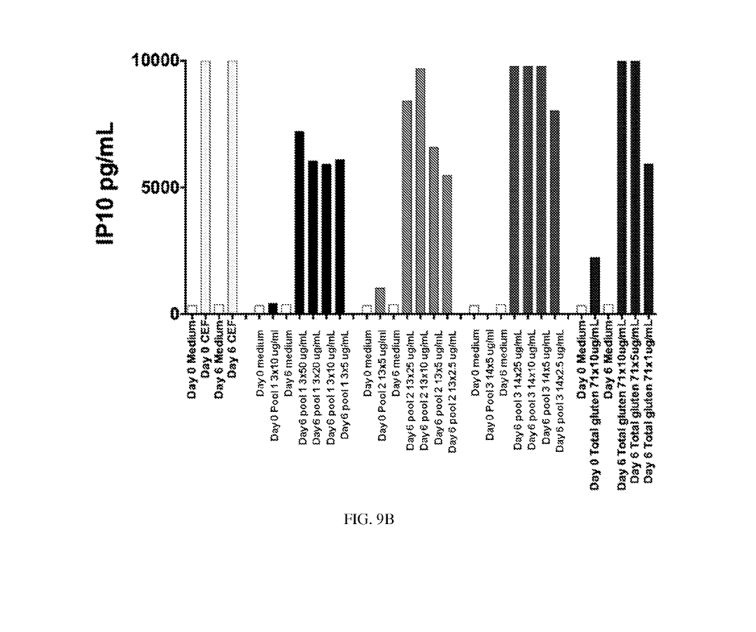

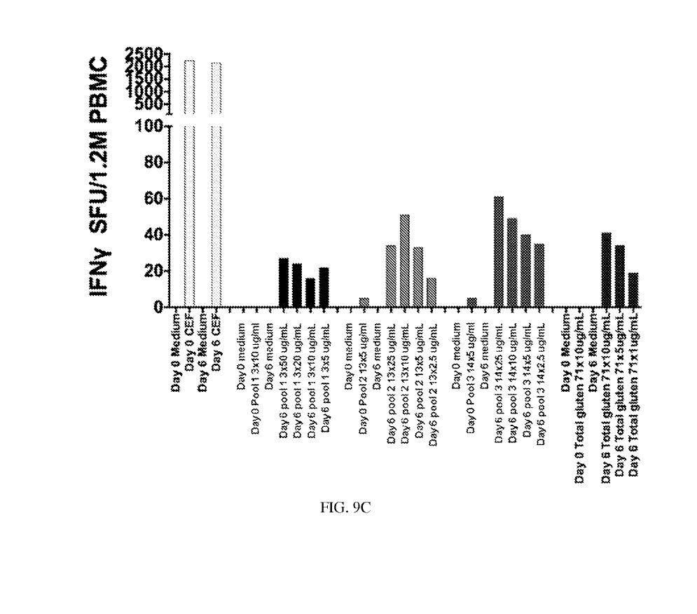

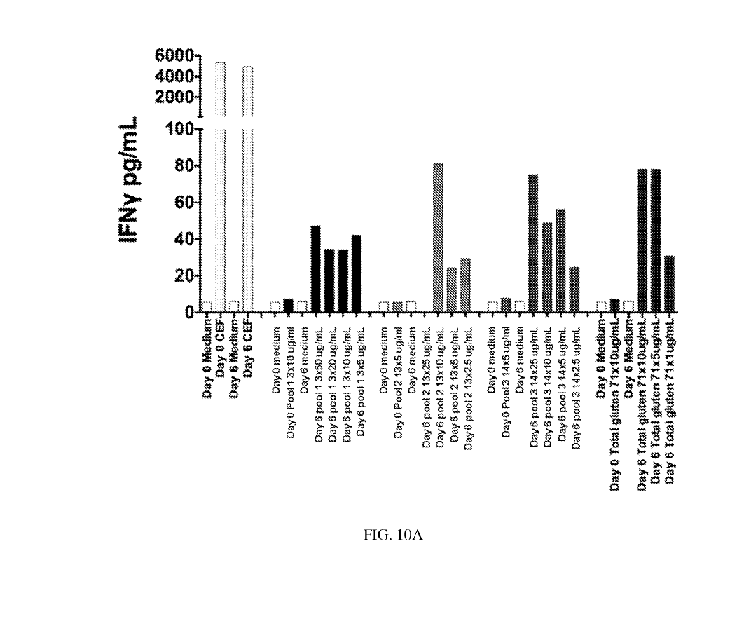

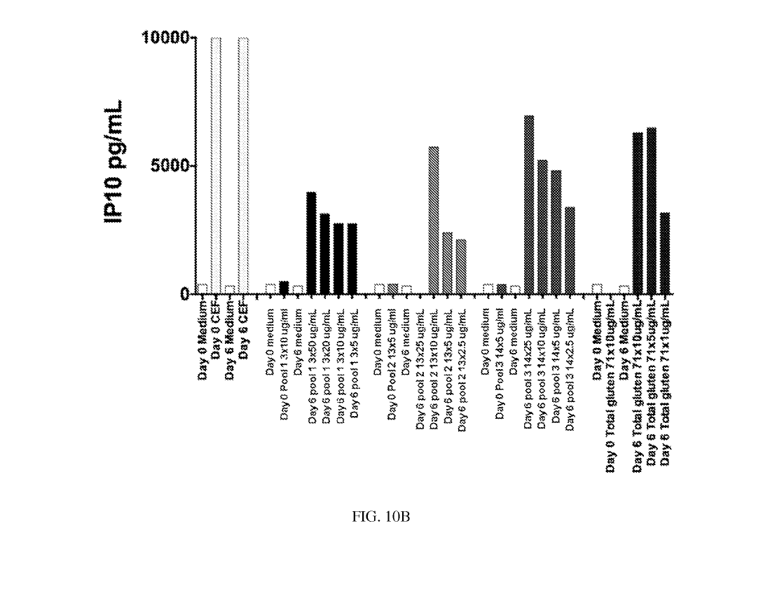

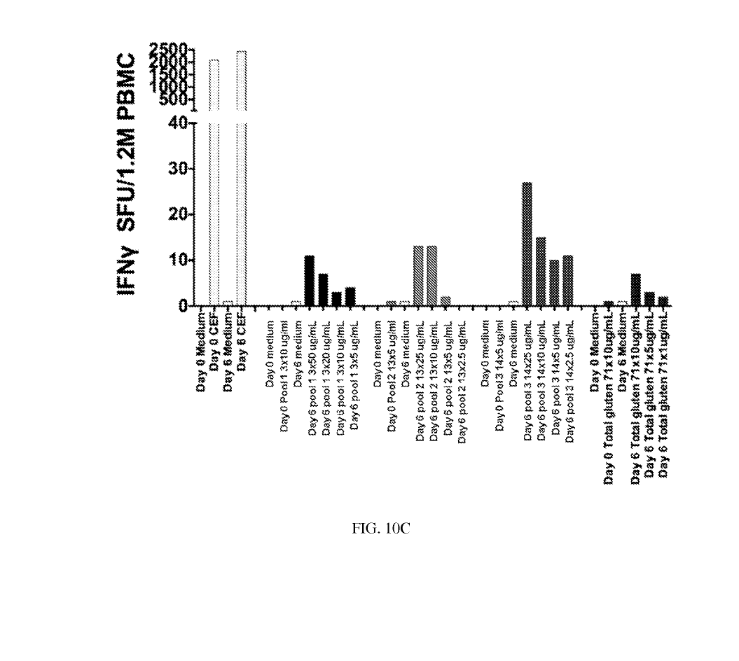

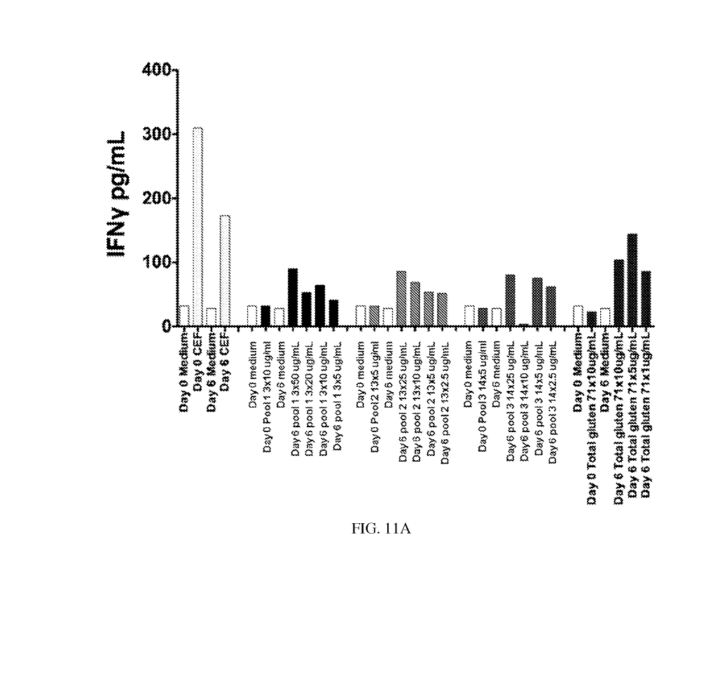

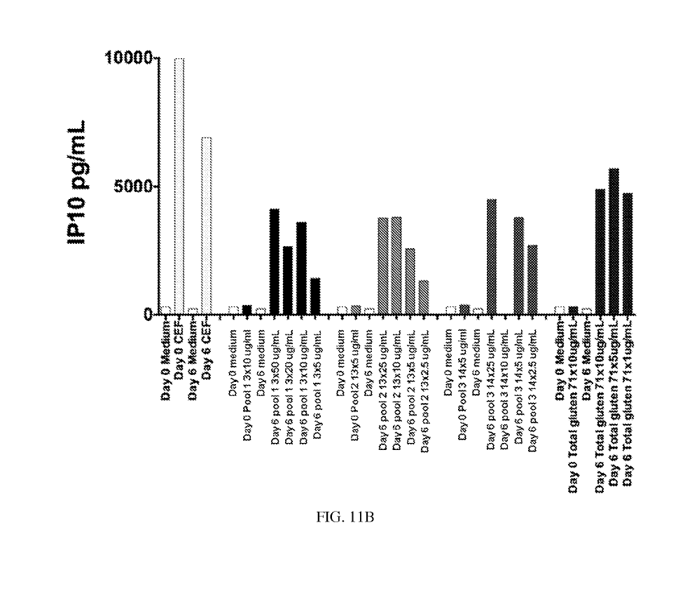

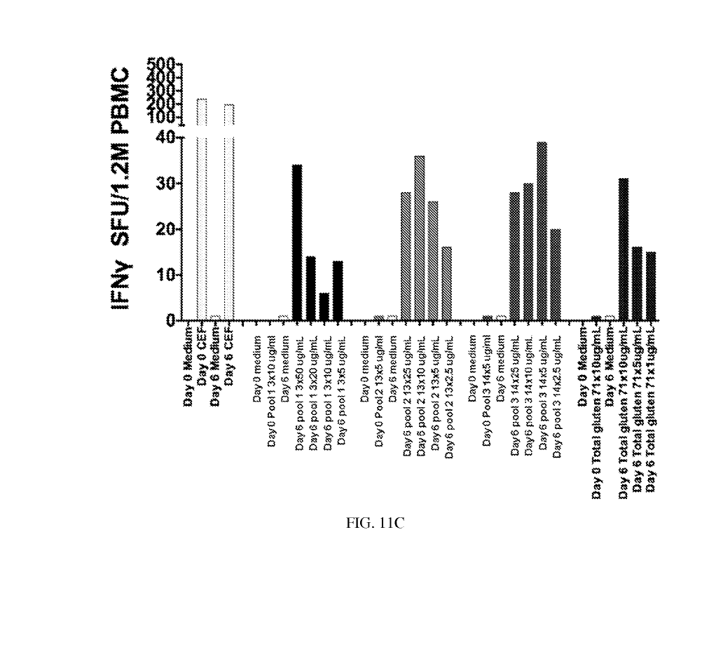

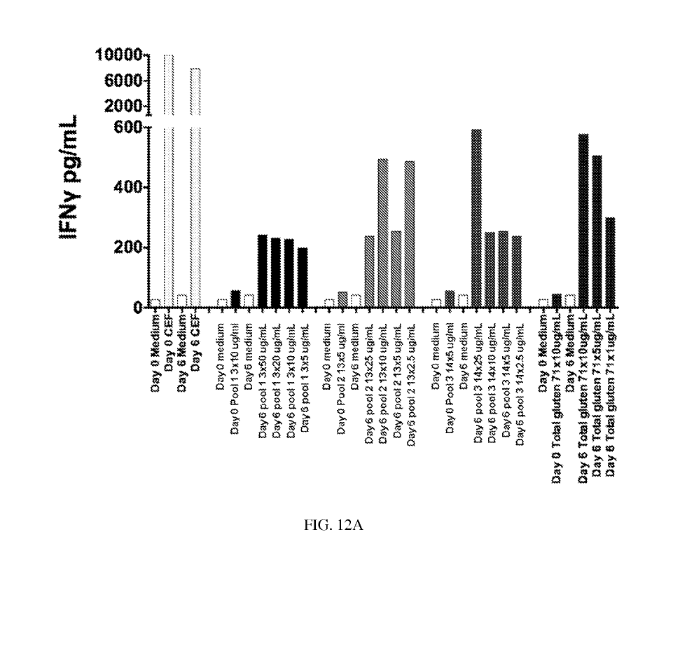

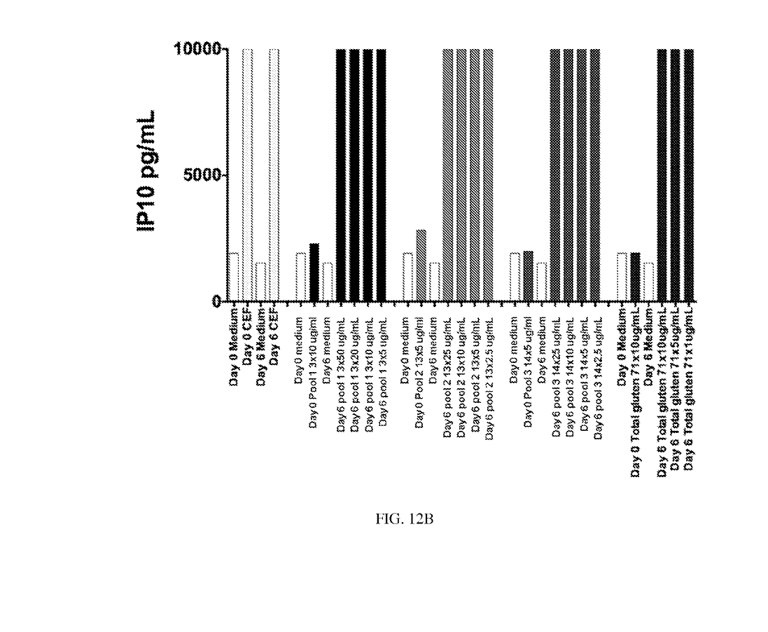

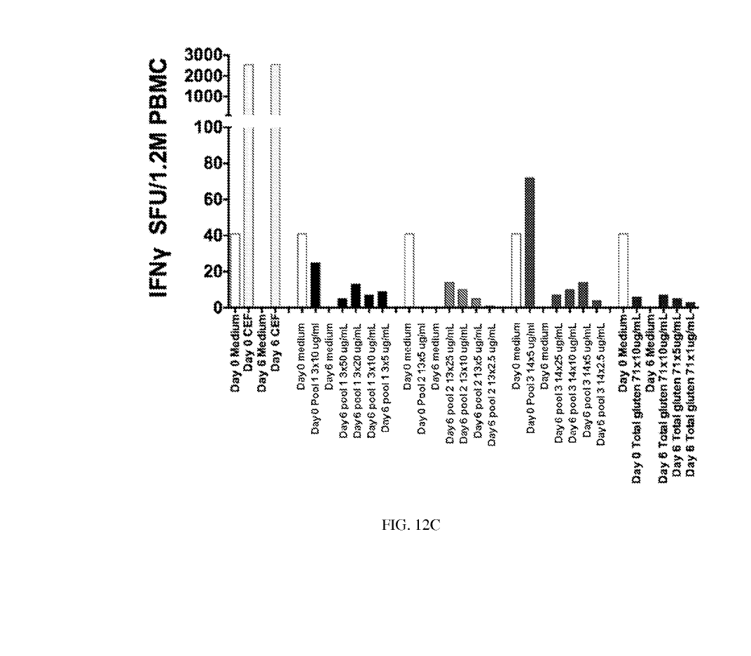

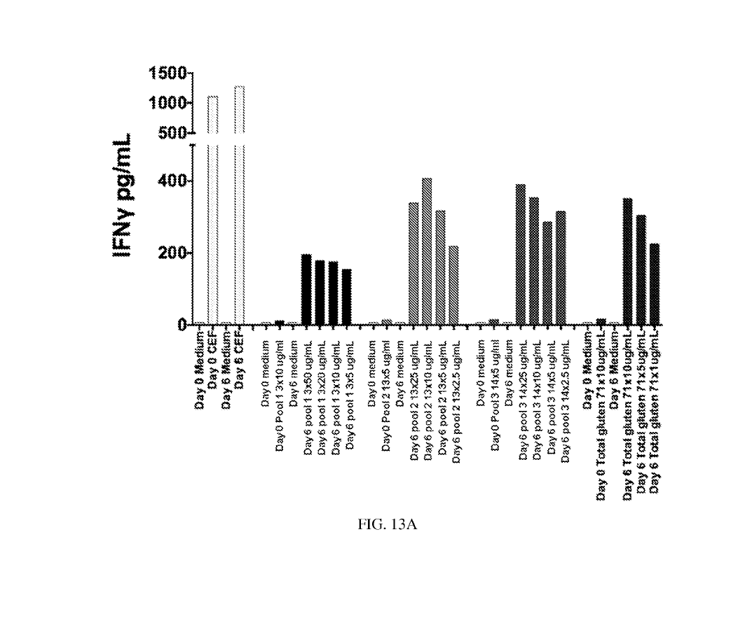

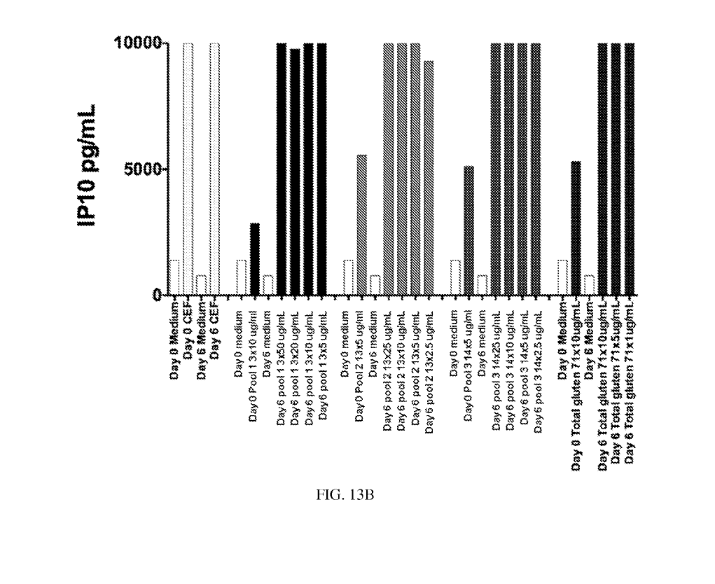

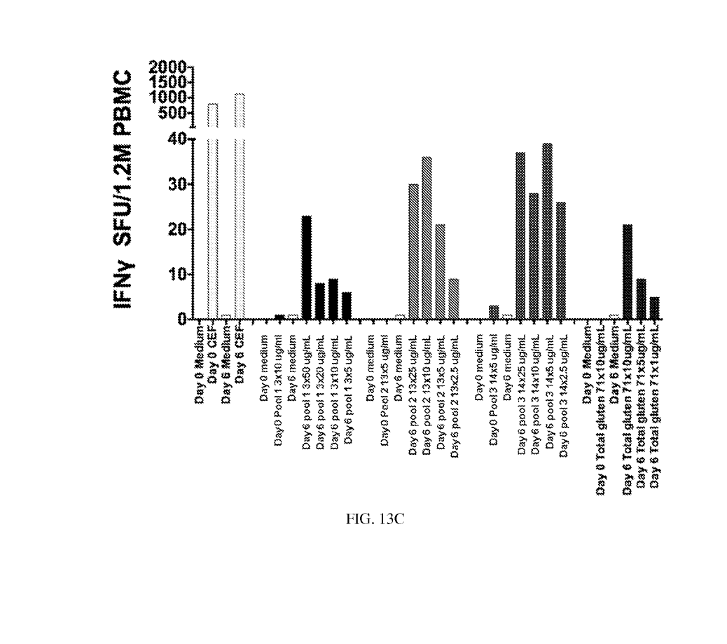

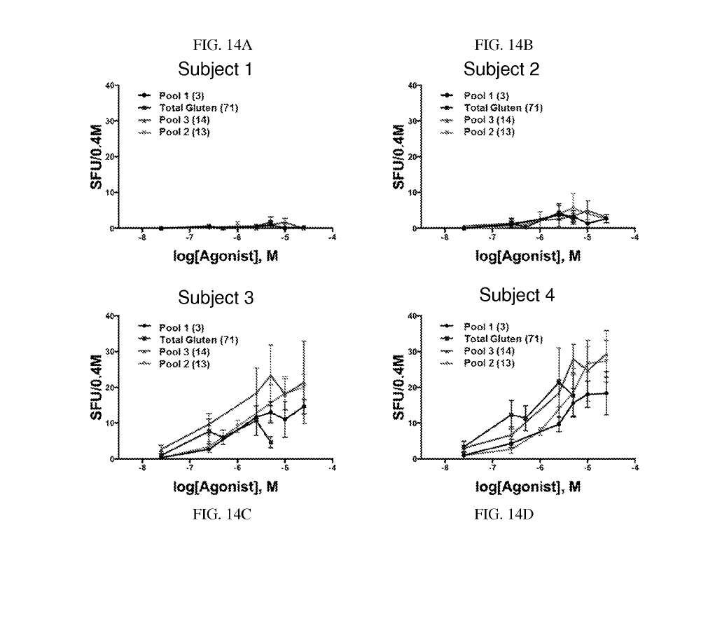

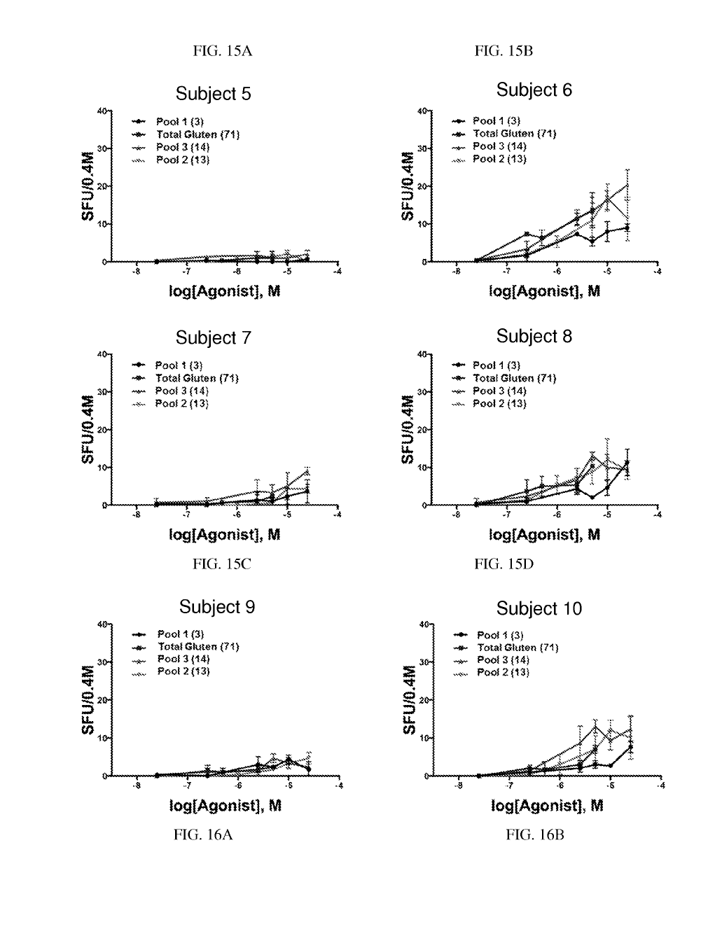

T cell responses to peptide pools 1, 2, and 3 were assessed in blood samples from subjects with DQ2.5+ Celiac disease. Elevated levels of IFNγ and IP-10, compared to a negative control level, were used as a readout for induction of a T cell response. Peptide pools 2 and 3, which contained 13 and 14 peptides, respectively, both stimulated whole blood secretion of IP-10 and IFNγ, and also IFNγ ELISpot responses, that were consistently equal to or greater than peptide pool 1, which contained 3 peptides (FIGS. 4-13 and Table 5). Surprisingly, peptide pools 2 and 3 also induced T cell responses generally similar to the total gluten pool, which contained 71 peptides capturing the majority of T cell epitopes in gluten (FIGS. 14-16 and Table 5). Thus, compositions containing fewer than the majority of gluten T cell epitopes were able to stimulate T cell responses robustly and to a similar degree as a composition comprising 71 peptides. It is expected that these compositions are effective for use in diagnosing Celiac disease (e.g., using a T cell response) and also for treating Celiac disease (e.g., by inducing tolerance to the T cell epitopes in the peptides, and to gluten, which contains the same T cell epitopes).

TABLE 5
Peptide-specific IFN-gamma ELISpot responses Day-6 after gluten challenge DQ2.5+
CD (spot forming units per 0.8 million PBMC)

| | Subject 1 | Subject 2 | Subject 3 | Subject 4 | Subject 5 | Subject 6 | Subject 7 | Subject 8 | Subject 9 | Subject 10 |
|---|---|---|---|---|---|---|---|---|---|---|
| Peptide pool 1 50 ug/ml | 0.67 | 5.33 | 29.3 | 36.7 | 1.33 | 18 | 7.33 | 22.7 | 22.7 | 15.3 |
| Peptide pool 2 25 uM | 0.67 | 5.33 | 40 | 54.7 | 0.67 | 22.7 | 8.67 | 18.7 | 18.7 | 20 |
| Peptide pool 3 25 uM | 0 | 6 | 42.7 | 58.7 | 4 | 40.7 | 18 | 18.7 | 18.7 | 24.7 |
| Total gluten pool 10 ug/ml | 3.33 | 5.33 | 9.33 | 35.3 | 2 | 27.3 | 4.67 | 20.7 | 20.7 | 14 |
| Medium | 0.25 | 0.25 | 0.75 | 2.5 | 0 | 1.25 | 0.5 | 0.75 | 0.5 | 0 |
| Peptide 1 10 ug/mL | 2 | 4 | 2 | 8 | 0 | 4 | 1 | 0 | 0 | 1 |
| Peptide 2 10 ug/mL | 0 | 1 | 2 | 17 | 0 | 2 | 2 | 4 | 1 | 0 |
| Peptide 3 10 ug/mL | 0 | 1 | 9 | 30 | 0 | 11 | 1 | 9 | 3 | 1 |
| Peptide 4 5 uM | 1 | 1 | 18 | 36 | 0 | 16 | 6 | 17 | 0 | 11 |
| Peptide 5 5 uM | 1 | 2 | 3 | 16 | 0 | 3 | 1 | 2 | 0 | 3 |
| Peptide 6 5 uM | 0 | 2 | 3 | 7 | 0 | 0 | 1 | 3 | 0 | 6 |
| Peptide 7 5 uM | 0 | 1 | 2 | 20 | 0 | 5 | 1 | 0 | 3 | 1 |
| Peptide 8 5 uM | 0 | 1 | 0 | 4 | 0 | 1 | 0 | 0 | 0 | 1 |
| Peptide 9 5 uM | 1 | 0 | 8 | 23 | 1 | 5 | 1 | 3 | 2 | 3 |
| Peptide 10 5 uM | 0 | 7 | 2 | 6 | 0 | 5 | 0 | 1 | 0 | 0 |
| Peptide 11 5 uM | 0 | 2 | 4 | 11 | 0 | 3 | 2 | 4 | 0 | 3 |
| Peptide 12 5 uM | 1 | 1 | 0 | 0 | 1 | 0 | 0 | 1 | 0 | 0 |
| Peptide 13 5 uM | 0 | 0 | 3 | 1 | 1 | 1 | 0 | 1 | 1 | 1 |
| Peptide 14 5 uM | 0 | 0 | 0 | 1 | 0 | 1 | 2 | 1 | 0 | 0 |
| Peptide 15 5 uM | 0 | 0 | 1 | 0 | 0 | 50 | 0 | 1 | 0 | 0 |
| Peptide 16 5 uM | 1 | 2 | 1 | 2 | 0 | 0 | 0 | 0 | 0 | 0 |

### Example 2

### Peptide Compositions and Detection Assays for Gluten-Reactive T Cells in Celiac Disease

Peptide selection is important to the design of epitope-specific immunotherapy (ESIT) and epitope-specific immunodiagnostics (ESID).¹ ESIT's and ESID's require selection of peptides with epitopes that are recognized by a substantial proportion of the CD4+ T cells responsible for pathology.

The frequency and hierarchy of gluten-specific T cells in vivo can be quantified in overnight IFNγ ELISpot assays using freshly isolated peripheral blood mononuclear cells (PBMC) collected after patients undergo oral gluten challenge.^{2,3} Optimizing minimal peptide compositions generally requires not only quantitative assays, but also establishing that epitopes are mostly non-redundant, i.e. that each peptide targets distinct T cell populations that together consistently account for a substantial proportion of pathogenic T cells in patients. In celiac disease, circulating gluten-reactive T cells are extremely rare and so far have not been detected by quantitative cytokine release assays except after oral gluten challenge.² However, oral challenge with wheat, barley, and rye reactivates gluten-reactive T cell populations with subtly different specificities that allowed selection of the three peptides included in P3.

The three peptides in pool P3 (containing Peptides 1, 2, and 3 in Table 9) constitute at least five, mostly non-redundant HLA-DQ2.5-restricted epitopes. IFNγ ELISpot studies using blood collected after oral gluten challenge indicate that ex vivo T cell responses to an optimal concentration of P3 (3×50 μg/mL) is about ⅔ of that elicited by peptic digests of semi-purified gliadin, hordein or the most active secalin fraction (ω-secalin) pre-treated with transglutaminase.³

In vitro studies with T cell clones specific for P3 indicate that the five defined epitopes in P3 are relatively non-redundant, and recognize over two-thirds of the 90 wheat, barley and rye prolamin-derived peptides confirmed to be T-cell stimulatory in HLA-DQ2.5+CD patients.³ However, there are some relatively potent T-cell stimulatory gluten-derived peptides that are not recognized by T cell clones specific for P3. This suggests that additional peptides could be added to P3 to increase the size of the responding gluten-reactive T cell population present in HLA-DQ2.5+ celiac disease patients.

Two strategies would be expected to increase the population of responding T cells: (1) selecting non-redundant HLA-DQ2.5 epitopes not already covered by P3, and (2) selecting peptides with gluten-derived epitopes restricted by celiac disease-associated HLA-DQ molecules apart from HLA-DQ2.5 (e.g. HLA-DQ8, HLA-DQ2.2 or transdimers formed between HLA-DQA and DQB chains of HLA-DQ2.5 and DQ8). The prevalence of HLA-DQ2.5 in patients confirmed to have celiac disease is typically about 90% (Table 6). But HLA-DQ2.2 or HLA-DQ8 is also present in about ⅓ of HLA-DQ2.5+ patients, and in patients not carrying HLA-DQ2.5, HLA-DQ8 or HLA-DQ2.2 are usually present.

TABLE 6
HLA-DQ genetics in celiac disease

| | %⁴ | %⁵ | %⁶ | %⁷ |
|---|---|---|---|---|
| HLA-DQ^{2.5+/any} | 91 | 91 | 94 | 88 |
| HLA-DQ^{2.5+/2.5+} | 13 | | 20 | |
| HLA-DQ^{2.5+/2.2+} | 28 | | 20 | |
| HLA-DQ^{2.5+/8+} | 4 | 9 | | |
| HLA-DQ^{2.5-/8+} | 7 | 5 | 2 | 6 |
| HLA-DQ^{2.5-/8-/2.2+} | 4 | 2 | 1 | |
| HLA-DQ^{2.5-/8+/2.2+} | 2 | | | |
| HLA-DQ^{2.5-/8-/2.2+} | 2 | | | 4 |
| HLA-DQ^{2.5-/8+/2.2-} | 5 | | | |

The hierarchy of wheat gluten peptides recognized by circulating T cells in HLA-DQ8+ 2.5-patients after wheat challenge has been reported,⁸ and is relatively consistent with in vitro studies using intestinal T cell lines.⁹⁻¹¹ Amongst the peptides recognized by circulating T cells in HLA-DQ8+ patients, three HLA-DQ8-restricted epitopes are efficiently presented by transdimers of HLA-DQ2.5 and 8.^{11,12} Only one HLA-DQ2.2-restricted epitope has been reported,¹³ but earlier the same sequence had also been claimed to activate T cell clones from HLA-DQ2.5+ donors.¹⁴ As described herein, addition of further epitopes to those in the three peptides in P3 was expected to increase T cell responses in patients who have HLA-DQ2.2 and/or 8 whether or not they also carry HLA-DQ2.5.

In principle, the activity of new peptide pools compared to P3 is readily measurable using optimal concentrations of peptides in ex vivo cytokine release assays with fresh polyclonal T cells circulating in blood after oral gluten challenge.

Quantifying the proportion of the pathogenic T cell population targeted is also likely to predict therapeutic efficacy and diagnostic accuracy of peptide compositions. In celiac disease, all pathogenic T cells are specific for gluten and the specificities of gluten-reactive T cells circulating after oral challenge with wheat, barley and rye have been exhaustively mapped.³

Although IFNγ ELISpot using fresh PBMC from patients following oral gluten challenge has been the mainstay of studies mapping and quantifying the importance of epitopes for circulating gluten-reactive T cells, other assay formats may be more sensitive for detection of rare antigen-specific T cells.¹⁵ The median frequency of effector memory T cells in blood from patients with treated celiac disease that are stained by tetramers for either the DQ2.5-gliaα1a or DQ2.5-gliaα2 epitopes is 5 per million CD4 T cells and in untreated patients 15 per million.¹⁶ The frequency of CD4 T cells in blood is 0.30-1.50 million/mL, implying that the frequency of T cells specific for Peptide 1, which contains the DQ2.5-gliaα1a or DQ2.5-gliaα2 epitopes (Peptide 1), is in the range 1.5-7.5/mL in treated celiac disease patients. Peptide 1 stimulates IFNγ secretion by cognate T cells, and in blood both monocytes and neutrophils are known to secrete the chemokine IP-10 when incubated with IFNγ.^{17,18} Monocytes and neutrophils are abundant in blood (0.20-0.90 million/mL and 2.09-5.97 million/mL, respectively). IP-10 plays an important role in the recruitment of T cells and monocytes to sites of inflammation.¹⁹ In principle, IP-10 in whole blood incubated with gluten peptides could be a sensitive, and more robust biomarker than IFNγ for the presence of activated gluten-specific T cells. Whole blood release of IP-10 is as sensitive as IFNγ for detection of T cells specific for Peptide 1 and Peptide 2 in celiac disease patients after oral gluten.¹⁵ Whole blood release of IP-10 may also be more sensitive than IFNγ to support the diagnosis of mycobacterium tuberculosis infection.^{20,21}

The primary objective of the current study was to test whether adding peptides to P3 could increase IFNγ and IP-10 secretion in cytokine release assays using fresh blood or PBMC. A secondary objective was to compare the sensitivity of whole blood IFNγ and IP-10 release assays for detection of gluten-reactive T cells before and after oral gluten challenge.

### Methods

### Clinical

Ten HLA-DQ2.5+ adults with celiac disease diagnosed according to the National Institutes of Health Consensus Statement 2004 were enrolled (Table 7).²² Subjects were required to have followed gluten-free diet for at least one year and without known gluten exposures within the previous two-months. Celiac disease-specific serology was also required to be no greater 50% above the upper level of normal, but in fact all subjects showed transglutaminase (tTG)-IgA (INOVA 704605 QUANTA Lite® R h-tTG IgA or 708760 QUANTA Lite® h-tTG IgA, San Diego, Calif. 92131) and deamidated gliadin peptide (DGP)-IgG (704520 QUANTA Lite® Gliadin IgG II (DGP)) within the normal range. Full inclusion and exclusion criteria are described in Table 8. Each of 3 consecutive days subjects consumed 3 cookies that were prepared from approximately 4.5 g wheat gluten, 3 g barley flour protein, and 1.5 g rye flour protein. Blood was collected before and six days after commencing the oral challenge. Blood was collected using a 21G butterfly needle directly into 10 mL lithium heparin tubes (BD Vacutainer® Heparin tube #367880) and QuantiFERON® NIL (0591-0205, Cellestis Ltd., Chadstone VIC 3148 Australia) and QuantiFERON® MITOGEN (0593-0201, Cellestis Ltd.).

TABLE 7
HLA-DQ and serology status of subjects

| | | HLA- | HLA- | | | | |
|---|---|---|---|---|---|---|---|
| | | DQA1 | DQB1 | HLA- | HLA- | tTG- | DGP- |
| Sex | Age | alleles | alleles | DQ#1 | DQ#2 | IgA¹ | IgG¹ |
| F | 51 | | 02:01, | 2.5 | 6 | <5* | <20 |
| | | | 06:03 | | | | |
| F | 49 | | 02:01, | 2.5 | 2.2 | <5* | <20 |
| | | | 02:02 | | | | |
| F | 41 | | 02:01/ | 2.5 | 2.2 | 1.2 | 2 |
| | | | 02:02 | | | | |
| M | 45 | 05:01 | 02:01, | 2.5 | 6 | 5.2 | 6 |
| | | | 06:09 | | | | |
| F | 48 | | 02:01, | 2.5 | 2.2 | <2* | 5 |
| | | | 02:02 | | | | |
| M | 47 | 02:01, 05:01 | 02:01, | 2.5 | 2.2 | 2.6 | 2 |
| | | | 02:02 | | | | |
| M | 43 | 01:02/08/09, | 02:01, | 2.5 | 6 | 7.6 | 4 |
| | | 05:01 | 06:02G | | | | |
| M | 36 | 01, 05 | 02, 06 | 2.5 | 6 | 12.5 | 3 |
| F | 42 | 01, 05 | 02, 06 | 2.5 | 6 | 5.3 | 1 |
| F | 48 | | 02, 0302 | 2 | 8 | 5.7 | 4 |

TABLE 8
Entry Criteria

Inclusion criteria:
a. Aged between 18 and 50 years.
b. HLA-A1*05 and HLA-B1*02 present (HLA-2.5+).
c. Celiac disease without Type-1 diabetes. Celiac disease diagnosed according to
National Institutes of Health Consensus Statement 2004 (Department of Health and
Human Services, 2004): small bowel histology showing at least villous atrophy, and
serology showing elevated transglutaminase IgA or abnormal endomysial
immunofluorescence while gluten is being regularly consumed.
d. Following strict gluten free diet.
e. Willing to consume an amount of gluten equivalent to approximately 4 slices of
bread daily for three days.
f. Provide written informed consent.
Exclusion criteria
a. Individual has not been prescribed and/or has not followed a GFD for at least 12
months or has had known gluten exposure within two months prior to screening.
b. Subject with elevation in transglutaminase [tTG]-IgA, deamidated gliadin peptide
[DGP]-IgA or IgG to a level ≧50% above upper limit of normal range for that assay.
c. Individual has uncontrolled complications of celiac disease or unstable
autoimmune disease which, in the opinion of the investigator, would impact the immune
response or pose an increased risk to the patient.
d. Individual has had treatment with systemic biological agents (e.g., adalimumab,
etanercept, infliximab, certolizumab pegol) less than six months prior to screening.
e. Individual has taken systemic immunomodulatory agents (e.g., azathioprine,
methotrexate) less than 30 days prior to screening.
f. Human immuno-deficiency virus-1 and -2 (HIV1 + 2) infection or active, untreated
hepatitis B virus (HBV) or hepatitis C virus (HCV) infection.

### Peptides

The P3 pool was prepared in sterile normal saline from acetate salts of Peptides 1, 2 and 3 (purities >98%; CSBio Menlo Park, Calif. 94025) to yield a stock equimolar solution (0.7 mL/vial stored at −20° C.) with constituent peptides at a concentration of 15.6 mM (MicroTest Laboratories, Inc.; Agawam Mass. 01001) (Table 9). The P14 pool (Table 9) included trifluoroacetate (TFA) salts of 14 peptides between 14 and 19 amino-acids (Mᵣ median: 1801.6, range: 1601.7-2228.6 g/mol) (Pepscan Presto BV, 8243 RC Lelystad, The Netherlands). Identities of constituent peptides were confirmed by LC/MS. Median purity assessed by HPLC was 97.4% (range: 95.0-99.8%). P14 was constituted as a lyophilized mixture in vials containing 0.2 μmol of each peptide that was stored at −20° C. until being dissolved directly in sterile normal saline yielding 5 mM per peptide. The P13 (Table 9) stock solution (13×1.556 mM) consisted of P3 diluted by the addition of the other 10 constituent peptides (Pepscan) individually dissolved in normal saline. P71 was prepared as a PepMix™ Peptide Pool from TFA salts of 71 individual peptides 14 to 19 amino acids in length (Mᵣ median: 1688.85, range: 1423.51-2229.48 g/mol) (JPT Peptide Technologies GmbH, 12489 Berlin, Germany). The identity of constituent peptides was confirmed by LC/MS, and median purity assessed by HPLC was 86% (range: 71-98.8%). Individual vials of lyophilized P71 containing 0.1 mg of each peptide were stored at 20° C. until being first dissolved in dimethylsulfoxide (DMSO) (10 mg/mL per peptide) then diluted to 1 mg/mL in sterile normal saline. The CEF pool of 23 peptides consisting of MHC class I-restricted T-cell epitopes from human cytomegalovirus, Epstein Barr virus and influenza virus was purchased from Mabtech (#3615-1; Nacka Strand, Sweden). Each vial of CEF contained 0.1 mL of a 10% DMSO aqueous solution with each peptide at a concentration of 0.2 mg/mL (individual peptide purities were >95%) that was stored at −20° C. Individual gluten peptides were dissolved directly in normal saline to 5 mM. Individual peptides and pools were further diluted in PBS and DMSO to achieve a DMSO concentration of 1%, and 10× the final assay concentration of peptide. All peptides referred to as “Peptide X” discussed in Example 2 refer to those in Table 9.

TABLE 9
Peptide Pools

| | P3 | P13 | P14 | P13alt | | HLA-DQ | Epitope |
|---|---|---|---|---|---|---|---|
| Peptide | pool | pool | pool | pool | Sequence | Restriction | sequences |
| 1 | Present | Present | Absent | Present | (pE)LQPFPQPE | DQ2.5 | PFPQPELPY |
| | | | | | LPYPQPQ-amide | | (SEQ ID NO: 1), |
| | | | | | (SEQ ID NO: 66) | | PQPELPYPQ |
| | | | | | | | (SEQ ID NO: 2) |
| 2 | Present | Present | Present | Present | (pE)QPFPQPEQ | DQ2.5 | PFPQPEQPF |
| | | | | | PFPWQP-amide | | (SEQ ID NO: 3), |
| | | | | | (SEQ ID NO: 67) | | PQPEQPFPW |
| | | | | | | | (SEQ ID NO: 4) |
| 3 | Present | Present | Absent | Present | (pE)PEQPIPEQP | DQ2.5 | EQPIPEQPQ |
| | | | | | QPYPQQ-amide | | (SEQ ID NO: 5), |
| | | | | | (SEQ ID NO: 68) | | PIPEQPQPY |
| | | | | | | | (SEQ ID NO: 6) |
| 4 | Absent | Present | Present | Present | (pE)QPFPQPEQ | DQ2.5/2.5 + 8/8 | PFPQPEQPI |
| | | | | | PIPVQPEQS-amide | | (SEQ ID NO: 7), |
| | | | | | (SEQ ID NO: 69) | | PQPEQPIPV |
| | | | | | | | (SEQ ID NO: 8), |
| | | | | | | | EQPIPVQPE |
| | | | | | | | (SEQ ID NO: 9) |
| 5 | Absent | Present | Present | Present | (pE)QPFPQPEQ | DQ2.5/2.5 + 8/8 | PFPQPEQPT |
| | | | | | PTPIQPEQP-amide | | (SEQ ID NO: 10), |
| | | | | | (SEQ ID NO: 70) | | PQPEQPTPI |
| | | | | | | | (SEQ ID NO: 11), |
| | | | | | | | EQPTPIQPE |
| | | | | | | | (SEQ ID NO: 12) |
| 6 | Absent | Present | Present | Present | (pE)QPFPQPEQ | DQ2.5/2.5 + 8/8 | PFPQPEQPF |
| | | | | | PFPLQPEQP-amide | | (SEQ ID NO: 3), |
| | | | | | (SEQ ID NO: 72) | | PQPEQPFPL |
| | | | | | | | (SEQ ID NO: 13), |
| | | | | | | | EQPFPLQPE |
| | | | | | | | (SEQ ID NO: 14) |
| 7 | Absent | Present | Present | Absent | (pE)QPFPQPEQ | DQ2.5 | PFPQPEQPF |
| | | | | | PFSQQ-amide | | (SEQ ID NO: 3), |
| | | | | | (SEQ ID NO: 73) | | PQPEQPFSQ |
| | | | | | | | (SEQ ID NO: 15) |
| 8 | Absent | Present | Present | Absent | (pE)PQPYPEQP | DQ2.5 | PYPEQPQPF |
| | | | | | QPFPQQ-amide | | (SEQ ID NO: 16) |
| | | | | | (SEQ ID NO: 74) | | |
| 9 | Absent | Present | Present | Present | (pE)SGEGSFQP | DQ8/2.5 + 8/8 | EGSFQPSQE |
| | | | | | SQENPQ-amide | | (SEQ ID NO: 17) |
| | | | | | (SEQ ID NO: 77) | | |
| 10 | Absent | Present | Present | Present | (pE)GQQGYYP | DQ2.5/2.5 + 8/ | QGYYPTSPQ |
| | | | | | TSPQQSG-amide | 8 | (SEQ ID NO: 18) |
| | | | | | (SEQ ID NO: 78) | | |
| 11 | Absent | Present | Present | Present | (pE)PEQPEQPF | DQ2.5/2.5 + 8/ | EQPEQPFPE |
| | | | | | PEQPQQ-amide | 8/2.2 + 8 | (SEQ ID NO: 19), |
| | | | | | (SEQ ID NO: 79) | | EQPFPEQPQ |
| | | | | | | | (SEQ ID NO: 20) |
| 12 | Absent | Present | Present | Absent | (pE)QPFPEQPE | DQ2.5 | PFPEQPEQI |
| | | | | | QIIPQQP-amide | | (SEQ ID NO: 21) |
| | | | | | (SEQ ID NO: 80) | | |
| 13 | Absent | Present | Present | Present | (pE)QPPFSEQE | DQ2.2 | PFSEQEQPV |
| | | | | | QPVLPQ-amide | | (SEQ ID NO: 22) |
| | | | | | (SEQ ID NO: 81) | | |
| 14 | Absent | Absent | Present | Absent | (pE)PEQPFPEQ | DQ2.5 | EQPFPEQPI |
| | | | | | PIPEQPQPYP- | | (SEQ ID NO: 23), |
| | | | | | amide | | PFPEQPIPE |
| | | | | | (SEQ ID NO: 69) | | (SEQ ID NO: 24), |
| | | | | | | | EQPIPEQPQ |
| | | | | | | | (SEQ ID NO: 5), |
| | | | | | | | PIPEQPQPY |
| | | | | | | | (SEQ ID NO:6) |
| 15 | Absent | Absent | Present | Present | (pE)QPYPQPEL | DQ2.5 | PYPQPELPY |
| | | | | | PYPQPQ-amide | | (SEQ ID NO: 25), |
| | | | | | (SEQ ID NO: 75) | | PQPELPYPQ |
| | | | | | | | (SEQ ID NO: 2) |
| 16 | Absent | Absent | Present | Absent | (pE)QPFPQPEL | DQ2.5 | PFPQPELPY |
| | | | | | PYPYPQ-amide | | (SEQ ID NO: 1), |
| | | | | | (SEQ ID NO: 76) | | PQPELPYPY |
| | | | | | | | (SEQ ID NO: 26) |
| 17 | Absent | Absent | Absent | Present | (pE)PQEQPFPE | DQ2.5 | EQPFPEQPI |
| | | | | | QPIPEQP-amide | | (SEQ ID NO: 23), |
| | | | | | (SEQ ID NO: 82) | | PFPEQPIPE |
| | | | | | | | (SEQ ID NO: 24) |
| 18 | Absent | Absent | Absent | Present | (pE)QPQPYPEQ | DQ2.5 | PQPYPEQPQ |
| | | | | | PQPFPQQ-amide | | (SEQ ID NO: 27), |
| | | | | | (SEQ ID NO: 83) | | PYPEQPQPF |
| | | | | | | | (SEQ ID NO: 16) |

Peptide = Peptide identifier, (pE) = pyroglutamate, Present = present in the pool listed in the top row
(P3, P13, P14, or P13alt), Absent = not present in the pool listed in the top row (P3, P13, P14, P13alt).

### Cytokine Release Assays

### 96-Well Plate Whole Blood Multiplex Cytokine Release

Gluten peptide solutions for addition to whole blood were arranged according to standardized templates in sterile 96-well U-bottom plates (30 μL/well). Plates were sealed with adhesive ELISA plate cover slips before the plastic lid was replaced and being frozen at −80° C. Whole blood assay (WBA) “medium” (1% DMSO in 90% PBS and 9% normal saline), mitogen (PHA-L, Sigma-Aldrich #L2769; St Louis Mo.) 100 μg/ml in WBA medium, and CEF diluted to 10 μg/ml in PBS with a final concentration of 1% DMSO were frozen separately in sterile cryovials. 96-well plates and cryovials with frozen incubation solutions were shipped on dry ice to the clinical site where blood was collected and incubations performed. Solutions were thawed at room temperature for 10 min just before being added to whole blood. 96-well plates were thawed while being centrifuged at 300 g to avoid surface condensation. A multi-channel pipette was used to efficiently transfer 25 μL of peptide solutions from the original 96-well plate to corresponding wells in a fresh sterile 96-well U-bottom plate. Once incubation solutions had been added, 225 μL of whole blood was added to each well and the plate transferred to incubate for 24 h at 37° C. 5% CO₂. DMSO was present in all assays at a final concentration of 0.1%, the highest concentration tested that did not reduce antigen-stimulated whole blood IFNγ or IP-10 secretion (data not shown). Individual peptides were incubated at a final assay concentration of 5 μM. Final concentrations of individual peptides assessed in P3 was 0.05-50 μg/mL, and in P14 and P13 pools 0.025-25 μM. The P71 pool was tested at between 0.005-10 μg/mL, the highest tested concentration being the maximal possible not exceeding 0.1% DMSO. Peptide pools, mitogen, and medium only were assessed in triplicate wells, and on Day-6 individual peptides were assessed in duplicate wells. Incubations were terminated after 24 h, and 96-well plates centrifuged at 300 g for 10 min. A total of approximately 90 to 120 μL of plasma was removed from each well taking care to avoid red cell contamination, and transferred into corresponding wells of two further 96-well plates (one with 30 μL/well and the residual in the second plate). Plasmas were frozen at −80° C. and later shipped on dry ice to the central lab where IP-10 and IFNγ multiplex bead assays were performed according to manufacturer's instructions using 25 μL plasma per well (Milliplex® MAP Human Cytokine/Chemokine Magnetic Bead Panel #HCYTOMAG-60k-02; EMD Millipore Corp., Billerica, Mass. 01821) and analysed with the Luminex® MAGPIX® System xPONENT® software (Luminex Corporation, Austin Tex. 78727). Plasma cytokine levels for each assay condition were expressed as the mean analyte concentration of each of the three replicate incubations. Laboratory staff were unaware of the arrangement of peptide solutions incubated with during assay setup and plate counting.

### In-Tube Whole Blood IFNγsecretion

Aliquots of PBS, P3 peptides diluted in PBS (final concentration in blood 3×50 μg/mL), or CEF to a final concentration 1 μg/mL were prepared in sterile cryovials (0.11 mL/vial) and stored frozen (−80° C.) until immediately before use. P3, CEF and two PBS aliquots were drawn up in separate sterile 0.3 mL insulin syringes with attached 29G× ½″ needle (Terumo, SS*30M2913; Elkton Md. 21921). P3, CEF and one PBS aliquot was injected through the stoppers of three separate QuantiFERON® NIL tubes already containing 1 mL blood, and one aliquot of PBS was injected into a QuantiFERON® MITOGEN tube already containing 1 mL blood. All four tubes containing blood and incubation solution were gently inverted ten-times and transferred to incubate at 37° C. 5% CO₂. After for 24 h, tubes were centrifuged 300 g 10 min. Plasma was separated and equal volumes placed in two 1.5 mL cryovials. Frozen plasmas were transferred to the central lab where IFNγ levels were measured in triplicate 50 μL samples by ELISA (MABtech Human IFN-γ ELISA development kit HRP, 3420-1H-6; capture mAb 1-D1K). IFNγ responses were considered elevated if levels were more than 7.2 pg/mL greater than in the medium only tube, and the ratio between IFNγ levels in the NIL tube with P3 or CEF to the NIL tube with PBS only (stimulation index, SI) was >1.25.

### ELISpot Assay

Overnight IFNγ ELISpot assays (Human IFN-γ ELISpotPRO kit, transparent, ALP; Mabtech AB, #3420-2APT-10) were performed using PBMC freshly separated from heparinized blood diluted 1:1 in PBS with 2% fetal bovine serum (Stemcell Technologies #07905; Vancouver, BC, V5Z 1B3, Canada) overlaid on density gradient medium (Ficoll-Paque™ PLUS; GE Healthcare Lice Sciences #:17-1440-02) in SepMate™-50 tubes (Stemcell Technologies #15460). PBMC (8 million/mL) were resuspended in serum-free medium with gentamicin and phenol red (X-VIVO™15; Lonza, Walkersville Mass. 21793). PBMC (0.4 million/50 μL/well) and incubated with 50 μL 80% X-vivol5, 0.2% DMSO and 20% PBS in three triplicate wells (“medium only”), or with 50 μL PHA-L 20 μg/ml or peptide at 2× final concentration in 80% X-vivol5, 0.2% DMSO and 20% PBS. Peptide solutions to be added to individual ELISpot wells were prepared in 96-well U-bottom plates (60 μL/well) that were then sealed with adhesive ELISA plate cover slips before replacing the plastic lid and being frozen at −80° C. 96-well plates containing peptide solutions were thawed at room temperature for 10 min while being centrifuged at 300 g immediately prior to being added to ELISpot wells. Peptide pools and mitogen were assessed in triplicate wells, and individual peptides were assessed in duplicate wells. X-VIVO™15 (50 μL) was incubated with 80% X-vivol5, 0.2% DMSO and 20% PBS (50 μL) in triplicate wells (“no PBMC” control). A Zeiss automated ELISpot counter was used to determine spot forming units (SFU) per well (Zellnet Inc., Fort Lee, N.J. 07024). Laboratory staff were unaware of the arrangement of peptide solutions in ELISpot wells during assay setup and plate counting.

### Results

### Design and Preparation of Gluten Peptide Pools

There is presently no functional assay that enumerates all gluten-reactive T cells relevant to celiac disease. Wheat gluten is a variable mixture of aqueous insoluble proteins that requires digestion by proteases and deamidation to be efficiently activate T cells in patients with celiac disease. Hordeins from barley and secalins from rye are also complex mixtures of proteins closely related to wheat gluten that harbor potent CD4+ T cell epitopes that are not represented in wheat gluten. Furthermore gluten contains other proteins such as amylase trypsin inhibitors (ATI's) that activate innate immune cells and may compromise interpretation of functional immuno-assays.²³ Whole protein and mixtures of overlapping peptide spanning a protein antigen's primary sequence both efficiently reactivate recall CD4+ and CD8+ T-cell responses to CMV and HIV antigens in vitro.^{24,25} A pool of synthetic peptides including a comprehensive set of epitopes implicated in celiac disease could be used to stimulate virtually all gluten-reactive T-cells.

After comprehensive, unbiased epitope screening, only 90 14-16mer peptides were confirmed to be stimulatory for T cells circulating in blood after oral challenge with wheat, barley or rye in HLA-DQ2.5+ patients with celiac disease.³ A pool consisting of 71 peptides (P71) was designed to include each of the core immunogenic sequences in the 90 immunogenic peptides implicated in HLA-DQ2.5+ celiac disease as well as three HLA-DQ8- and one HLA-DQ2.2-restricted epitopes. Peptides were 14 to 19 amino acids in length with their N-terminals “capped” by N-acetylation or by the presence of N-pyroglutamate, and C-terminals were amidated. All glutamine residues in native gluten-derived sequences that were predicted to be susceptible to human transglutaminase-2 were replaced with glutamate residues.²⁶ Peptides were combined to yield a final lyophilized mixture of all 71 peptides. Unlike the smaller gluten peptide pools, the P71 was not immediately soluble in normal saline but did dissolve in sterile DMSO to 10 mg per peptide/mL before being diluted in phosphate buffered saline to desired concentrations. Amongst the 71 peptide sequences in P71, 14 were synthesized separately to greater than 95% purity and combined in three different mixtures: P3 (3 peptides, 15-16 amino acids), P14 (14 peptides, 14-19 amino acids) and P13 (13 peptides, 14-18 amino acids) (Table 9). An alternative equimolar solution of 13 peptides corresponding to Peptides 1, 2, and 3 and 10 of the peptides in P14 was prepared from individual peptide solutions initially in normal saline (P13alt).

Peripheral Blood Responses to Gluten Peptide Pools after Oral Gluten Challenge

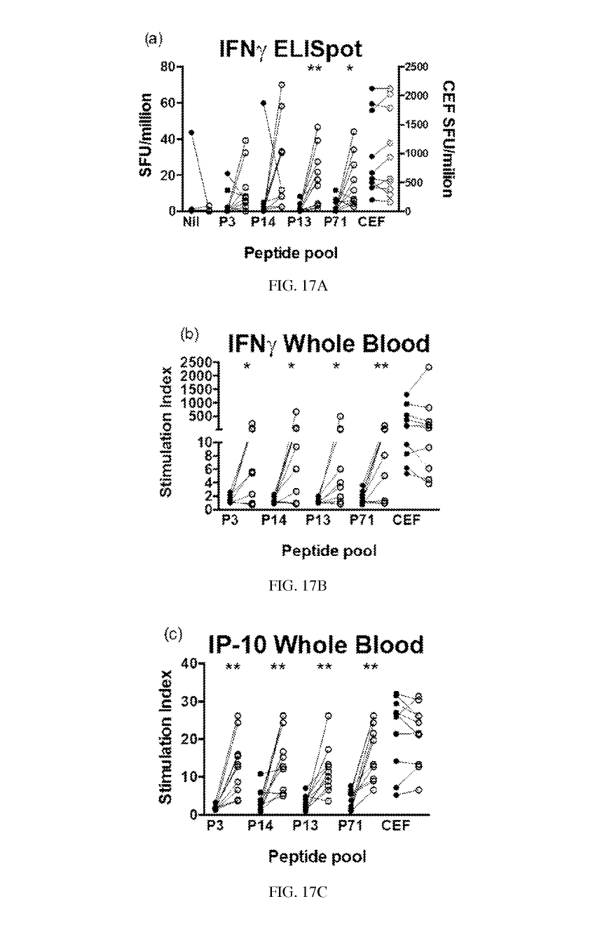

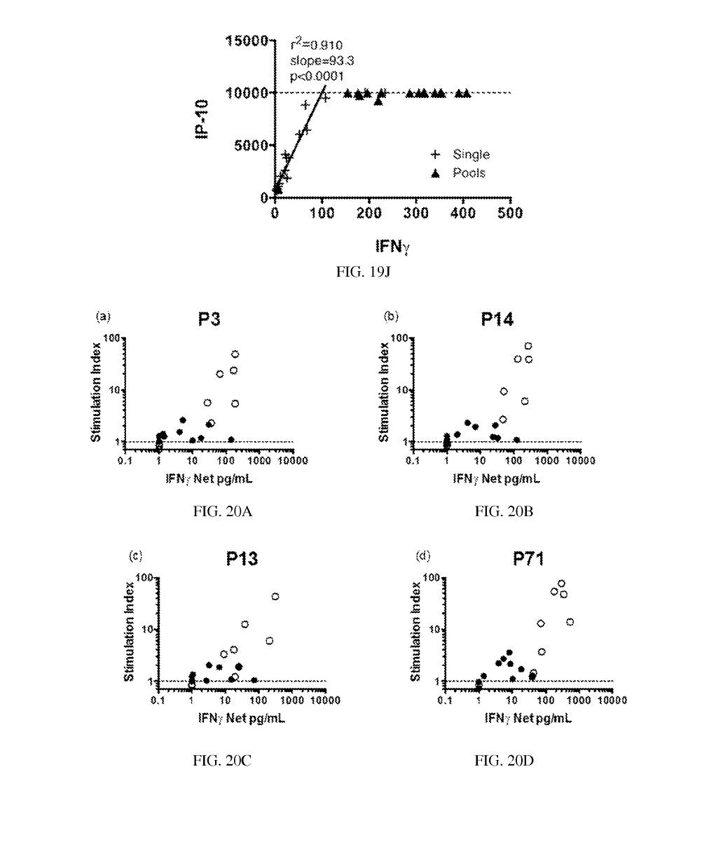

Whole blood IP-10 release stimulated by each of the four gluten peptide pools (P3, P13, P14, and P71) was increased in all ten subjects after oral gluten challenge (FIG. 17C, Table 10, FIG. 20E-H) and consistently reached statistical significance. In contrast, the change in IFNγ responses to gluten peptide pools after oral challenge were not as pronounced or as consistent as IP-10 (FIGS. 17A and B). Four subjects failed to mount IFNγ ELISpot responses greater than 10 SFU/1.2 million PBMC or show an increased response after oral gluten challenge (subjects 1, 2, 5, and 7), and three showed no increase in IFNγ response to gluten peptide pools in 96-well whole blood assay formats (1, 2 and 5). Subjects 1 and 2 were also negative when IFNγ release was measured by ELISA in plasma from whole blood collected after gluten challenge that had been incubated with P3 in QuantiFERON® NIL tubes (Table 11). All subjects responded strongly to the recall MHC Class I epitope pool CEF in all four of IFNγ and IP-10 assay formats, but overall there was no statistically significant change after oral gluten challenge (FIG. 17).

TABLE 10
Responses to gluten peptide pools in whole blood cytokine release assays
Response relative to medium only - Stimulation Index (median, 95% confidence interval)

| Assay | Sample | N | P3 10 μg/mL | P14 5 μM | P13 5 μM | P71 10 μg/mL |
|---|---|---|---|---|---|---|
| IFNγ | Day-0 | 10 | 1.3 (1.1-2.1) | 1.3 (1.1-2.1) | 1.2 (1.0-2.0) | 1.5 (1.0-2.7) |
| IFNγ | Day-6 | 10 | 5.5 (0.8-49) | 7.7 (0.92-70) | 3.7 (0.9-43) | 13 (1.2-76) |
| IP-10 | Day-0 | 10 | 1.5 (1.2-2.0) | 3.1 (1.0-5.9) | 2.7 (1.1-4.8) | 4.6 (1.1-6.6) |
| IP-10 | Day-6 | 10 | 13 (3.8-24) | 14 (5.6-26) | 10.69 (6.5-17) | 17 (8.9-26) |

TABLE 11
Day-6 whole blood IFNγ release by ELISA

| | NIL | P3^{#} | P3- | P3 | P3 | CEF^{##} | CEF |
|---|---|---|---|---|---|---|---|
| Subject | pg/mL | pg/mL | NIL | SI | response* | pg/mL | SI |
| 1 | 2.0 | 5.7 | 3.8 | 2.9 | − | 297 | 152 |
| 2 | 9.6 | 12 | 1.4 | 1.1 | − | 654 | 68 |
| 3 | 6.5 | 130 | 124 | 20 | + | 198 | 30 |
| 4 | 2.0 | 100 | 98 | 51 | + | 226 | 116 |
| 5 | 4.6 | 17 | 12 | 3.6 | + | 925 | 203 |
| 6 | 2.6 | 30 | 28 | 12 | + | 1810 | 707 |
| 7 | 5.6 | 28 | 23 | 5.0 | + | 2000 | 357 |
| 8 | 2.2 | 48 | 45 | 21 | + | 93 | 42 |
| 9 | 28 | 102 | 74 | 3.6 | + | 1110 | 39 |
| 10 | 2.0 | 65 | 63 | 33 | + | 354 | 181 |

^{#}P3 50 μg/mL;
^{##}CEF 1 μg/mL;
*Positive response to P3 is defined as P3-NIL pg/mL >7.2 and P3/NIL >1.25

### Peripheral Blood Responses to Individual Gluten Peptides

The baseline frequency of memory T cells specific for gluten epitopes and the relative dose of epitopes presented after oral challenge would be expected to determine the number and relative frequencies of gluten epitope-specific T cells circulating after oral gluten challenge. Cytokine release responses to the 16 constituent peptides in P3, P14 and P13 were compared using blood collected after oral gluten challenge (Table 12 and 13).

TABLE 12
Peptide pool compositions and responses to individual gluten peptides

| | | | | IFNγ WB | IP-10 WB | |
|---|---|---|---|---|---|---|
| | | | | MAGPIX | MAGPIX | IFNγ ELISpot |
| | | | | % max | % max | % max |
| | | | HLA-DQ | Mean | Mean | Mean |
| P3 | P14 | P13 | restriction | (rank)/n = 8 | (rank)/n = 10 | (rank)/n = 5 |
| | Peptide | | DQ2.5 | 94 (1) | 82 (1) | 100 (1) |
| | 14 | | | | | |
| Peptide 3 | | Peptide 3 | DQ2.5 | 62 (2) | 65 (2) | 53 (2) |
| Peptide 1 | | Peptide 1 | DQ2.5 | 33 (3) | 58 (3) | 13 (10) |
| | Peptide | | DQ2.5 | 31 (4) | 42 (7) | 24 (5) |
| | 16 | | | | | |
| | Peptide 6 | Peptide 6 | DQ2.5/2.5 + 8/8 | 28 (5) | 43 (6) | 21 (8) |
| | Peptide | | DQ2.5 | 27 (6) | 49 (4) | 25 (4) |
| | 2* | | | | | |
| | Peptide | | DQ2.5 | 25 (7) | 33 (10) | 13 (11) |
| | 15 | | | | | |
| | Peptide 8 | Peptide 8 | DQ2.5 | 25 (8) | 42 (8) | 37 (3) |
| Peptide | | Peptide 2 | DQ2.5 | 23 (9) | 43 (5) | 19 (9) |
| 2* | | | | | | |
| | Peptide 4 | Peptide 4 | DQ2.5/2.5 + 8/8 | 15 (10) | 38 (9) | 24 (6) |
| | Peptide 5 | Peptide 5 | DQ2.5/2.5 + 8/8 | 11 (11) | 25 (11) | 22 (7) |
| | Peptide 7 | Peptide 7 | DQ2.5 | 9 (12) | 12 (12) | 5 (13) |
| | Peptide | Peptide | DQ2.2 | 7 (13) | 10 (13) | 2 (16) |
| | 13 | 13 | | | | |
| | Peptide | Peptide | DQ2.5/2.5 + 8/8 | 4 (14) | 1 (15) | 8 (12) |
| | 10 | 10 | | | | |
| | Peptide 9 | Peptide 9 | DQ8/2.5 + 8/8 | 2 (15) | 0 (17) | 1 (17) |
| | Peptide | Peptide | DQ2.5 | 2 (16) | 3 (14) | 3 (15) |
| | 12 | 12 | | | | |
| | Peptide | Peptide | DQ2.5/2.5 + 8/8/ | 0 (17) | 1 (16) | 3 (14) |
| | 11 | 11 | 2.2 + 8 | | | |

*Peptide 2 was re-synthesised by Pepscan for inclusion in P14 and assessed at a concentration of 10 μg/mL (5.45 μM). Peptide 2 prepared by CSBio was tested at 5 μM, and used to prepare P3 and P13 pools. The two versions of Peptide 2 were assessed separately. Individual peptides are those identified in Table 9.

TABLE 13
Percent of maximal response to individual gluten peptides by subjects mounting
elevated responses in cytokine assays

| | Subject | | | | | | | | | | |
|---|---|---|---|---|---|---|---|---|---|---|---|
| | 3 | 3 | 3 | 10 | 10 | 10 | 4 | 4 | 4 | 2 | 2 |
| | Cytokine | | | | | | | | | | |
| | IFNg | IP-10 | ELISp | IFNg | IP-10 | ELISp | IFNg | IP-10 | ELISp | IFNg | IP-10 |
| | Units | | | | | | | | | | |
| | pg/mL | pg/mL | SFU | pg/mL | pg/mL | SFU | pg/mL | pg/mL | SFU | pg/mL | pg/mL |
| Max. | 799 | 9492 | 18 | 229 | 8922 | 11 | 136 | 9463 | 36 | 116 | 3646 |
| Peptide | 100 | 100 | 100 | 100 | 100 | 100 | 100 | 100 | 100 | 53 | 31 |
| 14 (% of | | | | | | | | | | | |
| max) | | | | | | | | | | | |
| Peptide 3 | 92 | 100 | 50 | 81 | 100 | 9 | 66 | 100 | 83 | 35 | 14 |
| (% of | | | | | | | | | | | |
| max) | | | | | | | | | | | |
| Peptide 1 | 14 | 100 | 11 | 7 | 34 | 9 | 26 | 96 | 22 | 100 | 100 |
| (% of | | | | | | | | | | | |
| max) | | | | | | | | | | | |
| Peptide | 13 | 100 | 22 | 2 | 11 | 27 | 16 | 69 | 31 | 65 | 45 |
| 16 (% of | | | | | | | | | | | |
| max) | | | | | | | | | | | |
| Peptide 6 | 10 | 100 | 11 | 20 | 56 | 9 | 54 | 100 | 56 | 58 | 21 |
| (% of | | | | | | | | | | | |
| max) | | | | | | | | | | | |
| Peptide 2 | 17 | 100 | 0 | 26 | 87 | 18 | 51 | 100 | 89 | 33 | 33 |
| (% of | | | | | | | | | | | |
| max) | | | | | | | | | | | |
| Peptide | 5 | 100 | 11 | 7 | 17 | 0 | 24 | 67 | 17 | 46 | 30 |
| 15 (% of | | | | | | | | | | | |
| max) | | | | | | | | | | | |
| Peptide 8 | 24 | 100 | 44 | 45 | 94 | 27 | 39 | 100 | 64 | 35 | 5 |
| (% of | | | | | | | | | | | |
| max) | | | | | | | | | | | |
| Peptide 2 | 20 | 100 | 11 | 8 | 30 | 0 | 48 | 100 | 47 | 15 | 35 |
| (% of | | | | | | | | | | | |
| max) | | | | | | | | | | | |
| Peptide 4 | 13 | 100 | 17 | 11 | 30 | 27 | 24 | 100 | 44 | 33 | 19 |
| (% of | | | | | | | | | | | |
| max) | | | | | | | | | | | |
| Peptide 5 | 5 | 100 | 17 | 27 | 60 | 55 | 13 | 46 | 19 | 39 | 13 |
| (% of | | | | | | | | | | | |
| max) | | | | | | | | | | | |
| Peptide 7 | 2 | 60 | 0 | 1 | 3 | 9 | 8 | 20 | 11 | 60 | 7 |
| (% of | | | | | | | | | | | |
| max) | | | | | | | | | | | |
| Peptide | 3 | 70 | 6 | 0 | 0 | 0 | 0 | 0 | 6 | 47 | 16 |
| 13 (% of | | | | | | | | | | | |
| max) | | | | | | | | | | | |
| Peptide | 0 | 0 | 17 | 9 | 9 | 9 | 0 | 0 | 3 | 23 | 0 |
| 10 (% of | | | | | | | | | | | |
| max) | | | | | | | | | | | |
| Peptide 9 | 0 | 0 | 0 | 0 | 0 | 0 | 0 | 0 | 0 | 19 | 0 |
| (% of | | | | | | | | | | | |
| max) | | | | | | | | | | | |
| Peptide | 0 | 9 | 6 | 1 | 0 | 0 | 0 | 5 | 0 | 13 | 3 |
| 12 (% of | | | | | | | | | | | |
| max) | | | | | | | | | | | |
| Peptide | 0 | 6 | 0 | 0 | 0 | 0 | 0 | 2 | 3 | 0 | 0 |
| 11 (% of | | | | | | | | | | | |
| max) | | | | | | | | | | | |

Individual peptides are those identified in Table 9.

| | Subject | | | | | | | | | | | |
|---|---|---|---|---|---|---|---|---|---|---|---|---|
| | 6 | 6 | 6 | 9 | 9 | 8 | 8 | 8 | 7 | 7 | 5 | 1 |
| | Cytokine | | | | | | | | | | | |
| | IFNg | IP-10 | ELISp | IFNg | IP-10 | IFNg | IP-10 | ELISp | IFNg | IP-10 | IP-10 | IP-10 |
| | Units | | | | | | | | | | | |
| | pg/mL | pg/mL | SFU | pg/mL | pg/mL | pg/mL | pg/mL | SFU | pg/mL | pg/mL | pg/mL | pg/mL |
| Max. | 72 | 5863 | 16 | 67 | 8349 | 47 | 4019 | 17 | 17 | 2330 | 9336 | 2331 |
| Peptide | 100 | 100 | 100 | 98 | 100 | 100 | 91 | 100 | 99 | 100 | 100 | 2 |
| 14 (% of | | | | | | | | | | | | |
| max) | | | | | | | | | | | | |
| Peptide | 63 | 43 | 69 | 60 | 85 | 89 | 100 | 53 | 6 | 5 | 53 | 51 |
| 3 (% of | | | | | | | | | | | | |
| max) | | | | | | | | | | | | |
| Peptide | 25 | 23 | 25 | 43 | 75 | 6 | 3 | 0 | 43 | 39 | 14 | 100 |
| 1 (% of | | | | | | | | | | | | |
| max) | | | | | | | | | | | | |
| Peptide | 10 | 4 | 19 | 39 | 60 | 0 | 1 | 24 | 100 | 800 | 35 | 16 |
| 16 (% of | | | | | | | | | | | | |
| max) | | | | | | | | | | | | |
| Peptide | 4 | 0 | 31 | 69 | 100 | 6 | 8 | 0 | 2 | 1 | 8 | 34 |
| 6 (% of | | | | | | | | | | | | |
| max) | | | | | | | | | | | | |
| Peptide | 18 | 30 | 0 | 33 | 70 | 39 | 32 | 18 | 3 | 3 | 31 | 0 |
| 2 (% of | | | | | | | | | | | | |
| max) | | | | | | | | | | | | |
| Peptide | 5 | 0 | 31 | 100 | 69 | 4 | 5 | 6 | 6 | 6 | 33 | 4 |
| 15 (% of | | | | | | | | | | | | |
| max) | | | | | | | | | | | | |
| Peptide | 6 | 2 | 31 | 5 | 31 | 42 | 39 | 18 | 0 | 0 | 45 | 0 |
| 8 (% of | | | | | | | | | | | | |
| max) | | | | | | | | | | | | |
| Peptide | 3 | 3 | 13 | 72 | 100 | 2 | 6 | 24 | 14 | 11 | 16 | 29 |
| 2 (% of | | | | | | | | | | | | |
| max) | | | | | | | | | | | | |
| Peptide | 1 | 2 | 19 | 29 | 48 | 9 | 7 | 12 | 1 | 3 | 51 | 18 |
| 4 (% of | | | | | | | | | | | | |
| max) | | | | | | | | | | | | |
| Peptide | 1 | 1 | 0 | 0 | 10 | 0 | 4 | 18 | 6 | 2 | 13 | 0 |
| 5 (% of | | | | | | | | | | | | |
| max) | | | | | | | | | | | | |
| Peptide | 0 | 0 | 6 | 0 | 17 | 0 | 3 | 0 | 0 | 0 | 12 | 0 |
| 7 (% of | | | | | | | | | | | | |
| max) | | | | | | | | | | | | |
| Peptide | 5 | 5 | 0 | 0 | 0 | 0 | 0 | 0 | 1 | 0 | 6 | 0 |
| 13 (% of | | | | | | | | | | | | |
| max) | | | | | | | | | | | | |
| Peptide | 0 | 0 | 6 | 0 | 17 | 0 | 3 | 0 | 0 | 0 | 12 | 0 |
| 10 (% of | | | | | | | | | | | | |
| max) | | | | | | | | | | | | |
| Peptide | 0 | 0 | 0 | 0 | 0 | 0 | 0 | 6 | 0 | 0 | 0 | 0 |
| 9 (% of | | | | | | | | | | | | |
| max) | | | | | | | | | | | | |
| Peptide | 0 | 0 | * | 0 | 1 | 0 | 0 | 6 | 0 | 0 | 4 | 11 |
| 12 (% of | | | | | | | | | | | | |
| max) | | | | | | | | | | | | |
| Peptide | 0 | 0 | 6 | 0 | 0 | 0 | 0 | 6 | 0 | 0 | 0 | 0 |
| 11 (% of | | | | | | | | | | | | |
| max) | | | | | | | | | | | | |

* indicates that the ELISpot response to Peptide 12 was negative in one of two duplicate ELISpot assays in this subject

The most active peptide tested was a barley-derived 19mer, Peptide 14, which corresponds to the partially deamidated sequence of B1 hordein (Genbank CAA60681.1) residues 21 to 37 with the addition of pyroGlu-Pro (ZP) at the N-terminal and an amide group at the C-terminal (*): ZPEQPFPEQPIPEQPQPYP*. Peptide 3 was consistently the second most active peptide after Peptide 14. IFNγ ELISpot responses to Peptide 3 10 μg/mL (5.3 μM) were only one-half of those to Peptide 14 5 μM. In whole blood incubations with Peptide 3, IP-10 release was one-quarter less and IFNγ release one-third less than that stimulated by Peptide 14. Peptide 14 is closely related to Peptide 3. Peptide 14 differs from Peptide 3 by the insertion of EQPFP following N-pyroGlu-Pro and by removal of the C-terminal di-glutamine. The most likely explanation for Peptide 14's greater activity is that it comprises two additional epitopes (EQPFPEQPI (SEQ ID NO: 23), and PFPEQPIPE (SEQ ID NO: 24)) further to those in Peptide 3 (EQPIPEQPQ (SEQ ID NO: 5) and PIPEQPQPY (SEQ ID NO: 6)). Indeed, deamidation of the peptide named B16 QQQPFPQQPIPQ (SEQ ID NO: 155) by transglutaminase was confirmed to generate an immunogenic sequence recognized by circulating T cells after oral challenge of HLA-DQ2.5 CD subjects with pure barley.³ The sequences EQPFPEQPI (SEQ ID NO: 23) and PFPEQPIPE (SEQ ID NO: 24) were also predicted to be candidate HLA-DQ2.5-restricted epitopes.²⁷

Half the daily prolamin dose in the oral gluten challenge was wheat gluten (approximately half as gliadins and half as glutenins), one third was from barley and one sixth was from rye. Despite the substantial load of wheat gluten, peptides (Peptide 1, 15 and 16) comprising overlapping epitopes dominant after oral wheat challenge (DQ2.5-glia-α1a, DQ2.5-glia-α1b and DQ2.5-glia-α2) were no more than two-thirds as active as Peptide 14. Peptide 1, which includes DQ2.5-glia-α1a and DQ2.5-glia-α2 epitopes, was the most active of this group of related peptides.

Peptides 2, 6 and 8 were each approximately one-half to one-fifth as active as Peptide 14 in the three cytokine release assays. These peptides comprised sequences known to be HLA-DQ2.5-restricted epitopes. Peptide 6 also encompassed sequences recognized by T cells in blood after oral challenge in HLA-DQ8+ celiac disease subjects. Peptides 4 and 5 were from one-half to one-eighth as active as Peptide 14. They had been included in the larger pools because of their contribution to the T cell response after oral gluten challenge in patients with celiac disease who are HLA-DQ2.5+ and/or HLA-DQ8+. Peptides 7 and 12 stimulated cytokine release that was on average no more than 10% of that to Peptide 14. This finding was at odds with previous findings following oral challenges with pure wheat or rye.

Cytokine release stimulated by peptides comprising important HLA-DQ8- or HLA-DQ2.2-restricted epitopes (Peptides 9, 10, 11, and 13) was weak or not distinguishable from medium alone. However, HLA-DQ8+ and DQ2.2+ subjects who do not also carry HLA-DQ2.5 will be required to fully assess the immunogenicity of these peptides.

In summary, the relative magnitude and rank order of responses to peptides in HLA-DQ2.5+ subjects was generally consistent across each of the cytokine release assays. With the exceptions of Peptides 7 and 12, peptides that had been selected for their importance in HLA-DQ2.5+CD were at least as active as Peptide 2. The most active peptide was Peptide 14, a hordein-derived 19mer that included up to four overlapping epitopes. Inclusion of Peptide 14 in P14, and its replacement by Peptide 3 in P13, is likely to account for P14 but not P13 being significantly more bioactive than P3.

Building upon these findings a further 13-peptide pool, P13alt, was designed to retain the higher T-cell stimulatory activity of P14, but reduce the number and length of constituent peptides as well as include the three peptides in P3. In the 13-peptide pool named P13alt, the highly immunogenic 19mer, Peptide 14 sequence is divided up between the 16mers Peptide 3 and Peptide 17 which both include overlapping 9mer cores predicted to be immunogenic in Peptide 14. In P13alt, only the more active peptide with the α-gliadin-derived epitopes DQ2.5-glia-α1 and DQ2.5-glia-α2 is included (Peptide 1) while Peptide 16 has been omitted. Peptide 8, which was included in P14, is replaced in P13alt by a closely related sequence frame-shifted by one amino-acid to ensure that the two overlapping core 9mers are flanked at both the N- and C-terminals by at least 2 amino-acids. Peptides 7 and 12 are omitted from P13alt because even though they included core sequences predicted to be HLA-DQ2.5-restricted epitopes their immunogenicity in blood collected from HLA-DQ2.5+ celiac disease subjects after gluten challenge was weak or absent. Peptides 2,4-7, 9-11, 13, and 15 present in P14 are also included in P13alt.

### Optimizing Gluten Peptide Pools for Maximal Cytokine Release

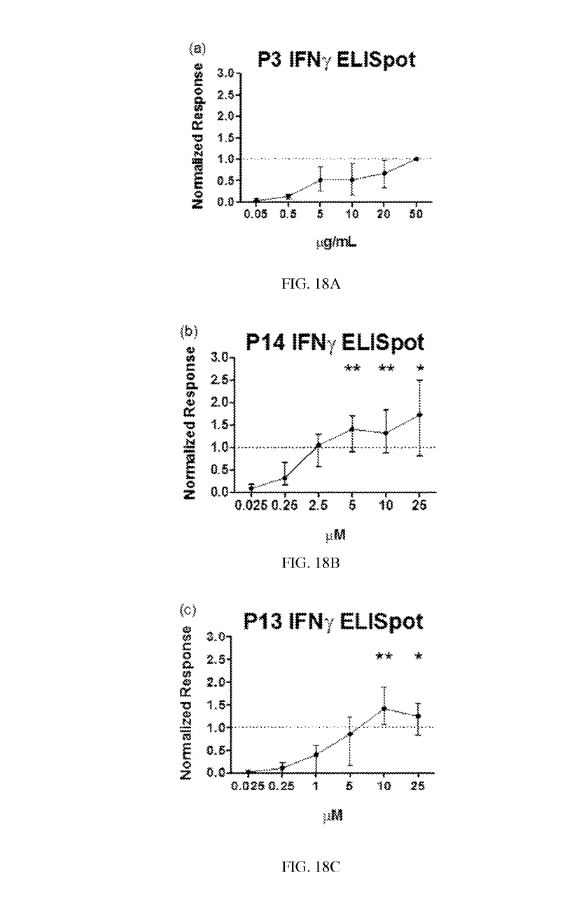

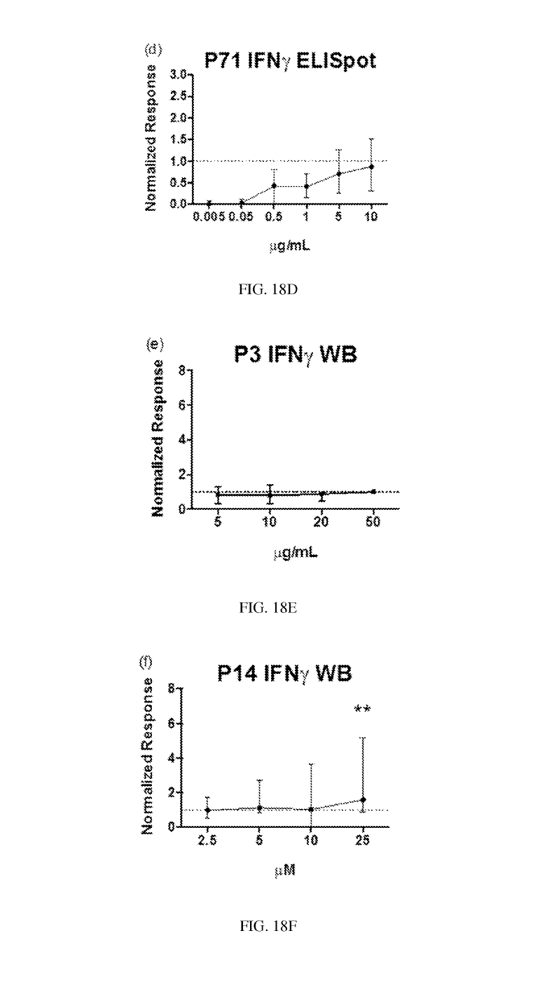

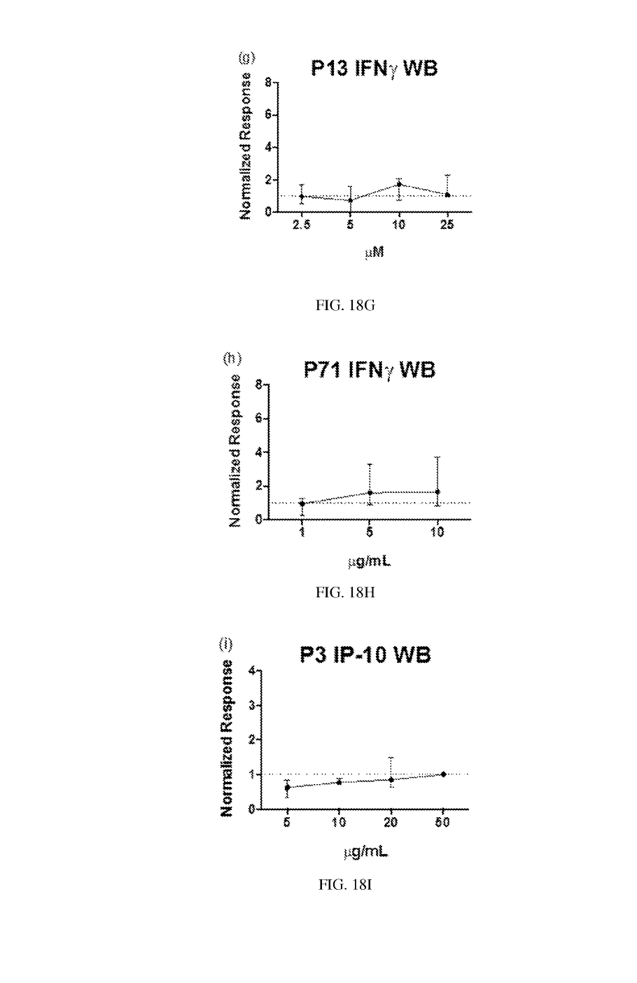

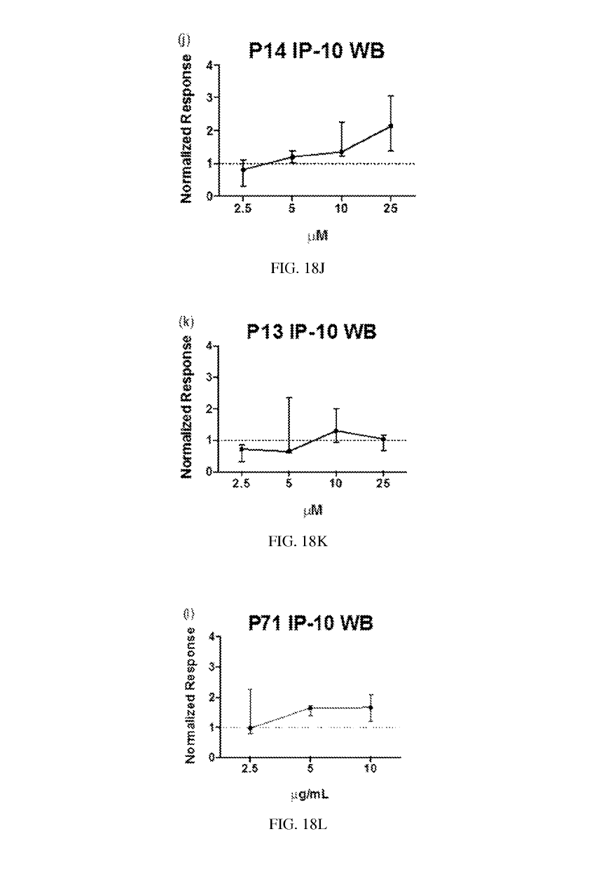

Before gluten challenge, whole blood IFNγ release was similar for P3 and the other gluten peptide pools, but IP-10 release stimulated by P3 10 μL/mL was only half that for P14 5 μM and a third that of P71 10 μL/mL (Table 14). Oral gluten challenge was followed by significant increases in cytokine release; in six subjects IP-10 levels in plasma after whole blood incubation with gluten peptides were at or above the maximal quantifiable concentration. However, after normalizing each subject's cytokine release assay responses against their own response to the highest tested concentration of P3 (50 μg/mL), median responses to the highest tested concentration of P14 (25 μM) were 60% greater than P3 50 μg/mL in both the IFNγ ELISpot and whole blood IFNγ release assays (p<0.01, and p<0.05, respectively) (Table 14 and FIG. 18). Statistical significance was not formally tested in the whole blood IP-10 release assay because six subjects mounted supra-maximal responses, but in the four subjects whose levels were within the dynamic range of the assay, responses to P14 (25 μM) were a median of 2.1× (range: 1.4-3.1) greater than to P3 50 μg/mL. The increase in responses to P13 and P71 compared to P3 were not as pronounced as for P14, but did reach statistical significance for P13 (25 μM) in the IFNγ ELISpot. Responses to P71 (10 μg/mL) were 1.7× higher than P3 (50 μg/mL) in the whole blood IFNγ and IP-10 release assays, but neither was statistically significant. To test whether normalization of data against P3 50 μg/mL was valid, data were also normalized against P3 20 μg/mL. According to this analysis whole blood IFNγ release stimulated by P14 25 uM was 70% greater than P3 50 ug/mL (p<0.014, two-tail Wilcoxon matched-pairs signed rank test). Furthermore, whole blood IFNγ release stimulated by P14 101.1M was greater than P3 20 μg/mL (p<0.04), and P14 5 μM was greater than P3 10 μg/mL (p<0.006). In conclusion, responses to P14 in cytokine release assays were significantly greater than P3.

TABLE 14
Responses to gluten peptide pools in cytokine release assays
Response on Day-6 relative to P3 (median, range)

| Cytokine | Assay | P3 50 μg/mL | P14 25 μM | P13 25 μM | P71 10 μg/mL |
|---|---|---|---|---|---|
| IFNγ | ELISpot 6¹ | 1 | 1.6 (0.82-2.3) | 1.3 (0.82-1.5) | 0.91 (0.31-1.5) |
| | | | p < 0.05 | p < 0.05 | |
| IFNγ | WB 7² | 1 | 1.6 (0.9-5.2) | 1.1 (0.95-2.3) | 1.7 (0.84-3.7) |
| | | | p < 0.01 | | |
| IP-10 | WB 4³ | 1 | 2.1 (1.4-3.1) | 1.0 (0.68-1.2) | 1.7 (1.2-2.1) |

¹Data from subjects with a P3 (50 μg/mL)-specific response <3x above medium alone or <10 SFU (sum of three wells with 0.4 million PBMC/well) were excluded from analysis.
²Data from subjects with a P3 (50 μg/mL)-specific response <1.5x medium alone were excluded from analysis.
³All subjects showed a P3 (50 μg/mL)-specific response <1.5x medium alone, but 6 were excluded from analysis because responses were above 10,000 pg/mL, the maximal level of quantitation.

### Comparison of Whole Blood IFNγ and IP-10 Release Measured by Bead Assays

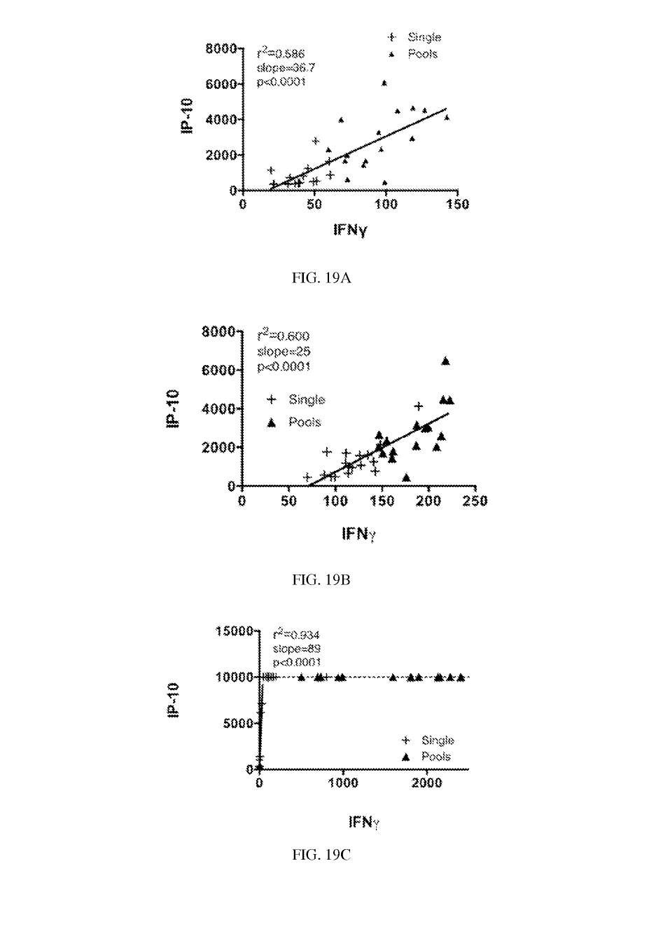

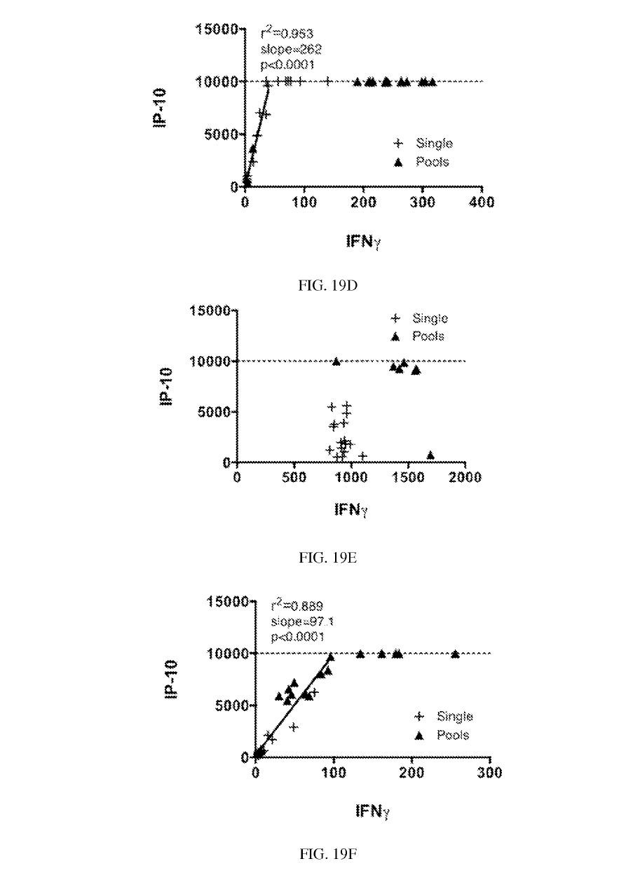

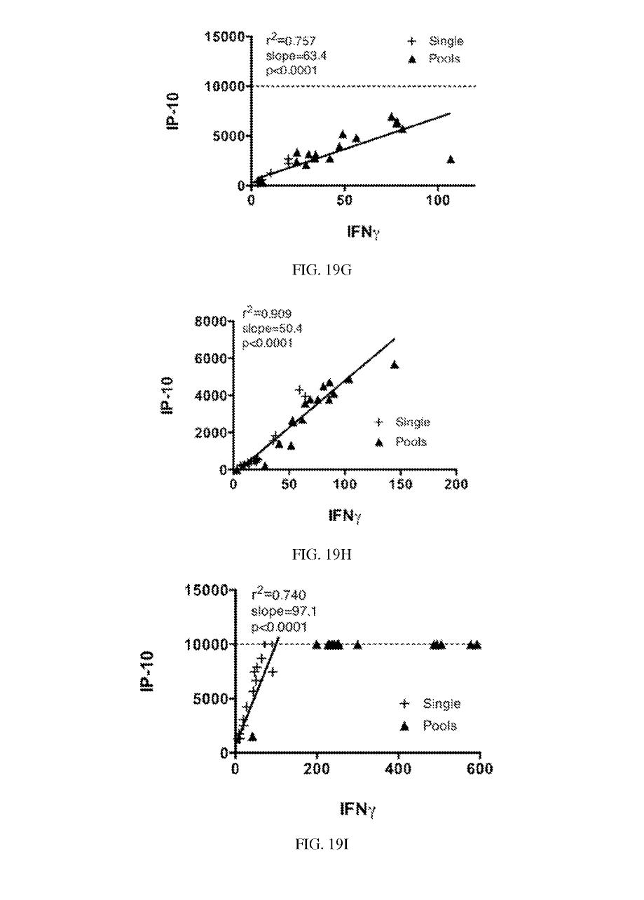

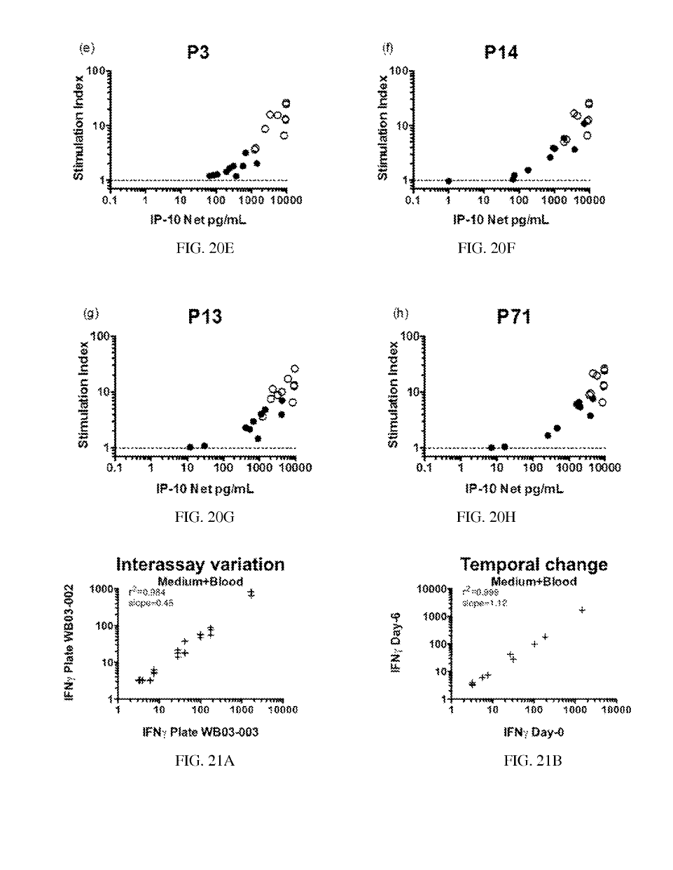

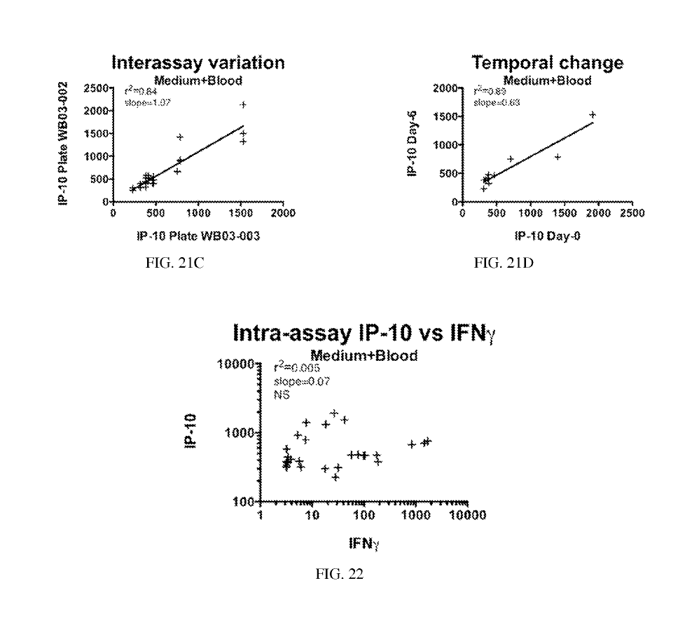

Within the dynamic range of the cytokine bead assay, there was a close correlation between individual subject's whole blood IP-10 and IFNγ release stimulated by pools or single gluten peptides (FIG. 19). Furthermore, IP-10 and IFNγ levels in plasma samples from blood collected from the same subject on Day-0 or Day-6 that were incubated with medium alone and later measured in the same or separate assays were also closely correlated (FIG. 21). However, the linear relationship between IP-10 and IFNγ was not found when data from the cohort of all ten subjects was pooled (FIG. 22), and in one subject (Subject 5) with substantially higher measured levels of plasma IFNγ in blood incubated with medium alone there was no significant correlation.

These findings were consistent with IP-10 secretion being in direct proportion to IFNγ during whole blood incubation. To test whether whole blood release of IP-10 may be more sensitive than IFNγ for detection of rare gluten-specific T cells, IP-10 levels corresponding to the threshold for “positive” whole blood IFNγ release measured by ELISA were applied to data for IFNγ and IP-10 release measured by bead assay (Table 15).

TABLE 15
IFNγ secretion required for “positive” IFNγ or IP-10 response¹

| | Subject | | | | | | | | | | Median |
|---|---|---|---|---|---|---|---|---|---|---|---|
| | 1 | 2 | 3 | 4 | 5 | 6 | 7 | 8 | 9 | 10 | (range) |
| IFNγ Day-6 medium | 76 | 125 | 3 | 4 | 1234 | 3 | 5 | 23 | 33 | 6 | 15 |
| only | | | | | | | | | | | (3-1234) |
| IFNγ Day-6 medium | 84 | 132 | 10 | 11 | 1241 | 11 | 12 | 30 | 40 | 14 | 22 |
| only +7.2 (A) | | | | | | | | | | | (10-1241) |
| IFNγ Day-6 medium | 95 | 156 | 4 | 4 | 1542 | 4 | 6 | 29 | 42 | 8 | 18 |
| only*1.25 (B) | | | | | | | | | | | (4-1542) |
| IFNγ “positive” | 95 | 156 | 10 | 11 | 1542 | 11 | 12 | 30 | 42 | 14 | 22 |
| threshold (the | | | | | | | | | | | (10-1542) |
| greater A or B) | | | | | | | | | | | |
| IP-10 Day-6 medium | 458 | 476 | 445 | 474 | 709 | 385 | 331 | 256 | 1591 | 932 | 466 |
| only | | | | | | | | | | | (256-1591) |
| IP-10 Day-6 *1.25 = | 572 | 595 | 556 | 592 | 886 | 481 | 414 | 320 | 1988 | 1165 | 582 |
| “positive” threshold | | | | | | | | | | | (320-1988) |
| Slope IP-10/IFNg | 37 | 25 | 231 | 252 | ND | 97 | 63 | 50 | 97 | 93 | 93 |
| Day-6 | | | | | | | | | | | (25-252) |
| Net increase in IP-10 | 114 | 119 | 111 | 118 | 177 | 96 | 83 | 64 | 398 | 233 | 116 |
| at IP-10 positive | | | | | | | | | | | (64-398) |
| “threshold”² | | | | | | | | | | | |

Elevation in IFNγ to reach threshold:

| | | | | | | | | | | | |
|---|---|---|---|---|---|---|---|---|---|---|---|
| For positive IP-10 | 3.1 | 4.8 | 0.5 | 0.5 | NA | 1.0 | 1.3 | 1.3 | 4.1 | 2.5 | 1.3 |
| | | | | | | | | | | | (0.5-4.8) |
| For positive IFNγ | 19 | 31 | 7.2 | 7.2 | 308 | 7.2 | 7.2 | 7.2 | 8.3 | 7.2 | 7.2 |
| | | | | | | | | | | | (7.2-308) |

¹Assay positive threshold for IFNγ was based on that for ELISA: net elevation of 7.2 pg/mL above medium alone (NIL), and stimulation index (measured IFNγ pg/mL/NIL) >1.25.
²Elevation in IP-10 stimulated by secretion of IFNγ was determined according to the slope of the regression line comparing IP-10 and IFNγ (FIG. 20)

Plasma IFNγ concentration preferably should be at least 7.2 pg/mL greater than blood incubated with medium only (NIL), and the concentration ratio to NIL preferably should be greater than 1.25 for a “positive” whole blood IFNγ ELBA. Applying these criteria to IFNγ release measured by bead assay on Day-6, the threshold concentration for positive IFNγ responses was between 10 and 42 pg/mL in five subjects, but substantially higher (95, 156 and 1542 pg/mL) in three subjects (1, 2 and 5) with elevated responses to medium only. Indeed, these three subjects were regarded as having negative IPNγ responses to P3 on both Day-0 and Day-6, in contrast to the other seven who were regarded as being “positive” on Day-6.

The slope of the regression line linking bead assay IP-10 to IFNγ levels on Day-6 for nine subjects indicated that for every pg/mL elevation of IFNγ the concentration of IP-10 increased by a median of 96 pg/mL (range: 78-479). Median NIL levels of IP-10 were 466 pg/mL (range: 256-1591), if a positive response for IP-10 was regarded as being 25% above the response to NIL, then a median elevation in IP-10 concentration of at least 116 pg/mL (range: 64-398) should indicate a “positive” response. The median elevation in IFNγ levels above NIL that would be predicted to translate to elevating IP-10 levels to the threshold for “positive” is therefore 1.3 pg/mL (range: 0.5-4.8), corresponding to a median of 6.1 (range: 2.0-15.3) times lower than that required for a positive IFNγ response. This outcome is consistent with only one subject being positive for IFNγ release to P3 before oral gluten challenge (median P3-NIL: 4.625, range: −0.1-143; P3/NIL: median 1.27, range: 1.0-2.6) compared to 7 of 10 subjects having IP-10 stimulation indices >1.25 for P3 10 pg/mL on Day-0 (P3-NIL median: 275 pg/mL, range: 654453; P3/NIL: 1.5, 1.2-3.2). Interestingly, P14 elicited responses that were more pronounced than those to P3 on Day-0 (IP-10 P14-NIL median: 839 pg/mL, range: −13-6936; P14/NIL: 3.1, 1.040.8), but still only 7 of 10 subjects had IP-10 stimulation indices >1.25.

### Conclusions

In summary, whole blood IP-10 release is tightly correlated to IFNγ release stimulated by gluten peptides in celiac disease. According to the regression line linking IFNγ and IP-10 release, measuring plasma levels of IP-10 in whole blood incubated with gluten peptides is capable of detecting rare gluten-reactive T cells that are not detected by measuring IFNγ. Although the three peptides in P3 are confirmed to be potent stimuli for circulating T cells in celiac disease, expanding the diversity of epitopes in enlarged peptide pools can further enhance ex vivo detection and therapeutic targeting of gluten-reactive T cells. The findings that IP-10 but not IFNγ responses to P14 were more pronounced than those to P3 on Day-0 suggests that measuring whole blood IP-10 release stimulated by peptide pools based on P3 but expanded by additional peptides may overcome the need for oral gluten challenge to detect circulating gluten-reactive T cells in many patients with celiac disease. An assay such as this could provide an attractive diagnostic test for celiac disease.

### REFERENCES

1. Anderson, R. P. & Jabri, B. Vaccine against autoimmune disease: antigen-specific immunotherapy. Current opinion in immunology 25, 410-417 (2013).
2. Anderson, R. P., Degano, P., Godkin, A. J., Jewell, D. P. & Hill, A. V. In vivo antigen challenge in celiac disease identifies a single transglutaminase-modified peptide as the dominant A-gliadin T-cell epitope. Nature medicine 6, 337-342 (2000).
3. Tye-Din, J. A., et al. Comprehensive, quantitative mapping of T cell epitopes in gluten in celiac disease. Science translational medicine 2, 41ra51 (2010).
4. Koskinen, L., et al. Cost-effective HLA typing with tagging SNPs predicts celiac disease risk haplotypes in the Finnish, Hungarian, and Italian populations. Immunogenetics 61, 247-256 (2009).
5. Anderson, R. P., et al. A novel serogenetic approach determines the community prevalence of celiac disease and informs improved diagnostic pathways. BMC medicine 11, 188 (2013).
6. Murray, J. A., et al. HLA DQ gene dosage and risk and severity of celiac disease. Clinical gastroenterology and hepatology: the official clinical practice journal of the American Gastroenterological Association 5, 1406-1412 (2007).
7. Karell, K., et al. HLA types in celiac disease patients not carrying the DQA1*05-DQB1*02 (DQ2) heterodimer: results from the European Genetics Cluster on Celiac Disease. Human immunology 64, 469-477 (2003).
8. Henderson, K. N., et al. A structural and immunological basis for the role of human leukocyte antigen DQ8 in celiac disease. Immunity 27, 23-34 (2007).
9. van de Wal, Y., et al. Selective deamidation by tissue transglutaminase strongly enhances gliadin-specific T cell reactivity. Journal of immunology 161, 1585-1588 (1998).
10. van de Wal, Y., et al. Glutenin is involved in the gluten-driven mucosal T cell response. European journal of immunology 29, 3133-3139 (1999).
11. Tollefsen, S., et al. HLA-DQ2 and -DQ8 signatures of gluten T cell epitopes in celiac disease. The Journal of clinical investigation 116, 2226-2236 (2006).
12. Kooy-Winkelaar, Y., et al. Gluten-specific T cells cross-react between HLA-DQ8 and the HLA-DQ2alpha/DQ8beta transdimer. Journal of immunology 187, 5123-5129 (2011).
13. Bodd, M., Kim, C. Y., Lundin, K. E. & Sollid, L. M. T-cell response to gluten in patients with HLA-DQ2.2 reveals requirement of peptide-MHC stability in celiac disease. Gastroenterology 142, 552-561(2012).
14. Vader, W., et al. The gluten response in children with celiac disease is directed toward multiple gliadin and glutenin peptides. Gastroenterology 122, 1729-1737 (2002).
15. Ontiveros, N., Tye-Din, J. A., Hardy, M. Y. & Anderson, R. P. Ex-vivo whole blood secretion of interferon (IFN)-gamma and IFN-gamma-inducible protein-10 measured by enzyme-linked immunosorbent assay are as sensitive as IFN-gamma enzyme-linked immunospot for the detection of gluten-reactive T cells in human leucocyte antigen (HLA)-DQ2.5(+)-associated coeliac disease. Clinical and experimental immunology 175, 305-315 (2014).
16. Christophersen, A., et al. Tetramer-visualized gluten-specific CD4+ T cells in blood as a potential diagnostic marker for coeliac disease without oral gluten challenge. United European gastroenterology journal 2, 268-278 (2014).
17. Luster, A. D. & Ravetch, J. V. Biochemical characterization of a gamma interferon-inducible cytokine (IP-10). The Journal of experimental medicine 166, 1084-1097 (1987).
18. Cassatella, M. A., et al. Regulated production of the interferon-gamma-inducible protein-10 (IP-10) chemokine by human neutrophils. European journal of immunology 27, 111-115 (1997).
19. Dufour, J. H., et al. IFN-gamma-inducible protein 10 (IP-10; CXCL10)-deficient mice reveal a role for IP-10 in effector T cell generation and trafficking. Journal of immunology 168, 3195-3204 (2002).
20. Ruhwald, M., et al. CXCL10/IP-10 release is induced by incubation of whole blood from tuberculosis patients with ESAT-6, CFP10 and TB7.7. Microbes and infection/Institut Pasteur 9, 806-812 (2007).
21. Wang, S., et al. Evaluation of the diagnostic potential of IP-10 and IL-2 as biomarkers for the diagnosis of active and latent tuberculosis in a BCG-vaccinated population. PloS one 7, e51338 (2012).
22. National Institutes of Health Consensus Development Conference Statement on Celiac Disease, Jun. 28-30, 2004. Gastroenterology 128, S1-9 (2005).
23. Junker, Y., et al. Wheat amylase trypsin inhibitors drive intestinal inflammation via activation of toll-like receptor 4. The Journal of experimental medicine 209, 2395-2408 (2012).
24. Kern, F., et al. Analysis of CD8 T cell reactivity to cytomegalovirus using protein-spanning pools of overlapping pentadecapeptides. European journal of immunology 30, 1676-1682 (2000).
25. Maecker, H. T., et al. Use of overlapping peptide mixtures as antigens for cytokine flow cytometry. Journal of immunological methods 255, 27-40 (2001).
26. Vader, L. W., et al. Specificity of tissue transglutaminase explains cereal toxicity in celiac disease. The Journal of experimental medicine 195, 643-649 (2002).
27. Shan, L., et al. Identification and analysis of multivalent proteolytically resistant peptides from gluten: implications for celiac sprue. Journal of proteome research 4, 1732-1741 (2005).

### Example 3

### Whole Blood Cytokine Release Stimulated by Gluten Peptides in Seronegative CD Patients Compared to Seronegative Patients with Non-Celiac Gluten Sensitivity with Reduced Intake of Dietary Gluten

### Aim:

To assess gluten-peptide pool stimulated whole blood cytokine release assays for celiac disease (CD) patients negative for CD-specific serology (tTG-IgA and DGP-IgG).

### Endpoints:

### Primary

Sensitivity and specificity of whole blood cytokine release detected by IP-10 ELISA for tTG-IgA/DGP-IgG seronegative CD vs non-celiac gluten-sensitive (NCGS) patients

### Secondary

(1) Sensitivity and specificity of whole blood cytokine release detected by IP-10 ELISA for CD patients vs NCGS patients who carry HLA-DQ genotypes associated with celiac disease (HLA-DQ2.5+ or DQ2.2+ or DQ8+).
(2) Sensitivity and specificity of cytokine release detected by IP-10 compared to IFNγ ELISAs for CD patients vs NCGS patients who carry HLA-DQ genotypes associated with celiac disease (HLA-DQ2.5+ or DQ2.2+ or DQ8+).

### Patients:

### Inclusion:

(1) Celiac disease on gluten-free diet—diagnosis of CD established and documented according to Expert Clinical Guidelines (e.g. World Gastroenterology Organisation Global Guidelines on Celiac Disease. 2013) who self report being generally compliant with gluten-free diet

### OR

Non-celiac gluten-sensitive—established by normal tTG-IgA serology and/or small bowel histology while regularly consuming gluten who self report being generally compliant with gluten-free diet
(2) No medical contradiction to blood collection by standard venepuncture with a 21G butterfly needle
(3) tTG-IgA (INOVA rhtTG-IgA) and DGP-IgG (INOVA Gliaden II IgG) within the laboratory normal range
(4) Aged 18 or older

### Screening Tests and Information:

EDTA-anticoagulated blood for comprehensive HLA-DQA and HLA-DQB allele determination
Serum tTG-IgA (INOVA rhtTG-IgA) and DGP-IgG (INOVA Gliaden II IgG) assessment
Documentation of medical tests establishing or excluding a diagnosis of celiac disease
Symptoms at diagnosis and current GI symptoms
Duration of gluten-free diet
Co-morbidities (if any)
Medications (if any)

### Age and sex

### Procedure:

Subjects attend a single visit to the trial site for collection of blood to perform:
1. HLA-DQ gene test (Lavender-top EDTA 5 mL, Melbourne Pathology, SONIC)
2. CD serology (Brown-top serum tube 5 mL, Dorevitch Pathology),
3. Whole blood release—subjects will have ONE tube (1 mL blood/tube) for each whole blood incubation condition (9 Quantiferon-GoldTB NIL and 1 MITO tube). In addition, 10 of 30 CD subjects will have 27 additional Cellestis NIL tubes drawn to determine inter- and intra-assay variability (the first 10 CD subjects). After blood is drawn, 0.1 mL volumes of aliquots (listed below) are added by 0.5 mL insulin syringe to NIL tubes containing 1 mL blood, and PBS is added to MITOGEN tube containing 1 mL blood. All Quantiferon tubes are placed in 37° C. incubator. After 24 h incubation, plasma is separated from blood in the Quantiferon tubes and placed in appropriately labeled cryovials then frozen −80° C. Frozen plasma samples then used for ELISA determination of IP-10 and IFNγ.
   Tubes and aliquots are prepared containing one of the following:

### PBS

### PBS+0.5% DMSO,

CEFT 11 ug/mL,
Pool 1—P3 pool 550 μg/mL in PBS (see Example 2 for P3 pool peptides)
Pool 2—P14 pool 275 μM in PBS (see Example 2 for P14 pool peptides)
Pool 3—Total Gluten 110 μg/mL in PBS 0.5% DMSO
Pool 4a—P16 pool 110 μM in PBS
Pool 4b—P16 pool 275 μM in PBS
Pool 4c—P16 pool 550 μM in PBS
P16 pool

| Peptide | Epitope(s) |
|---|---|
| (pE)PFPQPELPYPQP-amide | PFPQPELPY (SEQ ID NO: 1), |
| (SEQ ID NO: 84) | PQPELPYPQ (SEQ ID NO: 2) |
| (pE)PFPQPEQPFPWQ-amide | PFPQPEQPF (SEQ ID NO: 3), |
| (SEQ ID NO: 85) | PQPEQPFPW (SEQ ID NO: 4) |
| (pE)EQPIPEQPQPYP-amide | EQPIPEQPQ (SEQ ID NO: 5), |
| (SEQ ID NO: 86) | PIPEQPQPY (SEQ ID NO: 6) |
| (pE)PFPQPEQPIPVQ-amide | PFPQPEQPI (SEQ ID NO: 7), |
| (SEQ ID NO: 87) | PQPEQPIPV (SEQ ID NO: 8) |
| (pE)PEQPIPVQPEQS-amide | EQPIPVQPE (SEQ ID NO: 9) |
| (SEQ ID NO: 88) | |
| (pE)PFPQPEQPTPIQ-amide | PFPQPEQPT (SEQ ID NO: 10), |
| (SEQ ID NO: 89) | PQPEQPTPI (SEQ ID NO: 11) |
| (pE)PEQPTPIQPEQP-amide | EQPTPIQPE (SEQ ID NO: 12) |
| (SEQ ID NO: 90) | |
| (pE)PFPQPEQPFPLQ-amide | PQPEQPFP (SEQ ID NO: 13) |
| (SEQ ID NO: 91) | |
| (pE)PEQPFPLQPEQP-amide | EQPFPLQPE (SEQ ID NO: 14) |
| (SEQ ID NO: 92) | |
| (pE)GEGSFQPSQENP-amide | EGSFQPSQE (SEQ ID NO: 17) |
| (SEQ ID NO: 93) | |
| (pE)QQGYYPTSPQQS-amide | QGYYPTSPQ (SEQ ID NO: 18) |
| (SEQ ID NO: 94) | |
| (pE)PEQPEQPFPEQP-amide | EQPEQPFPE (SEQ ID NO: 19) |
| (SEQ ID NO: 95) | |
| (pE)PPFSEQEQPVLP-amide | PFSEQEQPV (SEQ ID NO: 22) |
| (SEQ ID NO: 96) | |
| (pE)PYPQPELPYPQP-amide | PYPQPELPY (SEQ ID NO: 25), |
| (SEQ ID NO: 97) | (PQPELPYPQ (SEQ ID NO: 2)) |
| (pE)EQPFPEQPIPEQ-amide | EQPFPEQPI (SEQ ID NO: 23), |
| (SEQ ID NO: 98) | PFPEQPIPE (SEQ ID NO: 24) |
| (pE)PQPYPEQPQPFP-amide | PYPEQPQPF (SEQ ID NO: 16), |
| (SEQ ID NO: 99) | PQPYPEQPQ (SEQ ID NO: 27) |

(pE) = pyroglutamate

Validated ELISAs and/or bead-based multiplex assays will be used for determination of IP-10 and IFN-γ and will be used to establish an upper limit for stimulation index and concentration of each analyte using plasma collected from NCGS who are not genetically susceptible to celiac disease. In the initial analysis, data points will be determined to be elevated or not according to this threshold (e.g. Stimulated blood minus NIL with PBS only >7.2 pg/mL and Stimulated blood/NIL with PBS only >1.25 for IFNγ). Threshold values to optimize sensitivity and specificity differentiating CD vs NCGS will be further refined according receiver operating characteristic (ROC) curve analysis and area under the curve (AUC) analysis. Data from subjects with CD who are excluded because of being seropositive for tTG-IgA or DGP-IgG will be reported and analyzed separately according to the same cutoffs as applied to other subjects.

### Sample Size Estimation

Celiac disease—approximately ⅓ of treated CD subjects show elevated CD-serology and >99% are HLA-DQ2.5+ or DQ8+ or DQ2.2+
NCGS—all have normal CD serology and 60% are HLA-DQ2.5+ or DQ8+ or DQ2.2+
To enroll ˜20 seronegative CD subjects, 30 total should be enrolled
To enroll ˜20 HLA-DQ2.5+ or DQ8+ or DQ2.2+ NCGS subjects, 30 total should enrolled

### Example 4

### Whole Blood Cytokine Release Stimulated by Gluten Peptides in Seronegative CD Patients

### Aim:

To determine 24 h IP-10 release stimulated by gluten-peptide pools in whole blood collected from subjects with celiac disease (CD) on strict gluten-free diet who were negative for celiac disease-specific serology (tTG-IgA and DGP-IgG).

Subjects were Included if they Met the Following Criteria:

(1) Celiac disease on gluten-free diet—diagnosis of CD established and documented according to Expert Clinical Guidelines (e.g. World Gastroenterology Organisation Global Guidelines on Celiac Disease. 2013) who self-report being generally compliant with gluten-free diet

(2) No medical contradiction to blood collection by standard venepuncture with a 21G butterfly needle

(3) tTG-IgA (INOVA rhtTG-IgA) and DGP-IgG (INOVA Gliaden II IgG) within the laboratory normal range

(4) Aged 18-70 years

(5) Provide written informed consent

### Procedure:

Blood (1 mL) was collected directly into heparinized tubes and peptide solutions in phosphate buffered saline (PBS) or PBS alone (0.1 mL) was injected into the blood collection tube. Blood was incubated for 24 h at 37° C. After incubation, plasma was separated and frozen until being thawed and the concentration of IP-10 determined by magnetic bead assay (Luminex, Millipore). Peptide solutions consisted of the following from Example 3: Gluten Pool 1 (2 16mers and 1 15mer peptides), Pool 2 (14 peptides 13-19mers) and Pool 4 (16 13mer peptides). Pool 1 included at least five HLA-DQ2.5-restricted epitopes, Pool 2 and Pool 4 were designed to include the same core 9mer sequences recognized by gluten-reactive CD4+ T cells from HLA-DQ2.5+, HLA-DQ8+, and/or HLA-DQ2.2+ donors with celiac disease.

### Results:

Demographics of subjects included in the study are shown in Table 16. Seven donors aged between 35 and 56 yrs were studied, five were HLA-DQ2.5+, and the two other subjects were either HLA-DQ8+ or HLA-DQ2.2+.

TABLE 16
Demographics of subjects

| | | | | | | CD | | | | |
|---|---|---|---|---|---|---|---|---|---|---|
| | HLA serotype | | | | | Serology | | Year of | | |
| | HLA- | HLA- | HLA- | HLA alleles | | tTg- | DGP- | CD | GFD | |
| Subject | DQ | DR | DQ | DQA1 | DQB1 | IgA | IgG | diagnosis | since | Age/yrs |
| A | 2.5, 2.2 | 3 7 | 2, 2 | 02 05 | 02 02 | <1 | 1 | 2002 | 2002 | 49 |
| B | 2.5 | 3 3 | 2, 2 | 05 05 | 02 02 | 1 | 4 | 2009 | 2009 | 56 |
| | homozygote | | | | | | | | | |
| C | 2.5 | 1 3 | 2, 5 | 01 05 | 02 05 | <1 | 5 | 1990 | 1990 | 51 |
| D | 2.5 | 3 15 | 2, 6 | 01 05 | 02 06 | <1 | 3 | 2010 | 2010 | 44 |
| E | 2.5/8 | 3 4 | 2, 8 | 03 05 | 02 0302 | 3 | 98 | 2003 | 2004 | 49 |
| F | 8 | 4 13 | 6, 8 | 01, 03 | 0302, 06 | 1 | 2 | 2010 | 2010 | 35 |
| G | 2.2 | 7 13 | 2, 6 | 01, 02 | 02, 06 | <1 | 2 | 2004 | 2004 | 50 |

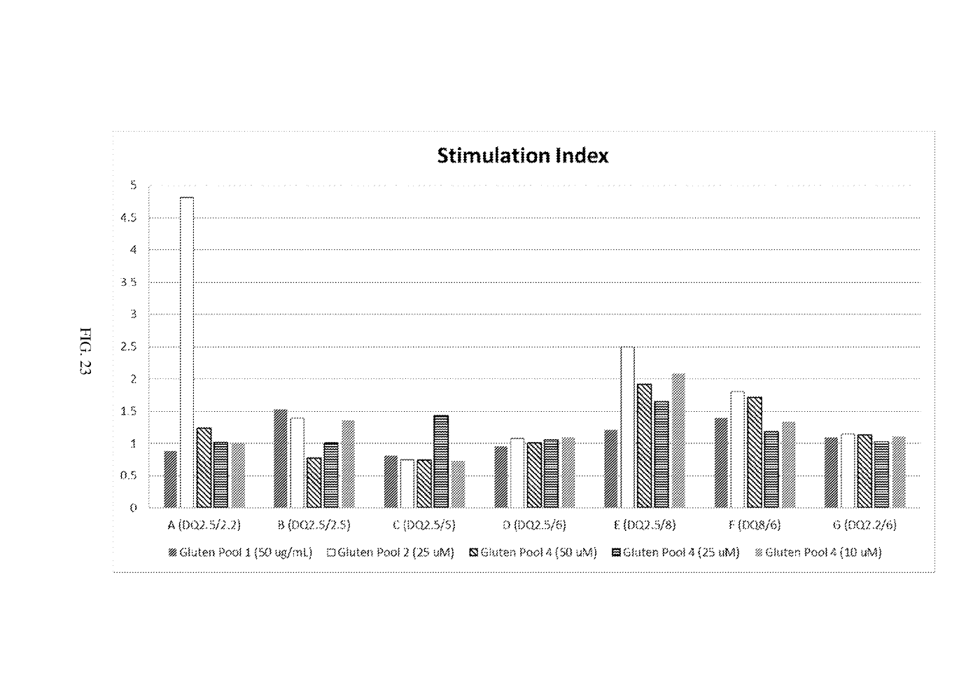

Peptide Pool 2 and Pool 4 showed stimulation indices higher than Pool 1, and above one, i.e. IP-10 plasma concentrations were greater than in blood incubated with PBS alone (FIG. 23). Notably, the subject with the genotype HLA-DQ8/6+ showed a stimulation index above one for Pool 2 and Pool 4, but not Pool 1 indicating the potential for the expanded peptide pools to activate T cells in donors who were not HLA-DQ2.5+(FIG. 23).

### Conclusion:

Expanded peptide pools that include gluten-derived epitopes additional to those represented in the 3-peptide composition (PFPQPELPY (SEQ ID NO: 1), PQPELPYPQ (SEQ ID NO: 2), PFPQPEQPF (SEQ ID NO: 3), PQPEQPFPW (SEQ ID NO: 4), PIPEQPQPY (SEQ ID NO: 6), and EQPIPEQPQ (SEQ ID NO: 5)) have, in some embodiments, the capacity to increase IP-10 release in blood from celiac disease donors who are HLA-DQ2.5+ and those who are negative for HLA-DQ2.5.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A composition comprising at least one peptide, the at least one peptide comprising at least one amino acid sequence selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), PQPEQPFSQ (SEQ ID NO: 15), PYPEQPQPF (SEQ ID NO: 16), EGSFQPSQE (SEQ ID NO: 17), QGYYPTSPQ (SEQ ID NO: 18), EQPEQPFPE (SEQ ID NO: 19), EQPFPEQPQ (SEQ ID NO: 20), PFPEQPEQI (SEQ ID NO: 21), PFSEQEQPV (SEQ ID NO: 22), EQPFPEQPI (SEQ ID NO: 23), PFPEQPIPE (SEQ ID NO: 24), PYPQPELPY (SEQ ID NO: 25), PQPELPYPY (SEQ ID NO: 26), and PQPYPEQPQ (SEQ ID NO: 27).

2. The composition of claim 1, comprising at least one peptide, the at least one peptide comprising at least eight amino acid sequences selected from PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), PIPEQPQPY (SEQ ID NO: 6), PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), PQPEQPFSQ (SEQ ID NO: 15), PYPEQPQPF (SEQ ID NO: 16), EGSFQPSQE (SEQ ID NO: 17), QGYYPTSPQ (SEQ ID NO: 18), EQPEQPFPE (SEQ ID NO: 19), EQPFPEQPQ (SEQ ID NO: 20), PFPEQPEQI (SEQ ID NO: 21), PFSEQEQPV (SEQ ID NO: 22), EQPFPEQPI (SEQ ID NO: 23), PFPEQPIPE (SEQ ID NO: 24), PYPQPELPY (SEQ ID NO: 25), PQPELPYPY (SEQ ID NO: 26), and PQPYPEQPQ (SEQ ID NO: 27).

3. The composition of claim 2, comprising at least one peptide comprising the amino acid sequences PFPQPELPY (SEQ ID NO: 1), PQPELPYPQ (SEQ ID NO: 2), PFPQPEQPF (SEQ ID NO: 3), PQPEQPFPW (SEQ ID NO: 4), EQPIPEQPQ (SEQ ID NO: 5), and PIPEQPQPY (SEQ ID NO: 6); and at least two amino acid sequences selected from PFPQPEQPI (SEQ ID NO: 7), PQPEQPIPV (SEQ ID NO: 8), EQPIPVQPE (SEQ ID NO: 9), PFPQPEQPT (SEQ ID NO: 10), PQPEQPTPI (SEQ ID NO: 11), EQPTPIQPE (SEQ ID NO: 12), PQPEQPFPL (SEQ ID NO: 13), EQPFPLQPE (SEQ ID NO: 14), PQPEQPFSQ (SEQ ID NO: 15), PYPEQPQPF (SEQ ID NO: 16), EGSFQPSQE (SEQ ID NO: 17), QGYYPTSPQ (SEQ ID NO: 18), EQPEQPFPE (SEQ ID NO: 19), EQPFPEQPQ (SEQ ID NO: 20), PFPEQPEQI (SEQ ID NO: 21), PFSEQEQPV (SEQ ID NO: 22), EQPFPEQPI (SEQ ID NO: 23), PFPEQPIPE (SEQ ID NO: 24), PYPQPELPY (SEQ ID NO: 25), PQPELPYPY (SEQ ID NO: 26), and PQPYPEQPQ (SEQ ID NO: 27).

4. The composition of any one of claims 1 to 3, wherein the composition comprises at least five peptides.

5. The composition of claim 1, wherein the composition comprises at least one peptide selected from:
(a) a first peptide comprising the amino acid sequence PFPQPELPY (SEQ ID NO: 1) and the amino acid sequence PQPELPYPQ (SEQ ID NO: 2);
(b) a second peptide comprising the amino acid sequence PFPQPEQPF (SEQ ID NO: 3) and the amino acid sequence PQPEQPFPW (SEQ ID NO: 4);
(c) a third peptide comprising the amino acid sequence EQPIPEQPQ (SEQ ID NO: 5) and the amino acid sequence PIPEQPQPY (SEQ ID NO: 6);
(d) a fourth peptide comprising the amino acid sequence PFPQPEQPI (SEQ ID NO: 7), the amino acid sequence PQPEQPIPV (SEQ ID NO: 8), and the amino acid sequence EQPIPVQPE (SEQ ID NO: 9);
(e) a fifth peptide comprising the amino acid sequence PFPQPEQPT (SEQ ID NO: 10), the amino acid sequence PQPEQPTPI (SEQ ID NO: 11), and the amino acid sequence EQPTPIQPE (SEQ ID NO: 12);
(f) a sixth peptide comprising the amino acid sequence PFPQPEQPF (SEQ ID NO: 3), the amino acid sequence PQPEQPFPL (SEQ ID NO: 13), and the amino acid sequence EQPFPLQPE (SEQ ID NO: 14);
(g) a seventh peptide comprising the amino acid sequence PFPQPEQPF (SEQ ID NO: 3) and the amino acid sequence PQPEQPFSQ (SEQ ID NO: 15);
(h) an eighth peptide comprising the amino acid sequence PYPEQPQPF (SEQ ID NO: 16);
(i) a ninth peptide comprising the amino acid sequence PFPEQPEQI (SEQ ID NO: 21);
(j) a tenth peptide comprising the amino acid sequence EGSFQPSQE (SEQ ID NO: 17);
(k) an eleventh peptide comprising the amino acid sequence QGYYPTSPQ (SEQ ID NO: 18);
(l) a twelfth peptide comprising the amino acid sequence EQPEQPFPE (SEQ ID NO: 19) and the amino acid sequence EQPFPEQPQ (SEQ ID NO: 20);
(m) a thirteenth peptide comprising the amino acid sequence PFSEQEQPV (SEQ ID NO: 22);
(n) a fourteenth peptide comprising the amino acid sequence EQPFPEQPI (SEQ ID NO: 23), the amino acid sequence PFPEQPIPE (SEQ ID NO: 24), the amino acid sequence EQPIPEQPQ (SEQ ID NO: 5), and the amino acid sequence PIPEQPQPY (SEQ ID NO: 6);
(o) a fifteenth peptide comprising the amino acid sequence PQPELPYPQ (SEQ ID NO: 2) and the amino acid sequence PYPQPELPY (SEQ ID NO: 25);
(p) a sixteenth peptide comprising the amino acid sequence PFPQPELPY (SEQ ID NO: 1) and the amino acid sequence PQPELPYPY (SEQ ID NO: 26);
(q) a seventeenth peptide comprising the amino acid sequence EQPFPEQPI (SEQ ID NO: 23) and the amino acid sequence PFPEQPIPE (SEQ ID NO: 24); and
(r) an eighteenth peptide comprising the amino acid sequence PQPYPEQPQ (SEQ ID NO: 27) and the amino acid sequence PYPEQPQPF (SEQ ID NO: 16).

6. The composition of claim 5, wherein:
(a) the first peptide comprises the amino acid sequence LQPFPQPELPYPQPQ (SEQ ID NO: 28);
(b) the second peptide comprises the amino acid sequence QPFPQPEQPFPWQP (SEQ ID NO: 29);
(c) the third peptide comprises the amino acid sequence PEQPIPEQPQPYPQQ (SEQ ID NO: 30);
(d) the fourth peptide comprises the amino acid sequence QPFPQPEQPIPVQPEQS (SEQ ID NO: 31);
(e) the fifth peptide comprises the amino acid sequence QPFPQPEQPTPIQPEQP (SEQ ID NO: 32);
(f) the sixth peptide comprises the amino acid sequence QPFPQPEQPFPLQPEQP (SEQ ID NO: 33);
(g) the seventh peptide comprises the amino acid sequence QPFPQPEQPFSQQ (SEQ ID NO: 34);
(h) the eighth peptide comprises the amino acid sequence PQPYPEQPQPFPQQ (SEQ ID NO: 35);
(i) the ninth peptide comprises the amino acid sequence QPFPEQPEQIIPQQP (SEQ ID NO: 36);
(j) the tenth peptide comprises the amino acid sequence SGEGSFQPSQENPQ (SEQ ID NO: 37);
(k) the eleventh peptide comprises the amino acid sequence GQQGYYPTSPQQSG (SEQ ID NO: 38);
(l) the twelfth peptide comprises the amino acid sequence PEQPEQPFPEQPQQ (SEQ ID NO: 39);
(m) the thirteenth peptide comprises the amino acid sequence QPPFSEQEQPVLPQ (SEQ ID NO: 40);
(n) the fourteenth peptide comprises the amino acid sequence PEQPFPEQPIPEQPQPYP (SEQ ID NO: 41);
(o) the fifteenth peptide comprises the amino acid sequence QPYPQPELPYPQPQ (SEQ ID NO: 42);
(p) the sixteenth peptide comprises the amino acid sequence QPFPQPELPYPYPQ (SEQ ID NO: 43);
(q) the seventeenth peptide comprises the amino acid sequence PQEQPFPEQPIPEQP (SEQ ID NO: 44); and
(r) the eighteenth peptide comprises the amino acid sequence QPQPYPEQPQPFPQQ (SEQ ID NO: 45).

7. The composition of claim 5 or 6, wherein the composition comprises at least four of the peptides.

8. The composition of claim 7, comprising:
(i) the first, second, and third peptides or the second, fourteenth, fifteenth, and sixteenth peptides; and
(ii) at least one of the fourth, fifth, sixth, seventh, eighth, ninth, tenth, eleventh, twelfth, thirteenth, sixteenth, seventeenth, and eighteenth peptides.

9. The composition of claim 8, comprising at least two of the fourth, fifth, sixth, seventh, eighth, ninth, tenth, eleventh, twelfth, thirteenth, sixteenth, seventeenth, and eighteenth peptides.

10. The composition of claim 9, comprising at least three of the fourth, fifth, sixth, seventh, eighth, ninth, tenth, eleventh, twelfth, thirteenth, sixteenth, seventeenth, and eighteenth peptides.

11. The composition of claim 10, comprising at least four of the fourth, fifth, sixth, seventh, eighth, ninth, tenth, eleventh, twelfth, thirteenth, sixteenth, seventeenth, and eighteenth peptides.

12. The composition of claim 11, comprising at least five of the fourth, fifth, sixth, seventh, eighth, ninth, tenth, eleventh, twelfth, thirteenth, sixteenth, seventeenth, and eighteenth peptides.

13. The composition of claim 12, comprising at least six of the fourth, fifth, sixth, seventh, eighth, ninth, tenth, eleventh, twelfth, thirteenth, sixteenth, seventeenth, and eighteenth peptides.

14. The composition of claim 13, comprising at least seven of the fourth, fifth, sixth, seventh, eighth, ninth, tenth, eleventh, twelfth, thirteenth, sixteenth, seventeenth, and eighteenth peptides.

15. The composition of claim 14, comprising at least eight of the fourth, fifth, sixth, seventh, eighth, ninth, tenth, eleventh, twelfth, thirteenth, sixteenth, seventeenth, and eighteenth peptides.

16. The composition of claim 15, comprising at least nine of the fourth, fifth, sixth, seventh, eighth, ninth, tenth, eleventh, twelfth, thirteenth, sixteenth, seventeenth, and eighteenth peptides.

17. The composition of claim 16, comprising at least ten of the fourth, fifth, sixth, seventh, eighth, ninth, tenth, eleventh, twelfth, thirteenth, sixteenth, seventeenth, and eighteenth peptides.

18. The composition of any one of claims 5 to 8, comprising the first, second, third, fourth, fifth, sixth, seventh, eighth, ninth, tenth, eleventh, twelfth, and thirteenth peptides.

19. The composition of any one of claims 5 to 8, comprising the second, fourth, fifth, sixth, seventh, eighth, ninth, tenth, eleventh, twelfth, thirteenth, fourteenth, fifteenth, and sixteenth peptides.

20. The composition of any one of claims 5 to 8, comprising the first, second, third, fourth, fifth, sixth, tenth, eleventh, twelfth, thirteenth, fifteenth, seventeenth, and eighteenth peptides.

21. The composition of claim 1, wherein the composition comprises at least one of:
(a) a first peptide comprising the amino acid sequence PFPQPELPY (SEQ ID NO: 1) and the amino acid sequence PQPELPYPQ (SEQ ID NO: 2);
(b) a second peptide comprising the amino acid sequence PFPQPEQPF (SEQ ID NO: 3) and the amino acid sequence PQPEQPFPW (SEQ ID NO: 4);
(c) a third peptide comprising the amino acid sequence EQPIPEQPQ (SEQ ID NO: 5) and the amino acid sequence PIPEQPQPY (SEQ ID NO: 6);
(d) a fourth peptide comprising the amino acid sequence PFPQPEQPI (SEQ ID NO: 7) and the amino acid sequence PQPEQPIPV (SEQ ID NO: 8);
(e) a fifth peptide comprising the amino acid sequence EQPIPVQPE (SEQ ID NO: 9);
(f) a sixth peptide comprising the amino acid sequence PFPQPEQPT (SEQ ID NO: 10) and the amino acid sequence PQPEQPTPI (SEQ ID NO: 11);
(g) a seventh peptide comprising the amino acid sequence EQPTPIQPE (SEQ ID NO: 12);
(h) an eighth peptide comprising the amino acid sequence PQPEQPFPL (SEQ ID NO: 13);
(i) a ninth peptide comprising the amino acid sequence EQPFPLQPE (SEQ ID NO: 14);
(j) a tenth peptide comprising the amino acid sequence EGSFQPSQE (SEQ ID NO: 17);
(k) a eleventh peptide comprising the amino acid sequence QGYYPTSPQ (SEQ ID NO: 18);
(l) a twelfth peptide comprising the amino acid sequence EQPEQPFPE (SEQ ID NO: 19);
(m) a thirteenth peptide comprising the amino acid sequence PFSEQEQPV (SEQ ID NO: 22);
(n) a fourteenth peptide comprising the amino acid sequence PYPQPELPY (SEQ ID NO: 25);
(o) a fifteenth peptide comprising the amino acid sequence EQPFPEQPI (SEQ ID NO: 23) and the amino acid sequence PFPEQPIPE (SEQ ID NO: 24); and
(p) a sixteenth peptide comprising the amino acid sequence PYPEQPQPF (SEQ ID NO: 16) and the amino acid sequence PQPYPEQPQ (SEQ ID NO: 27).

22. The composition of claim 21, wherein
(a) the first peptide comprises the amino acid sequence PFPQPELPYPQP (SEQ ID NO: 46);
(b) the second peptide comprises the amino acid sequence PFPQPEQPFPWQ (SEQ ID NO: 47);
(c) the third peptide comprises the amino acid sequence EQPIPEQPQPYP (SEQ ID NO: 48);
(d) the fourth peptide comprises the amino acid sequence PFPQPEQPIPVQ (SEQ ID NO: 49);
(e) the fifth peptide comprises the amino acid sequence PEQPIPVQPEQS (SEQ ID NO: 50);
(f) the sixth peptide comprises the amino acid sequence PFPQPEQPTPIQ (SEQ ID NO: 51);
(g) the seventh peptide comprises the amino acid sequence PEQPTPIQPEQP (SEQ ID NO: 52);
(h) the eighth peptide comprises the amino acid sequence PFPQPEQPFPLQ (SEQ ID NO: 53);
(i) the ninth peptide comprises the amino acid sequence PEQPFPLQPEQP (SEQ ID NO: 54);
(j) the tenth peptide comprises the amino acid sequence GEGSFQPSQENP (SEQ ID NO: 55);
(k) the eleventh peptide comprises the amino acid sequence QQGYYPTSPQQS (SEQ ID NO: 56);
(l) the twelfth peptide comprises the amino acid sequence PEQPEQPFPEQP (SEQ ID NO: 57);
(m) the thirteenth peptide comprises the amino acid sequence PPFSEQEQPVLP (SEQ ID NO: 58);
(n) the fourteenth peptide comprises the amino acid sequence PYPQPELPYPQP (SEQ ID NO: 59);
(o) the fifteenth peptide comprises the amino acid sequence EQPFPEQPIPEQ (SEQ ID NO: 60); and
(p) the sixteenth peptide comprises the amino acid sequence PQPYPEQPQPFP (SEQ ID NO: 61).

23. The composition of claim 21 or 22, wherein the composition comprises the first, second, third, fourth, fifth, sixth, seventh, eighth, ninth, tenth, eleventh, twelfth, thirteenth, fourteenth, fifteenth, and sixteenth peptides.

24. The composition of any one of claims 1 to 23, wherein at least one of the peptides comprises an N-terminal pyroglutamate and/or a C-terminal amide group.

25. The composition of claim 24, wherein each of the peptides comprises an N-terminal pyroglutamate and/or a C-terminal amide group.

26. The composition of any one of claims 1 to 25, wherein each of the peptides is independently between 8 to 50 amino acids in length.

27. The composition of claim 26, wherein each of the peptides is independently between 10 to 30 amino acids in length.

28. The composition of claim 27, wherein each of the peptides is independently between 14 to 20 amino acids in length.

29. The composition of any one of claims 1 to 28, wherein the composition further comprises a pharmaceutically acceptable carrier.

30. A composition comprising one or more polynucleotides encoding the peptides of the composition of any one of claims 1 to 28.

31. An isolated antigen presenting cell comprising the composition of any one of claims 1 to 28.

32. The composition of any one of claims 1 to 28, wherein at least one of the peptides is bound to a) an HLA molecule, or b) a fragment of an HLA molecule, capable of binding the peptide.

33. A kit comprising the composition of any one of claims 1 to 28 and means to detect binding of one or more of the peptides in the composition to T cells.

34. The kit of claim 33, wherein the means to detect binding of one or more of the peptides in the composition to T cells is an antibody specific for a cytokine.

35. The kit of claim 34, wherein the cytokine is selected from IFN-gamma or IP-10.

36. A method for treating Celiac disease in a subject, the method comprising:
administering to a subject having Celiac disease an effective amount of a composition of any one of claims 1 to 30 or the antigen presenting cell of claim 31.

37. A method for identifying a subject as having or at risk of having Celiac disease, the method comprising:
determining a T cell response to a composition of any one of claims 1 to 30 or the antigen presenting cell of claim 31 in a sample comprising a T cell from the subject; and assessing whether or not the subject has or is at risk of having Celiac disease.

38. The method of claim 37, wherein the assessing comprises:
identifying the subject as
(i) having or at risk of having Celiac disease if the T cell response to the composition is elevated compared to a control T cell response, or
(ii) not having or not at risk of having Celiac disease if the T cell response to the composition is reduced compared to the control T cell response or the same as the control T cell response.

39. The method of claim 37 or 38, wherein the step of determining comprises contacting the sample with the composition and measuring a T cell response to the composition.

40. The method of claim 39, wherein measuring a T cell response to the composition comprises measuring a level of a cytokine in the sample.

41. The method of claim 40, wherein the cytokine is IFN-gamma or IP-10.

42. The method of any one of claims 39 to 41, wherein measuring comprises an enzyme-linked immunosorbent assay (ELISA), an enzyme-linked immunosorbent spot (ELISpot) assay, or a multiplex bead-based immunoassay.

43. The method of any one claims 37 to 42, wherein the sample comprises whole blood or peripheral blood mononuclear cells.

44. The method of any one of the claims 37 to 43, wherein the method further comprises administering a composition comprising wheat, rye, or barley, or one or more peptides thereof, to the subject prior to determining the T cell response.

45. The method of claim 44, wherein the composition comprising wheat, rye, or barley, or one or more peptides thereof, is administered to the subject more than once prior to determining the T cell response.

46. The method of claim 45, wherein the composition comprising wheat, rye, or barley, or one or more peptides thereof, is administered to the subject at least once a day for three days.

47. The method of any one of claims 44 to 46, wherein the sample comprising the T cell is obtained from the subject after the administration of the composition comprising wheat, rye, or barley, or one or more peptides thereof.

48. The method of any one of claims 44 to 47, wherein the composition comprising wheat, rye, or barley, or one or more peptides thereof, is administered to the subject via oral administration.

49. The method of claim 48, wherein the composition comprising wheat, rye, or barley, or one or more peptides thereof, is a foodstuff.

50. The method of claim 48 or 49, wherein the sample is obtained from the subject 6 days after the oral administration.

51. The method of any one of claims 37 to 50, wherein the method further comprises treating the subject if identified as having or at risk of having Celiac disease or providing information to the subject about a treatment.

52. The method of any one of claims 37 to 50, where the method further comprises a step of recommending a gluten-free diet if the subject is identified as having or at risk of having Celiac disease or providing information to the subject about such a diet.

53. The method of any one of claims 36 to 52, wherein the subject is HLA-DQ2.2 positive and/or HLA-DQ8 positive.

54. The method of any one of claims 36 to 52, wherein the subject is HLA-DQ2.5 positive and either HLA-DQ2.2 positive or HLA-DQ8 positive.

55. A composition comprising at least one peptide selected from:
(a) a first peptide comprising the amino acid sequence PFPQPELPY (SEQ ID NO: 1) and the amino acid sequence PQPELPYPQ (SEQ ID NO: 2);
(b) a second peptide comprising the amino acid sequence PFPQPEQPF (SEQ ID NO: 3) and the amino acid sequence PQPEQPFPW (SEQ ID NO: 4);
(c) a third peptide comprising the amino acid sequence PIPEQPQPY (SEQ ID NO: 6);
(d) a fourth peptide comprising the amino acid sequence PFPQPEQPIP (SEQ ID NO: 62) and the amino acid sequence EQPIPVQPE (SEQ ID NO: 9);
(e) a fifth peptide comprising the amino acid sequence PFPQPEQPTPI (SEQ ID NO: 65) and the amino acid sequence EQPTPIQPE (SEQ ID NO: 12);
(f) a sixth peptide comprising the amino acid sequence PQPEQPFPL (SEQ ID NO: 13) and the amino acid sequence EQPFPLQPE (SEQ ID NO: 14);
(g) a seventh peptide comprising the amino acid sequence PFPQPEQPF (SEQ ID NO: 3) and the amino acid sequence PQPEQPFSQ (SEQ ID NO: 15);
(h) an eighth peptide comprising the amino acid sequence PYPEQPQPF (SEQ ID NO: 16);
(i) a ninth peptide comprising the amino acid sequence PFPEQPEQIIP (SEQ ID NO: 63);
(j) a tenth peptide comprising the amino acid sequence EGSFQPSQE (SEQ ID NO: 17);
(k) an eleventh peptide comprising the amino acid sequence QGYYPTSPQ (SEQ ID NO: 18);
(l) a twelfth peptide comprising the amino acid sequence EQPEQPFPEQPQ (SEQ ID NO: 64);
(m) a thirteenth peptide comprising the amino acid sequence PFSEQEQPV (SEQ ID NO: 22);
(n) a fourteenth peptide comprising the amino acid sequence EQPFPEQPI (SEQ ID NO: 23) and PIPEQPQPY (SEQ ID NO: 6);
(o) a fifteenth peptide comprising the amino acid sequence PQPELPYPQ (SEQ ID NO: 2) and the amino acid sequence PYPQPELPY (SEQ ID NO: 25);
(p) a sixteenth peptide comprising the amino acid sequence PFPQPELPY (SEQ ID NO: 1) and the amino acid sequence PQPELPYPY (SEQ ID NO: 26); and
(q) a seventeenth peptide comprising the amino acid sequence EQPFPEQPI (SEQ ID NO: 23).

56. The composition of claim 55, wherein:
(a) the first peptide comprises the amino acid sequence LQPFPQPELPYPQPQ (SEQ ID NO: 28);
(b) the second peptide comprises the amino acid sequence QPFPQPEQPFPWQP (SEQ ID NO: 29);
(c) the third peptide comprises the amino acid sequence PEQPIPEQPQPYPQQ (SEQ ID NO: 30);
(d) the fourth peptide comprises the amino acid sequence QPFPQPEQPIPVQPEQS (SEQ ID NO: 31);
(e) the fifth peptide comprises the amino acid sequence QPFPQPEQPTPIQPEQP (SEQ ID NO: 32);
(f) the sixth peptide comprises the amino acid sequence QPFPQPEQPFPLQPEQP (SEQ ID NO: 33);
(g) the seventh peptide comprises the amino acid sequence QPFPQPEQPFSQQ (SEQ ID NO: 34);
(h) the eighth peptide comprises the amino acid sequence PQPYPEQPQPFPQQ (SEQ ID NO: 35);
(i) the ninth peptide comprises the amino acid sequence QPFPEQPEQIIPQQP (SEQ ID NO: 36);
(j) the tenth peptide comprises the amino acid sequence SGEGSFQPSQENPQ (SEQ ID NO: 37);
(k) the eleventh peptide comprises the amino acid sequence GQQGYYPTSPQQSG (SEQ ID NO: 38);
(l) the twelfth peptide comprises the amino acid sequence PEQPEQPFPEQPQQ (SEQ ID NO: 39);
(m) the thirteenth peptide comprises the amino acid sequence QPPFSEQEQPVLPQ (SEQ ID NO: 40);
(n) the fourteenth peptide comprises the amino acid sequence PEQPFPEQPIPEQPQPYP (SEQ ID NO: 41);
(o) the fifteenth peptide comprises the amino acid sequence QPYPQPELPYPQPQ (SEQ ID NO: 42);
(p) the sixteenth peptide comprises the amino acid sequence QPFPQPELPYPYPQ (SEQ ID NO: 43); and
(q) the seventeenth peptide comprises the amino acid sequence EQPFPEQPI (SEQ ID NO: 23).

57. The composition of claim 55 or 56, wherein the composition comprises at least four of the peptides.

58. The composition of claim 57, comprising:
(i) the first, second, and third peptides or the second, fourteenth, fifteenth, and sixteenth peptides; and
(ii) at least one of the fourth, fifth, sixth, seventh, eighth, ninth, tenth, eleventh, twelfth, and thirteenth peptides.

59. The composition of claim 58, comprising at least two of the fourth, fifth, sixth, seventh, eighth, ninth, tenth, eleventh, twelfth, and thirteenth peptides.

60. The composition of claim 59, comprising at least three of the fourth, fifth, sixth, seventh, eighth, ninth, tenth, eleventh, twelfth, and thirteenth peptides.

61. The composition of claim 60, comprising at least four of the fourth, fifth, sixth, seventh, eighth, ninth, tenth, eleventh, twelfth, and thirteenth peptides.

62. The composition of claim 61, comprising at least five of the fourth, fifth, sixth, seventh, eighth, ninth, tenth, eleventh, twelfth, and thirteenth peptides.

63. The composition of claim 62, comprising at least six of the fourth, fifth, sixth, seventh, eighth, ninth, tenth, eleventh, twelfth, and thirteenth peptides.

64. The composition of claim 63, comprising at least seven of the fourth, fifth, sixth, seventh, eighth, ninth, tenth, eleventh, twelfth, and thirteenth peptides.

65. The composition of claim 64, comprising at least eight of the fourth, fifth, sixth, seventh, eighth, ninth, tenth, eleventh, twelfth, and thirteenth peptides.

66. The composition of claim 65, comprising at least nine of the fourth, fifth, sixth, seventh, eighth, ninth, tenth, eleventh, twelfth, and thirteenth peptides.

67. The composition of any one of claims 55 to 58, comprising the first, second, third, fourth, fifth, sixth, seventh, eighth, ninth, tenth, eleventh, twelfth, and thirteenth peptides.

68. The composition of any one of claims 55 to 58, comprising the second, fourth, fifth, sixth, seventh, eighth, ninth, tenth, eleventh, twelfth, thirteenth, fourteenth, fifteenth, and sixteenth peptides.

69. The composition of any one of claims 55 to 58, comprising the first, second, third, fourth, fifth, sixth, eighth, ninth, tenth, eleventh, twelfth, and thirteenth peptides.

70. The composition of any one of claims 55 to 58, comprising the second, fourth, fifth, sixth, eighth, ninth, tenth, eleventh, twelfth, thirteenth, fourteenth, fifteenth, and sixteenth peptides.

71. The composition of any one of claims 55 to 70, wherein at least one of the peptides comprises an N-terminal pyroglutamate and/or a C-terminal amide group.

72. The composition of claim 71, wherein each of the peptides comprises an N-terminal pyroglutamate and/or a C-terminal amide group.

73. The composition of any one of claims 55 to 72, wherein each of the peptides is independently between 8 to 50 amino acids in length.

74. The composition of claim 73, wherein each of the peptides is independently between 10 to 30 amino acids in length.

75. The composition of claim 74, wherein each of the peptides is independently between 14 to 20 amino acids in length.

76. The composition of any one of claims 55 to 75, wherein the composition further comprises a pharmaceutically acceptable carrier.

77. A composition comprising one or more polynucleotides encoding the peptides of the composition of any one of claims 55 to 75.

78. An isolated antigen presenting cell comprising the composition of any one of claims 55 to 75.

79. The composition of any one of claims 55 to 75, wherein at least one of the peptides is bound to a) an HLA molecule, or b) a fragment of an HLA molecule, capable of binding the peptide.

80. A kit comprising the composition of any one of claims 55 to 75 and means to detect binding of one or more of the peptides in the composition to T cells.

81. The kit of claim 80, wherein the means to detect binding of one or more of the peptides in the composition to T cells is an antibody specific for a cytokine.

82. The kit of claim 81, wherein the cytokine is selected from IFN-gamma or IP-10.

83. A method for treating Celiac disease in a subject, the method comprising:
administering to a subject having Celiac disease an effective amount of a composition of any one of claims 55 to 77 or the antigen presenting cell of claim 78.

84. A method for identifying a subject as having or at risk of having Celiac disease, the method comprising:
determining a T cell response to a composition of any one of claims 55 to 77 or the antigen presenting cell of claim 78 in a sample comprising a T cell from the subject; and
assessing whether or not the subject has or is at risk of having Celiac disease.

85. The method of claim 84, wherein the assessing comprises:
identifying the subject as
(i) having or at risk of having Celiac disease if the T cell response to the composition is elevated compared to a control T cell response, or
(ii) not having or not at risk of having Celiac disease if the T cell response to the composition is reduced compared to the control T cell response or the same as the control T cell response.

86. The method of claim 84 or 85, wherein the step of determining comprises contacting the sample with the composition and measuring a T cell response to the composition.

87. The method of claim 86, wherein measuring a T cell response to the composition comprises measuring a level of a cytokine in the sample.

88. The method of claim 87, wherein the cytokine is IFN-gamma or IP-10.

89. The method of any one of claims 86 to 88, wherein measuring comprises an enzyme-linked immunosorbent assay (ELISA), an enzyme-linked immunosorbent spot (ELISpot) assay, or a multiplex bead-based immunoassay.

90. The method of any one claims 84 to 89, wherein the sample comprises whole blood or peripheral blood mononuclear cells.

91. The method of any one of the claims 84 to 90, wherein the method further comprises administering a composition comprising wheat, rye, or barley, or one or more peptides thereof, to the subject prior to determining the T cell response.

92. The method of claim 91, wherein the composition comprising wheat, rye, or barley, or one or more peptides thereof, is administered to the subject more than once prior to determining the T cell response.

93. The method of claim 92, wherein the composition comprising wheat, rye, or barley, or one or more peptides thereof, is administered to the subject at least once a day for three days.

94. The method of any one of claims 91 to 93, wherein the sample comprising the T cell is obtained from the subject after the administration of the composition comprising wheat, rye, or barley, or one or more peptides thereof.

95. The method of any one of claims 91 to 94, wherein the composition comprising wheat, rye, or barley, or one or more peptides thereof, is administered to the subject via oral administration.

96. The method of claim 95, wherein the composition comprising wheat, rye, or barley, or one or more peptides thereof, is a foodstuff.

97. The method of claim 95 or 96, wherein the sample is obtained from the subject 6 days after the oral administration.

98. The method of any one of claims 84 to 97, wherein the method further comprises treating the subject if identified as having or at risk of having Celiac disease or providing information to the subject about a treatment.

99. The method of any one of claims 84 to 97, where the method further comprises a step of recommending a gluten-free diet if the subject is identified as having or at risk of having Celiac disease or providing information to the subject about such a diet.

100. The method of any one of claims 83 to 99, wherein the subject is HLA-DQ2.2 positive and/or HLA-DQ8 positive.

101. The method of any one of claims 83 to 99, wherein the subject is HLA-DQ2.5 positive and either HLA-DQ2.2 positive or HLA-DQ8 positive.
